(19) United States
(12) Patent Application Publication
(10) Pub. No.: US 2024/0081300 A1
(43) Pub. Date: Mar. 14, 2024

(54) ADRENERGIC MECHANISMS OF AUDIOGENIC SEIZURE-INDUCED DEATH IN MOUSE MODEL OF SCN8A ENCEPHALOPATHY

(71) Applicant: University of Virginia Patent Foundation, Charlottesville, VA (US)

(72) Inventors: Manoj K. Patel, Charlottesville, VA (US); Ian C. Wenker, Charlottesville, VA (US); Eric R. Wengert, Charlottesville, VA (US)

(73) Assignee: University of Virginia Patent Foundation, Charlottesville, VA (US)

(21) Appl. No.: 18/271,911

(22) PCT Filed: Jan. 12, 2022
(86) PCT No.: PCT/US2022/012203

§ 371 Date: Jul. 12, 2023

Related U.S. Application Data
(60) Provisional application No. 63/136,439, filed on Jan. 12, 2021.

(51) Int. Cl. A01K 67/027

(52) U.S. Cl. CPC A01K 67/0278; A01K 2217/072; A01K 2227/105; A01K 2267/0356

(57) ABSTRACT

Provided are compositions and methods for treating and/or preventing seizure-induced death in subjects in need thereof. In some embodiments, the methods include methods for inducing an audiogenic seizure and/or seizure-induced death, treating and/or preventing death associated with seizures in subjects, preventing sudden unexpected death in epilepsy (SUDEP) in subjects, preventing and/or reducing the risk of death in subjects having SCN8A gain-of-function mutations, preventing or reducing the risk of death associated with tonic seizures in subjects, and preventing or reducing the risk of death associated with epileptic seizures in subjects. Also provided are animals that have been modified to carry gain-of-function SCN8A mutations and methods for using the same to identify compounds that have activity in treating and/or preventing seizures and/or seizure-induced death in subjects.

### CROSS REFERENCE TO RELATED APPLICATION

The presently disclosed subject matter claims the benefit of U.S. Provisional Patent Application Ser. No. 63/136,439, filed Jan. 12, 2021, the disclosure of which incorporated herein by reference in its entirety.

### GOVERNMENT INTEREST

This invention was made with government support under Grant No. NS103090 awarded by The National Institutes of Health. The government has certain rights in the invention.

### REFERENCE TO SEQUENCE LISTING

The Sequence Listing associated with the instant disclosure has been electronically submitted to the United States Patent and Trademark Office as a 546 kilobyte ASCII text file created on Jan. 12, 2022 and entitled “3062_144_PCT_ST25.txt”. The Sequence Listing submitted via EFS-Web is hereby incorporated by reference in its entirety.

### TECHNICAL FIELD

The presently disclosed subject matter relates in some embodiments to compositions and methods for treating and/or preventing seizure-induced death in subjects in need thereof. In particular embodiments, the presently disclosed subject matter relates to providing compositions comprising one or more alpha-1 adrenergic receptor activators, optionally in conjunction with mechanical ventilation, to the subject, optionally wherein the intervention occurs prior to the termination of a tonic phase of the seizure in the subject.

### BACKGROUND

Sudden unexpected death in epilepsy (SUDEP) is defined as the sudden, unexpected, nontraumatic, and non-drowning death of a person with epilepsy for which postmortem examination does not reveal an anatomical or pathological cause of death (Nashef et al., 2012). SUDEP is the most common cause of death associated with epilepsy, accounting for between 8 and 17% of all epilepsy-related deaths (Terra et al., 2013), and rising to 50% for patients with poorly controlled seizures (Devinsky, 2011; Terra et al., 2013). SUDEP primarily affects the young; among neurological conditions it takes a close second only to stroke for number of life-years lost (Devinsky et al., 2016).

Due to the unpredictable nature for instances of SUDEP, clinical mechanisms of death remain elusive. To circumvent this issue, several rodent models have been developed in recent years to gain mechanistic insights into SUDEP. Most clinical SUDEP cases are believed to occur after generalized tonic-clonic seizures (Dasheiff & Dickinson, 1986; Bird et al., 1997; Nilsson et al., 1999; So et al., 2000; Ryvlin et al., 2013). Thus, animal models in which death occurs immediately after convulsive seizures are used to study SUDEP. Such models include the DBA/1&2, Cacnala^{S218L}, Scn1a^{R1407X}, RyR2^{R176Q}, Scn1a KO, and Kcna1 KO mouse models as well as inducible kainic acid and maximal electroshock seizure models. These approaches have fueled various hypotheses concerning the mechanisms of SUDEP, including brainstem spreading depolarization (Aiba & Noebels, 2015; Aiba et al., 2016; Jansen et al., 2019; Loonen et al., 2019), autonomic dysregulation and cardiac arrhythmias (Glasscock et al., 2010; Auerbach et al., 2013; Kalume et al., 2013), and respiratory arrest due to central (Faingold et al., 2010; Buchanan et al., 2014a; Kim et al., 2018; Kruse et al., 2019), or obstructive apnea (Nakase et al., 2016; Villiere et al., 2017; Irizarry et al., 2020). Of these models, only the Dravet Syndrome models (i.e. Scn1a^{R1407X} and Scn1a KO) directly represent a patient population that is susceptible to SUDEP (Escayg & Goldin, 2010; Kim et al., 2018). The genetic etiologies of DBA/1&2, Cacna1a^{S218L}, RyR2^{R176Q}, Lmx1b^{f/f/p}, and Kcna1 KO mice are either unknown, identified from non-epilepsy patient populations, or are manipulations that lead to loss of an entire cell population, receptor subtypes, or ion channels, which are not known to occur in epilepsy patients.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to germline modified animals, in some embodiments germline modified mice. In some embodiments, the germline of the transgenic animal (e.g., mouse) comprises a nucleotide sequence encoding a gain-of-function SCN8A mutation. In some embodiments, the gain-of-function SCN8A mutation encodes a substitution selected from the group consisting of a leucine to valine substitution at amino acid 257 of SEQ ID NO: 12, a leucine to valine substitution at amino acid 862 of SEQ ID NO: 12, a glutamine to histidine substitution at amino acid 1468 of SEQ ID NO: 12, a glycine to arginine substitution at amino acid 1473 of SEQ ID NO: 12, an alanine to valine substitution at amino acid 1489 of SEQ ID NO: 12, a methionine to threonine substitution at amino acid 1643 of SEQ ID NO: 12, an alanine to threonine substitution at amino acid 1648 of SEQ ID NO: 12, and an asparagine to aspartic acid substitution at amino acid 1766 of SEQ ID NO: 12, or any combination thereof. In some embodiments, the germline modified animal (e.g., mouse) comprises a genome comprising an endogenous SCN8A coding sequence with an asparagine to aspartic acid substitution at amino acid 1766 of SEQ ID NO: 12.

In some embodiments, the presently disclosed subject matter also relates to methods for inducing audiogenic seizures and/or seizure-induced death in an animal such as a mouse. In some embodiments, the methods comprise, consist essentially of, or consist of subjecting a germline modified animal as disclosed herein to an audiogenic stimulus of sufficient intensity and duration to induce an audiogenic seizure and/or seizure-induced death in the animal. In some embodiments, the audiogenic stimulus comprises sound of at least about 12 kHz at an intensity of at least about 100 dB for a duration of at least about 10 seconds.

In some embodiments, the presently disclosed subject matter also relates to methods for treating and/or preventing death associated with a seizure in subjects. In some embodiments, the methods comprise, consist essentially of, or consist of stimulating breathing of the subject via mechanical ventilation and/or by administering a composition comprising an effective amount of an alpha-1 adrenergic receptor activator to the subject. In some embodiments, the seizure is an epileptic seizure. In some embodiments, the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline. In some embodiments, the mechanical ventilation of the subject is continued until the subject is able to breath unassisted. In some embodiments, the alpha-1 adrenergic receptor activator is administered to the subject during a tonic phase of the seizure, optionally within 1, 2, 3, 4, or 5 minutes from the onset of the seizure. In some embodiments, the composition is provided to the subject as an injectable, optionally in the form of a pen delivery device.

In some embodiments, the presently disclosed subject matter also relates to methods for identifying compounds that have activity in treating and/or preventing seizure and/or seizure-induced death in subjects. In some embodiments, the methods comprise, consist essentially of, or consist of inducing an audiogenic seizure in a germline modified animal (e.g., mouse) as disclosed herein; (b) administering a compound to be tested to the germline modified animal (e.g., mouse); and (c) determining whether the compound treats and/or prevents a seizure and/or seizure-induced death in the subject, whereby a compound that has activity in treating and/or preventing a seizure and/or seizure-induced death in a subject is identified.

In some embodiments, the presently disclosed subject matter also relates to methods for preventing sudden unexpected death in epilepsy (SUDEP) in subjects in need thereof. In some embodiments, the methods comprise, consist essentially of, or consist of stimulating breathing of the subject via mechanical ventilation and/or by administering a composition comprising an effective amount of an alpha-1 adrenergic receptor activator to the subject. In some embodiments, the SUDEP is associated with a gain-of-function mutation in an SCN8A gene product in the subject. In some embodiments, the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof. In some embodiments, the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline. In some embodiments, the alpha-1 adrenergic receptor activator is administered to the subject as soon as possible after the onset of the a tonic phase of the seizure, optionally within 1, 2, 3, 4, or 5 minutes from the onset of the tonic phase of the seizure. In some embodiments, the presently disclosed subject matter methods further comprise providing mechanical ventilation to the subject. In some embodiments, the mechanical ventilation is provided to the subject until the subject is able to breath unassisted.

In some embodiments, the presently disclosed subject matter also relates to methods for preventing and/or reducing the risk of death in subjects having one or more gain-of-function mutations in an SCN8A gene product. In some embodiments, the methods comprise, consist essentially of, or consists of stimulating breathing of the subject via mechanical ventilation and/or by administering a composition comprising an effective amount of an alpha-1 adrenergic receptor activator to the subject. In some embodiments, the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof. In some embodiments, the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline. In some embodiments, the alpha-1 adrenergic receptor activator is administered to the subject during a tonic phase of the seizure, optionally within 1, 2, 3, 4, or 5 minutes from the onset of the seizure and/or the tonic phase thereof. In some embodiments, the presently disclosed methods further comprise providing mechanical ventilation to the subject. In some embodiments, the mechanical ventilation is provided to the subject until the subject is able to breath unassisted.

In some embodiments, the presently disclosed subject matter also relates to methods for preventing or reducing the risk of death associated with a tonic seizure in a subject in need thereof. In some embodiments, the methods comprise, consist essentially of, or consist of stimulating breathing of the subject via mechanical ventilation and/or by administering a composition comprising an effective amount of an alpha-1 adrenergic receptor activator to the subject. In some embodiments, the subject has a genome comprising a gain-of-function mutation in an SCN8A gene product. In some embodiments, the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof. In some embodiments, the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline. In some embodiments, the alpha-1 adrenergic receptor activator is administered to the subject during a tonic phase of the seizure, optionally within 1, 2, 3, 4, or 5 minutes from the onset of the seizure and/or of the tonic phase thereof. In some embodiments, the presently disclosed methods further comprise providing mechanical ventilation to the subject. In some embodiments, the mechanical ventilation is provided to the subject until the subject is able to breath unassisted.

In some embodiments, the presently disclosed subject matter also relates to methods for preventing or reducing the risk of death associated with an epileptic seizure in a subject in need thereof. In some embodiments, the methods comprise, consist essentially of, or consist of stimulating breathing of the subject via mechanical ventilation and/or by administering a composition comprising an effective amount of an alpha-1 adrenergic receptor activator to the subject. In some embodiments, the subject has a genome comprising a gain-of-function mutation in an SCN8A gene product. In some embodiments, the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof. In some embodiments, the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline. In some embodiments, the alpha-1 adrenergic receptor activator is administered to the subject during a tonic phase of the seizure, optionally within 1, 2, 3, 4, or 5 minutes from the onset of the seizure and/or of the tonic phase thereof. In some embodiments, the presently disclosed methods further comprise providing mechanical ventilation to the subject. In some embodiments, the mechanical ventilation is provided to the subject until the subject is able to breath unassisted. In some embodiments, the composition comprising the alpha-1 adrenergic receptor activator, the mechanical ventilation, if administered, or both are administered to the subject subsequent to development of apnea but prior to the end of a tonic phase experienced by the subject.

In some embodiments of the presently disclosed methods, the alpha-1 adrenergic receptor activator is provided to the subject as an injectable, optionally in the form of a pen delivery device.

In some embodiments of the presently disclosed methods, the alpha-1 adrenergic receptor activator is administered to the subject via a route selected from the group consisting of intraperitoneal, intramuscular, intravenous, and intranasal, or any combination thereof.

In some embodiments of the presently disclosed methods, the subject is a human.

Accordingly, it is an object of the presently disclosed subject matter to provide compositions and methods for treating and/or preventing seizure-induced death in subjects in need thereof. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIGS. 1A-1I: Audiogenic seizures and sudden death in D/+ mice. FIG. 1A. In response to high-intensity acoustic stimulation, D/+ mice exhibited wild-running, a tonic phase characterized by hindlimb extension, a clonic stage characterized by rapid kicking of the hindlimbs, and either recovery as the animal righted itself and resumed normal movement throughout the cage or sudden death. See FIGS. 1B-1E, particularly the differential hatching provided therein which is maintained throughout. Audiogenic seizure behavioral progression in each mouse at developmental time points postnatal day 15 (P15; FIG. 1B), postnatal day 20/21 (P20-21; FIG. 1C), postnatal day 32 (P32; FIG. 1D), and postnatal days 49-69 (P49-P69; FIG. 1E). FIG. 1B. Only 2 of 8 D/+ mice at P15 exhibited audiogenic seizures. FIG. 1C. All 13 D/+ mice at P20-21 experienced audiogenic seizures, and 11 of 13 died directly following the tonic phase. The two that recovered did so after a relatively extended clonic phase. FIG. 1D. At P32, all 8 D/+ mice had audiogenic seizures and recovered. FIG. 1E. In adult D/+ mice (P49-P69), all mice experienced seizures followed by recovery. FIGS. 1F-1I. Duration of seizure phases, latency to seizure onset (FIG. 1F), wild-running (FIG. 1G), tonic phase (FIG. 1H), and clonic phase (FIG. 1I) for D/+ mice ages P20-21 (n=13), P32 (n=8), and P49-P69 (n=14). **, ***, ****, and NS indicate p<0.01, p<0.001, p<0.0001, and p>0.2, respectively, for post hoc comparison between age groups.

FIG. 2: Intensity-dependence of audiogenic seizures in D/+ mice. Cumulative probability of adult D/+ with audiogenic seizures relative to the intensity of acoustic stimulation at 14 kHz. All mice exhibited seizures by 100 dB.

FIGS. 3A-3F: D/+ mice exhibit mild hearing impairment. FIG. 3A. Example auditory brainstem response (ABR) traces for a WT mouse at P56 in response to a 32 kHz tone. ABR threshold is indicated with a broken line at 35 dB. FIG. 3B. D/+ ABR example traces indicated a threshold of 55 dB (broken line trace). FIG. 3C. Average ABR thresholds for WT (closed circles; n=10) and D/+(open circles; n=10) mice at P56. D/+ mice had significantly elevated ABR thresholds at 22.4 and 32 kHz. FIG. 3D. Example ABR traces for a WT mouse at P112 in response to 32 kHz tone. ABR threshold is indicated at 60 dB (broken line trace). FIG. 3E. Example ABR traces for a D/+ mouse at P112 in response to a 32 kHz tone with the ABR threshold indicated at 70 dB. FIG. 3F. Average ABR thresholds for WT (closed circles; n=6) and D/+(open circles; n=5) revealed significantly elevated ABR thresholds in D/+ mice. * indicates p<0.05.

FIGS. 4A-4G: Audiogenic seizures resemble spontaneous seizures in D/+ mice. FIGS. 4A and 4B. Example traces of electrocorticogram activity (ECoG), electrocardiogram activity (ECG/EMG), and breathing activity (Pleth) during example spontaneous (FIG. 4A) and audiogenic seizures (FIG. 4B). Stages of seizure behavior are indicated above the traces: Wild-running, tonic phase, and recovery phases are indicated with differential hatching (see also FIGS. 1A-1E). ECoG ictal activity, bradycardia, and apnea are each highlighted in brackets. Arrow indicates onset of 15 kHz acoustic stimulation (FIG. 4B). FIGS. 4A₁ and 4A₂. Expanded data traces from regions indicated in FIG. 4A. FIGS. 4B₁ and 4B₂. Expanded data traces from regions indicated in FIG. 4B. Note the high degree of tonic muscle activity and apnea in FIG. 4A₂ and FIG. 4B₂. FIG. 4C. Average breathing frequency (Rf) binned every second during spontaneous (solid line; n=16 seizures recorded from 4 mice) and audiogenic (dashed line; n=15 seizures recorded from 7 mice) seizures. FIG. 4D. Average heart rate (HR) binned every second during spontaneous (solid line; n=16 seizures recorded from 4 mice) and audiogenic (dashed line; n=12 seizures recorded from 7 mice) seizures. FIG. 4E. ECoG ictal duration for spontaneous (open circles; n=15 seizures recorded from 4 mice) and audiogenic (closed black circles; n=15 seizures recorded from 6 mice) seizures. FIG. 4F. Apnea duration for spontaneous (open circles; n=16 seizures recorded from 4 mice) and audiogenic (closed black circles; n=18 seizures recorded from 7 mice) seizures. FIG. 4G. Time of ictal onset relative to tonic phase. Audiogenic seizures (closed black circles; n=15 seizures recorded from 6 mice) exhibited delayed onset of ECoG ictal activity relative to tonic phase compared to spontaneous seizures (open circles; n=15 seizures recorded from 4 mice) in D/+ mice. **** indicates p<0.0001 comparison between genotypes. Hatching is as in FIG. 1.

FIGS. 5A-5D: Seizure-induced respiratory arrest and sudden death in P20-21 D/+ mice. FIGS. 5A and 5B. Plethysmography recordings during audiogenic seizures in a P21 D/+ mouse (FIG. 5A) and a P25 D/+ mouse (FIG. 5B). Behavioral seizure stages shown above traces. Wild running precedes a tonic phase followed by either death (FIG. 5A; left to right wide descending hatching) or recovery (FIG. 5B; left to right narrow ascending hatching). FIG. 5A. At the onset of the tonic phase of the audiogenic seizure, the P21 D/+ mouse ceased breathing (Rf near zero) and never recovered. FIG. 5B. In a P25 D/+ mouse, breathing ceased during the tonic phase but recovered shortly after and the mouse survived. FIG. 5C. Image of mechanical ventilation intervention to stimulate breathing after onset of an audiogenic seizure in P20-21 D/+ mice. A breathing tube is depicted covering the mouse's nose and mouse to deliver oxygen. FIG. 5D. Bar chart of survival of control non-ventilated (left bar; n=15) and ventilated (right bar; n=8) P20-21 D/+ mice. Mechanical ventilation significantly improved rate of survival (*p<0.05; one-sided Fisher's exact test). Hatching is as in FIG. 1.

FIGS. 6A-6E: Acute activation of alpha-1 adrenergic receptors rescues seizure-induced sudden death in P20-21 D/+ mice. FIG. 6A. P20-21 D/+ mice were injected (i.p.) with saline, 1 mg/kg epinephrine (Epi), 2 mg/kg norepinephrine (NE), or 3 mg/kg phenylephrine (PE) 1-minute before stimulating an audiogenic seizure. FIGS. 6B and 6C. Example plethysmography recordings of breathing during audiogenic seizures in P20-21 D/+ mice injected with saline control (FIG. 6B) or PE (FIG. 6C) 1 minute before acoustic stimulation. In the saline-treated mouse, breathing ceased during the tonic phase and never recovered leading to death. In the PE-treated mouse, breathing also ceased during the tonic phase but recovered and the mouse survived. FIG. 6D. Average breathing rates binned every second for saline-treated (upper panel; n=5) and PE-treated (lower panel; n=3) P20-21 D/+ mice. FIG. 6E. Bar chart of survival of control saline-treated mice (n=13), Epi-treated mice (n=5), NE-treated mice (n=13), and PE-treated mice (n=8) revealed significantly elevated rates of survival in NE (p<0.05) and PE-treated (**p<0.01) mice. Statistical comparisons made using a one-sided Fisher's exact test. Hatching is as in FIG. 1.

FIGS. 7A-7E: Inhibition of adrenergic receptors leads to seizure-induced respiratory arrest and sudden death in adult D/+ mice. FIG. 7A. Adult D/+ mice were injected (i.p.) with saline, prazosin (1 mg/kg) and/or sotalol (10 mg/kg) 15 minutes before stimulation of an audiogenic seizure. FIGS. 7B and 7C. Example plethysmography recordings of breathing during audiogenic seizures in D/+ mice treated with saline (FIG. 7B) or prazosin and sotalol (FIG. 7C) ˜15 minutes before acoustic stimulation. Breathing recovered in the saline-treated adult mouse. However, breathing never recovered in the combined prazosin and sotalol-treated adult mouse after the audiogenic seizure. FIG. 7D. Average breathing rates for saline-treated (upper panel; n=4) and prazosin/sotalol-treated mice (lower panel; n=5). FIG. 7E. Bar chart of survival of adult D/+ mice treated with saline (SAL; n=10), sotalol (SOT; n=12), prazosin (PRAZ; n=11), and prazosin/sotalol (PRAZ & SOT; n=14). Prazosin alone significantly reduced survival rate compared to saline injection (***p<0.001; n=11), with a similarly strong effect observed in mice treated with both prazosin and sotalol (***p<0.001; n=14). Statistical comparisons made using one-sided Fisher's exact tests. Hatching is as in FIG. 1.

FIGS. 8A-8D: Mechanical ventilation does not require adrenergic receptor function to prevent seizure-induced sudden death. FIG. 8A. Adult mice were injected (i.p.) with prazosin (1 mg/kg) and sotalol (10 mg/kg) 15 minutes before simulation of an audiogenic seizure. Some mice were either mechanical ventilated (MV) or not (No MV). FIG. 8B. MV increased survival compared to No MV (***p<0.001). FIG. 8C. P20-21 D/+ mice were either non-injected or injected with a combination of prazosin (1 mg/kg) and sotalol (10 mg/kg) 15 minutes prior to stimulation of an audiogenic seizure. All mice received mechanical ventilation (MV). FIG. 8D. 6 of 8 P20-21 D/+ mice that received MV only survived and were not statistically different from mice injected with a combination of prazosin (1 mg/kg) and sotalol (10 mg/kg) 15 minutes prior to stimulation of an audiogenic seizure that also received MV (6 of 6 mice). Statistical comparisons were made using one-sided Fisher's exact tests.

FIGS. 9A-9C: Neither pre-ictal breathing rate nor extended duration of tonic phase cause seizure-induced death. FIG. 9A. Pre-ictal breathing rates in survival (gray) and fatal seizures (pink) in both P20-21 and adult D/+ mice. FIG. 9B. Tonic phase duration in P20-21 and adult D/+ mice for both survival and fatal seizures. FIG. 9C. Graphic summary: At the onset of tonic phase of seizures, breathing stopped. Recovery of breathing from this apnea determined whether or not the animal recovered and survived. Mechanistically, alpha-1 receptors function upstream to drive respiration and prevent seizure-induced death. **, ***, ****, and NS indicate p<0.01, p<0.001, p<0.0001, and p>0.2, respectively, for main effect of survival vs. fatal and post hoc comparisons between age groups.

### BRIEF DESCRIPTION OF THE SEQUENCE LISTING

SEQ ID NOs: 1 and 2 are the nucleotide sequences of exemplary oligonucleotides that can be employed for genotyping D/+ mice with respect to the presence of an N1768D-encoding transgene.

SEQ ID NOs: 3-10 are the nucleotide and amino acid sequences of exemplary human SCN8A gene products. Within SEQ ID NOs: 3-10, odd numbered SEQ ID NOs. represent nucleotide sequence and even numbered SEQ ID NOs. represent amino acid sequences encoded by the immediately preceding SEQ ID NO.

SEQ ID NOs: 11 and 12 are the nucleotide and amino acid sequences of exemplary mouse SCN8A gene products.

SEQ ID NO: 13 is the genomic DNA sequence of the human SCN8A genetic locus, and corresponds to nucleotides 51,591,233-51,812,864 of Accession No. NC_000012.12 of the GENBANK® biosequence database.

### DETAILED DESCRIPTION

Described herein in some embodiments is a transgenic mouse model of SCN8A epileptic encephalopathy, also known as Early Infantile Epileptic Encephalopathy 13 (EIEE13; Online Mendelian Inheritance in Man (OMIM) No. 614558; see Hamosh et al., 2005). Patients with this recently recognized syndrome have de novo, gain-of-function SCN8A mutations and a high incidence of SUDEP ranging between 5 and 10%, although this is possibly an underestimate due to the young average patient age (Hammer et al., 1993; Ohba et al., 2014; Blanchard et al., 2015; Larsen et al., 2015; Gardella et al., 2018; Johannesen et al., 2018). This Scn8a mouse model harbors the germline knock-in mutation N1768D (D/+), a mutation identified in an SCN8A epilepsy patient that died of SUDEP (Wagnon et al., 2015; Lopez-Santiago et al., 2017; Ottolini et al., 2017; Sprissler et al., 2017; Wengert et al., 2019). The D/+ mice recapitulate the key hallmarks of SCN8A encephalopathy and SUDEP: chronic spontaneous tonic-clonic seizures and seizure-induced deaths (Wagnon et al., 2015). While the D/+ mice have been used to unravel mechanisms of disease etiology (Wagnon et al., 2015; Lopez-Santiago et al., 2017; Ottolini et al., 2017; Sprissler et al., 2017; Wengert et al., 2019), their utility as a SUDEP model has been hampered by the inability to temporally control the occurrence of seizures and seizure-induced death.

As disclosed herein, the ability to evoke seizures on command in D/+ mice using high-intensity sound is demonstrated. These evoked seizures are nearly identical to spontaneous seizures with respect to behavioral, electrographic, and cardiorespiratory parameters. Also described is a developmental window where audiogenic seizures almost always (˜85%) lead to seizure-induced death. Using the audiogenic seizure model disclosed herein, it is demonstrated that: (1) the primary cause of seizure-induced death is respiratory arrest that is initiated during the tonic phase; (2) non-fatal seizures also present with transient apnea but breathing recovers after the tonic phase; (3) peri-ictal alpha-1 adrenergic receptor activity is both necessary and sufficient for survival after a tonic-clonic seizure; and (4) the mechanism of action for adrenergic stimulation is breathing recovery after initial tonic phase apnea.

### I. General Considerations

Sudden unexpected death in epilepsy (SUDEP) is the leading cause of death amongst patients whose seizures are not adequately controlled by current therapies. Patients with SCN8A encephalopathy have an elevated risk for SUDEP. While transgenic mouse models have provided insight into the molecular mechanisms of SCN8A encephalopathy etiology, our understanding of seizure-induced death has been hampered by the inability to reliably trigger both seizures and seizure-induced death in these mice. Here, it is demonstrated that mice harboring an Scn8a allele with the patient-derived mutation N1768D (D/+) are susceptible to audiogenic seizures and seizure-induced death. In adult D/+ mice, audiogenic seizures are non-fatal and have nearly identical behavioral, electrographical, and cardiorespiratory characteristics as spontaneous seizures. In contrast, at postnatal days 20-21 (P20-21), D/+ mice exhibit the same seizure behavior, but have a significantly higher incidence of seizure-induced death following an audiogenic seizure. Seizure-induced death was prevented by either stimulating breathing via mechanical ventilation or by acute activation of alpha-1 adrenergic receptors. Conversely, in adult D/+ mice inhibition of alpha-1 adrenergic receptors converted normally non-fatal audiogenic seizures into fatal seizures. Taken together, described herein are data demonstrating that in the presently disclosed audiogenic seizure-induced death model, adrenergic receptor activation was necessary and sufficient for recovery of breathing and prevention of seizure-induced death.

### II. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

As use herein, the terms “administration of” and/or “administering” a compound should be understood to refer to providing a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

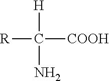

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

An “antagonist” is a composition of matter which when administered to a mammal such as a human, inhibits a biological activity attributable to the level or presence of a compound or molecule of interest in the subject.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group. In the context of a peptide or polypeptide sequence, a “derivative” is a peptide or polypeptide that has one or more modifications to its amino acid sequence such that it differs in at least one respect from a reference sequence (e.g., a naturally occurring peptide or polypeptide) either with respect to amino acid sequence (e.g., resulting from one or more additions, deletions, and/or amino acid substitutions) or with respect to some modification thereof. Exemplary non-limiting modifications include N- and/or C-terminal additions of one or more amino acids, in some embodiments functional amino acids (e.g., cysteine), N- and/or C-terminal amidation, N- and/or C-terminal acylation, N- and/or C-terminal acetylation, and N- and/or C-terminal pegylation.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to cells, refers to a single cell of interest, or a population of cells of interest, at least partially isolated from other cell types or other cellular material with which it occurs in a culture or a tissue of origin. A sample is “substantially pure” when it is in some embodiments at least 60%, in some embodiments at least 75%, in some embodiments at least 90%, and, in certain cases, in some embodiments at least 99% free of cells or other cellular material other than the cells of interest.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject. In some embodiments, a pharmaceutically-acceptable carrier is pharmaceutically-acceptable for use in a human.

As used herein, the term “physiologically acceptable” ester or salt means an ester or salt form of the active ingredient which is compatible with any other ingredients of the pharmaceutical composition, which is not deleterious to the subject to which the composition is to be administered.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

The term “prevent”, as used herein, means to stop something from happening, or taking advance measures against something possible or probable from happening, to decrease the chance and/or risk of it happening at least some degree. In the context of medicine, “prevention” and grammatical variations thereof generally refers to action taken to decrease the chance and/or risk of getting or suffering from a disease or condition, to at least some degree.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, a “subject in need thereof” is a patient, animal, mammal, human, or other subject who will benefit from a method or compositions of the presently disclosed subject matter.

The term “substantially pure” describes a compound, molecule, or the like, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, such as but not limited to in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

As used herein, the term “SCN8A” refers to the sodium voltage-gated channel alpha subunit 8 gene and its gene products. The human SCN8A genetic locus is present on chromosome 12 at nucleotides 51,591,233-51,812,864 of Accession No. NC 000012.12 of the GENBANK® biosequence database (SEQ ID NO: 13). Exemplary gene products derived from the human SCN8A genetic locus include Accession Nos. NM_014191.4 (SEQ ID NO: 3), NM_001177984.3 (SEQ ID NO: 5), NM_001330260.2 (SEQ ID NO: 7), and NM_001369788.1 (SEQ ID NO: 9) of the GENBANK® biosequence database, which encode Accession Nos. NP_055006.1 (SEQ ID NO: 4), NP_001171455.1 (SEQ ID NO: 6), NP_001317189.1 (SEQ ID NO: 8), and NP_001356717.1 (SEQ ID NO: 10) of the GENBANK® biosequence database, respectively. These GENBANK® Accession Nos. as well as all annotations presented therewith are expressly incorporated by reference herein in their entireties.

### III. Animal Models of SCN8A Epilepsy

In some embodiments, the presently disclosed subject matter relates to animal models of SCN8A Epilepsy. Particularly, in some embodiments the presently disclosed subject matter relates to germline modified animals, including but not limited to mice, the germlines of which carry nucleotide sequences encoding one or more gain-of-function SCN8A mutations.

By way of example and not limitation, Wagnon et al., 2014 discloses the production of a transgenic mouse that was modified to carry the Scna8^{N1768D} mutation by genetic knock-in. The mouse line was generated by TALEN targeting in which the codon encoding an asparagine at amino acid 1766 of the mouse Scna8 gene product (e.g., SEQ ID NO: 12) was modified to a codon encoding an aspartic acid. This codon, AAC, which corresponds to nucleotides 5449-5451 of SEQ ID NO: 11, can be modified to the codon GAC, which encodes an aspartic acid residue. This modification can be produced by targeted mutagenesis using TALEN, the Crispr/Cas system, or as described, for example, in U.S. Pat. No. 9,738,908 and U.S. Patent Application Publication No. 2019/0153430, each of which is incorporated by reference in its entirety. Mice bearing other gain-of-function SCN8A mutations can be produced similarly, including but not limited to mice carrying a leucine to valine substitution at amino acid 257 of SEQ ID NO: 12 by modification of the codon at nucleotides 922-924 of SEQ ID NO: 11, a leucine to valine substitution at amino acid 862 of SEQ ID NO: 12 by modification of the codon at nucleotides 2737-2739 of SEQ ID NO: 11, a glutamine to histidine substitution at amino acid 1468 of SEQ ID NO: 12 by modification of the codon at nucleotides 4555-4557 of SEQ ID NO: 11, a glycine to arginine substitution at amino acid 1473 of SEQ ID NO: 12 by modification of the codon at nucleotides 4570-4572 of SEQ ID NO: 11, an alanine to valine substitution at amino acid 1489 of SEQ ID NO: 12 by modification of the codon at nucleotides 4618-4620 of SEQ ID NO: 11, a methionine to threonine substitution at amino acid 1643 of SEQ ID NO: 12 by modification of the codon at nucleotides 5080-5082 of SEQ ID NO: 11, and/or an alanine to threonine substitution at amino acid 1648 of SEQ ID NO: 12 by modification of the codon at nucleotides 5095-5097 of SEQ ID NO: 11, or any combination thereof. Codon changes of interest can be determined by referring to the Codon Table presented herein below:

Amino Acid Codes and Functionally Equivalent Codons

| | 3- | 1- | |
|---|---|---|---|
| | Letter | Letter | |
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

### IV. Methods for Inducing Audiogenic Seizures and Seizure-Induced Death

In some embodiments, the presently disclosed subject matter provides approaches for inducing audiogenic seizures and seizure-induced death in a mouse animal model of SCN8A epilepsy that uses a transgenic mouse and sonicator.

Patients with SCN8A epilepsy often have apparent apnea and bradycardia during tonic seizures (see e.g., Trivisano et al., 2019; Negishi et al., 2021; Wenker et al., 2021). These studies included patients with several SCN8A mutations: L257V, L864V, Q1470H, G1475R, A1491V, M1645T, A1650T, and N1768D. This is precisely what was observed during the tonic seizures of both W/+ and D/+ mice (Wengert et al., 2021; Wenker et al., 2021). Thus, it stands to reason that alpha-1 receptor stimulation and mechanical ventilation would increase seizure survival for SCN8A epilepsy caused by any mutation. In addition, because SUDEP is a risk factor for patients with tonic seizures, use of alpha 1 adrenergic receptor agonists to prevent SUDEP would be applicable to epilepsy patients experiencing tonic seizures

As disclosed herein, various mutations in SCN8A gene products have been associated with human diseases including, but not limited to epilepsy. With reference to the amino acid numbering provided in Accession No. NP_055006.1 of the GENBANK® biosequence database (i.e., SEQ ID NO: 4), exemplary such mutations associated with human SCN8A epilepsy include the amino acid substitutions L257V, L864V, Q1470H, G1475R, A1491V, M1645T, A1650T, and N1768D. With particular reference to the human N1768D amino acid substitution, a review of the human and mouse SCN8A sequences indicates that the amino acid that corresponds to amino acid N1768 in humans is amino acid N1766 in the mouse. Similarly, L257 in the human SCN8A protein corresponds to L257 in the mouse SCN8A protein of SEQ ID NO: 12, L864 in the human SCN8A protein corresponds to L862 in the mouse SCN8A protein of SEQ ID NO: 12, Q1470 in the human SCN8A protein corresponds to Q1468 in the mouse SCN8A protein of SEQ ID NO: 12, G1475 in the human SCN8A protein corresponds to G1473 in the mouse SCN8A protein of SEQ ID NO: 12, A1491 in the human SCN8A protein corresponds to A1489 in the mouse SCN8A protein of SEQ ID NO: 12, M1645 in the human SCN8A protein corresponds to M1643 in the mouse SCN8A protein of SEQ ID NO: 12, and A1650 in the human SCN8A protein corresponds to A1648 in the mouse SCN8A protein of SEQ ID NO: 12. Nonetheless, because the amino acid substitutions generally relate to human mutations, the human numbering system as set forth in SEQ ID NO: 4 is employed herein. For example, as used herein the terms “N1768” and “N1768D” refer polypeptides comprising the human N1768D amino acid substitution as well as transgenic and non-transgenic animals that express the same in at least one cell.

Thus, in some embodiments the presently disclosed subject matter relates to methods for inducing audiogenic seizures and/or seizure-induced death in subjects such as but not limited to mice, the methods comprising, consisting essentially of, or consists of subjecting a germline modified animal such as a mouse as disclosed herein to an audiogenic stimulus of sufficient intensity and duration to induce an audiogenic seizure and/or seizure-induced death in the mouse. Exemplary audiogenic stimuli that can be employed for inducing audiogenic seizures and/or seizure-induced death include sound of at least about 12 kHz at an intensity of at least about 100 dB for a duration of at least about 10 seconds.

V. Methods for Identifying Compounds that have Activity in Treating and/or Preventing Seizures and/or Seizure-Induced Death

Using the animal models and the methods disclosed herein, compounds that have activity in treating and/or preventing a seizure and/or seizure-induced death in subjects can be identified. For example, in some embodiments the presently disclosed subject matter relates to methods for identifying compounds that have activity in treating and/or preventing a seizure and/or seizure-induced death in subjects, the methods comprising, consisting essentially of, or consisting of inducing an audiogenic seizure in the germline modified animal (e.g., mouse) as described herein; (b) administering a compound to be tested to the germline modified animal; and (c) determining whether the compound treats and/or prevents a seizure and/or seizure-induced death in the subject, whereby a compound that has activity in treating and/or preventing a seizure and/or seizure-induced death in a subject is identified. Techniques that can be employed for assessing the compounds are described in the EXAMPLES and also would be apparent to one of ordinary skill in the art upon a review of the disclosure herein of an animal model.

### VI. Compositions for Preventing Seizure-Induced Death

In some embodiments of the presently disclosed subject matter, a composition comprising, consisting essentially of, or consisting of an alpha-1 adrenergic receptor activator is employed. Exemplary alpha-1 adrenergic receptor activators include cirazoline (2-[(2-Cyclopropylphenoxy)methyl]-4,5-dihydro-1H-imidazole), methoxamine (2-amino-1-(2,5-dimethoxyphenyl)propan-1-ol), synephrine (4-[1-Hydroxy-2-(methylamino)ethyl]phenol), etilefrine (3-[2-(ethylamino)-1-hydroxyethyl]phenol), metaraminol (3-[(1R,2 S)-2-amino-1-hydroxypropyl]phenol), midodrine (2-amino-N-[2-(2,5-dimethoxyphenyl)-2-hydroxyethyl]acetamide), naphazoline (2-(naphthalen-1-ylmethyl)-4,5-dihydro-1H-imidazole), norepinephrine (4-[(1R)-2-amino-1-hydroxyethyl]benzene-1,2-diol), oxymetazoline (6-tert-butyl-3-(4,5-dihydro-1H-imidazol-2-ylmethyl)-2,4-dimethylphenol), phenylephrine (3-[(1R)-1-hydroxy-2-(methylamino)ethyl]phenol), pseudoephedrine ((1 S,2 S)-2-(methylamino)-1-phenylpropan-1-ol), tetrahydrozoline (2-(1,2,3,4-tetrahydronaphthalen-1-yl)-4,5-dihydro-1H-imidazole), and xylometazoline (2-[(4-tert-butyl-2,6-dimethylphenyl)methyl]-4,5-dihydro-1H-imidazole), and pharmaceutically acceptable salts thereof (e.g., hydrochlorides, phosphates, sodium salts, etc.).

In some embodiments, the presently disclosed subject matter provides, within the same model, an ˜80% chance of seizure-induced death at one timepoint and a ˜0% chance of seizure-induced death at another timepoint. This aspect of the model provides a feature of any experimental model aimed to examine mechanisms of seizure-induced sudden death.

The presently disclosed subject matter provides, with respect to the utility of phenylephrine as a potential SUDEP therapeutic intervention, at least two aspects: (1) it is believed that phenylephrine could be utilized as an interventional medication administered by a caregiver after seizure onset, particularly during clonic/tonic seizures, since these types of seizures have been observed in the few recorded cases of SUDEP in human (Mortemus study: Ryvlin et al., 2013); and (2) it is believed that phenylephrine could be utilized in potential closed-loop seizure-detection and medication delivery approaches which are currently being actively investigated. Unlike mice which have a short time between seizure onset and sudden death (<16 seconds in D/+ mice), human epilepsy patients have a wider window which would render therapeutic interventions possible (evidenced by various rescue medication approaches already used by epilepsy patients). Of interest, phenylephrine is readily used for clinical applications.

The presently disclosed methods are applicable to other epilepsy etiologies that experience tonic seizures. First, a number of SUDEP mouse models, including Scn1a^{R1407X}, DBA1/2, Kcna1^{-/-}, 129/SvTer, Cacna1a^{S218L} mice, experience fatal seizures that are classified as tonic, generally based on the observation of hindlimb extension (Faingold et al., 2010, 2016; Kim et al., 2018; Dhaibar et al., 2019; Jansen et al., 2019; Loonen et al., 2019; Martin et al., 2020). Furthermore, in the maximal electroshock, PTZ-induced, and inbred DBA/1 and LMX1f/f/p mouse models of SUDEP, death was prevented by enhancement of the noradrenergic system. Second, tonic seizures are common to many types of epilepsy, including the very fatal Lennox-Gastaut Syndrome (Autry et al., 2009).

VI.A. Formulations

The compositions (e.g., alpha-1 adrenergic receptor activators) of the presently disclosed subject matter can be administered in any formulation or route that would be expected to deliver the compositions to whatever target site might be appropriate.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

VI.D. Dosages

An effective dose of a composition of the presently disclosed subject matter is administered to a subject in need thereof. An “effective amount”, “treatment effective amount” or a “therapeutic amount” is an amount of a therapeutic composition sufficient to produce a measurable response (e.g., a biologically or clinically relevant response in a subject being treated, such as but not limited to a reduction in seizure activity and/or in the incidence of death, particularly as compared to the same subject had the subject not received the composition). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

VI.C. Routes of Administration

Suitable methods for administration of the compositions of the presently disclosed subject matter include, but are not limited to intravenous administration and delivery directly to a target tissue or organ. Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated. By way of example and not limitation, in some embodiments a composition of the presently disclosed subject matter is administered to the subject via a route selected from the group consisting of intraperitoneal, intramuscular, intravenous, and intranasal, or any combination thereof.

The presently disclosed subject matter uses mechanical ventilation and/or acute activation of alpha-1 adrenergic receptors to prevent seizure-induced death. Based on these findings, the presently disclosed subject matter relates in some embodiments to activation of these receptors using an interventional “Epi pen” style therapy stimulating the recovery of breathing function to prevent seizure-induced death following a seizure.

In addition, with respect to subcutaneous delivery, a pen delivery device readily has applications in delivering a pharmaceutical composition of the presently disclosed subject matter. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, United Kingdom), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Indiana, United States of America), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, New Jersey, United States of America), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, California, United States of America), the PENLET™ (Haselmeier, Stuttgart, Germany), the EPI PEN (Dey, L. P.), and the HUMIRA™ Pen (Abbott Labs, Abbott Park, Illinois, United States of America), to name only a few. See e.g., U.S. Pat. Nos. 7,762,994; 8,409,149; 8,556,864; 8,579,869; 9,011,391; and 9,265,893, the disclosure of each of which is incorporated herein by reference in its entirety.

### VII. Methods for Preventing Seizure-Induced Death

In some embodiments, the presently disclosed subject matter relates to methods for treating and/or preventing death associated with seizures in subjects. In some embodiments, the methods comprise, consist essentially of, or consist of stimulating breathing of the subject via mechanical ventilation and/or by administering a composition comprising an effective amount of an alpha-1 adrenergic receptor activator to the subject. In some embodiments, the seizure is an epileptic seizure.

In some embodiments, the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline. The alpha-1 adrenergic receptor activator can be administered at any time, including before the onset of and/or during a seizure. In some embodiments, the alpha-1 adrenergic receptor activator is administered to the subject during a tonic phase of the seizure, optionally within 1, 2, 3, 4, or 5 minutes from the onset of the seizure.

When mechanical ventilation is provided, the mechanical ventilation of the subject is initiated as early in the seizure phase as possible, including just after the subject's own breathing fails. It can be continued until the subject is able to breath unassisted.

In some embodiments, the seizure-induced death is sudden unexpected death in epilepsy (SUDEP). As is known in the art, in some embodiments SUDEP is associated with a gain-of-function mutation in an SCN8A gene product in the subject. Exemplary gain-of-function SCN8A mutation include an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof.

As such, with respect to SUDEP, in some embodiments the presently disclosed subject matter relates to methods for preventing or reducing the incidence of SUDEP comprising, consisting essentially of, or consisting of stimulating breathing of the subject via mechanical ventilation and/or by administering a composition comprising an effective amount of an alpha-1 adrenergic receptor activator to the subject. As used herein, the phrase “preventing or reducing the incidence of” refers to an outcome that is characterized by a lower incidence of a particular outcome when a particular intervention is employed as compared to what might have been expected had the intervention not occurred. By way of example and not limitation, “preventing or reducing the incidence of SUDEP” means that a particular treatment (e.g., stimulating breathing of the subject via mechanical ventilation and/or by administering a composition comprising an effective amount of an alpha-1 adrenergic receptor activator to the subject) results in the subject surviving the seizure when, in the absence of the treatment, the subject would have been expected to have died.

The alpha-1 adrenergic receptor activator can be administered to the subject during a tonic phase of the seizure. In some embodiments, the alpha-1 adrenergic receptor activator can be administered to the subject as soon as possible after the onset of the seizure, which can be in some embodiments within 1, 2, 3, 4, or 5 minutes from the onset of the seizure.

Here as well, mechanical ventilation can be provided to the subject. In some embodiments, the mechanical ventilation is provided to the subject as soon as possible after the subject's own unassisted breathing fails or is compromised, and can in some embodiments continue until the subject is able to breath unassisted.

Accordingly, in some embodiments the presently disclosed subject matter relates to methods for preventing and/or reducing the risk of death in subjects having one or more a gain-of-function mutations in an SCN8A gene product. Here as well, the phrase “preventing and/or reducing the risk of death” refers to an outcome that is characterized by a lower incidence of death when a particular intervention is employed as compared to what might have been expected had the intervention not occurred. As such, “preventing and/or reducing the risk of death” means that a particular treatment (e.g., stimulating breathing of the subject via mechanical ventilation and/or by administering a composition comprising an effective amount of an alpha-1 adrenergic receptor activator to the subject) results in the subject being more likely to survive the seizure when, in the absence of the treatment, the subject would have been expected to have died.

In some embodiments, the methods comprise, consist essentially of, or consist of stimulating breathing of the subject via mechanical ventilation and/or by administering a composition comprising an effective amount of an alpha-1 adrenergic receptor activator to the subject. SCN8A gain-of-function mutations are disclosed herein, and include but are not limited to an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof.

Furthermore, in some embodiments the presently disclosed subject matter relates to methods for preventing or reducing the risk of death associated with tonic seizures in subjects in need thereof. In some embodiments, the methods comprise, consist essentially of, or consist of stimulating breathing of the subject via mechanical ventilation and/or by administering a composition comprising an effective amount of an alpha-1 adrenergic receptor activator to the subject. In some embodiments, the subject' genome comprises a gain-of-function mutation in an SCN8A gene product, including but not limited to those disclosed herein. It is noted, however, that tonic seizures can occur in the absence of SCN8A mutations, and the instant methods can still be employed in such subjects.

More generally, in some embodiments the presently disclosed subject matter relates to methods for preventing or reducing the risk of death associated with epileptic seizures resulting from any underlying cause. As such, in some embodiments the methods comprise, consist essentially of, or consist of stimulating breathing of the subject via mechanical ventilation and/or by administering a composition comprising an effective amount of an alpha-1 adrenergic receptor activator to the subject. In some embodiments, the subject's genome carries a gain-of-function mutation in an SCN8A gene product, and in some embodiments the subject's genome does not carry a gain-of-function mutation in an SCN8A gene product. In those embodiments where the subject's genome carries a gain-of-function mutation in an SCN8A gene product, the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof. Here as well, in some embodiments the alpha-1 adrenergic receptor activator is administered to the subject during a tonic phase of the seizure, in some embodiments as early as possible subsequent to the onset of the tonic phase of the seizure, optionally within 1, 2, 3, 4, or 5 minutes from the onset of the seizure. The presently disclosed methods can also comprise providing mechanical ventilation to the subject, with or without the administration of an alpha-1 adrenergic receptor activator.

In some embodiments of any of the presently disclosed methods, the composition comprising the alpha-1 adrenergic receptor activator, the mechanical ventilation, if administered, or both can be administered to the subject subsequent to development of apnea but prior to the end of a tonic phase experienced by the subject.

In some embodiments of any of the presently disclosed methods, the subject is a human.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the Examples

Mice. All mice were housed and maintained in accordance with the Animal Care and Use Committee standards of the University of Virginia in a temperature and humidity-controlled vivarium with a standard 12-hour light/dark cycle with food and water ad libitum. Both male and female mice were used in roughly equal numbers, and no sex differences were observed for any of the experiments based on seizure behavior or risk of sudden death.

Genotyping. Genotyping of transgenic mice was done using standard PCR techniques with DNA acquired from tail biopsies. Genotyping of D/+ mice was performed as previously described (Wagnon et al., 2015), using the primers 5′-TGACTGCAGCTTGGACAAGGAGC-3′ (SEQ ID NO: 1) and 5′-TCGATGGTGTTGGGCTTGGGTAC-3′ (SEQ ID NO: 2). The resulting PCR product, a 327 bp genomic fragment derived from the SCN8A genetic locus and containing the mutation, was then digested with HincII, which generates a single fragment of 327 bp for the wild type allele and two fragments of 209 and 118 bp for the mutant allele.

Audiogenic seizure assessment. To test for audiogenic seizures mice were taken from their home cage and transferred to a clean test cage where they were allowed to acclimate for ˜20 seconds before the onset of the acoustic stimulus. Similar to a method described previously (Martin et al., 2020), a sonicator (Branson 200 ultrasonic cleaner) was used to produce the audiogenic stimulus directly adjacent to the test cage. The stimulus duration lasted for 50 seconds or until the animal had a behavioral seizure.

Audiogenic seizures were recorded using a laptop webcam. Duration of seizure phases were analyzed by taking the time in seconds that the mouse spent in each of the phases: a wild-running phase characterized by fast circular running throughout the cage, a tonic phase characterized by hindlimb extension and muscle rigidity, a clonic phase typified by myoclonic jerking of the hindlimbs, and recovery exemplified when the mouse ceased myoclonic jerking and righted itself. In cases where death occurred, the end of the tonic phase was apparent at the point of hindlimb muscle relaxation. For all experiments involving rescue of seizure-induced sudden death, at least one control mouse from the experimental litter was confirmed to experience seizure-induced sudden death before conducting any rescue experiments on remaining littermates.

Intensity-dependence of audiogenic seizures. Intensity-dependent sensitivity of the audiogenic seizures was tested using adult D/+, placed in a custom wooden chamber and exposed to ˜20 seconds of pure tone acoustic stimulation (Audacity open-source digital audio editor) at 14 kHz using a JBL speaker (Model #2446H; JBL Incorporated, Stamford, Connecticut, United States of America). Tones were delivered beginning at 50 dB and manually increased in increments of ˜5 dB until the D/+ mouse exhibited an audiogenic seizure.

Auditory brainstem responses. Auditory Brainstem Responses (ABRs) were recorded from WT and D/+ mice at P56 and P112 in a blinded manner, as previously described (Ruhl et al., 2019). The mice were anesthetized with a single intraperitoneal injection of 100 mg/kg ketamine hydrochloride (Fort Dodge Animal Health, Overland Park, Kansas, United States of America) and 10 mg/kg xylazine hydrochloride (Lloyd Laboratories Inc., Peterborough, Ontario, Canada). Body temperature was maintained with a Deltaphase isothermal heating pad (Braintree Scientific Inc., Braintree, Massachusetts, United States of America) during the procedure. The ABRs were performed in a sound-attenuating booth (Med-Associates, St. Albans, Vermont, United States of America) using equipment and software (Smart-EP) from Intelligent Hearing Systems of Miami, Florida, United States of America. Recordings were collected through subdermal needle electrodes (Intelligent Hearing Systems). A noninverting electrode was placed at the vertex of the midline, an inverting electrode was placed over the mastoid of the right ear, and a ground electrode was placed on the upper thigh. Pure tone stimuli of 31.3 microseconds were presented at the rate of 21.1/seconds through a high frequency transducer (Intelligent Hearing Systems). Responses were filtered at 300-3000 Hz and threshold levels were determined from averages of 1024 stimulus presentations. Stimulus intensity at each tested frequency (8, 11.3, 16, 22.4, and 32 kHz) was decreased from the maximum intensity in 5-10 dB steps until a waveform response could no longer be identified. The maximum intensities used were 120, 120, 110, 100, and 90 dB for 8, 11.3, 16, 22.4, and 32 kHz, respectively. The lowest intensity at which a response was observed was recorded as the threshold. If a waveform could not be identified at the maximum output of the transducer, a value of 5 dB was added to the maximum output as the threshold.

Surgical preparation. Custom ECoG/ECG headsets (PlasticsOne, Inc., Roanoke, Virginia, United States of America; or Pinnacle Technology Inc., Lawrence, Kansas, United States of America) were implanted in 6-10-week-old D/+ mice using standard aseptic surgical techniques. Anesthesia was induced with 5% and maintained with 0.5%-3% isoflurane. Adequacy of anesthesia was assessed by lack of toe-pinch reflex. A midline skin incision was made over the skull, and burr holes were made at the lateral/rostral end of both the left and right parietal bones to place EEG leads, and at the interparietal bone for a reference and ground electrodes. Two ECG leads were passed subcutaneously to the left abdomen and right shoulder and sutured into place to approximate a lead II arrangement. The headsets were attached to the skull with dental acrylic (Jet Acrylic; Lang Dental, Wheeling, Illinois, United States of America). Mice received postoperative analgesia with meloxicam (0.5-1 mg/kg, i.p.) or ketoprofen (5 mg/kg, i.p.) and 0.9% saline (0.5 ml i.p.) and were allowed to recover a minimum of 2-5 days prior to experiments.

Recording of ECoG, ECG, and breathing for spontaneous seizures. After recovery from surgery, mice were individually housed in custom-fabricated plethysmography chambers and monitored 24 hours a day. Plethysmography chambers were built to comply with requirements for continuous housing described in the Guide for the Care and Use of Laboratory Animals (Council, 2011). The floor of the chambers had approximate dimensions of 4.5×4.5 inches (>20 sq. inches) and 7 inches tall. There were ports for air in and air out, and for pressure monitoring. The chamber was supplied with a continuous flow of room air at approximately 400 ml/min via supply and exhaust air pumps (MK-1504 Aquarium Air Pump; AQUA Culture, Bentonville, Arkansas, United States of America) balanced to maintain chamber pressure near atmospheric. Mice had access to a continuous supply of water and food. The surgically implanted headsets were attached to a custom low torque swivel cable, allowing mice to move freely in the chamber.

To assess breathing frequency, the pressure of the epilepsy monitoring unit chamber was measured with an analogue pressure transducer (SDP1000-L05; Sensirion Inc., Chicago, Illinois, United States of America). ECoG and ECG signals were amplified at 2000 and bandpass filtered between 0.3-100 Hz and 30-300 Hz, respectively, with an analogue amplifier (Neurodata Model 12, Grass Instruments Co., West Warwick, Rhode Island, United States of America). Biosignals were digitized with a Powerlab 16/35 and recorded using LabChart 7 software (AD Instruments, Inc., Milford, Massachusetts, United States of America) at 1 kS/s. Video acquisition was performed by multiplexing four miniature night vision-enabled cameras and then digitizing the video feed with a Dazzle Video Capture Device (Corel, Inc., Ottawa, Ontario, Canada) and recording at 30 fps with LabChart 7 software in tandem with biosignals.

Recording of ECoG, ECG, and breathing for audiogenic seizures. For simultaneous ECoG, ECG, and breathing during audiogenic seizures, the same surgical procedure and experimental setup described above was used. Although mice remained in the chambers 24 hours a day, recording only took place during periods of audiogenic seizure stimulation. To induce audiogenic seizures, a 15 kHz signal was generated using Tone Generator software (NCH Software, Inc., Greenwood Village, Colorado, United States of America), amplified using a Kinter K3118 stereo amplifier (Kinter USA, Waukegan, Illinois, United States of America), and converted to sound using a small 3-watt speaker lowered into the plethysmography chamber.

For recording of only breathing in pharmacological experiments, non-implanted mice were placed in the chambers immediately after injection of pharmacological agents. Stimulation of audiogenic seizures and recording of breathing were performed as described above.

Breathing and heart rate detection. Individual breaths and heart beats were identified as inspiratory deflections in the pressure transducer signal and R waves in the ECG signal, respectively, using Spike2 software (Cambridge Electronic Design, Ltd., Cambridge, United Kingdom). A breath was scored when the downward deflection went below a certain hysteresis value determined by the experimenter and rose back above a threshold of 0 mV. The minimum time between breaths was set to 0.05 s. Similarly, an R wave was identified when an upward deflection crossed a threshold value determined by the experimenter. The minimum time between R waves was set to 0.02 s. All breaths and R waves were inspected by the experimenter.

Mechanical ventilation. For the mechanical ventilation of P20-21 D/+ mice during an audiogenic seizure, we quickly placed a custom-made 3 mL pipet which fit snuggly over the mouse's nose and mouth and provided regular pulses of air (˜1 mL at 2 Hz). The ventilation was terminated after the mouse initiated gasping behavior or 20 s after the animal experienced sudden death.

Adrenergic receptor pharmacology. All chemicals were purchased from Sigma Aldrich and were either of pharmaceutical grade or were sterile filtered prior to injection. Injections were given intraperitoneal in a volume of 50-100 ul sterile saline per mg of mouse weight (e.g. 0.1 mL for a 20 mg adult mouse). The concentration of each drug was based on the achieving the desired dosages of 2 mg/kg epinephrine HCl, 2 mg/kg norepinephrine HCl, and 3 mg/kg phenylephrine HCl, 10 mg/kg sotalol HCl, and 1 mg/kg prazosin HCl. Injection of 50-100 ul sterile saline per mg of mouse weight was used as control.

Statistical analysis. All interventions were conducted and analyzed with the experimenter blinded, except for mechanical ventilation. All data points denote biological replicates (i.e. no animal was used more than once for the same test), except for data comparing spontaneous and audiogenic seizures, which are technical replicates and the animal numbers are reported in the figure legend. All average data values are mean±SEM. Statistics were computed using GraphPad Prism version 7 or later (GraphPad Software, Inc., San Diego, California, United States of America) and comparisons were considered statistically detectable when p<0.05. Differences between two groups were assessed by unpaired, two-tailed Student's t-test when distributions passed the D'Agnostino-Pearson Omnibus normality test and Mann-Whitney non-parametric test when any distribution failed to pass the normality test. Differences between more than two groups were assessed by one-Way or two-Way ANOVA followed by Holm-Sidak's or Sidak's multiple comparison tests, respectively. For the few cases where residuals of one-Way ANOVAs failed the D'Agnostino-Pearson Omnibus normality test (p<0.05), we used the Kruskal-Wallis nonparametric test followed by Dunn's multiple comparisons test. Comparison of survival proportions was done using a one-sided Fisher's exact test.

### Example 1

### Characterization of Audiogenic Seizure Behavior in D/+ Mice

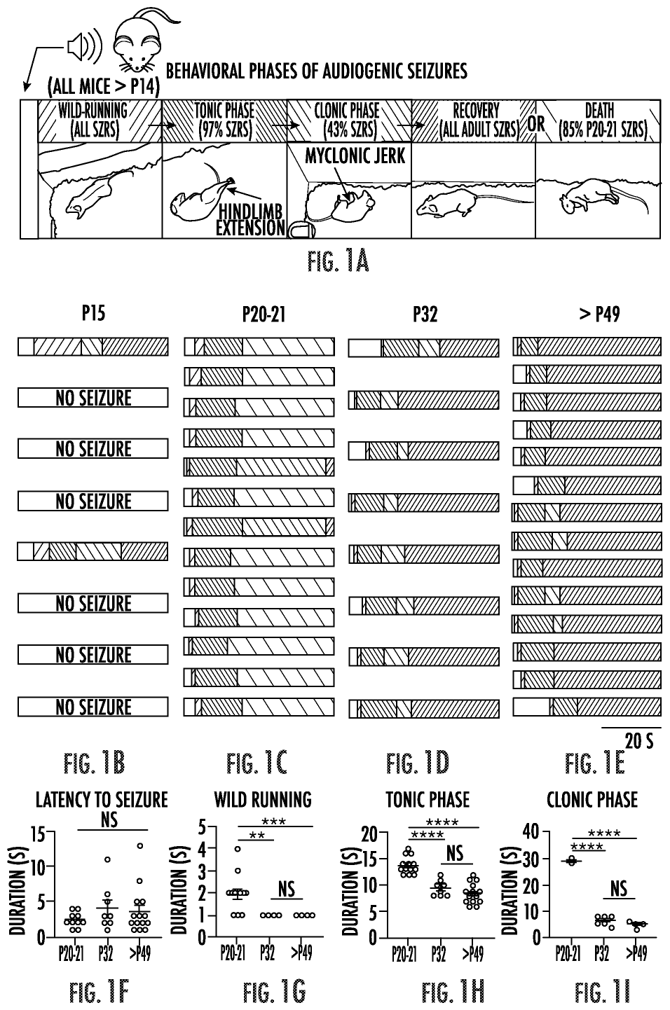

It was discovered that D/+ mice were susceptible to audiogenic seizures when a sonicator (e.g., a Branson 200 Ultrasonic cleaner) was turned on in close proximity to D/+ mice. Further investigation using the same acoustic stimulus revealed that D/+ mice at ages P20-21, P32, and P49-P69 exhibited stereotyped seizure behaviors: wild-running followed by a tonic phase with hindlimb extension that was, in some cases, followed by a clonic phase consisting of myoclonic leg jerking (FIG. 1A). Only 2 of 8 D/+ mice were sensitive to audiogenic seizures at P15 (FIG. 1B), which we attributed to the fact that mice at this age are likely still developing their auditory system. Strikingly, all 13 D/+ mice tested at P20-21 experienced audiogenic seizures, 11 of which succumbed to sudden death immediately following the seizure (FIG. 1C). In contrast, no deaths were observed in D/+ mice at P32 (n=8) or P49-69 (n=14) even though all mice experienced an audiogenic seizure (FIGS. 1D and 1E). Further characterization of the audiogenic seizures revealed that there were no differences in latency to seizure (p=0.25; F_{2,32}=1.460; FIG. 1F) although P20-21 D/+ mice experienced seizures with longer wild-miming (**p=0.0017 and ***p<0.0002, respectively; Dunn's multiple comparisons test; FIG. 1G), tonic (****p<0.0001 for both comparisons; F_{2,32}=32.99, FIG. 1H), and clonic (****p<0.0001 for both comparisons; F_{2,32}=211.5, FIG. 1I) phases compared to D/+ mice of ages P32 and P49-69. No seizures were observed in wild type (WT) littermates exposed to the same acoustic stimulus (n=37). It should be noted that due to the high incidence of death at P20-21, only two instances of a clonic phase were observed.

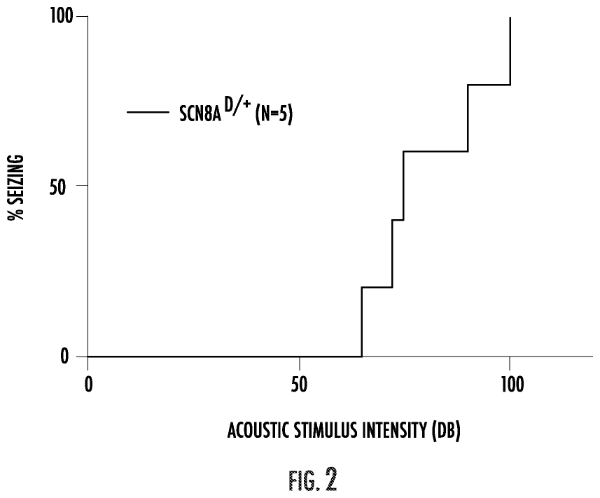

The intensity-sensitivity of audiogenic seizures was characterized in adult D/+ mice using a custom speaker/microphone feedback system (see Materials and Methods for the EXAMPLES section herein above). To determine intensity-sensitivity, D/+ mice were exposed to pulses of ˜20 s, 14 kHz acoustic stimuli, increasing each consecutive pulse by ˜5 dB until an audiogenic seizure was triggered. All 5 D/+ had audiogenic seizures by 100 dB (see FIG. 2).

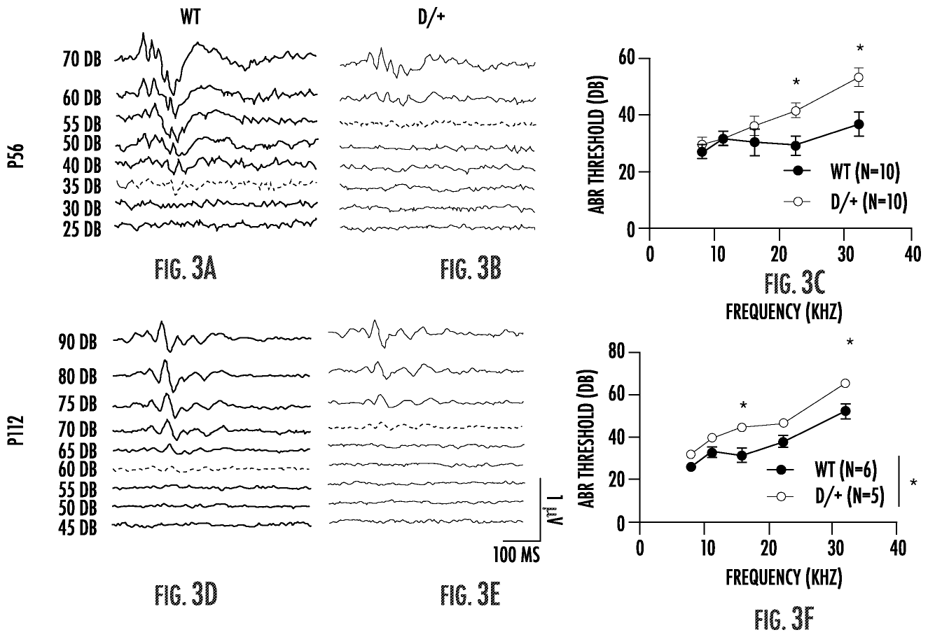

Other mouse models that exhibit audiogenic seizures, as well as mice with different Scn8a mutations, have impaired hearing (Willott et al., 1995; Willott & Bross, 1996; Koay et al., 2002; Mackenzie et al., 2009; Heffner et al., 2019). Thus, hearing sensitivity in D/+ and WT littermate control mice was measured using auditory brainstem response (ABR) testing. Hearing thresholds at a range of frequencies (8, 11.3, 16, 22.4, and 32 kHz) were determined at two developmental time points; P56 and P112. Mild hearing impairment was found at both ages (FIG. 3). At P56, ABR thresholds of D/+ mice were elevated compared to those of WT mice at 22.4 and 32 kHz (*p=0.0479 and 0.0322, respectively; F_{1,18}=3.929; FIG. 3C). At P112, ABR thresholds observed in D/+ were elevated at 16 and 32 kHz and across the entire frequency range tested compared to WT mice (**p=0.0034, **p=0.003, *p=0.0156; F_{1,9}=8.844; FIGS. 3D-3F).

### Example 2

### Audiogenic and Spontaneous Seizures Have Similar Semiology

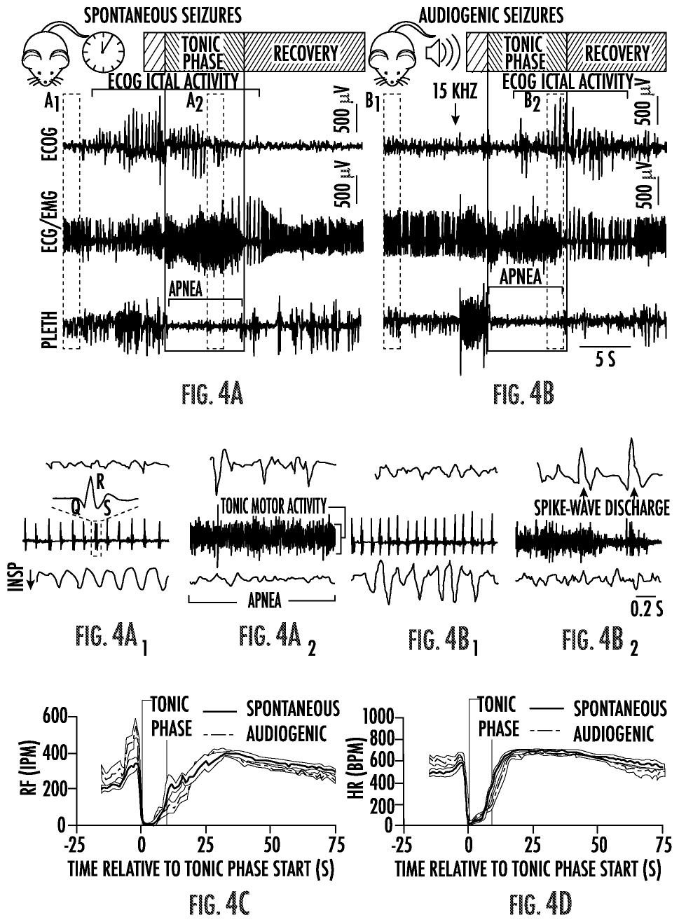

Spontaneous and audiogenic seizures from adult D/+ mice were recorded with standard rodent video/Electrocorticogram (ECoG) techniques. In addition, muscle/cardiac function and breathing frequency were simultaneously recorded via electrocardiogram (ECG)/electromyogram (EMG) activity and plethysmography, respectively (FIG. 4). Both spontaneous and audiogenic seizures presented with a series of cortical spike-wave discharges in addition to a period noted by a large amount of tonic muscle activity, apnea, and bradycardia that coincided with the behavioral tonic phase (FIGS. 4A and 4B). No differences in ictal or postictal breathing (FIG. 4C) or heart rate (FIG. 4D), nor ECoG spike-wave discharge (FIG. 4E) or apnea duration (Mann-Whitney Test; FIG. 4F) were detected between spontaneous and audiogenic seizures. A robust increase in respiratory frequency was observed upon audio stimulation that was not observed in the spontaneous seizure type (FIG. 4C, −5 to 0 s). Interestingly, we found that the onset of ECoG spike-wave discharge always occurred after the initiation of the tonic phase in audiogenic seizures, whereas it typically preceded the tonic phase in spontaneous seizures (****p=0.0001; Mann-Whitney Test; FIG. 4G). These results suggested that audiogenic and spontaneous seizures in D/+ mice are comparable, sharing similar mechanisms.

### Example 3

### Audiogenic Seizure-Induced Death is Due to Lack of Breathing Recovery after the Tonic Phase

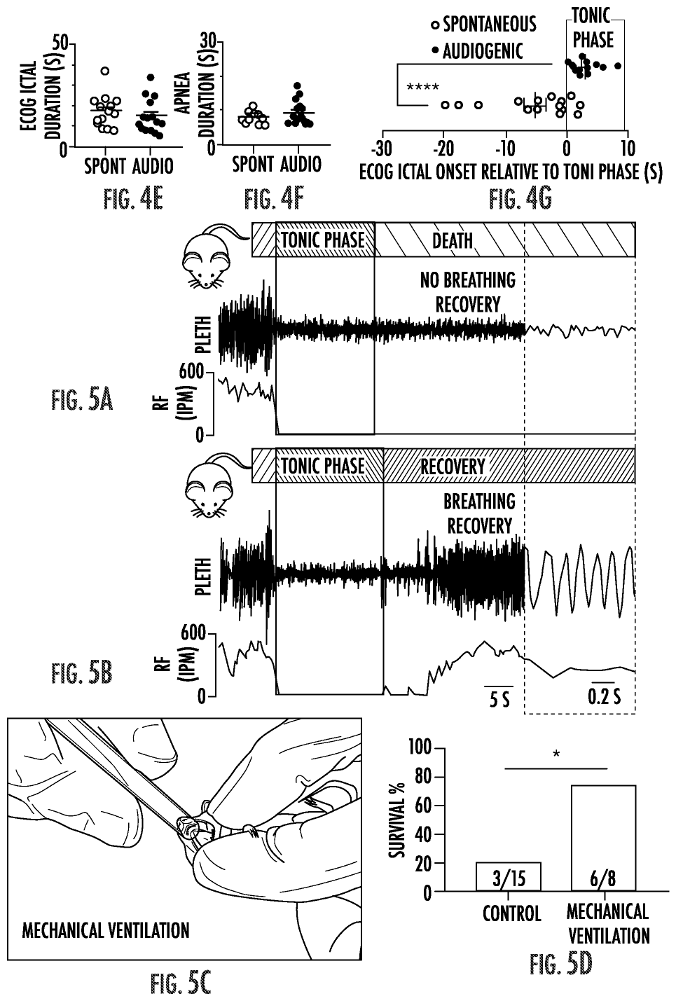

While both cardiac and respiratory arrest have been suggested as mechanisms of SUDEP, the primary mechanism of death remains debated (Massey et al., 2014; Devinsky et al., 2016). To examine breathing activity during terminal seizures, we conducted plethysmography recordings of P20-21 D/+ mice during audiogenic seizures. Audiogenic seizure-induced death observed in P20-21 D/+ mice occurred when apnea was initiated during the tonic phase and breathing failed to recover (FIG. 5A). In mice only a few days older (P25), breathing resumed immediately after the tonic phase and all mice tested survived (n=7; FIG. 5B). These data supported the notion that at a critical time point of P20-21, a lack of breathing recovery caused seizure-induced death.

Stimulation of breathing via mechanical ventilation has been shown to rescue seizure-induced respiratory arrest in other mouse models of SUDEP (Faingold et al., 2010; Kim et al., 2018). The ability to evoke seizures on command disclosed herein allowed us to assess whether this intervention could rescue the P20-21 D/+ mice. Mechanical ventilation (approximately 1 mL at 2 Hz delivered manually) was delivered to mice immediately after the onset of the tonic phase (FIG. 5C). While only three of 15 non-ventilated mice survived, six of the eight mice that were ventilated survived (*p<0.05; One-sided Fisher's exact test; FIG. 5D), demonstrating that respiratory arrest was the primary cause of seizure-induced death in P20-21 D/+ mice.

### Example 4

### Exogenous Alpha-1 Adrenergic Receptor Stimulation is Sufficient for Recovery Breathing and Survival of Audiogenic Seizures in Adult D/+ Mice

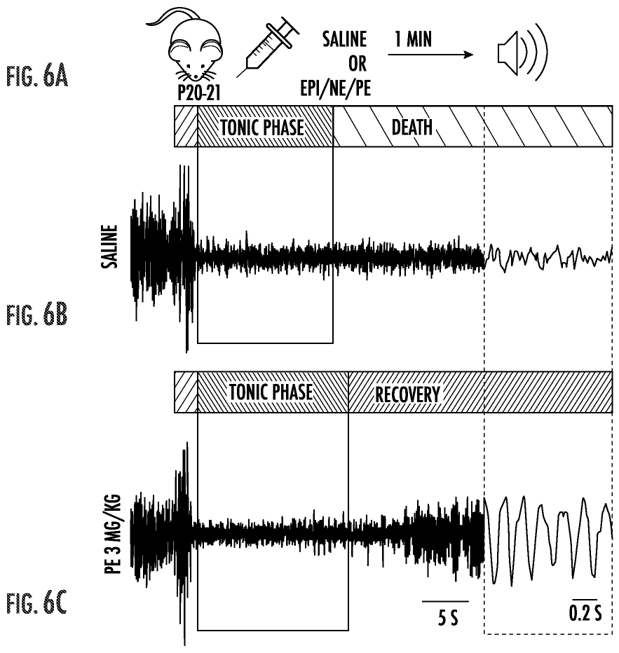

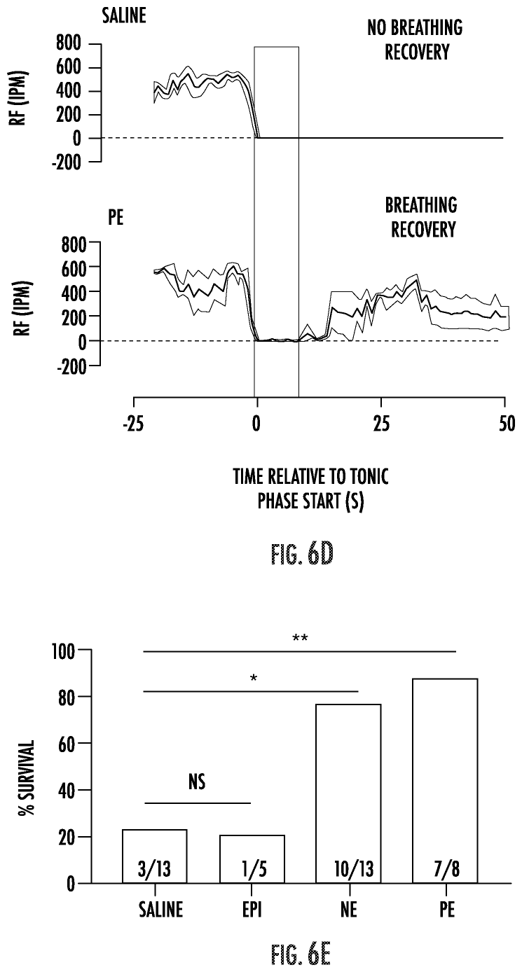

Activation of adrenergic receptors has been shown to stimulate respiration in animals and humans (Whelan & Young, 1953; Zhang et al., 2017; Zhao et al., 2017). To determine if acute activation of adrenergic receptors could stimulate breathing and prevent audiogenic seizure-induced death, P20-21 D/+ mice were injected with either 2 mg/kg norepinephrine (NE) or an equal volume of sterile saline via intraperitoneal injection (i.p.) 1 minute prior to audiogenic seizure stimulation (FIG. 6A). Saline injection had no effect on survival and was not different from non-injected mice (n=13; p=0.6; One-sided Fisher's exact test). In contrast, injection of NE resulted in breathing recovery and increased survival (10 of 13 mice) compared to saline injection (3 of 13 mice; *p<0.05; One-sided Fisher's exact test; FIG. 6E). Interestingly, injection of 2 mg/kg epinephrine (Epi) rescued only one of the five mice tested (FIG. 6E).

Based on the fact that NE primarily stimulates the alpha adrenergic receptors (Motiejunaite et al., 2020), whether selective alpha-1 receptor agonism might prevent seizure-induced death was tested. Injection of 3 mg/kg phenylephrine (PE) promoted postictal breathing recovery and survival in 7 out of 8 mice (**p<0.01; One-sided Fisher's exact test; FIGS. 6A-6E). Thus, alpha-1 adrenergic receptor activity was sufficient to promote postictal recovery of breathing and survival of audiogenic seizures in P20-21 D/+ mice.

### Example 5

### Endogenous Adrenergic Receptor Function is Required for Recovery Breathing and Survival of Audiogenic Seizures in Adult D/+ Mice

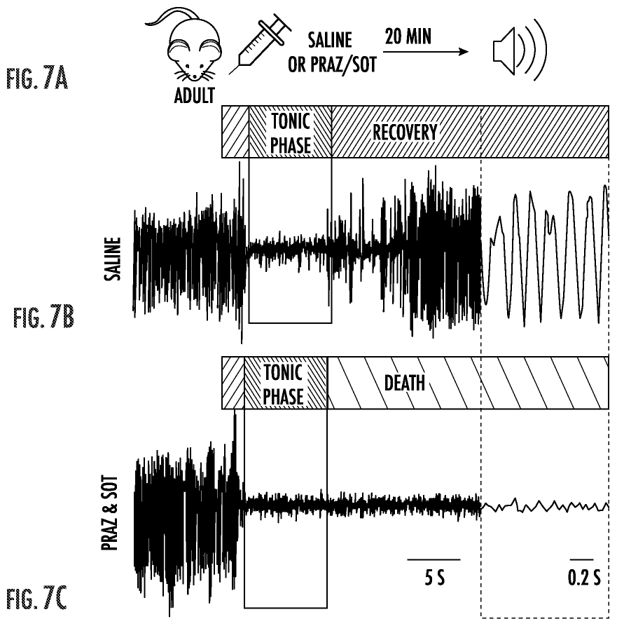

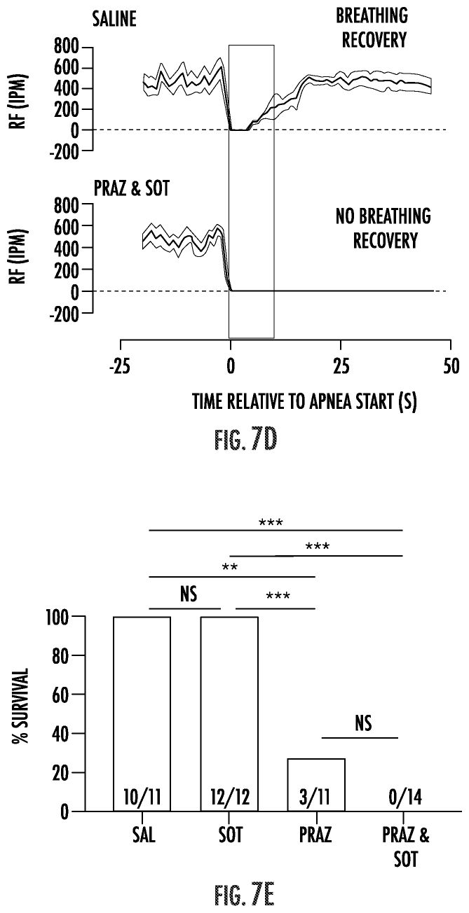

In order to test the necessity of adrenergic receptor function for breathing recovery and survival, we inhibited adrenergic receptor subtypes in adult D/+ mice which experience only non-fatal audiogenic seizures. Adult D/+ mice were injected (i.p.) 15 minutes prior to audiogenic seizure stimulation with either 1 mg/kg prazosin (an alpha-1 receptor antagonist), 10 mg/kg sotalol (a beta receptor antagonist), a combination of the two, or saline as a control (FIG. 7A). As expected, all 10 saline-injected adult D/+ mice had audiogenic seizures followed by recovery of breathing and survival (FIGS. 7B, 7D, and 7E). 12 out of 12 sotalol-treated mice survived, whereas only three of 11 prazosin-treated mice survived (**p<0.01 compared to saline-injected controls; ***p<0.001 compared to sotalol; FIG. 7E). Interestingly, all 14 D/+ mice treated with the combination of prazosin and sotalol died immediately following an audiogenic seizure (***p<0.001; FIGS. 7C-7E). Plethysmography recordings for prazosin/sotalol-treated adult D/+ mice revealed absence of breathing recovery (FIGS. 7C and 7D) similar to that of P20-21 D/+ mice (FIGS. 5A and 5B and FIGS. 6A and 6B). These results indicated that activity of alpha-1 adrenergic receptors was required for the normal recovery of breathing and survival following audiogenic seizures of adult D/+ mice.

### Example 6

### Prevention of Seizure-Induced Death with Mechanical Ventilation does not Require Adrenergic Receptor Function

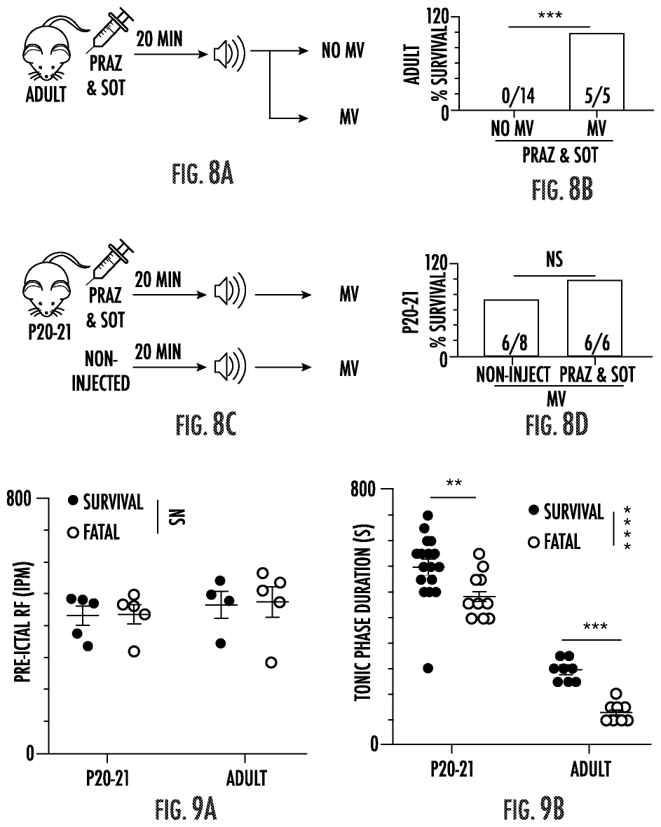

Our results suggested that adrenergic receptor activity prevented death primarily via stimulation of breathing. It was reasoned that even in the absence of functional adrenergic receptor signaling, any procedure to stimulate breathing would be able to rescue D/+ mice from seizure-induced death. To test this, we performed two sets of experiments: First, mice were injected (i.p.) with the combination of 10 mg/kg sotalol and 1 mg/kg prazosin 15 minutes prior to audiogenic seizure stimulation of adult D/+ mice, and either mechanically ventilated immediately after the start of the tonic phase or performed no intervention as a control (FIG. 8A). Although the adrenergic antagonist cocktail caused audiogenic seizure-induced death in adult D/+ mice (0 out 14 survived; FIG. 7E), all mice that received mechanical ventilation in addition to the adrenergic cocktail recovered breathing and survived (5 out of 5 survived; ***p=0.001; FIG. 8B).

Second, we either injected (i.p.) P20-21 D/+ mice with the same adrenergic antagonist cocktail or gave no injection prior to audiogenic seizure-stimulation, and mechanically ventilated both groups immediately after the start of the tonic phase (FIG. 8C). Adrenergic receptor blockade in P20-21 D/+ mice did not prevent the ability of mechanical ventilation to promote survival (6 of 6 survived; FIGS. 8C and 8D). This evidence supported the notion that adrenergic receptors function, likely indirectly, to stimulate breathing recovery after the tonic phase. However, any process that can directly stimulate breathing recovery will promote survival, even in the absence of adrenergic receptor function.

### Example 7

### Neither Suppressed Pre-Ictal Breathing Rate Nor Extended Tonic Phase Cause Seizure-Induced Death

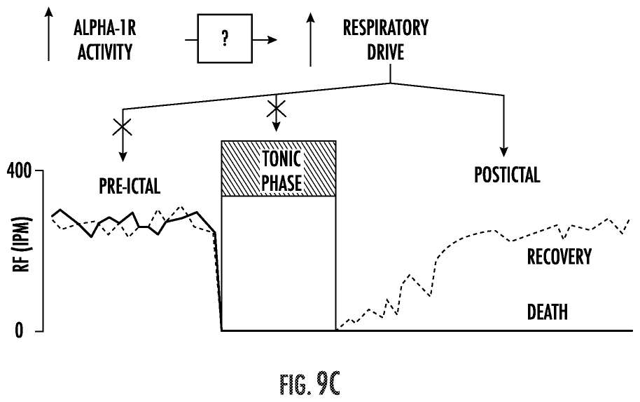

It was found that apnea occurs during all audiogenic seizures in D/+ mice older than P15, and whether an individual seizure was fatal or not depended on recovery of breathing after this initial apnea. It was possible that adrenergic receptor activity promoted basal breathing, and this increased the likelihood of survival. No differences in pre-ictal breathing rates between fatal and survival seizures (F_{1,15}=0.0351; p=0.8539; FIG. 9A) were observed.

It was also possible that a long tonic phase apnea could preclude the ability of respiratory neural circuitry to reinstate breathing. Contrary to this idea, we found that the behavioral tonic phase (i.e., duration of hindlimb extension) for fatal audiogenic seizures was actually shorter in duration than in mice that survived (p<0.0001, F_{1,49}=30.80; FIG. 9B). Taken together, these data suggested that the effect of adrenergic receptor-mediated seizure survival was due solely to increased respiratory drive immediately postictal (FIG. 9C).

### Discussion of the Examples

While it is not desired to be bound by any particular theory of operation, with regard to the age-dependent response to audiogenic seizure tests, it could be related to hearing impairment, developmental increases in Nav1.6 expression, and/or adrenergic receptor expression.

In FIG. 4 we show changes in heart rate during a seizure. Our findings demonstrated that bradycardia occurred at the same time as apnea during both audiogenic and spontaneous seizures; this is quantified in FIGS. 4C and 4D. All of these adult seizures were non-fatal, and thus did not lend well to interpretation of mechanisms of SUDEP. Technical difficulties due to young age and narrow developmental window prevented us from recording cardiac activity during the fatal seizures of the 3 week old D/+ mice. Implanting ECG headsets at this age is difficult due to the small size of the mice and the fact that they must be returned to their mothers, who often cannibalize mice with ECG headset attachments. This and the possibility of cardiac dysfunction as a contributor to seizure-induced death in the presently disclosed model is further discussed herein.

While it is not desired to be bound by any particular theory of operation, it is interpreted that i.p. phenylephrine does not directly stimulate the CNS because it does not cross the blood brain barrier. While it is not desired to be bound by any particular theory of operation, it is speculated that the blood pressure-maintaining effect of peripheral norepinephrine release could be the mechanism of rescue. It is noted that peripheral alpha-1 stimulation has minimal effect on heart (e.g., ECG).

Male and female D/+ mice in seizure behavior progressions (i.e., time spent in each phase of audiogenic seizure) and rates of survival in the various treatments both pharmacological and mechanical ventilation were compared and no evidence for sex differences was observed. Both sexes were chosen because the SCN8A encephalopathy affects both males and females in equal proportion with no obvious sex differences in clinical presentation.

At low doses, epinephrine primarily affects beta receptors whereas norepinephrine primarily affects alpha receptors. However, at higher doses there is no difference in the ability of epinephrine or norepinephrine to stimulate alpha receptors (Motiejunaite et al., 2020). Since the investigations described herein used high doses, it is noted that norepinephrine primarily stimulates alpha receptors, since norepinephrine has limited effect on beta receptors.

Selective beta-2 agonists like Albuterol or Terbutaline can impact bronchodilation. The effect of a beta-2 agonist in the P20-21 mice was not initially explored because the effect of phenylephrine was so robust emphasizing the sufficiency of alpha-1 adrenergic receptors in driving breathing recovery and survival. Additionally, in adults, sotalol treatment appeared to have only a small effect, and only as an additive of prazosin. Based on the data presented herein and while it is not desired to be bound by any particular theory of operation, the vast majority of the effect described appeared to be due to prazosin, indicating that alpha-1 receptors played the larger role in seizure survival.

Norepinephrine/phenylephrine and prazosin are competitive agonists/antagonists. If we were to observe that a dose of norepinephrine given 10-15 minutes after prazosin protected against death, this could be simply due to norepinephrine outcompeting prazosin at the doses/timing employed rather than working independently of alpha-1 adrenergic receptors. The use of phenylephrine as a selective alpha-1 agonist is a standard pharmacological approach, which, in addition to the use of prazosin in adults, allowed us to interpret the presently disclosed results to indicate a role for alpha-1 receptors in seizure survival.

The immediately preceding three paragraphs can be summarized as follows: (1) phenylephrine, an alpha-1 agonist, rescued death; (2) prazosin, an alpha-1 antagonist, caused death; and (3) sotalol, a beta-2 antagonist, had no affect on death rate. It is noted that a beta-2 agonist was not tested because of the robust responses described in points (1)-(3). Taken together, alpha-1 receptor function played a role in seizure survival in the presently disclosed model. The fact that epinephrine, at such a high dose, did not seem to result in rescue is a bit of an anomaly, but could have been due to some kind of pharmacokinetic/pharmacodynamic phenomena and/or a bioavailability difference.

In the experiments with adult D/+ mice, death after an audiogenic seizure was never observed. Based on other results, we confirm that while D/+ mice do occasionally die from spontaneous seizures, spontaneous seizure-induced death after any single seizure is relatively improbable, and we have recorded the relevant biosignals (breathing etc.) for these events.

Respiration in the P20-21 D/+ mice was recorded, which showed that apnea started proximal to the behavioral tonic phase, and did not recover postictally (FIG. 5A). This sequence of events was the same for adult D/+ mice when alpha-1 or alpha-1 & beta-2 receptors were blocked (FIG. 7D). In addition, when seizures were survived (norepinephrine/phenylephrine rescue in P20-21 and control experiments in adults), apnea still occurred during the tonic phase, but breathing was able to recover postictally. This is believed to be the first description of this type of behavior.

Implanting ECoG and ECG leads in P20-21 D/+ mice is particularly challenging due to the small size of the mice and the fact that surgery has to be done at pre-weaning ages. As such, mice are typically returned to their mothers and this increases the risk of cannibalization of the pups. That invasive surgery could potentially alter the developmental timeline of seizure-induced death was also considered. This aspect is discussed elsewhere herein, as is the possibility of cardiac dysfunction as a contributor to seizure-induced in the presently disclosed model. However, it is not believed that this detracts from the present disclosure regarding showing that breathing recovery played a role in seizure survival and this could be stimulated through alpha-1 adrenergic receptors.

The apparent anomaly of fatal audiogenic seizures in young but not old mice, and the converse for spontaneous seizures, is also discussed herein. Briefly, D/+ mice did not experience spontaneous seizures prior to 5-6 weeks of age and, in our hands, they experienced dozens of non-fatal spontaneous seizures as adults prior to death, which indicated that the probability of sudden death given any single seizure was relatively low. Thus, while it is not desired to be bound by any particular theory of operation, it is possible that if D/+ had spontaneous seizures at P20-21 and/or if we stimulated audiogenic seizures dozens of times in adult D/+ mice the rates of survival (and sudden death) would be similar.

Audiogenic seizures were induced in a different room from where the animals were typically housed in order to prevent unintended induction of audiogenic seizures in mice throughout the entire colony. Audiogenic seizures were observed in D/+ mice in different rooms within the lab as well as different rooms in other labs and vivaria, with no noticeable differences in seizure behavior. Thus, there was no evidence that the particular context influenced the susceptibility to audiogenic seizures and corresponding behavior.

A 20-second acclimation period might be viewed be short for many behavioral experiments. However, it is believed that a strong behavioral phenotype (audiogenic seizure is not contingent upon animal habituation) does not require additional acclimation time. Due to the inclusion of appropriate controls which all underwent the same 20-second acclimation period and the fact that we conducted as many experiments as possible in a blinded fashion, we do not believe that our short acclimation period significantly impacted our experimental results.

As such, as disclosed herein we present the finding that mice expressing the patient derived N1768D SCN8A mutation (D/+) had audiogenic seizures that were nearly identical to spontaneous seizures. Furthermore, at the specific time point of P20-21, these audiogenic seizures resulted in sudden death, which could be rescued by mechanical ventilation immediately after the onset of an audiogenic seizure, indicating that breathing cessation was the primary cause of death. Also disclosed is that blockade of adrenergic receptors resulted in seizure-induced death in adult mice, while activation of alpha-1 adrenergic receptors rescued seizure-induced death in P20-21 mice. These results indicated that alpha-1 adrenergic receptor activity was critical for breathing recovery after tonic seizures and represents a potential therapeutic intervention for SUDEP.

Scn8a mutant mice have audiogenic seizures. SCN8A encephalopathy is a severe genetic epilepsy syndrome and neurodevelopmental disorder characterized by refractory seizures, cognitive and motor dysfunction, and a substantial risk for SUDEP (Veeramah et al., 2012; de Kovel et al., 2014; Estacion et al., 2014; Ohba et al., 2014; Blanchard et al., 2015; Larsen et al., 2015; Gardella et al., 2018; Zaman et al., 2019). Scn8a alleles containing patient-derived mutations form Nav1.6 voltage-gated sodium channels that create aberrant neuronal excitability in various cortical neurons including hippocampal CA1 (Lopez-Santiago et al., 2017; Baker et al., 2018; Bunton-Stasyshyn et al., 2019), entorhinal cortex (Ottolini et al., 2017), subiculum (Wengert et al., 2019), and layer V somatosensory cortex (Bunton-Stasyshyn et al., 2019). The present findings that SCN8A mice experienced audiogenic seizures and seizure-induced sudden death suggested that additional regions, such as the inferior colliculus and amygdala, which have been previously implicated in audiogenic seizures and sudden death (Wada et al., 1970; Millan et al., 1986; Faingold et al., 1992, 2017; Coffey et al., 1996; Dlouhy et al., 2015; Kommajosyula et al., 2017; Ribak, 2017; Marincovich et al., 2019), could also be functionally impacted by SCN8A mutations. In particular, prior studies have revealed intrinsic hyperexcitability in inferior colliculus neurons from rats susceptible to audiogenic seizures (Li et al., 1994). Thus, the results presented herein encourage future examination of various additional brain regions in models of SCN8A encephalopathy.

Risk of death from audiogenic seizures in the D/+ mice was strongly age-dependent: sudden death due to audiogenic seizure occurred with high probability in P20-21 mice, but was never observed in adult mice. This is in contrast to the mortality rate from spontaneous seizures in D/+ mice, where premature death can occur starting around eight weeks of age and reaches 50% mortality by one year (Wagnon et al., 2015). The age-dependent differences in seizure phenotype and risk for sudden death were likely attributable to developmental changes in hearing, SCN8A expression, adrenergic receptor expression (particularly alpha-1 subtype), as well as additional unknown factors. Gain of function mutations in SCN8A could lead to altered wiring of auditory neural circuits that favor initiation of audiogenic seizures. Furthermore, alpha-1 adrenergic receptor function at P20-21 might be insufficient to rescue breathing and prevent seizure-induced sudden death. The fact that spontaneous death at P20-21 in D/+ mice was not observed suggested that although they were capable of having audiogenic seizures at this age, they likely did not experience spontaneous seizures, which is consistent with previous reports (Wagnon et al., 2015).

As to the low mortality of audiogenic seizures in adult D/+ mice, we have observed that D/+ mice have many non-fatal spontaneous seizures prior to death. However, as disclosed herein, we never induced audiogenic seizures more the three times in a single adult D/+ mouse. Thus, the likelihood of death from any single audiogenic versus spontaneous seizure was likely not different between spontaneous and audiogenic seizures. Future studies as to how developmentally-determined alpha-1 adrenergic signaling relates to seizure-induced sudden death are needed to further clarify the mechanism(s) of death in P20-21 D/+ mice. The present results demonstrating that norepinephrine and phenylephrine administration improved survival suggest that endogenous release of these alpha-1 adrenergic receptor-targeting monoamines at P20-21 was impaired in D/+ mice.

Similar to previous reports of other mouse models susceptible to audiogenic seizures, the recordings of auditory brainstem responses presented herein revealed that D/+ mice had impaired hearing (Willott et al., 1995; Willott & Bross, 1996; Koay et al., 2002; Mackenzie et al., 2009; Heffner et al., 2019). To date, there have been no reports of SCN8A encephalopathy patients that exhibit hearing abnormalities or audiogenic seizures. Further audiological evaluation of patients with SCN8A mutations is warranted to shed light on the importance of hearing impairment and seizures.

To our knowledge this is the first model which exhibited spontaneous seizures and sudden death events. Since seizure induced death can be reliably induced, the present model allows for a more in-depth examination of the cascade of events that lead to either fatal or non-fatal seizures, increasing the versatility of this clinically relevant model of epilepsy. Importantly, audiogenic seizures highly resembled spontaneous seizures in D/+ mice with respect to ECoG activity, heart rate, and breathing. The differences observed in relative timing of ECoG ictal activity and onset of tonic phase might indicate different focal regions between audiogenic and spontaneous seizures. Nonetheless, the presently disclosed data supported the notion that audiogenic seizures can be utilized as a model to understand mechanisms of seizure semiology and seizure-induced death.

Respiratory arrest contributes to seizure-induced death. There is increasing evidence that respiratory arrest is the primary cause of death in SUDEP. Most witnessed cases of SUDEP are preceded by convulsive seizures (Hesdorffer et al., 2011; Nashef et al., 2012; Ryvlin et al., 2013), and oxygen desaturation due to breathing complications is common during and after convulsive seizures (Bateman et al., 2008; Lacuey et al., 2018; Vilella et al., 2019a; Vilella et al., 2019b). SUDEP events where cardiorespiratory parameters are adequately recorded are understandably limited; however, in these few cases patients experienced respiratory arrest prior to terminal asystole (Ryvlin et al., 2013), suggesting the primacy of respiratory failure. Data obtained from mouse models of SUDEP support this notion. Death is due to respiratory arrest for the stimulated seizures of Lmx1b^{f/f/p} and DBA/1&2 mice, (Faingold et al., 2010; Buchanan et al., 2014; Irizarry et al., 2020), and the spontaneous seizure-induced deaths in Cacna1a^{S218L} mice and Scn1a^{R1407X}, a mouse model of Dravet Syndrome (Kim et al., 2018; Jansen et al., 2019; Loonen et al., 2019). Breathing dysfunction is also reported for seizures induced under urethane anesthesia using Kcna1 KO, RyR2^{R176Q}, Cacna1a^{S218L} mice, and Sprague-Dawley rats (Aiba & Noebels, 2015; Aiba et al., 2016; Loonen et al., 2019).

It has been previously reported that D/+ mice have cardiac arrhythmias and experience bradycardia prior to death (Frasier et al., 2016); however, breathing was not recorded in these studies and the sequence of events during seizure-induced death was not presented. Due to the young age of the mice in the presently described audiogenic model of seizure-induced death, cardiac function was not recorded. Thus, bradycardia, or other cardiac abnormalities, could still play a role in the mortality of D/+ mice. However, the observation that mechanical ventilation prevents seizure-induced death suggested that breathing cessation was clearly an important factor in death.

Tonic phase apnea and failure of breathing recovery. Many studies concerned with breathing cessation as a mechanism of SUDEP speak of seizure-induced respiratory arrest (S-IRA). Often, S-IRA is synonymous with seizure-induced death; drugs that prevent death also prevent S-IRA (Faingold et al., 2010; Zeng et al., 2015; Zhang et al., 2017; Irizarry et al., 2020). In D/+ mice, we found that S-IRA occurred during all audiogenic seizures and coincided with the behavioral and electrographic tonic phase, which we refer to as tonic phase apnea. D/+ mice only experienced seizure-induced death when breathing did not recover immediately after the tonic phase. This discrepancy could be attributed to the fact that for most audiogenic seizure-induce death experiments, respiratory activity was assessed by visualization, which could make it difficult to ascertain breathing, or the lack thereof, during convulsions. Another factor could be that tonic seizures in DBA1/2J mice, which are used in the majority of preclinical SUDEP research, almost always produce death, making S-IRA and death coincident with one another (Martin et al., 2020). Considering tonic seizures are associated with apnea in humans (Gastaut et al., 1963; Wyllie, 2015), it is likely that most, if not all, tonic seizures in mouse models produce apnea.

It is unclear whether tonic phase apnea is necessary for postictal apnea and death, as there is no method to selectively prevent the tonic phase from occurring. However, it has been shown that DBA1/2J and 129/SvTer mice die from tonic, but not clonic, seizures (Martin et al., 2020), implying the tonic phase is important and perhaps necessary for seizure-induced death. Thus, determining cellular and molecular underpinnings of both tonic phase apnea and failure of breathing recovery could be important foci for future SUDEP research.

Adrenergic signaling and seizure survival. Mechanisms of seizure-induced death are poorly understood. Many proposed mechanisms involve impaired brainstem neural activity occurring due to synaptic seizure spread or spreading depolarization that impairs function of respiratory centers in the medulla producing central (Aiba & Noebels, 2015; Aiba et al., 2016; Salam et al., 2017; Ellis et al., 2018; Jansen et al., 2019; Loonen et al., 2019) or obstructive (Weissbrod et al., 2011; Nakase et al., 2016; Villiere et al., 2017) apnea. Seizure spread to non-medullary sites, such as the amygdala, also suppresses breathing and is proposed to cause breathing cessation (Dlouhy et al., 2015; Marincovich et al., 2019; Nobis et al., 2019).

A body of work demonstrates the impairment of neuromodulator systems and their utility to rescue death in mouse models of SUDEP (Faingold et al., 2011, 2016; Buchanan et al., 2014; Zeng et al., 2015; Devinsky et al., 2016; Zhan et al., 2016; Zhao et al., 2017; Feng & Faingold, 2017; Zhang et al., 2017; Ellis et al., 2018; Kruse et al., 2019). Much of this work centers on the serotonergic system. Chronic administration of agents that increase serotonin levels can reduce the incidence of seizure-induced death (Faingold et al., 2011; Zeng et al., 2015; Feng & Faingold, 2017). In addition, genetic lesion of serotonergic raphe neurons elevates susceptibility of death after maximal electroshock seizures (Zhan et al., 2016), and optogenetic stimulation of the dorsal raphe reduces occurrence of seizure-induced death in DBA1 mice (Zhang et al., 2018). Noradrenaline has recently been shown to have a similar affect (Zhang et al., 2017; Zhao et al., 2017), and it appears that the ability of SSRIs to prevent seizure-induced death is dependent on functional adrenergic signaling (Kruse et al., 2019).

Acute alpha-1 adrenergic receptor stimulation was sufficient to prevent seizure-induced death in P20-21 D/+ mice. Functional adrenergic signaling was necessary for survival of seizures in adult D/+ mice, and this was largely dominated by alpha-1 receptor activity. Taken together with phenylephrine's ability to prevent death, alpha-1 receptors appear to be critical for seizure survival, similar to findings using maximal electroshock seizure-induced death (Kruse et al., 2019). For this reason, it appeared that activation of alpha-1 adrenergic receptors was a general requirement for survival after a seizure and that acute augmentation of alpha-1 receptors could also reduce risk of seizure-induced sudden death in other seizures models.

Precisely how adrenergic receptors promote breathing recovery and survival is still unclear. Previous studies have shown that intracerebroventricular injection of the norepinephrine reuptake inhibitor atomoxetine or alpha-1 receptor agonist phenylephrine prevents death from the audiogenic seizures of DBA1 mice (Zhao et al., 2017) or maximal electroshock seizures (Kruse et al., 2019), respectively. This could implicate a role in arousal neural circuitry, as a major function of noradrenergic signaling in the central nervous system is to increase arousal state (Broese et al., 2012). As described herein, norepinephrine and phenylephrine were given intraperitoneally. Considering monoamine analogues do not cross the blood brain barrier (Olesen, 1972; Hardebo & Owman, 1980), the presently disclosed data suggested that peripheral alpha-1 adrenergic receptor activity was responsible for rescuing breathing in D/+ mice. Peripheral stimulation of alpha-1 receptors has little effect on heart rate but does increase peripheral resistance, which is crucial for maintaining blood flow to the heart and brain during life-threatening conditions of hypoxia and hemorrhage (Bond, 1985; Schultz et al., 2007). It is feasible that in the already compromised state of a severe convulsive seizure, multiple systems would be needed to coordinate full recovery.

Taken together, the results presented herein highlight the D/+ mouse model of SCN8A encephalopathy as a new useful model for mechanistic investigation of SUDEP. The utility of this model was demonstrated by providing new evidence that respiratory arrest is the primary cause of death, and that interventional approaches to stimulate breathing including the augmentation of alpha-1 adrenergic receptor activity might be valuable for preventing SUDEP.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Aiba &Noebels (2015) Spreading depolarization in the brainstem mediates sudden cardiorespiratory arrest in mouse SUDEP models. Sci Transl Med 7:282ra46.
Aiba et al. (2016) Leaky RyR2 channels unleash a brainstem spreading depolarization mechanism of sudden cardiac death. Proc Natl Acad Sci USA 113(33):E4895-903.
Auerbach et al. (2013) Altered Cardiac Electrophysiology and SUDEP in a Model of Dravet Syndrome. PLoS One 8:e77843.
Autry et al. (2009) Increased Risk of Death Among Children With Lennox-Gastaut Syndrome and Infantile Spasms. J Child Neurol 25:441-447.
Baker et al. (2018) The novel sodium channel modulator GS-458967 (GS967) is an effective treatment in a mouse model of SCN8A encephalopathy. Epilepsia 59:1166-1176.
Bateman et al. (2008) Ictal hypoxemia in localization-related epilepsy: analysis of incidence, severity and risk factors. Brain 131:3239-3245.
Bird et al. (1997) Sudden Unexplained Death in Epilepsy: An Intracranially Monitored Case. Epilepsia 38:S52-S56.
Blanchard et al. (2015) De novo gain-of-function and loss-of-function mutations of SCN8A in patients with intellectual disabilities and epilepsy. J Med Genet 52(5):330-337.
Bond (1985) Vascular adrenergic interactions during hemorrhagic shock. Fed Proc 44:281-289.
Broese et al. (2012) α-Adrenergic Receptor Function, Arousal and Sleep: Mechanisms and Therapeutic Implications. Pharmacopsychiatry 45:209-216.
Buchanan et al. (2014) Serotonin neurones have anti-convulsant effects and reduce seizure-induced mortality. J Physiol 592:4395-4410.
Bunton-Stasyshyn et al. (2019) Prominent role of forebrain excitatory neurons in SCN8A encephalopathy. Brain 142(2):362-375.
Coffey et al. (1996) Amygdala Kindling of Forebrain Seizures and the Occurrence of Brainstem Seizures in Genetically Epilepsy-Prone Rats. Epilepsia 37:188-197.
Council (2011) Guide for the Care and Use of Laboratory Animals: Eighth Edition. National Academies Press, Washington, DC, United States of America.
Dasheiff & Dickinson (1986) Sudden Unexpected Death of Epileptic Patient due to Cardiac Arrhythmia After Seizure. Arch Neurol 43:194-196.
de Kovel et al. (2014) Characterization of a de novo SCN8A mutation in a patient with epileptic encephalopathy. Epilepsy Res 108(9): 1511-1518.
Devinsky (2011) Sudden, Unexpected Death in Epilepsy. N Engl J Med 365:1801-1811.
Devinsky et al. (2016) Sudden unexpected death in epilepsy: epidemiology, mechanisms, and prevention. Lancet Neurol 15:1075-1088.
Dhaibar et al. (2019) Cardiorespiratory profiling reveals primary breathing dysfunction in Kcna1-null mice: Implications for sudden unexpected death in epilepsy. Neurobiol Dis 127:502-511.
Dlouhy et al. (2015) Breathing Inhibited When Seizures Spread to the Amygdala and upon Amygdala Stimulation. J Neurosci 35:10281-10289.
Ellis et al. (2018) The ventrolateral medulla and medullary raphe in sudden unexpected death in epilepsy. Brain 141:1719-1733.
Escayg & Goldin (2010) Sodium channel SCN1A and epilepsy: Mutations and mechanisms. Epilepsia 51(9):1650-1658.
Estacion et al. (2014) A novel de novo mutation of SCN8A (Nav1.6) with enhanced channel activation in a child with epileptic encephalopathy. Neurobiol Dis 69:117-123.
Faingold et al. (1992) Glutamate in the inferior colliculus plays a critical role in audiogenic seizure initiation. Epilepsy Res 13:95-105.
Faingold et al. (2010) DBA/1 mice exhibit chronic susceptibility to audiogenic seizures followed by sudden death associated with respiratory arrest. Epilepsy Behav 17:436-440.
Faingold et al. (2011) Prevention of seizure-induced sudden death in a chronic SUDEP model by semichronic administration of a selective serotonin reuptake inhibitor. Epilepsy Behav 22:186-190.
Faingold et al. (2016) Susceptibility to seizure-induced sudden death in DBA/2 mice is altered by adenosine. Epilepsy Res 124:49-54.
Faingold et al. (2017) Chapter 31—Genetic Models of Reflex Epilepsy and SUDEP in Rats and Mice. Models of Seizures and Epilepsy (Second Edition), pages 441-453.
Feng & Faingold (2017) Abnormalities of serotonergic neurotransmission in animal models of SUDEP. Epilepsy Behav 71:174-180.
Frasier et al. (2016) Cardiac arrhythmia in a mouse model of sodium channel SCN8A epileptic encephalopathy. Proc Natl Acad Sci USA 113(45):12838-12843.
Gardella et al. (2018) The phenotype of &lt;em&gt;SCN8A&lt;/em&gt; developmental and epileptic encephalopathy. Neurology 91: e 1112.
Gastaut et al. (1963) An Electro-clinical Study of Generalized Epileptic Seizures of Tonic Expression. Epilepsia 4:15-44.
GENBANK® Accession Nos. NC_000012.12, NM_014191.4, NM_001177984.3, NM_001330260.2, NM_001369788.1, NP_055006.1, NP_001171455.1, NP_001317189.1, and NP_001356717.1.
Glasscock et al. (2010) Kv1.1 Potassium Channel Deficiency Reveals Brain-Driven Cardiac Dysfunction as a Candidate Mechanism for Sudden Unexplained Death in Epilepsy. J Neurosci 30:5167-5175.
Hammer et al. (1993) SCN8A-Related Epilepsy with Encephalopathy. In: GENEREVIEWS® [Internet]. Seattle (WA): University of Washington, Seattle; 1993-2021. 2016 Aug. 25. PMID: 27559564.
Hamosh et al. (2005) Online Mendelian Inheritance in Man (OMIM), a knowledgebase of human genes and genetic disorders. Nucleic Acids Res 33(Database Issue):D514-D517.
Hardebo & Owman (1980) Barrier mechanisms for neurotransmitter monoamines and their precursors at the blood-brain interface. Ann Neurol 8:1-11.
Harlow & Lane (1988) Antibodies: A Laboratory Manual, Cold Spring Harbor Laboratory Press, Cold Spring Harbor, New York, United States of America.
Heffner et al. (2019) Normal audiogram but poor sensitivity to brief sounds in mice with compromised voltage-gated sodium channels (Scn8a medJ) Hear Res 374:1-4.
Hesdorffer et al. (2011) Combined analysis of risk factors for SUDEP. Epilepsia 52:1150-1159.
Irizarry et al. (2020) Seizures induce obstructive apnea in DBA/2J audiogenic seizure-prone mice: Lifesaving impact of tracheal implants. Epilepsia 61:e13-e16.
Jansen et al. (2019) Apnea Associated with Brainstem Seizures in Cacna1a^{S218L} Mice Is Caused by Medullary Spreading Depolarization. J Neurosci 39:9633-9644.
Johannesen et al. (2018) Early mortality in SCN8A-related epilepsies. Epilepsy Res 143:79-81.
Kalume et al. (2013) Sudden unexpected death in a mouse model of Dravet syndrome. J Clin Invest 123:1798-1808.
Kim et al. (2018) Severe peri-ictal respiratory dysfunction is common in Dravet syndrome. J Clin Invest 128:1141-1153.
Koay et al. (2002) Behavioral audiograms of homozygous medJ mutant mice with sodium channel deficiency and unaffected controls. Hear Res 171:111-118.
Kommajosyula et al. (2017) Specific subcortical structures are activated during seizure-induced death in a model of sudden unexpected death in epilepsy (SUDEP): A manganese-enhanced magnetic resonance imaging study. Epilepsy Res 135:87-94.
Kruse et al. (2019) Effect of monoamine reuptake inhibition and al blockade on respiratory arrest and death following electroshock-induced seizures in mice. Epilepsia 60:495-507.
Lacuey et al. (2018) The incidence and significance of periictal apnea in epileptic seizures. Epilepsia 59:573-582.
Larsen et al. (2015) The phenotypic spectrum of SCN8A encephalopathy. Neurology 84:480-489. Li et al. (1994) Inferior colliculus neuronal membrane and synaptic properties in genetically epilepsy-prone rats. Brain Res 660:232-240.
Loonen et al. (2019) Brainstem spreading depolarization and cortical dynamics during fatal seizures in Cacna1a S218L mice. Brain 142:412-425.
Lopez-Santiago et al. (2017) Neuronal hyperexcitability in a mouse model of SCN8A epileptic encephalopathy. Proc Natl Acad Sci USA 114(9):2383-2388.
Mackenzie et al. (2009) Analysis of the mouse mutant Cloth-ears shows a role for the voltage-gated sodium channel Scn8a in peripheral neural hearing loss. Genes, Brain Behav 8:699-713.
Marincovich et al. (2019) Amygdala lesions reduce seizure-induced respiratory arrest in DBA/1 mice. Epilepsy Behav 121 (Part B):106440.
Martin et al. (2020) Audiogenic seizure as a model of sudden death in epilepsy: A comparative study between four inbred mouse strains from early life to adulthood. Epilepsia 61:342-349.
Massey et al. (2014) Mechanisms of sudden unexpected death in epilepsy: the pathway to prevention. Nat Rev Neurol 10(5):271-282.
Millan et al. (1986) Induction of audiogenic seizure susceptibility by focal infusion of excitant amino acid or bicuculline into the inferior colliculus of normal rats. Exp Neurol 91:634-639.
Motiejunaite et al. (2020) Adrenergic receptors and cardiovascular effects of catecholamines. Ann Endocrinol (Paris) 82(3-4):193-197.
Nakase et al. (2016) Laryngospasm, central and obstructive apnea during seizures: Defining pathophysiology for sudden death in a rat model. Epilepsy Res 128:126-139.
Nashef et al. (2012) Unifying the definitions of sudden unexpected death in epilepsy. Epilepsia 53:227-233.
Negishi et al. (2021) SCN8A-related developmental and epileptic encephalopathy with ictal asystole requiring cardiac pacemaker implantation. Brain Dev 43(7):804-808.
Nilsson et al. (1999) Risk factors for sudden unexpected death in epilepsy: a case control study. Lancet 353:888-893.
Nobis et al. (2019) The effect of seizure spread to the amygdala on respiration and onset of ictal central apnea. J Neurosurg 132(5): 1313-1323.
Ohba et al. (2014) Early onset epileptic encephalopathy caused by de novo SCN8A mutations. Epilepsia. 55(7):994-1000.
Olesen (1972) The effect of intracarotid epinephrine, norepinephrine, and angiotensin on the regional cerebral blood flow in man. Neurology 22:978-987.
Ottolini et al. (2017) Aberrant Sodium Channel Currents and Hyperexcitability of Medial Entorhinal Cortex Neurons in a Mouse Model of SCN8A Encephalopathy. J Neurosci 37(32):7643-7655.
Ribak (2017) An abnormal GABAergic system in the inferior colliculus provides a basis for audiogenic seizures in genetically epilepsy-prone rats. Epilepsy Behav 71:160-164.
Ruhl et al. (2019) Necroptosis and Apoptosis Contribute to Cisplatin and Aminoglycoside Ototoxicity. J Neurosci 39:2951-2964.
Ryvlin et al. (2013) Incidence and mechanisms of cardiorespiratory arrests in epilepsy monitoring units (MORTEMUS): a retrospective study. Lancet Neurol 12:966-977.
Salam et al. (2017) Mortality with brainstem seizures from focal 4-aminopyridine-induced recurrent hippocampal seizures. Epilepsia 58:1637-1644.
Schultz et al. (2007) Arterial Chemoreceptors and Sympathetic Nerve Activity. Hypertension 50:6-13.
So et al. (2000) Postictal Central Apnea as a Cause of SUDEP: Evidence From Near-SUDEP Incident. Epilepsia 41:1494-1497.
Sprissler et al. (2017) Altered gene expression profile in a mouse model of SCN8A encephalopathy. Exp Neurol 288:134-141.
Terra et al. (2013) Sudden unexpected death in epilepsy: From the lab to the clinic setting. Epilepsy Behav 26:415-420.
Trivisano et al. (2019) Generalized tonic seizures with autonomic signs are the hallmark of SCN8A developmental and epileptic encephalopathy. Epilepsy Behav 96:219-223.
Veeramah et al. (2012) De novo pathogenic SCN8A mutation identified by whole-genome sequencing of a family quartet affected by infantile epileptic encephalopathy and SUDEP. Am J Hum Genet 90(3):502-10.
U.S. Patent Application Publication No. 2019/0153430.
U.S. Pat. Nos. 7,762,994; 8,409,149; 8,556,864; 8,579,869; 9,011,391; 9,265,893; 9,738,908.
Vilella et al. (2019a) Incidence, Recurrence, and Risk Factors for Peri-ictal Central Apnea and Sudden Unexpected Death in Epilepsy. Front Neurol 10:166.
Vilella et al. (2019b) Post-convulsive central apnea as a biomarker for sudden unexpected death in epilepsy (SUDEP) Neurology 92: e171-e182.
Villiere et al. (2017) Seizure-associated central apnea in a rat model: Evidence for resetting the respiratory rhythm and activation of the diving reflex. Neurobiol Dis 101:8-15.
Wada et al. (1970) Inferior colliculus lesion and audiogenic seizure susceptibility. Exp Neurol 28:326-332.
Wagnon et al. (2015) Convulsive seizures and SUDEP in a mouse model of SCN8A epileptic encephalopathy. Hum Mol Genet 24(2):506-515.
Weissbrod et al. (2011) Quantity and Three-Dimensional Position of the Recurrent and Superior Laryngeal Nerve Lower Motor Neurons in a Rat Model. Ann Otol Rhinol Laryngol 120:761-768.
Wengert et al. (2019) Prax330 reduces persistent and resurgent sodium channel currents and neuronal hyperexcitability of subiculum neurons in a mouse model of SCN8A epileptic encephalopathy. Neuropharmacology 158:107699.
Wengert et al. (2021) Adrenergic Mechanisms of Audiogenic Seizure-Induced Death in a Mouse
Model of SCN8A Encephalopathy. Front Neurosci 15:581048.
Wenker et al. (2021) Postictal Death Is Associated with Tonic Phase Apnea in a Mouse Model of
Sudden Unexpected Death in Epilepsy. Ann Neurol 89:1023-1035.
Whelan & Young (1953) The effect of adrenaline and noradrenaline infusions on respiration in man.
Br J Pharmacol Chemother 8:98-102.
Willott & Bross (1996) Morphological changes in the anteroventral cochlear nucleus that accompany sensorineural hearing loss in DBA/2J and C57BL/6J mice. Dev Brain Res 91:218-226.
Willott et al. (1995) Genetics of age-related hearing loss in mice. II. Strain differences and effects of caloric restriction on cochlear pathology and evoked response thresholds. Hear Res 88:143-155.
Wyllie (2015) Wyllie's Treatment of Epilepsy: Principles and Practice. 6th ed. (Gidal et al. (eds)) Wolters Kluwer, Philadelphia, Pennsylvania, United States of America.
Zaman et al. (2019) A single-center SCN8A-related epilepsy cohort: clinical, genetic, and physiologic characterization. Ann Clin Transl Neurol 6:1445-1455.
Zeng et al. (2015) Fluoxetine prevents respiratory arrest without enhancing ventilation in DBA/1 mice. Epilepsy Behav 45:1-7.
Zhan et al. (2016) Impaired Serotonergic Brainstem Function during and after Seizures. J Neurosci 36:2711-2722.
Zhang et al. (2017) Atomoxetine, a norepinephrine reuptake inhibitor, reduces seizure-induced respiratory arrest. Epilepsy Behav 73:6-9.
Zhang et al. (2018) Optogenetic activation of 5-HT neurons in the dorsal raphe suppresses seizure-induced respiratory arrest and produces anticonvulsant effect in the DBA/1 mouse SUDEP model. Neurobiol Dis 110:47-58.
Zhao et al. (2017) The effect of atomoxetine, a selective norepinephrine reuptake inhibitor, on respiratory arrest and cardiorespiratory function in the DBA/1 mouse model of SUDEP. Epilepsy Res 137:139-144.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

What is claimed is:

1. A germline modified mouse, wherein the germline of the transgenic mouse comprises a nucleotide sequence encoding a gain-of-function SCN8A mutation.

2. The germline modified mouse of claim 1, wherein the gain-of-function SCN8A mutation encodes a substitution selected from the group consisting of a leucine to valine substitution at amino acid 257 of SEQ ID NO: 12, a leucine to valine substitution at amino acid 862 of SEQ ID NO: 12, a glutamine to histidine substitution at amino acid 1468 of SEQ ID NO: 12, a glycine to arginine substitution at amino acid 1473 of SEQ ID NO: 12, an alanine to valine substitution at amino acid 1489 of SEQ ID NO: 12, a methionine to threonine substitution at amino acid 1643 of SEQ ID NO: 12, an alanine to threonine substitution at amino acid 1648 of SEQ ID NO: 12, and an asparagine to aspartic acid substitution at amino acid 1766 of SEQ ID NO: 12, or any combination thereof.

3. The germline modified mouse of claim 1 or claim 2, wherein the germline modified mouse comprises a genome comprising an endogenous SCN8A coding sequence with an asparagine to aspartic acid substitution at amino acid 1766 of SEQ ID NO: 12.

4. A method for inducing an audiogenic seizure and/or seizure-induced death in a mouse, the method comprising subjecting the germline modified mouse of any one of claims 1-3 with an audiogenic stimulus of sufficient intensity and duration to induce an audiogenic seizure and/or seizure-induced death in the mouse.

5. The method of claim 4, wherein the audiogenic stimulus comprises sound of at least about 12 kHz at an intensity of at least about 100 dB for a duration of at least about 10 seconds.

6. A method for treating and/or preventing a death associated with a seizure in a subject, the method comprising stimulating breathing of the subject via mechanical ventilation and/or by administering a composition comprising an effective amount of an alpha-1 adrenergic receptor activator to the subject.

7. The method of claim 6, wherein the seizure is an epileptic seizure.

8. The method of claim 6 or claim 7, wherein the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline.

9. The method of any one of claims 6-8, wherein the mechanical ventilation of the subject is continued until the subject is able to breath unassisted.

10. The method of any one of claims 6-9, wherein the alpha-1 adrenergic receptor activator is administered to the subject during a tonic phase of the seizure, optionally within 1, 2, 3, 4, or 5 minutes from the onset of the seizure.

11. The method of any one of claims 6-10, wherein the composition is provided to the subject as an injectable, optionally in the form of a pen delivery device.

12. A method for identifying a compound that has activity in treating and/or preventing a seizure and/or seizure-induced death in a subject, the method comprising:
(a) inducing an audiogenic seizure in the germline modified mouse of any one of claims 1-3;
(b) administering a compound to be tested to the germline modified mouse; and
(c) determining whether the compound treats and/or prevents a seizure and/or seizure-induced death in the subject,
whereby a compound that has activity in treating and/or preventing a seizure and/or seizure-induced death in a subject is identified.

13. A method for preventing sudden unexpected death in epilepsy (SUDEP) in a subject in need thereof, the method comprising stimulating breathing of the subject via mechanical ventilation and/or by administering a composition comprising an effective amount of an alpha-1 adrenergic receptor activator to the subject.

14. The method of claim 13, wherein the SUDEP is associated with a gain-of-function mutation in an SCN8A gene product in the subject.

15. The method of claim 14, wherein the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof.

16. The method of any one of claims 13-15, wherein the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline.

17. The method of any one of claims 13-16, wherein the alpha-1 adrenergic receptor activator is administered to the subject during a tonic phase of the seizure, optionally within 1, 2, 3, 4, or 5 minutes from the onset of the seizure.

18. The method of any one of claims 13-17, further comprising providing mechanical ventilation to the subject.

19. The method of claim 18, wherein the mechanical ventilation is provided to the subject until the subject is able to breath unassisted.

20. A method for preventing and/or reducing the risk of death in a subject having a gain-of-function mutation in an SCN8A gene product, the method comprising stimulating breathing of the subject via mechanical ventilation and/or by administering a composition comprising an effective amount of an alpha-1 adrenergic receptor activator to the subject.

21. The method of claim 20, wherein the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof.

22. The method of claim 20 or claim 21, wherein the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline.

23. The method of any one of claims 20-22, wherein the alpha-1 adrenergic receptor activator is administered to the subject during a tonic phase of the seizure, optionally within 1, 2, 3, 4, or 5 minutes from the onset of the seizure.

24. The method of any one of claims 20-23, further comprising providing mechanical ventilation to the subject.

25. The method of claim 24, wherein the mechanical ventilation is provided to the subject until the subject is able to breath unassisted.

26. A method for preventing or reducing the risk of death associated with a tonic seizure in a subject in need thereof, the method comprising stimulating breathing of the subject via mechanical ventilation and/or by administering a composition comprising an effective amount of an alpha-1 adrenergic receptor activator to the subject.

27. The method of claim 26, wherein the subject has a genome comprising a gain-of-function mutation in an SCN8A gene product.

28. The method of claim 27, wherein the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof.

29. The method of any one of claims 26-28, wherein the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline.

30. The method of any one of claims 26-29, wherein the alpha-1 adrenergic receptor activator is administered to the subject during a tonic phase of the seizure, optionally within 1, 2, 3, 4, or 5 minutes from the onset of the seizure.

31. The method of any one of claims 26-30, further comprising providing mechanical ventilation to the subject.

32. The method of claim 31, wherein the mechanical ventilation is provided to the subject until the subject is able to breath unassisted.

33. A method for preventing or reducing the risk of death associated with an epileptic seizure in a subject in need thereof, the method comprising stimulating breathing of the subject via mechanical ventilation and/or by administering a composition comprising an effective amount of an alpha-1 adrenergic receptor activator to the subject.

34. The method of claim 33, wherein the subject has a genome comprising a gain-of-function mutation in an SCN8A gene product.

35. The method of claim 34, wherein the gain-of-function mutation in the SCN8A gene product in the subject comprises an amino acid substitution in an SCN8A polypeptide that is selected from the group consisting of a leucine to valine substitution at amino acid 257 of any one of SEQ ID NOs: 4, 6, 8, or 10; a leucine to valine substitution at amino acid 864 of any one of SEQ ID NOs: 4, 6, 8, or 10; a glutamine to histidine substitution at amino acid 1470 of any one of SEQ ID NOs: 4, 6, 8, or 10; a glycine to arginine substitution at amino acid 1475 of any one of SEQ ID NOs: 4, 6, 8, or 10; an alanine to valine substitution at amino acid 1491 of any one of SEQ ID NOs: 4, 6, 8, or 10; a methionine to threonine substitution at amino acid 1645 of any one of SEQ ID NOs: 4, 6, 8, or 10; an alanine to threonine substitution at amino acid 1650 of any one of SEQ ID NOs: 4, 6, 8, or 10; and an asparagine to aspartic acid substitution at amino acid 1768 of any one of SEQ ID NOs: 4, 6, 8, or 10; or any combination thereof.

36. The method of any one of claims 33-35, wherein the alpha-1 adrenergic receptor activator is selected from the group consisting of Cirazoline, Methoxamine, Synephrine, Etilefrine, Metaraminol, Midodrine, Naphazoline, Norepinephrine, Oxymetazoline, Phenylephrine, Pseudoephedrine, Tetrahydrozoline and Xylometazoline.

37. The method of any one of claims 33-36, wherein the alpha-1 adrenergic receptor activator is administered to the subject during a tonic phase of the seizure, optionally within 1, 2, 3, 4, or 5 minutes from the onset of the seizure.

38. The method of any one of claims 33-37, further comprising providing mechanical ventilation to the subject.

39. The method of claim 38, wherein the mechanical ventilation is provided to the subject until the subject is able to breath unassisted.

40. The method of any one of claims 6-39, wherein the composition comprising the alpha-1 adrenergic receptor activator, the mechanical ventilation, if administered, or both are administered to the subject subsequent to development of apnea but prior to the end of a tonic phase experienced by the subject.

41. The method of any one of claims 6-40, wherein the alpha-1 adrenergic receptor activator is provided to the subject as an injectable, optionally in the form of a pen delivery device.

42. The method of any one of claims 6-41, wherein the alpha-1 adrenergic receptor activator is administered to the subject via a route selected from the group consisting of intraperitoneal, intramuscular, intravenous, and intranasal, or any combination thereof.

43. The method of any one of claims 6-42, wherein the subject is a human.


SEQUENCE LISTING (13 sequences)

SEQ ID NO: 1 - DNA, 23 bp, Artificial Sequence
  Artificially synthesized oligonucleotide primer
tgactgcagcttggacaaggagc

SEQ ID NO: 2 - DNA, 23 bp, Artificial Sequence
  Artificially synthesized oligonucleotide primer
tcgatggtgttgggcttgggtac

SEQ ID NO: 3 - DNA, 11559 bp, Homo sapiens
  CDS (182)..(6124)
ggggagcgctccaagatggcgcccaccgcagtcccgcccgccgcatcctcggcgcctttgcagtccggccgcgcctcccgggccccgcgttagggccgccgctgcctccctcgccgccgccgctgccagctgacctgtcctggacgcagcataactaacgaagctgctgcaggatgagaagatggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggacccatggaactggttagatttcagtgtcatcatgatggcgtatataacagagtttgtaaacctaggcaatgtttcagctctacgcactttcagggtactgagggctttgaaaactatttcggtaatcccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtctctttagtcagccttatagctaatgccctgggctactcggaactaggtgccataaagtcccttaggaccctaagagctttgagacccttaagagccttatcacgatttgaagggatgagggtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacctattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttagaggagaacaaaaattcagtattatacagatctaaaactcgcaagtgaaagattgtttacaaacttcctgaatattatcaatgcagaacagctgtggagactctaacctgaagatctataccaaacgtcgtctgcttaccacgtaacacagctgcatcttgagcagtgacctgccaagggcaaaggaccccgctccctagacttacagattttctaatgcttgggcaggtggttactgcatgttccacatcagtcaatgcaacttaggacaaaactaaccagatacagaaacagaagagaggctgccgggaccagcatatttccgttgcagccaaatggattttattttttcattttattgattctcagaagcagaaagcatcactttaaaagttcgtttgttcatgcaaactatatttgcattcttacattagttaagctaagcagcaaaaagaaaacacacacacacactcacatttagcccatgtcatttaattgtcagtttctttgacataaagcgcatcttctccacatgggcttcacgtggtttggagatgggtgggggaaaacaatcaggtttcttcaggctgaggaggacttgctcaggccgattccaaacattgtgctcgttcaatgcgtagaaatgatttgcatgatggcatgccgtgatcagaagtcatgcatgagatccatacaccacaggacactactaatctagtcccttgcactgggtcagcctttggacaggacccagccctgcaccgttcactgtatttggagaaaatggtaagagttccataccggctacaattctttgagttcttaaaagtccttcatacaccttctgggtagggaaacaaccaactaattgactaacaccaccaacaacaaaaaacaaacccaatccaacaagcagatggatccgttgcgtgtatatgtttaacagacatctctaacatacagccattgttgcacattttgcaagatgaactatttaatgctgctctgtgtccagtacatgggggagactttgatcccaaatggcttgtactatttatgtcactgtaaaaccaaatcctagggctaaaaaaaaaattcatttgtatcttgcaatatttatgacgatcgtttagccttcatactggagaattgacacttatttggggtagagataaaagtgcttttcaaagtagcaccagttatctctaggggtaatttggggaacttaaaatgacctttcattgggagttatggggtgctcatttcatttcgtatcatgtcctctgcattgtggctttctccaggcatgggtcgataccgcgaggggttcagatattctgaactgacctctctcttctgcagaggggtcatcgcatactcacacactgcatggctccccctgcctccatcacatttctccactgagcagggcttcccttgcccacagaggtgggtgagggtagcagcattggggttacggctgtggtttctttcacttattattagaataatagtcactggtgagacctgaggtagagatggagccgcctctgaaccaagcccacttggtttctttactgcactggttttcatgagaaagcaatttaatattaattctttaggatgttccagcttccctcagtgcattcccttgcagccagttttgctatctgcttagcgacctgcccattgtcattgaagtgtggcttctgggggagcgtcatatcttccattccatctcctgctttaggaaagacagtttttagtccaggggtttctcacctaatgactggatttcaaagccaagatgctgcagttgaatgtgtcaggaagtctctatacaaagagtgaaaagagactttagcatgtgaaccaaatccaaataaattcttcctgaaccatacactagagggggaagaagaaccaacccataatcagacacaccgatctgccttgcagtagaagcactttgaaggtaatttcacagtccaccagactagttttgcatagaacaaaccacagtaagtcggcaagcagagctttctatcttgttggaccactagaaactgtccagctctcttaagcctgcttctgcagcagcatccgtagaatttttgtactacacccaataccagactgggaaggcctatctttcagtgaccctgctaactgctaggatgaatgtatagtaacatgtacaggctacattgtaaacagcacaaaacagaagctgacatgtgtggttattctttttaagagaattataaatactgtaatatataattttattttattggcatgcctttttgtaaatacaaattattaacttattaaataggaaatggcttctggcttatgccattgtcatgtttatttttattttttatacttttctttcttcttagaacttagatgctgtcagccaatgtacatccccaaaccagacagacagacaaaaaattttttattgctaatggtcttttccaaaacaacaaaaaatatatatttttgttccaatgtgcaataaattctaaccacgtctatgcaagatttggctaaacttaataattgttcttaggtcttgagatgggcaacagagctgtaagatcccgccaagtacaccatgtgctcatgctagtgatgtcattaagctactagggcatagtgatgagatttttctgagatctaaccgtttccctccgctagctcctgaccccagcccagcaggctgtctcctcagtaacacatgggccttggtatctggcacccatcagccagcctcataggaaagaagccacctccgctgtatttgaagtttcactttttaaaaaaaaaatgtttcccacctttttatccttcacccactgtccttccacctgctcactcactcactctctcacccatcctgctccacacttctttggtagtaaatgacaccatcaccagcagtttacacccgcagttaacaggcatcgaactgaaagaatcagtgatgacgtttttgtgcatcttcgctgagtgggtaagtggcaatgctttgccaaatattaacgaagccaatcaaagagcagcgcagccacagtatcactgcacatatatatatagatatataaatataatataaatatttattttttgtagccaggtccttggtgcagtatttatactccacaatccaccttttaaaaaaaggacaaaaagcaaaaagatgtgtgatgctgttaataatttaaaatgcagagccaaagccactgagcagcagctgacatggttgctggtttgtgtgtgaaaaacattttccccctcttctttccttaacctccttgttgcttaggatgaggagactctgtaatttaaagcagaggacgagaggaggaagaaacttccatgttaacatggcctactctcactcctttctaaacgagccacttttggcgtcagaagttgactggagagagataaacaaactcccagtcaaagcccctaaagcgacagtgcccagagttcttttattttttgctgcaaccaacgtaaacctgtaaaagaccaacagtgaagattagtgtattagtgaatgcatggaggccaatgctgccctaatgagacgtgataaaaagtccatcactcctctaagccaaaaggaaacaaaataaaaatgaccctttttactgtacaagggaagcctgtcttggtgtgtttgttgcttgactatcccaattcttgagtcctgggaggggtctgggaattgggaccttagcacatttggggaaaggaggggaatagagggaagagaaatcaaaggggctctcgcaaagttctggctaagaaggagagattggaagaaaagcaaggtcctgaccccccccagatccccactcacccattgcatgtggcattccccactactggccggtgcttagaggcccataaaacctctcttcacctaatccacctggcagagccctcagagatgacgtcagccaacccagctggactggccttacttctgcctttttcacctgtatgttaaaaaacaaaaacaaaaaaaccctatccctgtgccccaaagctagggcatgtgcgatgcccaacagcctccactctgcaagctcatactgggccctgccctccatcgccccagacaggcccacagacgagggctggtctccttcacatttgtgtgggggtgggggcacgcttcctcttccttctctccctgtccccctttggggacatccctctctaggcctaggccatgtctcctggttcaggaacactctctcaggaggatctcgaagtgcccactctagtccgctccagtagctccactctggagcgagatggctggtaggtcagtccccatcctcctcccctcggcccccagcctccaggcctgctgtggaggtgagatcgccatgggtcatgactttgtgacttgagtcctgggcatcttctgtaatgatgcctctttcttttctcagatgttgcccacagtttagcaaaaagcttcgttcattttcaggtactccccaccccctaaagaattggctgcaacttgccagccagggggacatcaggagagaagaggaagtctgggttgggaggggcccaagttttgcaacagagtttccattgttcagctctgtgctttcttggattccctttccaagattaccaagatttcctccattaaaactcctgaggcaccccctgcccctgccccccactgctgtttgctgtacatagaggctaagtggggtggggtgggtgggtgtgcacatgtggtgtgtgtgtgtgtgtgtgaagaatgtatataggtaaaggggagtgtggtccagtggttccagaaaagggacagaactgtgttctgtccctagcttgaccgctggctcgctgtgtggccttgggcaagtcacttaacctctctgtgcctcagtttccccatctgtaaaatggggataataatactgacctacctcacaggggtgttgtgaggctttaactaaatgttttgtaaagcgttttgagatacagaggcaaaggcgctagggaagggcaaagtattattggacttaaggaagctgaaatggtaccataaatgctgctattctgagagccattttaaactgcactgctccaaccacccccgcttctcatcaccaggacagcaggtcgagaaaatgtctttcctttcacttgcttctggggtttgtgattattcaagacaattttttccccttgctgtatctccttccctcacctcctcgacttgttccctgttctgtttatgcggaaatggcagaaatgcttgagaaatgagaatgtgtaagtggattggaagtttatagtatgaaatttcaattttactttgtactgtacatctttttactttagaatttgcaataaagggttacgtcaatcttgttttcaactctc

SEQ ID NO: 4 - protein, 1980 aa, Homo sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYITEFVNLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 5 - DNA, 11436 bp, Homo sapiens
  CDS (182)..(6001)
ggggagcgctccaagatggcgcccaccgcagtcccgcccgccgcatcctcggcgcctttgcagtccggccgcgcctcccgggccccgcgttagggccgccgctgcctccctcgccgccgccgctgccagctgacctgtcctggacgcagcataactaacgaagctgctgcaggatgagaagatggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggacccatggaactggttagatttcagtgtcatcatgatggcgtatataacagagtttgtaaacctaggcaatgtttcagctctacgcactttcagggtactgagggctttgaaaactatttcggtaatcccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacctattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttagaggagaacaaaaattcagtattatacagatctaaaactcgcaagtgaaagattgtttacaaacttcctgaatattatcaatgcagaacagctgtggagactctaacctgaagatctataccaaacgtcgtctgcttaccacgtaacacagctgcatcttgagcagtgacctgccaagggcaaaggaccccgctccctagacttacagattttctaatgcttgggcaggtggttactgcatgttccacatcagtcaatgcaacttaggacaaaactaaccagatacagaaacagaagagaggctgccgggaccagcatatttccgttgcagccaaatggattttattttttcattttattgattctcagaagcagaaagcatcactttaaaagttcgtttgttcatgcaaactatatttgcattcttacattagttaagctaagcagcaaaaagaaaacacacacacacactcacatttagcccatgtcatttaattgtcagtttctttgacataaagcgcatcttctccacatgggcttcacgtggtttggagatgggtgggggaaaacaatcaggtttcttcaggctgaggaggacttgctcaggccgattccaaacattgtgctcgttcaatgcgtagaaatgatttgcatgatggcatgccgtgatcagaagtcatgcatgagatccatacaccacaggacactactaatctagtcccttgcactgggtcagcctttggacaggacccagccctgcaccgttcactgtatttggagaaaatggtaagagttccataccggctacaattctttgagttcttaaaagtccttcatacaccttctgggtagggaaacaaccaactaattgactaacaccaccaacaacaaaaaacaaacccaatccaacaagcagatggatccgttgcgtgtatatgtttaacagacatctctaacatacagccattgttgcacattttgcaagatgaactatttaatgctgctctgtgtccagtacatgggggagactttgatcccaaatggcttgtactatttatgtcactgtaaaaccaaatcctagggctaaaaaaaaaattcatttgtatcttgcaatatttatgacgatcgtttagccttcatactggagaattgacacttatttggggtagagataaaagtgcttttcaaagtagcaccagttatctctaggggtaatttggggaacttaaaatgacctttcattgggagttatggggtgctcatttcatttcgtatcatgtcctctgcattgtggctttctccaggcatgggtcgataccgcgaggggttcagatattctgaactgacctctctcttctgcagaggggtcatcgcatactcacacactgcatggctccccctgcctccatcacatttctccactgagcagggcttcccttgcccacagaggtgggtgagggtagcagcattggggttacggctgtggtttctttcacttattattagaataatagtcactggtgagacctgaggtagagatggagccgcctctgaaccaagcccacttggtttctttactgcactggttttcatgagaaagcaatttaatattaattctttaggatgttccagcttccctcagtgcattcccttgcagccagttttgctatctgcttagcgacctgcccattgtcattgaagtgtggcttctgggggagcgtcatatcttccattccatctcctgctttaggaaagacagtttttagtccaggggtttctcacctaatgactggatttcaaagccaagatgctgcagttgaatgtgtcaggaagtctctatacaaagagtgaaaagagactttagcatgtgaaccaaatccaaataaattcttcctgaaccatacactagagggggaagaagaaccaacccataatcagacacaccgatctgccttgcagtagaagcactttgaaggtaatttcacagtccaccagactagttttgcatagaacaaaccacagtaagtcggcaagcagagctttctatcttgttggaccactagaaactgtccagctctcttaagcctgcttctgcagcagcatccgtagaatttttgtactacacccaataccagactgggaaggcctatctttcagtgaccctgctaactgctaggatgaatgtatagtaacatgtacaggctacattgtaaacagcacaaaacagaagctgacatgtgtggttattctttttaagagaattataaatactgtaatatataattttattttattggcatgcctttttgtaaatacaaattattaacttattaaataggaaatggcttctggcttatgccattgtcatgtttatttttattttttatacttttctttcttcttagaacttagatgctgtcagccaatgtacatccccaaaccagacagacagacaaaaaattttttattgctaatggtcttttccaaaacaacaaaaaatatatatttttgttccaatgtgcaataaattctaaccacgtctatgcaagatttggctaaacttaataattgttcttaggtcttgagatgggcaacagagctgtaagatcccgccaagtacaccatgtgctcatgctagtgatgtcattaagctactagggcatagtgatgagatttttctgagatctaaccgtttccctccgctagctcctgaccccagcccagcaggctgtctcctcagtaacacatgggccttggtatctggcacccatcagccagcctcataggaaagaagccacctccgctgtatttgaagtttcactttttaaaaaaaaaatgtttcccacctttttatccttcacccactgtccttccacctgctcactcactcactctctcacccatcctgctccacacttctttggtagtaaatgacaccatcaccagcagtttacacccgcagttaacaggcatcgaactgaaagaatcagtgatgacgtttttgtgcatcttcgctgagtgggtaagtggcaatgctttgccaaatattaacgaagccaatcaaagagcagcgcagccacagtatcactgcacatatatatatagatatataaatataatataaatatttattttttgtagccaggtccttggtgcagtatttatactccacaatccaccttttaaaaaaaggacaaaaagcaaaaagatgtgtgatgctgttaataatttaaaatgcagagccaaagccactgagcagcagctgacatggttgctggtttgtgtgtgaaaaacattttccccctcttctttccttaacctccttgttgcttaggatgaggagactctgtaatttaaagcagaggacgagaggaggaagaaacttccatgttaacatggcctactctcactcctttctaaacgagccacttttggcgtcagaagttgactggagagagataaacaaactcccagtcaaagcccctaaagcgacagtgcccagagttcttttattttttgctgcaaccaacgtaaacctgtaaaagaccaacagtgaagattagtgtattagtgaatgcatggaggccaatgctgccctaatgagacgtgataaaaagtccatcactcctctaagccaaaaggaaacaaaataaaaatgaccctttttactgtacaagggaagcctgtcttggtgtgtttgttgcttgactatcccaattcttgagtcctgggaggggtctgggaattgggaccttagcacatttggggaaaggaggggaatagagggaagagaaatcaaaggggctctcgcaaagttctggctaagaaggagagattggaagaaaagcaaggtcctgaccccccccagatccccactcacccattgcatgtggcattccccactactggccggtgcttagaggcccataaaacctctcttcacctaatccacctggcagagccctcagagatgacgtcagccaacccagctggactggccttacttctgcctttttcacctgtatgttaaaaaacaaaaacaaaaaaaccctatccctgtgccccaaagctagggcatgtgcgatgcccaacagcctccactctgcaagctcatactgggccctgccctccatcgccccagacaggcccacagacgagggctggtctccttcacatttgtgtgggggtgggggcacgcttcctcttccttctctccctgtccccctttggggacatccctctctaggcctaggccatgtctcctggttcaggaacactctctcaggaggatctcgaagtgcccactctagtccgctccagtagctccactctggagcgagatggctggtaggtcagtccccatcctcctcccctcggcccccagcctccaggcctgctgtggaggtgagatcgccatgggtcatgactttgtgacttgagtcctgggcatcttctgtaatgatgcctctttcttttctcagatgttgcccacagtttagcaaaaagcttcgttcattttcaggtactccccaccccctaaagaattggctgcaacttgccagccagggggacatcaggagagaagaggaagtctgggttgggaggggcccaagttttgcaacagagtttccattgttcagctctgtgctttcttggattccctttccaagattaccaagatttcctccattaaaactcctgaggcaccccctgcccctgccccccactgctgtttgctgtacatagaggctaagtggggtggggtgggtgggtgtgcacatgtggtgtgtgtgtgtgtgtgtgaagaatgtatataggtaaaggggagtgtggtccagtggttccagaaaagggacagaactgtgttctgtccctagcttgaccgctggctcgctgtgtggccttgggcaagtcacttaacctctctgtgcctcagtttccccatctgtaaaatggggataataatactgacctacctcacaggggtgttgtgaggctttaactaaatgttttgtaaagcgttttgagatacagaggcaaaggcgctagggaagggcaaagtattattggacttaaggaagctgaaatggtaccataaatgctgctattctgagagccattttaaactgcactgctccaaccacccccgcttctcatcaccaggacagcaggtcgagaaaatgtctttcctttcacttgcttctggggtttgtgattattcaagacaattttttccccttgctgtatctccttccctcacctcctcgacttgttccctgttctgtttatgcggaaatggcagaaatgcttgagaaatgagaatgtgtaagtggattggaagtttatagtatgaaatttcaattttactttgtactgtacatctttttactttagaatttgcaataaagggttacgtcaatcttgttttcaactctc

SEQ ID NO: 6 - protein, 1939 aa, Homo sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYITEFVNLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 7 - DNA, 11559 bp, Homo sapiens
  CDS (182)..(6124)
ggggagcgctccaagatggcgcccaccgcagtcccgcccgccgcatcctcggcgcctttgcagtccggccgcgcctcccgggccccgcgttagggccgccgctgcctccctcgccgccgccgctgccagctgacctgtcctggacgcagcataactaacgaagctgctgcaggatgagaagatggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggacccatggaactggttagatttcagtgtcatcatgatggcatatgtgacagagtttgtggacctgggcaatgtctcagcgctgagaacattcagggttctccgagctttgaaaactatctctgtaattccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtctctttagtcagccttatagctaatgccctgggctactcggaactaggtgccataaagtcccttaggaccctaagagctttgagacccttaagagccttatcacgatttgaagggatgagggtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacctattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttagaggagaacaaaaattcagtattatacagatctaaaactcgcaagtgaaagattgtttacaaacttcctgaatattatcaatgcagaacagctgtggagactctaacctgaagatctataccaaacgtcgtctgcttaccacgtaacacagctgcatcttgagcagtgacctgccaagggcaaaggaccccgctccctagacttacagattttctaatgcttgggcaggtggttactgcatgttccacatcagtcaatgcaacttaggacaaaactaaccagatacagaaacagaagagaggctgccgggaccagcatatttccgttgcagccaaatggattttattttttcattttattgattctcagaagcagaaagcatcactttaaaagttcgtttgttcatgcaaactatatttgcattcttacattagttaagctaagcagcaaaaagaaaacacacacacacactcacatttagcccatgtcatttaattgtcagtttctttgacataaagcgcatcttctccacatgggcttcacgtggtttggagatgggtgggggaaaacaatcaggtttcttcaggctgaggaggacttgctcaggccgattccaaacattgtgctcgttcaatgcgtagaaatgatttgcatgatggcatgccgtgatcagaagtcatgcatgagatccatacaccacaggacactactaatctagtcccttgcactgggtcagcctttggacaggacccagccctgcaccgttcactgtatttggagaaaatggtaagagttccataccggctacaattctttgagttcttaaaagtccttcatacaccttctgggtagggaaacaaccaactaattgactaacaccaccaacaacaaaaaacaaacccaatccaacaagcagatggatccgttgcgtgtatatgtttaacagacatctctaacatacagccattgttgcacattttgcaagatgaactatttaatgctgctctgtgtccagtacatgggggagactttgatcccaaatggcttgtactatttatgtcactgtaaaaccaaatcctagggctaaaaaaaaaattcatttgtatcttgcaatatttatgacgatcgtttagccttcatactggagaattgacacttatttggggtagagataaaagtgcttttcaaagtagcaccagttatctctaggggtaatttggggaacttaaaatgacctttcattgggagttatggggtgctcatttcatttcgtatcatgtcctctgcattgtggctttctccaggcatgggtcgataccgcgaggggttcagatattctgaactgacctctctcttctgcagaggggtcatcgcatactcacacactgcatggctccccctgcctccatcacatttctccactgagcagggcttcccttgcccacagaggtgggtgagggtagcagcattggggttacggctgtggtttctttcacttattattagaataatagtcactggtgagacctgaggtagagatggagccgcctctgaaccaagcccacttggtttctttactgcactggttttcatgagaaagcaatttaatattaattctttaggatgttccagcttccctcagtgcattcccttgcagccagttttgctatctgcttagcgacctgcccattgtcattgaagtgtggcttctgggggagcgtcatatcttccattccatctcctgctttaggaaagacagtttttagtccaggggtttctcacctaatgactggatttcaaagccaagatgctgcagttgaatgtgtcaggaagtctctatacaaagagtgaaaagagactttagcatgtgaaccaaatccaaataaattcttcctgaaccatacactagagggggaagaagaaccaacccataatcagacacaccgatctgccttgcagtagaagcactttgaaggtaatttcacagtccaccagactagttttgcatagaacaaaccacagtaagtcggcaagcagagctttctatcttgttggaccactagaaactgtccagctctcttaagcctgcttctgcagcagcatccgtagaatttttgtactacacccaataccagactgggaaggcctatctttcagtgaccctgctaactgctaggatgaatgtatagtaacatgtacaggctacattgtaaacagcacaaaacagaagctgacatgtgtggttattctttttaagagaattataaatactgtaatatataattttattttattggcatgcctttttgtaaatacaaattattaacttattaaataggaaatggcttctggcttatgccattgtcatgtttatttttattttttatacttttctttcttcttagaacttagatgctgtcagccaatgtacatccccaaaccagacagacagacaaaaaattttttattgctaatggtcttttccaaaacaacaaaaaatatatatttttgttccaatgtgcaataaattctaaccacgtctatgcaagatttggctaaacttaataattgttcttaggtcttgagatgggcaacagagctgtaagatcccgccaagtacaccatgtgctcatgctagtgatgtcattaagctactagggcatagtgatgagatttttctgagatctaaccgtttccctccgctagctcctgaccccagcccagcaggctgtctcctcagtaacacatgggccttggtatctggcacccatcagccagcctcataggaaagaagccacctccgctgtatttgaagtttcactttttaaaaaaaaaatgtttcccacctttttatccttcacccactgtccttccacctgctcactcactcactctctcacccatcctgctccacacttctttggtagtaaatgacaccatcaccagcagtttacacccgcagttaacaggcatcgaactgaaagaatcagtgatgacgtttttgtgcatcttcgctgagtgggtaagtggcaatgctttgccaaatattaacgaagccaatcaaagagcagcgcagccacagtatcactgcacatatatatatagatatataaatataatataaatatttattttttgtagccaggtccttggtgcagtatttatactccacaatccaccttttaaaaaaaggacaaaaagcaaaaagatgtgtgatgctgttaataatttaaaatgcagagccaaagccactgagcagcagctgacatggttgctggtttgtgtgtgaaaaacattttccccctcttctttccttaacctccttgttgcttaggatgaggagactctgtaatttaaagcagaggacgagaggaggaagaaacttccatgttaacatggcctactctcactcctttctaaacgagccacttttggcgtcagaagttgactggagagagataaacaaactcccagtcaaagcccctaaagcgacagtgcccagagttcttttattttttgctgcaaccaacgtaaacctgtaaaagaccaacagtgaagattagtgtattagtgaatgcatggaggccaatgctgccctaatgagacgtgataaaaagtccatcactcctctaagccaaaaggaaacaaaataaaaatgaccctttttactgtacaagggaagcctgtcttggtgtgtttgttgcttgactatcccaattcttgagtcctgggaggggtctgggaattgggaccttagcacatttggggaaaggaggggaatagagggaagagaaatcaaaggggctctcgcaaagttctggctaagaaggagagattggaagaaaagcaaggtcctgaccccccccagatccccactcacccattgcatgtggcattccccactactggccggtgcttagaggcccataaaacctctcttcacctaatccacctggcagagccctcagagatgacgtcagccaacccagctggactggccttacttctgcctttttcacctgtatgttaaaaaacaaaaacaaaaaaaccctatccctgtgccccaaagctagggcatgtgcgatgcccaacagcctccactctgcaagctcatactgggccctgccctccatcgccccagacaggcccacagacgagggctggtctccttcacatttgtgtgggggtgggggcacgcttcctcttccttctctccctgtccccctttggggacatccctctctaggcctaggccatgtctcctggttcaggaacactctctcaggaggatctcgaagtgcccactctagtccgctccagtagctccactctggagcgagatggctggtaggtcagtccccatcctcctcccctcggcccccagcctccaggcctgctgtggaggtgagatcgccatgggtcatgactttgtgacttgagtcctgggcatcttctgtaatgatgcctctttcttttctcagatgttgcccacagtttagcaaaaagcttcgttcattttcaggtactccccaccccctaaagaattggctgcaacttgccagccagggggacatcaggagagaagaggaagtctgggttgggaggggcccaagttttgcaacagagtttccattgttcagctctgtgctttcttggattccctttccaagattaccaagatttcctccattaaaactcctgaggcaccccctgcccctgccccccactgctgtttgctgtacatagaggctaagtggggtggggtgggtgggtgtgcacatgtggtgtgtgtgtgtgtgtgtgaagaatgtatataggtaaaggggagtgtggtccagtggttccagaaaagggacagaactgtgttctgtccctagcttgaccgctggctcgctgtgtggccttgggcaagtcacttaacctctctgtgcctcagtttccccatctgtaaaatggggataataatactgacctacctcacaggggtgttgtgaggctttaactaaatgttttgtaaagcgttttgagatacagaggcaaaggcgctagggaagggcaaagtattattggacttaaggaagctgaaatggtaccataaatgctgctattctgagagccattttaaactgcactgctccaaccacccccgcttctcatcaccaggacagcaggtcgagaaaatgtctttcctttcacttgcttctggggtttgtgattattcaagacaattttttccccttgctgtatctccttccctcacctcctcgacttgttccctgttctgtttatgcggaaatggcagaaatgcttgagaaatgagaatgtgtaagtggattggaagtttatagtatgaaatttcaattttactttgtactgtacatctttttactttagaatttgcaataaagggttacgtcaatcttgttttcaactctc

SEQ ID NO: 8 - protein, 1980 aa, Homo sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYVTEFVDLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 9 - DNA, 11436 bp, Homo sapiens
  CDS (182)..(6001)
ggggagcgctccaagatggcgcccaccgcagtcccgcccgccgcatcctcggcgcctttgcagtccggccgcgcctcccgggccccgcgttagggccgccgctgcctccctcgccgccgccgctgccagctgacctgtcctggacgcagcataactaacgaagctgctgcaggatgagaagatggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggacccatggaactggttagatttcagtgtcatcatgatggcatatgtgacagagtttgtggacctgggcaatgtctcagcgctgagaacattcagggttctccgagctttgaaaactatctctgtaattccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacctattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttagaggagaacaaaaattcagtattatacagatctaaaactcgcaagtgaaagattgtttacaaacttcctgaatattatcaatgcagaacagctgtggagactctaacctgaagatctataccaaacgtcgtctgcttaccacgtaacacagctgcatcttgagcagtgacctgccaagggcaaaggaccccgctccctagacttacagattttctaatgcttgggcaggtggttactgcatgttccacatcagtcaatgcaacttaggacaaaactaaccagatacagaaacagaagagaggctgccgggaccagcatatttccgttgcagccaaatggattttattttttcattttattgattctcagaagcagaaagcatcactttaaaagttcgtttgttcatgcaaactatatttgcattcttacattagttaagctaagcagcaaaaagaaaacacacacacacactcacatttagcccatgtcatttaattgtcagtttctttgacataaagcgcatcttctccacatgggcttcacgtggtttggagatgggtgggggaaaacaatcaggtttcttcaggctgaggaggacttgctcaggccgattccaaacattgtgctcgttcaatgcgtagaaatgatttgcatgatggcatgccgtgatcagaagtcatgcatgagatccatacaccacaggacactactaatctagtcccttgcactgggtcagcctttggacaggacccagccctgcaccgttcactgtatttggagaaaatggtaagagttccataccggctacaattctttgagttcttaaaagtccttcatacaccttctgggtagggaaacaaccaactaattgactaacaccaccaacaacaaaaaacaaacccaatccaacaagcagatggatccgttgcgtgtatatgtttaacagacatctctaacatacagccattgttgcacattttgcaagatgaactatttaatgctgctctgtgtccagtacatgggggagactttgatcccaaatggcttgtactatttatgtcactgtaaaaccaaatcctagggctaaaaaaaaaattcatttgtatcttgcaatatttatgacgatcgtttagccttcatactggagaattgacacttatttggggtagagataaaagtgcttttcaaagtagcaccagttatctctaggggtaatttggggaacttaaaatgacctttcattgggagttatggggtgctcatttcatttcgtatcatgtcctctgcattgtggctttctccaggcatgggtcgataccgcgaggggttcagatattctgaactgacctctctcttctgcagaggggtcatcgcatactcacacactgcatggctccccctgcctccatcacatttctccactgagcagggcttcccttgcccacagaggtgggtgagggtagcagcattggggttacggctgtggtttctttcacttattattagaataatagtcactggtgagacctgaggtagagatggagccgcctctgaaccaagcccacttggtttctttactgcactggttttcatgagaaagcaatttaatattaattctttaggatgttccagcttccctcagtgcattcccttgcagccagttttgctatctgcttagcgacctgcccattgtcattgaagtgtggcttctgggggagcgtcatatcttccattccatctcctgctttaggaaagacagtttttagtccaggggtttctcacctaatgactggatttcaaagccaagatgctgcagttgaatgtgtcaggaagtctctatacaaagagtgaaaagagactttagcatgtgaaccaaatccaaataaattcttcctgaaccatacactagagggggaagaagaaccaacccataatcagacacaccgatctgccttgcagtagaagcactttgaaggtaatttcacagtccaccagactagttttgcatagaacaaaccacagtaagtcggcaagcagagctttctatcttgttggaccactagaaactgtccagctctcttaagcctgcttctgcagcagcatccgtagaatttttgtactacacccaataccagactgggaaggcctatctttcagtgaccctgctaactgctaggatgaatgtatagtaacatgtacaggctacattgtaaacagcacaaaacagaagctgacatgtgtggttattctttttaagagaattataaatactgtaatatataattttattttattggcatgcctttttgtaaatacaaattattaacttattaaataggaaatggcttctggcttatgccattgtcatgtttatttttattttttatacttttctttcttcttagaacttagatgctgtcagccaatgtacatccccaaaccagacagacagacaaaaaattttttattgctaatggtcttttccaaaacaacaaaaaatatatatttttgttccaatgtgcaataaattctaaccacgtctatgcaagatttggctaaacttaataattgttcttaggtcttgagatgggcaacagagctgtaagatcccgccaagtacaccatgtgctcatgctagtgatgtcattaagctactagggcatagtgatgagatttttctgagatctaaccgtttccctccgctagctcctgaccccagcccagcaggctgtctcctcagtaacacatgggccttggtatctggcacccatcagccagcctcataggaaagaagccacctccgctgtatttgaagtttcactttttaaaaaaaaaatgtttcccacctttttatccttcacccactgtccttccacctgctcactcactcactctctcacccatcctgctccacacttctttggtagtaaatgacaccatcaccagcagtttacacccgcagttaacaggcatcgaactgaaagaatcagtgatgacgtttttgtgcatcttcgctgagtgggtaagtggcaatgctttgccaaatattaacgaagccaatcaaagagcagcgcagccacagtatcactgcacatatatatatagatatataaatataatataaatatttattttttgtagccaggtccttggtgcagtatttatactccacaatccaccttttaaaaaaaggacaaaaagcaaaaagatgtgtgatgctgttaataatttaaaatgcagagccaaagccactgagcagcagctgacatggttgctggtttgtgtgtgaaaaacattttccccctcttctttccttaacctccttgttgcttaggatgaggagactctgtaatttaaagcagaggacgagaggaggaagaaacttccatgttaacatggcctactctcactcctttctaaacgagccacttttggcgtcagaagttgactggagagagataaacaaactcccagtcaaagcccctaaagcgacagtgcccagagttcttttattttttgctgcaaccaacgtaaacctgtaaaagaccaacagtgaagattagtgtattagtgaatgcatggaggccaatgctgccctaatgagacgtgataaaaagtccatcactcctctaagccaaaaggaaacaaaataaaaatgaccctttttactgtacaagggaagcctgtcttggtgtgtttgttgcttgactatcccaattcttgagtcctgggaggggtctgggaattgggaccttagcacatttggggaaaggaggggaatagagggaagagaaatcaaaggggctctcgcaaagttctggctaagaaggagagattggaagaaaagcaaggtcctgaccccccccagatccccactcacccattgcatgtggcattccccactactggccggtgcttagaggcccataaaacctctcttcacctaatccacctggcagagccctcagagatgacgtcagccaacccagctggactggccttacttctgcctttttcacctgtatgttaaaaaacaaaaacaaaaaaaccctatccctgtgccccaaagctagggcatgtgcgatgcccaacagcctccactctgcaagctcatactgggccctgccctccatcgccccagacaggcccacagacgagggctggtctccttcacatttgtgtgggggtgggggcacgcttcctcttccttctctccctgtccccctttggggacatccctctctaggcctaggccatgtctcctggttcaggaacactctctcaggaggatctcgaagtgcccactctagtccgctccagtagctccactctggagcgagatggctggtaggtcagtccccatcctcctcccctcggcccccagcctccaggcctgctgtggaggtgagatcgccatgggtcatgactttgtgacttgagtcctgggcatcttctgtaatgatgcctctttcttttctcagatgttgcccacagtttagcaaaaagcttcgttcattttcaggtactccccaccccctaaagaattggctgcaacttgccagccagggggacatcaggagagaagaggaagtctgggttgggaggggcccaagttttgcaacagagtttccattgttcagctctgtgctttcttggattccctttccaagattaccaagatttcctccattaaaactcctgaggcaccccctgcccctgccccccactgctgtttgctgtacatagaggctaagtggggtggggtgggtgggtgtgcacatgtggtgtgtgtgtgtgtgtgtgaagaatgtatataggtaaaggggagtgtggtccagtggttccagaaaagggacagaactgtgttctgtccctagcttgaccgctggctcgctgtgtggccttgggcaagtcacttaacctctctgtgcctcagtttccccatctgtaaaatggggataataatactgacctacctcacaggggtgttgtgaggctttaactaaatgttttgtaaagcgttttgagatacagaggcaaaggcgctagggaagggcaaagtattattggacttaaggaagctgaaatggtaccataaatgctgctattctgagagccattttaaactgcactgctccaaccacccccgcttctcatcaccaggacagcaggtcgagaaaatgtctttcctttcacttgcttctggggtttgtgattattcaagacaattttttccccttgctgtatctccttccctcacctcctcgacttgttccctgttctgtttatgcggaaatggcagaaatgcttgagaaatgagaatgtgtaagtggattggaagtttatagtatgaaatttcaattttactttgtactgtacatctttttactttagaatttgcaataaagggttacgtcaatcttgttttcaactctc

SEQ ID NO: 10 - protein, 1939 aa, Homo sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYVTEFVDLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 11 - DNA, 11340 bp, Mus musculus
  CDS (154)..(6090)
ggcgcgcatccagtccggagcgccgggacgcaggccggggagctgggagcgaagaaggcaggcgagcgggcgagcgggcggccgggcgcgcccggcaggagccgacctgtcccggactcagcataactaacgaagctgctgcagaatgagaagatggcagcgcggctgctcgcaccaccaggccccgacagtttcaagcctttcacccccgagtcgctggcaaacatcgagaggcgcatcgccgagagcaagctcaagaaaccacccaaggcggatggcagccaccgggaggacgatgaagacagcaagcccaagccaaacagtgacctggaggcggggaagagtttgcctttcatctacggggacatcccgcaaggcctggttgcggttcccctggaggactttgacccgtactatttgacgcagaaaacttttgtagtattaaacagagggaaaactctcttcagatttagcgccactcctgccttgtacattctaagtccttttaacctgataagaagaatagctattaaaattttgatacattcagtcttcagcatgatcatcatgtgcaccatcctgaccaactgtgtattcatgacttttagtaaccctcccgaatggtccaagaatgtggagtacacattcacagggatttacacatttgaatcactggtgaaaatcatcgccagaggtttctgcatagacggcttcaccttcttacgagacccgtggaactggttagacttcagtgtcatcatgatggcgtatataacagagtttgtaaacctaggcaatgtttcagctctacgcactttcagggtactgagggctttgaaaactatttcggtaatcccaggcctgaagacaatcgtgggcgccctcatccagtctgtgaagaagctgtcagacgtgatgatcctgacggtgttctgcctgagtgtcttcgccctgattggcctgcagctcttcatggggaaccttcgaaacaagtgtgtcgtgtggcccataaacttcaacgagagctatctggagaacggcaccagaggcttcgactgggaggagtatatcaacaataaaacaaacttttacatggtccctggcatgctagaacccttgctctgcgggaacagctctgacgccgggcaatgcccagaggggttccagtgcatgaaagcaggaagaaatcccaactacggctacaccagcttcgacaccttcagctgggccttcctggcactgttccgcctcatgactcaggactactgggagaacttataccagctgaccttacgagcggctgggaaaacgtacatgatcttctttgtcttggtgatcttcgtgggttctttctatttggtgaacttgatcttggctgtggtggccatggcttacgaagagcagaaccaggcaacactggaggaggcagagcagaaagaggcagagttcaaggcgatgctggagcagctcaagaagcagcaggaggaggcgcaggctgctgcaatggccacttcggcgggcaccgtctcagaagatgccattgaagaagaaggggaagatggggtaggctctccgaggagctcatctgagctgtctaaactcagttccaagagcgcgaaggagcgtcggaaccgacggaagaagaggaagcagaaggagctctctgaaggggaggagaagggggaccccgagaaggtgtttaagtcagagtcggaagatggcatgagaaggaaggccttccggctgccagacaataggatagggaggaaattttccatcatgaatcagtcgctgctcagcatcccaggctcgcccttcctttcccgacacaacagcaaaagcagcatcttcagctttcggggtcctggacggttccgggaccccggttcggagaacgagttcgcagacgacgagcacagcacagtggaggaaagtgagggccggcgcgactcgctcttcatcccgatccgcgcccgcgagcgccgcagcagctacagcggctacagcggctacagccagtgcagccgctcgtcgcgcatcttccccagcctgcggcgcagcgtgaaacgcaacagcaccgtggactgcaatggcgtggtgtcgctcatcgggcctggctcgcacatcgggcggctcctgcctgaggcaacaactgaggtggagattaagaagaaaggccctggatcgctcttagtctccatggaacaactcgcctcctacggacggaaggacagaatcaacagtataatgagcgttgtcacaaacacactagtggaagagctggaagagtctcagagaaagtgcccaccatgctggtataagtttgccaatactttcctcatctgggagtgtcacccctactggataaaactgaaggagattgtgaacctgatcgtcatggaccctttcgtagacttagccataaccatctgcattgtcctgaacacactatttatggcaatggagcaccatcccatgacgccacaattcgaacatgtcctggccgtagggaacctggtgttcactgggatcttcacggcggaaatgttcctgaagctcatagccatggatccctactattacttccaagaaggctggaacatttttgacggatttatcgtctccctcagtttaatggagctgggccttgcagacgtggaggggctctcagtgctgcgatctttccgattgctccgagtcttcaaattggccaagtcctggcccaccctgaacatgctgatcaagatcattgggaactccgttggcgccctgggcaacctgaccctggtgctggccatcattgtcttcatctttgccgtggtggggatgcagctctttggaaagagctacaaggagtgcgtctgtaagatcagccaggagtgcaagctcccgcgctggcacatgaacgacttcttccactccttcctcatcgtcttccgagtgctgtgtggggagtggatcgagaccatgtgggattgcatggaagtggccggccaggccatgtgccttatcgtgttcatgatggtcatggtcatcggcaacctggtggtgctgaacttatttctggccttgcttctgagctccttcagcgcagacaatctggcggccacggacgacgacggggaaatgaacaacctgcagatatcggtgatccggatcaagaagggcgtggcctgggccaaagtgaaggtgcatgccttcatgcaggcacacttcaagcaacgggaggctgatgaagtgaaacctttagacgagctgtatgagaagaaggccaactgcatcgccaaccacaccggcgtggacattcacaggaacggcgacttccagaaaaatggaaatggcaccactagcggcatcggcagcagcgtggagaagtacatcatcgacgaggaccacatgtccttcattaacaacccaaacctgaccgtgcgggtgcccattgctgtgggcgagtctgatttcgagaacctcaacacagaggatgttagcagcgagtcagaccccgaaggcagcaaagacaaactggacgataccagctcctcagaagggagtaccatcgacatcaagcctgaggtggaagaagtcccagtggagcagcctgaggaatacttggatccagacgcctgcttcacggagggttgtgtccagaggttcaagtgctgccaggtcaacatcgaggaaggactaggcaagtcgtggtggatcctgcggaaaacctgcttcctcattgtggagcacaattggtttgagaccttcatcattttcatgattctcctcagcagtggagccctggccttcgaggacatctacattgagcagaggaagaccatccgtaccatcctggagtatgcggacaaggtcttcacctacatcttcatcctggagatgttgctcaaatggacagcctatggcttcgtcaagttcttcaccaatgcctggtgctggttggacttcctcattgtggctgtctctttagtcagccttatagctaatgccctgggctactcggaactaggtgccataaagtcccttaggaccctaagagctttgagacccttaagagccttatcacgatttgaagggatgagggtggtggtgaacgccttggtgggcgccatcccctccatcatgaacgtgctgctggtgtgtctcatcttctggctgattttcagcatcatgggcgttaacctgtttgctgggaaataccactactgcttcaacgagacttcagaaatccggttcgaaatcgacgaggtgaacaataaaaccgactgtgagaagctcatggagggcaacaacacagagatccgatggaagaacgtcaagatcaacttcgacaacgtcggggcaggctacctggcccttcttcaagtggcaaccttcaaaggctggatggacatcatgtatgcagctgtagattcccgaaagccggacgagcagcctgattatgagggcaacatctacatgtacatctacttcgtcatcttcatcatcttcggctccttcttcaccctcaacctgttcatcggtgtcatcatcgacaatttcaatcaacagaagaaaaagtttggaggtcaggacatcttcatgacagaggaacagaagaagtactacaacgccatgaagaagctaggctccaagaagccacagaagcccatcccccgacctttgaacaaaatccaagggattgtctttgatttcgtcacgcaacaagccttcgacatcgtgatcatgatgcttatctgccttaacatggtgaccatgatggtggagacagacacacagagcaagcagatggagaacattctctactggattaatctggtcttcgtcatcttcttcacctgcgagtgtgtgctcaaaatgtttgccttgagacactactatttcaccattggctggaacatctttgactttgtggtggtcattctctccattgtgggaatgttcctggctgatatcattgagaagtacttcgtctccccgaccctattccgcgtcatccgattggcccgcatcgggcgcatcttgcgtctgatcaagggcgccaaagggatccgcaccctgctctttgccttaatgatgtcgctgcccgccctgttcaacatcggcctcctgctcttcctggtcatgttcatcttctccatctttgggatgtccaacttcgcgtacgtgaagcacgaggccggcattgatgacatgttcaacttcgagacgtttggcaacagcatgatctgcctgttccagatcacgacctctgctggttgggatggcttactgctgccaatcctgaaccgcccccctgactgcagcttggacaaggagcacccaggaagtggcttcaaaggggactgcgggaacccctccgtgggcatcttcttcttcgtcagctacatcatcatctcctttctgatcgtggtaaacatgtacattgccatcatcttggagaacttcagcgtagccacagaggaaagcgccgatcctctgagcgaggacgacttcgagactttctatgagatctgggagaagtttgatcctgatgccacccagttcatcgagtactgtaagctggccgactttgccgacgccctggagcatccgctccgagtacccaagcccaacaccatcgagctcatcgccatggacctgcccatggtgagcggagatcgaatccactgcttggacatccttttcgccttcaccaagcgagtcctgggagacagtggggagttggacatcctgcggcagcagatggaggagcggttcgtggcgtccaatccttccaaagtgtcttacgagcctatcacgaccactctgcggcgcaagcaggaagaggtgtctgcagtggtcttgcagcgtgcttacaggggacacctggctaggcggggcttcatctgcagaaagatcacttccaacaagctggagaatggaggcacccaccgagagaagaaggagagcaccccgtccacagcctccctcccctcttacgacagcgtgacaaagcccgacaaggagaagcagcagcgggcagaggaaggcaggagggaaagagccaagaggcaaaaagaagtcagggagtccaagtgctagaggagagggtaagggaagcttacgctggctgaacacgggcaagtgaaagcttgtttacaaacttccgaatctcacggacacagagcagctgcggagatgctgtactggaagacctataccaaacatagtctgcttacacgtgacacggttgcatcctgagcggtgacctgctggggacaaaggaccctgctccctggactcacagattttttctatcgcttgggcagacgttactgcatgttccacactcggtcaatgcaacttaggagtaaacttaacaggatacaaacagaggcggctgccaggaccagcagatcgccgctgcagccaaatggattctattttttttttcattttgttgattctcagaagcagaaagcatcactttaaaagtttgtttgttcatgcaaacaatatttgcattcttacattagttaagctaagcagcaaaaagaaacacacacacatacagacacacaaagacacacacagacacatacacacacacacacacattcagcctatgtcactaatcgtctgtttctttaacataacagcatcttctccacgtgggtggcacatggtttggagacggtgggggaaacaatcagggtttcaggctgaggaggacttgctcaggccaattccaaatattgtgctcgttcaatgcgtagaaatgacttgcatgatggcatgctgtgttcagaagtcatgcatgaggtccatacaccaacagacactagcaatcctgtccctcgcattggctcagccttcggacaggacccagccctgcactgttcactgtatttggagaaatggtaagagttccataccggctgcaatcctctcagtgtacaagtctttcatacacctctgggtagggagacataattaaccaattgaccactaccaacaaaaacaacccaaccaaacaagcagatggatccgctgcatatacatgtttgacagacatctctgacatacagccattgtcgtacattttgctgctctgtccaatacatggggagactctggccccgaatggcttgtactattaatgtcactgtaaaaccaaatcctagggctaaaaaaaaaaaaaaaaagaaaaaagaaaaaaaaaagaaaaatactaatagaaaaaagagttcatttgtatcttgcaatatcctgttgattgtttagccttcatcctggagaactgaccctggggtagagataaaagtgctatgtattgtagcatgttacctctagggggcctttggggggcttacagaaaccttgtggggggtttggggtgcttattgcattcagtgtcatgtctgtctgcttttctccaggctcaggtagatacggaaaggggttcagatattctgaactgccctgtcctgcagaggggttgtcacaagctcctggctgcacggttcttcctgcttctgccatatccctccagccagcagggctccccttcccagccgctcagggctggcaaggacagcagtgttggctgagataaggctgtaggctcttccatttgtagaataacgctctctaacaggacctgatggaaagatggagctgcctctggacagagcccacttggtcttgtttctacgacggcttttgtgagaaagggactcagtagtcattctttagggtgtcacagcttcccctggcctttcctttcagctcaccttgctttctgcttagtggcctgcccactgttgttgggggatggcttctagaggggcgtcatatcttccattccatcccccactttatagaagacagttcttagtccagggtgtctcagctgaccacaaggtctcaaagccaagatgctgcactcgaatgccagaaagtctctctgaaccaaatcaacacaaactctccctgagcatagtatggagggaatcgagtggcccatcagtagacacatcagatgcaccagtccgccttgcagtagaagcactttggatgtaatctcacagtccacctgactagttttgcatagaacaaaccacagccagtcagtgagcagagctttctatcttcttggaccggtagacactgtccaactctcccagctcagagagtgtgtccgtgtcaccacagctaggacaattctagagcgccacctgcaggcaacatcgcaaacagcacaaaacaggagctgacctgtggttattctttttaagagaattataaatactgtaatatataattttattttattggcatgcctttttgtaaatacaaattactaacttattaaataggaaatggcttctggcttacgccattgtcatggttatttttattttttatactttcctttcttcttagaatttagaagctgtcagccaatgtatacccctaaccagacagacagacaaaaaattttttattgctaatggtattttccaaaacaacaaaatatatatatatttttgttccaatgtgcaataaattctaaccacttctatgcaagatttggctaaactcaatcattgttcttagatcttgagatgggcaacagctatttgatcctgctaggaacaccaagcgccatgctagtaatgttcagcgattagatctgaccctcctaccccagctcctgacccagcccagcaggctgtgcctcagtttctcacaccctccaacagcctcgcagggaagaagccacctccgagtctgcttgctctaactttcactctgatcagacctctcccagcaatgcccctcaccttccatctgtccacccagccactctctgcccgcctcgctctctctgtcccactccatgatcctttcttgtgaaccacaccatcaccagcagttcacacccgcagttaacaggcatcgaaccttaaagaatcagctacggcaggttttggttggtttggttttgttttgcgtcctcattgcgtgagcaagtggcagggctttgccaaatattcatgaagcctgtcaaagagcggcagcagccagtaccactgcacatatatatatagatatataaatataatataaatatttattttttgtagccgggtccttggtgcagtatttatattccacgatcctcctttaaaaataataataataataataaaaaggacaaaaaagcaaaaagtcatgtgatgctgttcatttaaaacgcagagccaaagcccctgagcagtagtcgccatgactactggttgtgtgaaaacactcccttcttccttaacttccctgtggcttagggtgagaggctgggagtcacagggcagagggtgggagcaggaaagccgagtctgagtgatctgctctcaccatctggaaatgagccacttgggcatctgaagctgcgcacagagagacaagctccccctcagggcccctgaagcgacatagagtgcccaaagggcttctctcatcaccacaacccacgtaaactgtaagagacccatagtgtgctagtgagtgcatggagcctgatgctgccctaaacgatacctgatctttagaaaagtaaatcactattagccaaaaggaaacaaaatttacaaaacaaacaaacaaacaaaaaatacggccctttttaaccacacaagggaagcccgtcttgctgtgtgtctgtttgactttccagttattgaggtctgggaactgggaccctggagctttggggaaggagaaaggaaaacagagccccccccccccaattctaagtaggtactgcagagaaaagccaggtcctgaccccccaggtccctgcttactgattgcatgtggcattaccccttagactggacccccctccctcaccgtttccacccagcagagccccccagagatgacttcaaccaacctagctggtctggccttacttctgccttgttcatggttatgatttaagaaagaaagaaaaaaaaaaaacctctcctgaccccccacctcccaaggctagcgcaagcacgctgcccggcagcttccagtccaccagctcgtgctggccctgccttccatccactcccattatcagaccggcagactggggctgtccccttcacatttgtgcaggggagggggcatcccttggggacatccctctccaggccatgtctcctggtctaggaacactcaggaggatcttgaagtgcccactctagtccactctagtagctgaactagagagagtcggctggtaggtcagtccctgtccttgtccctcagctccagcctccaggccttctgtggaggtgaaattgccatgggtccggacatggtgacctgaaccctgggcatcttctggaacgatgcctctttcttttctcagatgttgcccaaagttttgcaaaaagcttccttcattttcaggtactcccccgccccctaaagaattagctgccgtcagtcagccaggggggtcaggagaagaggaagtctgggttcagaggacaagacttgcaccccggcctgtgcttcttgcctcttctcaagactgccaagatttcctccattaaaacactcctgaggcacccctgcccctgaccccactgctgtttgctgtacatagaggctaagtggggtgaagtgggcaggtgtgcacatgtatgtgtgtgtgtgtgtgaagaatgtacataggtaaaggggagcgtggtccagtggttccagaagagggacagaactgtgttccatccctagcttgaccactggctcgctgtgtggccttgggcaagtcacttaacctctctgtgcctcagtttccccatctgtaaaatgggggtaataatactgacctacctcacaggggtgttgtgaggctttaactagatgttttgtaaagcgttctgagatacagagacaaaggcgccagggaagggcaaagtattctttggacttgaggaagaggaactagtaccataaacgctgctattctgagagccattttaaactgcactgctccaccaccccccgcttctcatcaccaggacagcaggtcgagaaatgtctttccttgcacttgcttctggggtttgtgattcttctgatttttccctttgctgtatctccttccttacccctccactcgttccctgttctgttaatgcggaaatggcagaaatgcttgagaaatgagaatgtataagtggattggaagtttatagtctggattttcaattttactttgtactgtacatctttttactttagaatttgcaataaagggttacgtcaatcttgttttcaactctcctcttggaa

SEQ ID NO: 12 - protein, 1978 aa, Mus musculus
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPEWSKNVEYTFTGIYTFESLVKIIARGFCIDGFTFLRDPWNWLDFSVIMMAYITEFVNLGNVSALRTFRVLRALKTISVIPGLKTIVGALIQSVKKLSDVMILTVFCLSVFALIGLQLFMGNLRNKCVVWPINFNESYLENGTRGFDWEEYINNKTNFYMVPGMLEPLLCGNSSDAGQCPEGFQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEDGVGSPRSSSELSKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQCSRSSRIFPSLRRSVKRNSTVDCNGVVSLIGPGSHIGRLLPEATTEVEIKKKGPGSLLVSMEQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELGLADVEGLSVLRSFRLLRVFKLAKSWPTLNMLIKIIGNSVGALGNLTLVLAIIVFIFAVVGMQLFGKSYKECVCKISQECKLPRWHMNDFFHSFLIVFRVLCGEWIETMWDCMEVAGQAMCLIVFMMVMVIGNLVVLNLFLALLLSSFSADNLAATDDDGEMNNLQISVIRIKKGVAWAKVKVHAFMQAHFKQREADEVKPLDELYEKKANCIANHTGVDIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIDEVNNKTDCEKLMEGNNTEIRWKNVKINFDNVGAGYLALLQVATFKGWMDIMYAAVDSRKPDEQPDYEGNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICRKITSNKLENGGTHREKKESTPSTASLPSYDSVTKPDKEKQQRAEEGRRERAKRQKEVRESKC

SEQ ID NO: 13 - DNA, 221632 bp, Homo sapiens
ggggagcgctccaagatggcgcccaccgcagtcccgcccgccgcatcctcggcgcctttgcagtccggccgcgcctcccgggccccgcgttagggccgccgctgcctccctcgccgccgccgctgccgtaagtcgccccagagcccccgccctagcgctgcttcctgccctccgggtttcccttctcgcctcgggacccttgagcggggctcgcccatctccagtcaggggctttgccgcagagtgcgcggcggggctccaggggccgtggagctggggctctgctctccttgttttggacaggtgtttggaactcttgcttccttctactgtgtgtaccgggggtctcttaagagaactgaggaccaccagtccccaccccggacaccctgctttctccgtctcggctacagtgagggacacggcccgtccttggtgccccttccgcggtgcagccctccatccctccctcagtgccgggcagagttggggcccctagccggcttgcactgggaagggcagcgctgtcgggatcttcctacagcagcccctcagccctccccctcccccatgtcttactgcatccttctccctcagccggcttcacactgctaaaaactgcagaagccaagggctcctgggttcttcccgcagccagcactcccgactccctctgtcctttactcggatgcgttcgtccccctcccaacagggtgctctggagttgtgatgggggctcccgggaccttcctcggcaggcgaggattcctgcatttggaagcacatcactttgagaatatccccttcccccatcccacccccacccccaccccccacccccacgccacccgatgcagagcttgttcctctcctctccctcccccaccttaccatcagcagcagcacccttggcttcttgttagcgttggggtacaggactgtttgcccagctccgtgggcttggaccctcctctctagttctcacttggtaccttggggcaccgcaaagcctctgcagtttctcctgagagctcagctccatcccctccattgccagcaacaaccaccggtggagacttggctaactttccaggggttagccaggactgtcagaccctttcaggggccccagcttcgagttgggatggaatgggaaccatttttcagcccgataaataggattcttcaaaccatgcattccatccgactactccaccctatgccctcttcctcacttacactgcagtttttgaagcatgagggaatacattttatgtccggagattccaaggttctgcatcagcgttcataataaatggatgtcatctgtttggggggagggtagaagaaactttggattgcatctggctaaagtcagggggagggagaaataattagcatggagcaagctctgccacacaaggaaaactgctttttatgcacagtgggcaccagagagtcagggatggaagcaggaagtgtgggtgggggccatgggggtaaagggggagagtgctttttataactgttttcccaagtttgaacctggagagcctttagagagaggtagcagtgtgttagggctgggggatgttttagctttccaatggagatttcgggtgtggtaggtttttcactgtctcaaaatccctctgcacttctcttttctccacgtgcattagaagatgtggagggggggatgttgtgcttttattcttttctcaaaaaaaattttttttttgatttgtgatgcaaatggctggataacattaccacacggtaccatcctggtaggaatcttgggggaatcagacttaaaatgctgaaagctttatagcacattacatgtaacctggttcttgaaatactgcagcatttttctttggagctcatgacccataaggccttcatttctccctgaaagaggatttggttgctggttggtgaggtggtggttgtgggtagtgatcctttggtgttcagcacgcattccctgtcctagaaattgaggtcctccctggagagggctgcaagcatgttccgtgttctctgaacatctgattcagggtatatgtaaatgtgttgtgtttctgagtagagctggtggtgggtattgcaaagcagttcataatggtgcagatcagttattggtttcttggcttcagtttgagcagctttggttttcatttggggttttgtcaacttttatcaaaatctttggaggattttgttttaaattaataaaggataatcaagtggaatactgcatccttaaaaacaaccatacggttgctgttgctttccttgtaggacccttaagaattttttctcctggtccagctttgggttgcctgtgtttaggcttgtattcaggtttcttgctgtggtgctgaaaggacagttccccgaccagttctgcggcgaagtgcacacacactggacatgattgagtttttttttgctttttattatcagcaggctttctcgtggatgtgtatgtacatgcgcagctgggtttgtacttgaggttgaggcctagttatcatattttattactcccacataactcttcattttggctgtccctcatggagacaatgttttacaggctgcaacagatccatatgtaagccataagtgacattttgctgactgcattatttcagcacacttcagtcatttcaaatagcaagcaactgttaatattcttggtctccacaggaagtaatccgatagctggctaaattaaactggatcaggagagcgtcttctgtgggttgatttgaatagtgaactgggaaaggcacttgacacagcagaggtcagtattactaaagtcgatttatccaaaaaactattttaaaatcataaaataggaaggaggccataaaattcacatggtaccacctcccacccaatacaggaatctcctttatgggagatctttgctttgaatacatccgttggtagggaacttactgccacataacgtagctcattccatttggattccactgtgatcaaagtattttaatctcattgaaaaggtaatatacataaagttgaaagacatataaattggagctatatcggggtaaatgccattcttgtgcttgtttccaagaaaagattgagaaccatttttctgtagagaacatacaagaaacttgcacttacttgaaaagaatgcaaaaatagagacatgtccacctaaagcaagaaaaacattctgtgaatggaaagaaaaataattatctttaatgtgctggtattatgtaacaaattgttctagaaggcacatgcttgaattaatgaatatttaattcctatggattatgtctaaggacactaaagctatactatatagaaagcgttaaaaatccaaagtatatccagagtgagatcagttaccataattataaaaatacatacttattgcagaaaaattttaatatatgtaaaaacagggaaaaagtcatccttagttttaccatccttaccatctttgcttctaccataagcaaaacaattatctgtgttttattatgtttttccagtctttttttttcccatagagagggaaagaagcgcttgtaaagttatccagaatgatagtattttaaaactgagccctttaagctcttaattttatagttcacttgctagtcatttagggacttaaacagattgttgtatctcaatttacctttttcttaatcctttagaaatgttccaaaaaaaactgtaatgtgaaagaattcactcatagaattggacttcactcgtaaagtctgtttgtttatatttagcacattaactgaggaatcaataaaccttttcttaatcaatgaatcaacaaatagtctcactctgcacctactatatataaggcactgaactgaacactagtacaatgcctctctaagctggcgagcacccgcttaaaaaataatactgtgctcaaaaactgtggctattaaggtgtcgaaggaaacagaactcttaccctgtaagtttattaattgcggaaatgtatggcagtctgttagggccagcccagtgatttaacctaaaaaagaatagtttgtgcaagaatgggaagtaaatgcagtcaaaagacaaaggagaaggtttctggaaaaattatccctttgtgaagttatggagacacagccaggacacatatttaagtactatgttagagattaaggtgaaatgaaaacccaagaaggttgctatagttacactattgagaagaaaatgtcagaagcagtatgggctttggaatcagacaaaactagattcaaatcccgactctgctatgtattggctttgtgaccttgggcaagttatttaatctattggagcttcagtgttcctatctataaaagtgggaataataatacctacgttgcagggtatttgtggggattaaatgaaatctctgtttccttatggtttctgacacatagtgcttaatatatattagcccccttgcctctagggatttagcagcttaatgtgtatttatgtatgtatttgacgtagtatatatgtactgtattgaatttcagtagtctgaaaagcagacggatgctttatcttcagtctggcatagaggatggaacccgtaggtaccctaacttggatggacattgctaagaaaagttttactgtacaattgctctgctggcttccaccaacacagtgcaaatgtggttaccaaagtacctcttgccttgtccattcccaaactacactagaataggactctttacccaaaaggaatgctttggcactctccagctgggctgtttttagctgtagcctgggatctgaaggtgaaatggactcaaaggctgaggcctgcctttatgctgacttgagctggatttttacattgtggtaagtgattatacatgtttggagttggttttgtcttcagcaccattttaggtttccccactgtatagtctttgaactgctttatttgaatgggtctcataggtgacctgccacctttggtaaatgttactctgtgtttgtctatttataaaaactttcaaaatccttctatcagtcgattgagaaggaggtaaagcaaatatcctacccactttatagctgaagaaactgagtatcgaagagcttaatgaaggaagttgtccaagctaaggactatctaactggtgtagtggagactggtacctaggttggtcgactcatgtttagtgacttctttctgttaaatggtgttatattctgatgttcatataaccaagtaatgtgaacaattttggttttattttaagatattattagcttaggtagctaaacatatcttagtgagtgggtataatgaaagattctgggatagacagagggagggcaaaagtgcctttgaaggaaagcattttggaagcaggaaggatgaggggaaattaacagaggatgtatcaataagtagaatatggatgaaaacaggaggcaccaaagtaacacacttgtgttccttctgtggctatcctagctacttttaaaaaggtcatatcttggtgtctgcttatctttcatccgtgtctcttgcatgtaaacacatatacttggctattcatgaaatctctcccattgccaacctaggttgtaaccaggactgtagggaattatgaccattgttacctggacttctttgcttatgcaaggctgaaactggtgtgagactcagagaacagaatatgctggggattggtgatgggtacgggcagcaagttggggccaaagaagttctgggaggactttgggctagaagtagtacaaaggttggagaggctaatagttaagattttccttgggtgtatggagatgtgtcatactgaggagaatagaatgaaataaaagccagaagcctgagggtctaagaggtacttttagtttggtgctgaagctctcttgatcctggaaggagttgattggggtgggttgactaaaataggcgatggagagatttggatcactttatattcactattgatggtaggcatcctgtattcactgggcatgtcccaggatctggaagtggaaagcactgaactccctcaaaaggcacattcacaggctcctttcatattcagctgattagtgtagttttttgtgctataaatttttgggggttcctggggagtaagaatgctagagaaatagtggatgcttacttatccagggatggatcatcaagcttgtgaattatacagttatttatttttctcctttttcctccattatgagcacttaactgatcattagcatttctaccagcgtgaacttgggaagggagaaaaagtcaaaattaaaatgttacttttactttttagataatgattgataaaggaagagatggcaactactttgtcaatgaaaaaacacctcatatttaatctgttcttctctccttattttaatagtatagacagttgaaggctgagcctctacaagtttaatgtccactgcaagctggtgtaaatagcatttttctttcattttagtgagcaacaaccctgtaaacaagtagggcttaatttattatatatttagctccttgtttcaggattaattcctcaattaaggataatgcaacaggcttttgtgttacattcactactgtttttttgtcttagcaggaagctgaatgcttttaagtgaaaaaaattaatgcattattaaattgcaggaaaaaaatcaagcaggttgcaaatgagactgatagggcattctcagaggtcagaatacccagaaatcacatttgctacagatttctcaattggatttcctgtgatttggtgaatactctaaagaggcataatgccttgtttgtaggaaagatgtcttgaagataattagtcattttttaagatgagtattgtgatagggagggaagaaagtgtttggagtgtgacgaatcatcatagggagaaagcaaaagaaggtgaatgacagagtgaaaatgggcaaggggatttggatgtgaacgtggggaactgctcatattttaagagtaggtattcactcccctccaacaattcctttgggtacacctccgtattactgaaccagatcctcatctgctggacagggcacagaaccttgggaaggtggtttttgttgttgttgtttagttttattttattcttagctctgctagaactttgctgcaaattgctttctttatctgatctctgcattgagaaaactggagaatattttcttgctttatggaaaaaaatgtaatattcttaaacgtttctttcatttcctcttgatttttagtcatttaatacaaaaacattttctcagaaggctctgttgacagtttctcttccattgtcttgaaatgtttgtttgaatatttaacagaggcacaaaattcaaaaatcaaaaagttcgaagttaagtcttcttttactgtctcccagtcatgcagtatgcactccataatagcaattactgttactaatttctcttgtaatgcttcctgcatacatacatgcacatatacatattgtctccccctgctcctcacagttagttccattgtcttttaaggaagacttaaaataatttgaggatttggattctcattctagatcatcttagatcagttagttaccccttttccttaagaaatacataatgcctctgttttatttttcttagtaaaatttatattctaatttcttggcttatttaatgtgcaagttagtttgggctgaagcagcctttcctactaacctacattgcttttagggttttccttatagaattacctgttaaaaataaaagtgcagggggagacataaccttgagaactattggttcattgctgtctatgagtgaattctaaatcgcctgcttgaccacatcagtcaacaaatatttagcgagtagcaactatgggatatataaaagatagaccttatgttctaggagcttatttattttgtgaaataagtcataaacacaaaatgttaaataagaaaagattttcataatggttgatgattaaaaagttggaaaggatttattgctaaataaatggtatagttaataaattgattttttaggaaagagagagcatttcaagttggtatgattagagacaggttgatggaagaagcatgatttgagctgggctttacagaatggttgggtagaacagtgtgagccaaagtgagaaggcaagaatgtacaaagtgttttgggggagcagtgagaatctccttgtggcagataagcctgcaaagataggatagagttagccaaatattaaggactttttgaagtttcattcataacatggagaatgtagaatttagagagtatagaatgacagatgatcaaagctatactgtagtgttttaggaatattgggcagcagtgtctaattggatggaatagagcaaggaatggacatccacttagttggctctttgatttgtcccagctgtgtagtgatgaagggttggagtagtgtagtggttataaggatgggaaggaaggaaagtaaatataactttgtatatgtgtgttacatgaatgtgtgtgtttaacgtttttctaatgttagaaatagtttataggaagactcagtagaactcagtgaatgactttacatgggagaaaagggagagggaataaagattattttactgttttaaagattagagatagagagtctcctattttgaaaatctggggaagtatgttctgtaatccatggtagtctggtccatagcattttggttaacaagctgtctttatttctaattttctaattgtttctctgcagagggttttcttgagcttttaatgttatttcttgtaggaaaacttgaatcttctgaactttattatctaagctctcactgataaagattatttaatcttcgtttgtgggtctctacccttgttttggagttttacattctgtccattcatagacacttaacttgtcattttcgttggcattctcaactcaccttcagtttggaggataaaggaacaagaattctatacctttttatagacttggtaaatgactcttttgtccttaccgaaactcagtatatttgattttttgattgtttgtttatatcccatgtgcttcatgacagccatactatttgaaattgcaggctgagttaaaggtagtaactgaaaggattttagtaggtgaaagcagagggaaagaaaaggccttatcagccagtgatgttgctttctgtttcaggtactgaatcttaggccgtgtatgaaaaatgtcagacttgacttgcatgatcttcttggtatagctggggtacaagacggtggccagagggccctaacgtacaacttgagtatacagtggtaacttagagcaaaggcagaactcagggagcttgggttctgctactgccctttctcgtcagaaatgatcattgaatgagaaaaaataaagggttactagattatcagaacatggacatcaaaataatagaatacaccaagagaaaaaaaattagaaatttaatgcaatttttaaatctcatttaatttgttaaaattatatttcttctcaccacgaaagttactgagttcttgcaaattccattacatttaccacagtttgcactcagacatttggcagaagtccccatggtttgctgtggaaccagtagagcaggcacagttcatgtacaagatgttaataaatttagcactctatttagggtattacccaaattacccttacagtaattctattaggagattctctgtgcaatcccattataagtgcatacctttctcctagtttgaatgctcaaagtaacagtaattaagaactgttatacaatagtctcttggaaaggcattcttaaggtagcctgggatgaggaaagtccataaacaccacttaactgaatgatggcaatctgttttattcatctggatgttctcatgcaagtaatattttagtagatctatagatttttcttatttcttaaccatctttttgtttgtagtatttcttcttccttgtctctagaaatgcaggagagggttgtgtaaaacccaagaagggttttactaagtgcacagactgaatgattaagaagtgtagtagggaaacaaagtacagtgatcatatgaatgtttcacttacctaacaaggccttttttttttttttcctgactcatgcaggaaacttcacccataagtgcccaaggggtaagacttattttgggatcttaaggtagacttctggcattagaaattgctcaaaagctttataggtcaaagaaaataaattcatctttgtgttatgagtggatatcaaagatttgctattaattagatatgttttccattttaattttaattagattaaaattaaagtctgacccttgggggctgatgtgctttgttcctagccagttgccttgtctttcacctcgcacttctccacaggctgccttgctggttccagtggtgaggctcagcagtcaaatggggatgtcacttacatgccccacttgggctctaccctggagccctacctttgccaagggtggtgctcagagaaagggtttttttttttttttttttttttttaagagcaatgcatttaatactcaatgcaatctgttgcaaaatctcaagaaaaaaactcattcccagagaggaaggagtgtgtatgttttttttctagccaggattctagaaccattagctccttataggcattgcttctgtttatatggatgttgagtggtcaggcctttccagtttttttgttttttccttccacagtttaatcctagaacctagaaagaactgttatttgtgggaattgaatgctgactttggtagtattaagaaaaatccagtgtggatctgtagagttcagagtattggtgctcaacacaggaatgataaatgtttgagattatggatatgcgaattaccctgatctgatcactatatatgaattgaaacatcactatgtaccccataaatatgtatagttattatgtgtcaattaaaaataaagcaaaggtaaaaatttgagtaagtttgtaacacagaaggccaaacattgtataattccacttatgtgagttacctagaatagtcaaattcacagacagaaagtagaatagtggctatcaagggttgtgagaaacgggtagtggagagctactggttaaggagtacagagtttcagcttgagaaaatgagattgttctggagatttgtagtgataatggttgtaccacaatgcatgtacttaatgttacatacttgtatacttaaaaaacggttaagatggtaaattttatgtatattttaccacaatcaatcaatcaatcaatcaatataaaacaaaaaatagagtatggatgctagtccagattttggacaagagggtatgtggtgtttttatggccaaaatatagggaccagaaaccctgctttctagtgtggctttgggccaatacacagttcccctgggatgagcagggtactctttcctagtgggagacctattcctattagagtattacagccaaataaaccactactttcttcacaagaataagctcaataaaactaatatttgaaaagattttgaagttctagctcatgaatggcaaatattattttcctttttttcttcttaatctacccaagatagtatttcatttttcccttttaacattttaaacagcttttgttttattttttcttatttttaaaattttacaaacagtaaaattgaaacaaatcttggtgtacagctctgaatttttacacatgtataggttcatgtaaccgtgccacaatcaggatacagaacagttccattaccccaaaatactccctcctgctgttcctttgcagtcaaactttccctaatccccagcccctggaaacctgattggttctccattcctgtggtgttaccttttccagaatgtcataaaagtagaatcatactacactatataaccctttgagactgacttcttttgctgagcataatactttggagatttaaccatgttgttgtatttttcaattgttcttttttattgctgagtagtattgtatgtaactgttccaatgcatatacattgtatataacagtttgtcaacctgttcgtctgttgaagaacatttggaatatgtccagtttctggtgattatgaatagtacttctgcaaacattcatgtacaggtttttgtgtgaccctaccttcatatttctcaaagctgatccctaagagtaggattgctgggtaatatagtaagtgtatgtttacctttataagcaactaacaactgtttttcagaatggttgtaccatttcgcattcccaacagcagtgtatgagagttcagcagcatatgcgagttcaggtgctctgtatcctcattggcacttggaattgtcaggtttaaatttttttttttttatcttagccattctaataggtgtgtagtggtatctcattgtgatttcagtttgcatttctgtaatggctaatgatgtctaatatcttttcatgtgctctctgtgtatctttgtgaagtgtctgttgaaatgttttgcccattaaaaaaaattggattgtttgtattcttactatgagttttgagatttttgtttttaaaaatgttgtttttgagatatcaaaacagatatctttatcagataacttacatgggcatattttctcccactctttagcttagcttttcttaatagtgtttttatagagctaaagttttaaattttgatttagtccaatttgtcaatttttttcatttatgggtcacgtttttgatgtcatatataagaactctttgtcaaattcaagatgttcttggttttctgttaaaaattttgtgtagttttacattttacctttagatctgtgatccattcaagttaatctgtgtattagatatgagatttaggcctaggtttatttttatttttttgctaatagatgtgcagttaactgtttgagcaccattgttgttcaaaagactgtctttctcctgaattgattttggacttttgtcaaaagtcaattgcctgtatttatgtgggtttatttctgggctctctcttctgttccatcagtctgtatgtgtgtctgcgccttcctcaataccacacggtctcttttttccccaggtttttttgtttgtttgtttgtttgttttttgtttttttaacgggtttttgtttcgttttgtttggttttctttagagatggagtttcgctcttgttgcccaggctggagtgcatgtcacgatctcagctcaccgcaacctccgcctccccttcaagcaattctcctgcctcagcctcagaagtagctgggattacaggcatgcgccaccacacctggctaattttgtatttttcttttttagtagagatggggtttctccatgttggtcaggctggtctcaaactcctgatctcaggtgatccgctgccttggcctcccaaagtgctgggattacaggcatgagccactgtaccaggctccagtgtttttttttttagcttttattttaggtttgggggtacatgtgcaggtttgttgtatagatagattacatatcactggggtttagcatacagattattttgtcacccaggttataagcatagtacctgatgggtgtttttttgtttctcaccctcctcctaacgtccaccctaaagtaggcccaggtgtctgttgttcccctcttcctttccatatgtactcagagttttaaatacgagtttttcatgttcctaatttagctattctattgtcatagctttcttttctttccccataagttattggggtacaggtggtatttggttacatcagtaagttctttagtggtgatatgtgagattttggtgtacccatcacctgagcagtacacactgcaccatatttgtagtcttttatcccctgccttcttccccaaagtccactgtatcattcttaggccttttcatcctcatagcttagctcccacctatcagtgagaacacatgatgtttggttttccattcctgagttacttcacttagaataatagtctccaatctcatctaggtcattgcaaatgccgttaattcattcctttttatggctgagtagtattccatcatatatacaccacagtttctttatccacttgttgattaatgggcatttgggttagttccacaatgttgcagttgtgaattgtgctgctataaacatgtgtgtgcaagtatctttttcatataatgacttcttttcctctgggtggatacccagtagtgggattgctggaccaaatggtagttctacttttagttttttaaggaatctccacactgttttccatagcagctgtactagtttacgttcccaccagcaatgtagaagtgttccctgttcactgcatccacaccaacatctactgttttttgattatttgattatggccattctttcaggagtaaggtggtattgcattgtggttttggttcccatttccctgatcattagtgatgttgagcattttttcatatatttgttggccatttgtatatcttcttttgagaattgtctgttcatgtccttagcccactttttgatgggattgtttgttttttttcttactgatttgtttgagttcattgtagattctggttattagtcctttttcagatatatagattgtcaagattttctcctctgtgggttgtctgtttactctgctgacttccttgtgccgtgcaaaagcttttagtttaattaagccccaactattatctttgtttttattgcatttgcttttgggttcttggccatgaaatccttgcctaagccaatgtctagaagggtttttcattgttatcttctagaatttttatagtttcaggttttagatttaagtccttaatccatcttgagttgatttttgtataaggtgagagatgaggatccaatttcattctcctacatgtggctagccaattatcccagcaccatttgttgaaaagggtgtctattccccgcttacgtttttgtttcctttgttgaagatcagttggctgtaagtatttgagtttacttctgggttctctattctgttccgttggtctatgtgcctatttttataccagtaccacactgttttggtgactatggccttatagtatagtttgaaatcaggtagtatgatgcctccagatttgttctttttgcttagtcttgctttggctatgtgggctcttttttggttccatatgaattttagaattgtttttctaattccgagaagagtgatggtggcatttttgtggggattgcattgcatttgtagattggttttggctgtgtggtcattttcacaatattgattgtacccatccatgagcatgggatgtgtttccatttatttgtgtcgtctatgatttctttcagcagttttgtagttttctttgtagaggtcttttgactccttggttaggtatattcctaagtattttattttattttattttattttattttgtggctattgtaagaggggttgagtttttatatatatttatttatttatttttttttttgagacagagtcttgctctgtcggccaggctggagttcagtggcacgatctcagctcactgcaacctctgcctcccgggctcaagcaattctcctgcctcagcctcccaagtagctgggattacaggggtatgccaccacacctggctatttttgtatttgcagtagagatggggtttcaccatgttggccaggctggtcttgaactcctgacctcagataatccacccaccttggcctcccaaagttctgggattacaggcgtgagccaccttgcctggccggggtttagttcttgatttgattctctgcttggttgctgttggtgtatagaagagttactgatttgcatacattaatcttgtatctggaaactttgttgaattcttttatcagttcttggagctttctggaggagtctttagggttttcaaggtaaatgatcatatcgtcagcaaatagtgacagtttcacttcctctttactgatttggatgtcctttatttctttctcttctctgattgctctggctaggacttccagtactatgttgaagacaagtggtgagagtaggcatccttgtcttcttgttccagttctcagagggaatgcgttcaacttttccccattcagtattgtgttggctgtcagtttgtcatagatggcttttattacattgaggtctgtcccttgtatgccgattttgctgagagggatcataaagcgatgctgcattttgtcgaatgctttttctgcatctgttgagatgatcatgtgatttttgtttttaattctgtttatgtgctgtatcacatttattgacttccatatgttaagccatccctgcatccttgatatgaaacccacttgatcatggtggattatctttttggtatgttgttggatttggttagctaatattttgtgaaggattttagcatctgtattcatcaaggatatctgtctatagttttcttttttggttatgtccttttctggttttggtattagggtgatgctggcttcatagaatgaattagggagggttccttctttctctgtcttgaggaatagtgtcaaaaggattggtaccaatttttctttttcctttttttttttttttttgagatggagtcttgctctgtcgcccaggctggagtgcagtggcgtgatctcggctcactgcaagctccgcctcccgggttcacaccattctcctgcctcagcctcctgagtagctggggctacaggcgcccaccaccgtgcccggctaattttttttgtatttttagtagagacggggtttcaccatggtctccatctcctgaccttgtgatccacccgcctcggcctccgaaagtgctgggattacaggcgtgagccaccgcgcctggcctggtaccaatttttctttcaatgtctggtagaattctgctgtgaatccttctggtcctggactttttttttattggtaatttttaaattactgtttcagtcttgctgcttgttattggtctgttcagggtatcctaattcttcctgatttaagctaggagggttgtatttttccagcaattgatccatctcttttaggttttctagtttatgtgcgtaaaagtgttcatagtagccttgaatgatcttttgtatttcagtgatatcagttgtaatacctccagttttgtttcttattgaggttatttggattttctctcctctcttctaggctaatcttgctaaaaggtctatcaattttatttatcctttgaaagaaccagctttttgtttcatttttgtattttttttgtttcaatttcatttagttctgctctgattttggttatttcctttcatctgctgtatttgggtttgatttgttcttgtttctctagttccttgaggtgtgaccttagatgtcagtttatgctcttacagtctttttgatgtaggcgtttagggccgtgaactttcctcttagcaccacctttgctgtatcccagaggttttgatagtttgtgtcattactgtcgttcagtttgaagaatttttaaatttccatcttgattttgtttttgactcaatgctgattcagaagcaggttacttaacttctatgtatttgcatggttttgaaggtttcttttggagttgatttctagttttattccactgtggtctgagagagtgcttgatacaatttcagttttcttaaatttattgagacttgttttatggcccatcatatggtctctcttggaggaagttctatacattgttgaatagaatgtgtattctgtggttgttggatgaaatgttctgaatgtatctgttaagtccatttattccaaggtattgtttaaatccgttgttaaatggattcatagtttaaatcccttgttaaatggatttgtagttaaatctgttgtttctttgttgactttctgtcttgatgacctgtgtagtgccgttagtattgaagtcccccactattattgtgttgctgtctcatttattaggtctattagtaattgttttataaatttgggagctccagtgttagatgcatatatgtttaggattgtgatagtttcctgttggacaaggccttttaccattttataatgtccctctttgtctcttttaactgctgttgctttaaagtttgttttgtctgatataagaatagctacccctaggctgggggcagtggctcacgcctgtaatcccagcactttgggaggcagaggcagacagatcacctgaggtcaggagttcgagatgagcctggccaacatggcaaaaccccgcctctactaaaaatacaaaaatactcataggtgggaattgaacaatgagaacacatggacacaggaaggggaacatcatacaccggggcctgttgtggggtgtggggaggggggaggggggagggatagcattaggagatatacctaatgttaaatgaagagttagtgggtgcagcacaccaacatggcacatgtatacatatgtaacaaacctgcacgttgtgcacatgtaccctaaaacttaaagtataataaaaaaaatacaataaaataaaataaataaaaaataaaaatacaaaaataagccgggcatggtggcgcatgcctgtaatcccagctactccggaggctgaggcaggagaactgcttgaacccaggaggcggaggttgcagtgagccgagatcacaccatggcactccagcctgggtgacagagcaaaactctgtctcaaaaaaaaaaaaaaaaaaaaaaaggaatagctctccctgctcactcttggtgtccggttgtatgaaatgcctttttccacctgtttacctgaagtttatgtgagtccttatgtgttaggtgagtctcctgaaggcagcagatggttggtgagttctgcagttttgtatcctttaagtggagcatttaggccatttatattcaatgttagtattgagatgtgaggtaccgttgcattcatcgtgcatttgttgcctgtgtactttggtttttttgttttttgtttttgctttttaacttgtatttttgttttataggtcctgtgtgatttatgctttaaagtggttctcttttaatgtgtttccaggatttgtttcaagatttagagctcgttttagcagttcttgtactggtggcttggtagtggcaaattctctcagcatttgtttgtctgaaaaagactgtatctttccttcatatatgatgcttagtttcactagatacaaaattcctggctgataattgttttgtttgaggaggcctgaagatagggtcccaatcccttctagctagtagggtttctgatgagaaatctgctgttaatctgataggttttcctttataggttacctgatgcttctgtctcacagctgttaagattctttcctttgtcttccacactgttgattactgtagcttcatagcaagacttaaaattggatattgtgacttttccatctttgttcttttttcaaagttgttttggccatttcagtccttttgcctttgcatataaatttcacagtcagcttttatatctgcaaaaactgctggcattttgattggaactgcattaaatctataaatcagtttgagaatttatatcttaactatatcaaatattccagttaatcaacatgatacatctcttcatttatttgggtaatattttattcctttcatcagcatcttatagttttcagcctatagattctacatgttttgttaggtttatacctaagtattttttttttttttttgagacagagtctctctgtcgcccaggctggagcaagctccgcctcccaggttcacgccattctcctgcctcagcctctagagtagctgggactacaggtgcccgccaccacgcctggctaatttctttgtatttttagtagagatggggtttcaccgggttagccaaggatggtctcgatctcctgaccttgtgatcgccttcctcagcctcccaaagtgctgggattacaggcgtgagccaccgcgcccagctatacctaagtatttcttaagctaatataatttgtttgtttgtttgtttgtttttagatggagtttcactcttgttgcccagtctggagtgcagtggtgtgatctcggctcactgcaacctctgcctcctgggttcaggtgattctcctgcctcagcctcccaagtagctgggattacatgcatccctatgcctggctaatttttgtatttttagtagagacagggtttcaccacattggtcaggctggtctcgaactccctgacctcaggtgatccacccaccctggcctcccaaagtgttgggattactgacgtgagccactgcacccggcctaatgtaaatttttctattttagatttcagtttctaatttatttcttgtatatagagatatgatgatttttttgtatgtgaacttgtatcttacaaccttgttaaagtcacttgttctagaagctttattataggtttcttgggatttgctatatagataatcatagtccatgaaaagtgatagttttatttcttcctgtctaatctatatgccttttctttgtttcttttttcccccatttttacagacttccagtaagatatcaaatgtgggtagcgagactggacatccatgcattcttcctgattttagggaaaaaacattcagtttttcactattaagtatgatattccctgcttgttttttttgttttgttttgtttttgagacaaggtgttactctgacagccaagctggagtgtagtggcgcgaacgcagctcactgcaacctctacctcccaggcttgagcaatcctcccacctcagccttcgaagtagctgggatcacaggcacatgccaccacacttggctaatttttaaaatttttttgtagagacagggttttgccatgttgcccaggctggtcttgaattcctgagctcaagtgatccactcaccttggcctaccaaagtactggattacaggcatgagccaccatgccctagctgctagttttagtacattcattttatcaggtcgggtaagttcccttctattcctagtttactgagaattttatcaggaatgaatgttgaattttgtcaaatgctttttctatatgaattgatatgatcatatgatttttttcttgtttagtctgttaatataatgaattacattggttttcagataatgaacctactttgcatttctggggtaaatgccacttggtggtgatgtattctcctatattgttggatatgatttgctaatattttttttgagatggagtttcactcttgttgaccaggctggagtgcaatggtacgatctcagctcactgcaacctccgccttctgggttcaagtgattctcctgcctcagcctcccgagtagctgggattacaggcacccaccaccatgctcagctaattttttgtatttttagtagagatggggtttcaccatattggccagactggtcttgaactcctgacatcccgtgatccaaccgccctgcctcccaaagtgctgaaattataggtgtgagccaccatacccagccaatttgctaatctttttgttgagaagttttgcattatctgtgttcactaggatattggtctgtagttttgtttttttcttgtcctgtctttgtttagttttggtatcaggataatactgaccttataagataagttgggatatgttccctttaacattaaaaaaatattttcggaaaaagattttgtagaattctccagggaaaccatctgagcctagggttttctttgtgtgaaggttttaaactataggttagatttctctaatagatatagaacaattcaggttgtcatttcttttggggtgagttttaatactttgtggctttcaaggaattggtccatttaatttttgttgtcaaatttatgtgtatagatttgtctgtagtatttccttattcttttaacatctatggaggtctatggtgatatactttcttttattcctgatattgatagtttgtatattcattcttttttttccctttggctagaggcttattaatgttattcatcttttcaaagaaccagcttttcctatattaattttcctctatttttctgttttcaatgtagttgatttctgttctttattttctttcttctgctttctttaatttactcttccttttctgtttttctaaggtggaaacttggattgttgatttgagacctttcttcttttctaatataatctcttaatgctacaaatttccctctaggtactgttgtccctgcattttacaaattttgatatgttgatttttgtttcccctgagactttctctttgacctgtggattatttagaagtatgttgtttggagtgttgggagatttgtcagtgtctttgttttattgatttctagtttattccattatgattagagagcatactttgtattatttcaatattttacaatttgttagggtttgttttatgaccaagatatgatctagcttggcgactgtacatgtgtacttgaagataatgtatattctgttgttaggtggaatgttctatgaatattgatatctattcaagttggttgatggtggtgtttagtttctctgtatccctgattattttctgtctacttgttttattgattactaagaaaggaaaattgactatacttgtggatttgtccattttatccttagttttgtccatcgactatacttgtggatttgtccattttatccttagttttgttagtttttattttgtatatttgaaggttcttttgttagttacatacacattttggattgttatgtcttttagtgaattgactttttaaaatcattatttagtgtctctatccctggtaattttccttgctttgaagtctactttatctgatattaatataatcatcctacctttctgtcggttcatattttcatggcatttattttccactttttacttaaaagacatttttcccttgaaataatttcagatttatagaagtgttgcaaaaatagtaaagagagttcttgtattttcttcacccagcttcccctaatgttagcatcttatataacagtgatacatttgttaaaactaagacatttacattggtataatactttattaggtaagctacagacttcatttggatttcaccaaattttctactaatgtccttttttctgatccaggatccggtccaggataccatattgcatttaacattctatttttaacttacatttatcgtatttaatgtgggtttctggtagatagcatactgtatgttctttttttttttttttaaatccattctgacaacctctcttcttcttattcctttttgaaaaaattgtattgtggtaagaacattttacatgagatgtaccctcttaacaaattttaagtgtacaatacagtattgttaattatatgttcaatgttgtacaggagatctccagaatttattcatcttacttaactgaaagtttatgcccattgattagtaactgcccatgacaatctcttttagttggtatgtttagaccaccttcatttaatgtagttattaatatgtttgggtggtctgtcattttatttgttttctatttccttttccttcttttggattatttttaatattctattttttttttactgttttcattatatagttattttagtgtagcactaggaattacaatgtacatacttaagtgttcagtctacttagaattaatatattaccactttaagtggaatatagaaatgttagtaccaccaggtgcagtggtgcacacctataatccaaacactttgggaggccaaggtgggcagatcgcttgagcccaggaatttgagactagcctgagcaacatggtaaaaccctatccctacaaaaaatacaaaaattagtgggcgtggtggctcatgcctgtggtcccagcttctctggaggctggggtgaaggattacttgagcctgggagtttgaggctgcagtgagctgtgatcacaccattgtcttccagctacggcagcagggtaaaaccctgtctgaaacaaaaacaaaagaaaaaaaaatgttagtaccgtgtagatctctttactgtctcccttttatgttgtaattttcatacatattgcatctacatgcatgaaaacccaatcaataatgttataatttgtgcttttcaaccatcatttatattttattattatttttaaaaattattttttgtggagacaaggtcttactctgtcacccaggctggagtgcagtggtacaatcgtagctcactgcagcctcaaacttctgggcttaagtgatcctctcacctcagcctcctgggtagataggactataggtgcatgtcatccttcatgcctggctaatttaaagaaaaaattttttttttgcagagatgagggtctgactgtgttgcccaggctggtcttaaactcttggcctccagcagtcctgtcatcttggcctcccaaaatggtgagactacaggcgtgagccaccacagttggcctcatcatttatattttaaagaccctaaaaggagtaaaatgggtattacatttaccttggtatttaccacttctattgctcttccttgtttcctgaagttctaaatttccctgtaatattattttccctccatctaaagaatttcctttagcatttcttttagagcagtctgctagtgatgaattattttggttttctttatatgagaatttctgtatttcatcatcatcttttaaaaatattttcactagatatacaattctgggttgacatttgtttcttttcattttcccccagcacttaaaagtgtttcactgggctaggccaggtgcagtggctcacacctggctcacggatcacttgaggtcaggagttcgagaccagcctggctaacatggtgaaacctgtctctactaaaaatacaaaaaaaaattagccagacatggtggtgggcacctgtagtcccagctactcaggaggttgaggcaggagaatcatttgaacccgggaggcggaggtacagtgagcaaagaatgcaccactgcaccccagcctgggcaacagagtgaaactgtctccaaacaaacaaacaaaaagtgtttcactgggttgggcatggtggttcctgcttgtcatctccgcactttgggaggctgacatgggaggactgcctgagcccaggagtttgagacaggctgggcaacatagggagacccccatctctacaaataataaaaaattagccaggcatggtggtgcatacctgtagtcccagctactcaggaggctgaggtgggaggatcacttgagcctgggaagttgaggctgcagtgagccatgatcatgccactgcactccagcctgagtgacagagtgagaccctgtctcaaaaaaaaaaaaaaagttttactgtcttttgttatgatttctgttgggaaatttcagtcattcaaattattgtttccctatgtgtaatgtatcctttttctcttactgctttcaagattttttattttagttttcattggtttgatcatgatgtttttgcacaaggatttctttgagtttatcctgtttggtgatcactgagtttcttaaatctgtaagtttatgtcttcttaaatttgggaagttttagccattgtttcttcaaatactttttgtgcactatactttctccttacctgttgggcctttgatacaaatgttaagtcttttggtgttgtcccatgggtcattgagattctgtgctttttttccttcgtttctaatttttttctctctctcttgttcagcttggataatttgtgtattttcaagttcatcaactattctgtcatctctattatactattgagcccatccaatgaattttaaaatttctgttattgtgttttctaatttaaaattttccatttggttcttttttaaaaaagtgttctatttcttcattaagactttttatttttccatttatttcaagactgtttgcctttccttctttgagcatacttagaatagctgcattaaagcctttgccaattctaacatcttgcattgatatctgttgattgtctttttccttgtgagatgttgagattttcaagtttttttgtatgttagtttgaatttgtatcctgaacactttcaaattgttagaagactttgggtcttgtttaaatcatatagagaatgtttcttgcttatttgttttagcaggcaatccagttagattcaggttgcaaattccttcctgccttctgtgggctggggtccaaaatcagttcagttttcaaagtatttgccattctatttctgtttattccaggtatgtcccagctagtggccagtttcagatctgggcagcagtatactccagagattagttgtcaaaacctttggtgtgctgtttagggccagatacatgcatgcacagcttggagatgagccccggaattcatatacagccttattgagctcctttctcttgttgtttcccagactttccagatctgaagggtttcccatcctgctaccctgcctagaagtcttggacttttagtttctctattctgccttttacttcctatgaccacatctcccttgagaaccaaatggcaagatacggagagtcaacgaagcaagggaggctttcctgtgctcttgggatcccagcttccctggtcagggaagaacttttgctattgactgccactgctgcttaactactgtgaggtagggcaggagagagcaggaaacacacacacaattcagattttctcccttccctttcgtgtctaggggctcccttttctgtttcttggaccagaaatggaggtcttctcctgtagctttttctgtctgtgcttggggcacagttctggatttggctgccttttttgagtccaggccaagagataccagagcaacacacacacacacacacacacacacacacacacacacacacacacacacagaaagaaaaaaaggcaaaactctgctggttttgtgatactttgaattgtgggtttcttttcctaaactatctgctatcaattatttttcagagtcattaagtaatggatggagcagctctgtccaaggattttagttgaatacagtgggagatacagggtagagtgggcttacttcatcttatctggagtgagaaactttggttatttgagagaggaacaaggctggcaggtgtgagaaaggtggtggctttgaggcaggttctcaaccttgagagatcaagtgactagattgacaggcctcgaggtcctgggaggagacaccctgaggaatgacttcagtatttttgatgcaggatgccctctcctcctcctttttaatttttaaatttaagtaaaatgttttttaaaatatataatatctgcacttagcaccagattggaaagctgtaaagggaacaaagtaagagaggtaagtcttcctcagttacctacctcctagccacacagttcctttccctggaggcaaccacttcccagtttctcatgtgtctttacagagactttgtacttattaccaaactggaatatggataagtgtgcatatattaacaaaaatggcagtgtactgtacacactattatataccttactttttctcttaatatgtcctggaggcctttccatatcagtgtccagagaaggcctcgttctttttagcagctgtgcagaatgccattaaatggatatactatcatttatttaataagtcctttattgatgggcatttatttcattaatgaatattcagtaaataaatatttattccattaatttcaaactctccttattatgggaaagttgcattcaatatccttgtatacatattttgcatatgtgaggctatatagaataaatacctgggattagaattgctgggctaatgatgtgtgtccatttaaaactgttaatagcatattgttaacttatttcatatcactacaaataatccatcaacttttaagccttgatatggtaaggaatattagaataggtaatagaagccttcatataatcttaaatctttattcaaactcaggttaacatttaactcaggttttaccaagtcttttaccaaaattatgtggaaaaaaataaaaaggattaactgacattttccttctttccttacccttctaattctagtaagagatgtgtcaacaagctgtgaaaagaatatcataaaatgactaaatagtaaacaaccaaaagggaaggatataacagtcaagaggatttagatgaccaattgattggacagtcagttcccaatcctcaaatgttggttggattaaagcttttcttctaagaacattttgtctatcatgtgttctcacctacacaagcatgtctgtattagtagagagggcaggtggaagttgttatttactgtactgatgtactgatggtggaatagaagccctgcaaaagtgataatcatttttttcctggtccctagatcaagacattcagtgtgaaaaaattttgtgcaagaaacagtcttagagaagtatttaatattatggagaccaaagagttgtcatttagggttgttttgatggattgtggtgataattttaagtcaaagctttcttggaaaatctgagatgcagatttttcaatacaatggagcattttcttaataacaatcattttaatgttatactagtgaccaaaaatcagatcagtggatgcttgcttaaagtttttcatgtttacttgggtatgattgtattggtttttacttcaattaatttcaataaatcatgaataaaattagctttaaaattaatctgaaaaagactatttttaagtgaataatgtgttctttatatgggaggactctacttggataaattgttaagtaataaaacaggttttggtagctcttcaaatagtcatctttaattttttctttatataactctatatttgagaaattgggatgagcacgtattagtttgtaaagaaatgcaatgcagtagaaactgtaaaagaaagaaaaacagctaacttggaaggctgtgcacttaatagaatgaagcaatcattgcataaaacattcttggaatttctattttgaattttgccttctgaacctgttccacatcagctagacttaaaaaaaaaaagaaaaaaactatgtgtggtgattcacgcctgtaatcccagcactttggagggccgaagcaagaggatcgcttgagctcaggagtttgagaccagtctgggcaacatagtgagacaccatctctatttaaaaaaaaaaaaaaattaggtgtggtagcacacgcctgtggtcccagctactcgggaggctgaggcgggaggatggattgagcccaggaggttgaggctgtagtgagccatggttacaccactgcattccagcctgggtgacagagtgagaccctgtctcaaaaaaacgtggggcctcataggatcagaaaccacggtggagcagactatgaaggagaaatatgatggttcttataatggcttcagtggggctggctttctggaagatgttcaggtatttatccttggtctctcctcagctgctttatagagtaatgcagtagaggaaagactgtcaagaaattttttattacttaggtgctatatcgtttgctttttaacttcataaaattttttggccatctctgcttagtatataaagcaagttgagcaagtgtggctcttgccaactataaacttaggtcacttgggtctggaaattccctcttgaatgctatttcctgaatttgatttagagatcagagtttacgtggtcaatgtacgtctttcccacaggatagccaatgaattcttagagttttaaaccatcactctaaagcatgctaatttgcatttagaaatacaaagtgcatcatattggtgaccccttttagatagtgggatgggtatcagatgttacccattagttctgatgtcttaggcagagcctatgcagttatttggccacagactaggacattgctagtatattatctattcatgtatatgatgatgattcttggaataatttcctctggatgtggagagactagggcagtacagatttctttaattctgtgtcagagcagtagtgagtaagaaggaaggggcagagtgtcagccacagctacaggcctcttacaggcagtctagggtccagaccagacacgatgagcatcccaacagaggcctgagttctgcgatgtaggcagaatgaccagagagacccagtttatctcatccggagatttggaagggaagaaaggagtccacatcaaagaaagaatgtgtacgcctgtgttttcaggattctgtctgcctctggtaaggaagcatctgaccgtgaatgtgccatggaagctttctcttcattaatagaagtatcattacaggagactcagttcacaagacagtatctcaagtgcactttgttctctgtagaaagaggtacccaataacatgatcatatacttctgtcacatagaaatgttgcaaataaatttttaataagttaaacagttaatttttctcgctaacttgaccttaaaccagcattagattttttaaaatcaactttttattttggaataattttaggtttacagcaaagttgcaaggggaatacggagaattcctgtatatgcctcacctggtttcccataatgttaatatatcaccatggtgtatttgtcagaactaagaaaccaatgttggtacattactattaactaaattccacactttatttggattttaccacttttcacactgatgtcctttttctgttccaggattcagtcaagagtattacattgcattgtcattatgtctccttagtctcttctggtgtgtgacagtttctcagacttcccttgttttttaataaccttggtaggtttgaggatactgatgaggtattttgtagaacacccctcaatttaggtttgtatgatgtttttctcatgattagactggggttatggatttttgggaagactactacagaggtgaagtgccttcctcatatcatattggaagaatatactaacatgacttattacagataatgttaattttgatcatttggttgtgttagtgtttgctaggtttctcactataaagttactattttttttctctttctatactgcattctttggcaatgggtcaccaagtccagcccacgcttgggggaggaggagtgaggattaagctttacctcctacagagggtattatctacttatattatctggaattcttctgcaaaggaagattttccccttctacctatttgtttatttattgagttgttaatttgtatctctatggacttgtatttattttataccttgggttataatacaataccatgctatttattttcttcctgaaattgttctagctttggcttctggggcctcttccaggttaacttctgtgtcactttgacatgtcctcatccttttgctacttgagcacctccctacttttttagaattagaagatgttccaggttcatcttgtattttccttgccccggctctagaatcagctttttctaaagagccctagttctttttattggagaaaccaagttctgagcattgggttgcatgctgctagtaggccctctcagtgaatagagttaggaaataaaagtatgtatgctaacccatgtatatatacatatttctaattatttctgtatctatcagtctgtatatatgctaagctaaacgtggatttgcactggtatctttgactccaactcagtactgcagagttcattctagcctttcccccttgcagatctgcatttcactctctggcaatgagaaacctgcctctcatcatccaccgtggtagatggggagtttacttatttgttcaaacccttacacatgtaaagcaggttcagatttgttaacttatactcctatgagctacgatttaccaattagagttccatgtttatgtacagattattttgtcattagcctatagttgtcagtcaaaacactattttctttttttttattattatactttaagttttagggtacatgtgcacaacgtgcaggttagttacatatgtaaacatgtgccatgttggtgtgctgcacccattaactcgtcatttaacattaggtatatcttctaatgctatcccttcctcctccccccaccccacaacaggccccggtgtgtgatgttccccttcctgtgtccatgtgttctcattgttcaattcccacctatgagtgagaacatgcggtgtttggttttttgtccttgtgatagtttgctgagaatgatggtttccagcttcatccatgtccctacaaaggacatgaactcatcgtttatggctgcatagtattccatggtgtatatgtgccacattttcttaatccagtctatcattgttggacatttgggttggttccaagtctttgctattgtgaatagtgccacaataaacatatgtgtgcatgtgtctttatagcagcatgatttatagtcctttgggtatatacccagtaatgggattgctgggtctaatggtatttctagttcaagatccctgaggaatcgccacactgacttccacaagggttgaactagtttacagtcccaccaacagtgtaaaagtgttcctatttctccacatcctctctagcacctgttgtttcctgactttttaatgatcgccattctaactggtgtgagatggtatctcactgtggttttgatttgcatttttctgatggccagtgatgatgagcattttttcatgtgttttttggctgcataaatgtcttcttttgagaagtgtctgttcatatccttcgcccactttttgatggggttgtttgtttttttcttgtaaatttgtttgagttcattgtagattctggatattagccctttgtcagatgagtagattgcaaaaattttctcccattctgtaggttgcctgttcactctgatggtagtttcttttgctgtgcagaagctctttagtttaattagatcccatttgtcaattttggcttttgttgccgttgcttttggtgttttagacatgaagtccttgcccatgcctatgtcctgaatggtattgcctaggttttgttttagggtttttatggttttaggtctaacgtttaagtctttaatccatcttgaattaatttttgtataaggtgtaaggaagggatccagtttcagctttctacatatggctagccagtttttccagcaccgtttattaaatagggaatcctttccccatttcttgttcaaaacactattttctaaggttatataggccagcttttttttttcctctacccccttcagtgagtttatgtcatacatttataatatgttaggttcatttgtcacagtctacatcctatcctgggattccctgacatactggttctcttgaaaaaacgtacataccttaaagttcactctttgtaaggtacagttctacggattttaacaaatgcatagagtaacgtatgttccaccctagtaacacatagaataattccgttatcctaaaagtttccctgtgcatctccctgtagtcaaacaccttccactgcaaaccctgataactagtaatctgtttttcatccctgtagttttgccttttccaaaatgtcacgtgagtggaatcatataatttgtagtctttcacctagcaaaatgtatttaagattcactcatattgttgcaagaatcaatggcttgttgctttttgttgttgactaatcgtccattctatggtataccacagtttctttattcagtcacctgtcaaaagacgttgttgcttccagttttggatgcttatacacaaagctgctataaatatttgtgtacaggtttttatgtgaacctggttttcagttaacttggatcaataactaggttgtgattgctgggtcatattttaagtttatatttaactttataggaagttgtcaaactgtgcggtattagagtttaatcacccctccccctcttttgtttttttggacagagggaataattaaacaatcgctaagattcaggccaaaaatgtcaactaattatctacttgtctgtgtaaaattcagcttatcaagtaactgctgtatacatagcattgaatttgttgagtggtagaagtttttaaaggagtttgctatctaatacggaaggtgagagtttgtaagcagtgatgtaagccaccatttaggattaagttgggttgggttctagttctttgtgtctggtggcaatagtagtggtagtatccagttgatttgtggccattgatgtaatttataaaggtgactcagtgactacacagagtggtcaatgtcattgaaaggaaagtttattacttacatttcccaagacaagagggcattctgcaccacacagggccgcagagggaagcaccagtttcagtcaggaggcagaaggagcgaggggaaagcatggtccagagctcttactatgttttccttgggaaaggcaaggcagggcaggagaaacaggttagaattggccaatttgaataattccagtgggctttagggctgtccttagttgtctggtacctggtcctgggttgatttagggcaggtgaaatgttggcttgatgtgtgagagttagataaaggaggtggttcagagaatggactttggctctcgggaggtttaaacaactttggccattagtttggccttgtgatctatggatgccagatagacaaatacagaatcagcaaaaacacagaatacccaacacgaccttcctgcatagagctctccgtacctaccagttgaattatttaattcaatgcattattgactgccaactttgaaatgggtacagaaatttatagatgaagactttgtcctcaaggagctcgtgatatatcatgaaaaaccattcattcattaattaagcaaacatttattgaatatctaccatgcaccaggaacttgctagtctttgaagattacagggctttaatcaaacagatggaaaatttataacaaaatagagtatgtttctcttccttataaacattagtatttaggacaaaaatacattctctcattttgctgtgttttctttctcttacctctcccacctccactctccaattgttttctggatttttaaaaagatttatttatttatattaaattattaattgatatttaattatatattctattatatgtaaattagtatttatttatttatttagatttttttagcttggattatggatttgagttttcacattcatgaacatgtaagcaaaagcatgtgattgacagattttcttttaaggatctggggcagatatttaaaaaaactcaaaagttttcatttagttgtctttttactattgcctctaacaggcagtaatgggggaagtagcatagttatatgtatgtgtggctgaaggagtgggaagatgaggtgctgtgggggaatattagcttgtgaggagagttagagactagggactaggagtaggagacttattctagtgttggttttgttgttaacatatacactttgggtagatgtcttcctctttctaaagcctcagtttccttgtttagagaacagggttcatgattacaacttgttctatcttatattattgttaaacatgaaatgagataataaatatcagccttaaagtgcaatacaattgtaaagtatgttataaggccttaaccataaaagcctcagaaaatattgagggaatgaatgagtgtatctctttgagacgcagtcttgctctgttgccagggaggagtacagtggcacgatctcggctcactgcaatctctgcctcccaggttcaagggattctcctgcctcagcctcccaagtagctgggattacaggcatgcaccaccacacctagctagtttttgtatttttcgtagagacaggatttcaccgtgttggccagggtggtctcgatctcctgacctcgtgattcccccagcctcggcttcccaaattgctgggattacaggcatgagccattgtgcccagccctcttttttcttctgtttctgacatttactacaagaaataagcactgagagcccttgatcttcttttctgaattgtctagtattaataatttatagctataatttagtaatcatgtgctttcttgtatttagttatatttttgaatgagttagtctcagcacttcaaggacagtaactagggagcattcatattttctgtctcctagagtgcaaagtacattatagttgcttatcaactatttgttgattcattgatacttgttgaatgtgtagaacatccaaataaaacctttggagactaatgctaaagcaggatatatgcaagtgaatgagaaattggtatacagactgggagagatcatcatggaatggagttgataaaaaggacttgatagaaggttcaagggagatcctgacacagacctttaagacattgtaggtttagtttagggaacagaagggcatgccaggtggaggattagaggcaggaatgagaatggcattagaagatgaatgactggtggaatgacagtaaaggtcaaattggtaaaggtcaaattggtgaatttttaagactagtttacatttagcatagacaagtgacctccaaattttgtttagaagtgactcattgtgattatgtggactggcccttcaaggttgagacttaatgcttacaaagtcccttgcttctgcagaagaaatctctgaggctactgatgtcactaataatataatccatgtagagtgtcagaggttttttagctgttttgtagcctggttaagcagatagggcaagaattagcaatctttatggagatgtgccaaagtttgggttactctccttctggagggtgaagaggattcttattctgtagggattcgcacaatctgggagtggtggggggaaaggggtggtggtgactgcatgctggccccagctgtaggtgacttatagagactgtttcaagtggaaagaagagttgagtattggtgtcaacaggctggtgtttgtgtgggccagcaatttggtacttccaccctgaaataaagatatctttcttattcaagaattatagcaagttggcagatacccacctgtttcatgttcgctttcaatctagttgtatattagtaagtactggttatatattattaagtgctagtctggataatatttttacggatgagctgggcaacatgatgtagacatgattacctggtcttgagaatttcagtccagggctcttaaacctagataaacatagaagtgtgtatagacaagtttctgaaagtctgtctggtgtgtttataatggaagatgcagtagctatcttctgactttgaagaactgccatgcagaacagggatttgactactgtttggcctcaaaaactagcagtagagtaattgatgggtgaaagctcgtagaggcaaattctaacagtacttgagaaatagatttctttattagcagatgaaatgggctgcttaggaggtcactgttctctgtctgtcatagctattgatatggcagaggctattttttcctggtaatttgtgaaggaagtaccagcatcagtcagtggtagagctatcagagtgtctcaatcttggcctttggcattattgacattttgaactagtaattcttccttgtggggctgttctgtgcattgtaagatgtttagtagcatccctggcctctaccactacttgcctgtggttccctcagcccccaagttgtgacactcaaaaatgtctccagacattgccagatgttccctgaaggacaaagtcacacaggtatagaacctctgggctaggtgaaattcaggtaccttgcaactctggtaatttatacttttttgttttgcaagtgaattctgcatcagttttgcaaaaaaaaaaaaaaaaaaaaaaagtgaaaatggtttagaggacgtacataattgatgtggtggtgttctgtttccttccaattcagggagactttatggggatggaagtttttcagaatctttgagtgatattgtgataagcttggaattagcaggatattctctgtggcatactcttgggagagggctaagaggtgggaaggagaagctgagatgaggtttgagtggtgacacctgggatgactactgtgagggagaagaggagagaggagaggtgtgaattagttgtggcagccagatcaggttatggccatgcagagcactgaggttctcttcctgatggctgtcttcatccttcttctctcccaaagcctgtgcaattctatggtggaggagggagtgccggggaaattataaggtttaatcttgtcttgaattttatctcctccatttcctagttttgacctggccaagtttcttaacttcactgtgccatgattttttcattttgaaaaattgtggtaaaatatacataatataaaatttaccatttttaccattttttaagtgtatggcttagtggcattaaatacattcacattgttatgtaaccatcagcaccatccatttccagaaggtttttatcttccctaacttaaactctagctattaaacatgaactccccattccctgctcccaagcctctgacaaccgccattctacttgctatctctatggatcgaccactctagggatctcatataagaagaattacacagtatttgtctttcttcaactggcgtattttacttagcataatgtcttcaagggtgatcgatgttgtagcatgtgtcatgatttccttcctttttgaggctgaataatattccgttgtgtgcatataccacattttgtttccattcatctgtcagtggacactttaggttacttccatctttggctattgtgaatgatgttgctgtggcattttgtttcattttcattgtaaaatgattttaaaaatgatgcttcttccacagcattctggcgtctgtccggaaaggctccttgtagagtgctgggcactggctcatgacaagtgacacttcctctcctcacctttttgatgccagttcattgcacttttgcttcttttatatctccctcctagtataagtaatcaaacccaagcagagactaatgaattttttttcctgccaattcaagtccattctaggactgacaatttccaatcaatgttctctgaactgtttgtttcaagacttgtcaatctttggaaatttgagtttgttctaatactgccttatatgttcagattctctttaatggctctgagagtccttcactacatctcattgtcacacttttctctaaaataatttgttacaaagccttgctaatggcagcttgctgtgcccagggtcctgaggtcccatcatcaagcgttttgattcatctgccattcaaaagactattgatatcccgagggcaagttttagatacacttggtgaatttcattgctttgttcctttgacttcttcctttaaacacccaggctccctccctctgtgggaaggcgtatttttggctggcccttttctatctgtgccatctgtcacaggggtcattgctcagaggctggcttcctccctgctgggagcaggtggtcaaaaccaatggctattgacagggtcaggttggggcagcttaccaatatgccccccaagcagtatgcatagttgttctccttcagaggaacaagagaaaaccttacttgggggtctttttctgcattaaagaggaaaagatgcagatgtgattctaagttgtttatttattcttgtggtccagccacttgtcaactgtcctgtctgtgctcttgtctctgcgtacttcaactggcactgtactttaagaccactacctgaagcagcctccagtgtagcaagaaagcaacatctgtgaaaattatggatgatagaattactcaagctggttcatgtctaagatgctagaggtgatgatcctgacccttgatcaaacaaaataaaatatccctgaagtcttttgatgatctctggtggtaaagacccttgtttcaaagtcttatttaaagagatcatacttccaagcacttaggattctgggcctgactcacaggagagccagcccagtgctgagtcacttagccaacttcattcattgatcatgtgcttgtgcacagcttggtggcaaggtgggagagaattactattttcaaatgttccttcctgaggtagcatagtccagaagtaagccgagagacaggatgctagagttctattcttggcccagctatgcaccttcagcaagatacatccccccactcccctgtctaatataggaggatccagggaaataagtaaaacaagtaaaacagataagaacagaagagtttttaaaaatgcttttcccagagagattgtggctcccaaacccatgtctacctggctggccaggttttgttgccaccacttgttcaaggaacttaccatagcctccactgataaggaagaatttcttctcaaaaaagaaattctggaattcctagtctttgttctttttcaaacctttaatgcttctttcctgcagtactcattacattatgcttgggtatattaatctttttaaaagcacaaatgatttttctgctaagtgggggaaataattcagaacagtttatagaataagtagtcttagatatgtagtctcaggttactgaaatcagcttgactcagtgtaaggcattgctggtttctttggggcctccctagttctcctttgagttctagtgttggtgacactcccctcccttgtggctgtactcttatgtctaacctcagtcttagacataattatgctttcacctcttctcatccctggagaggtaaaatcttttctacctagtcctcaagcattgtccacaacctagggtcgtatcataacagttcttttggctgacacctgaaccacactgtagtggtatctctaaatttgctcatagaatttatttttaatatgaaatataagaagcatacagcaaactacagagaataatataatatttactttggatctttcttttatgaacaagagaattcatgatgagtacacttgaagcctgaggcatcatgtcaacccctcttcccagcgcttttgaacaagccttttttgttcaaggcactttgcagcatgcttagcttccctgtaggaagtctgccttccatgacctatattcattattctgtttcctccgtgcttttacagcccccttcccacccctcactgtgaccttgggcaaattgctttacctctctgagctttatatttctgatctatacaataagaataataacaacatctgcctgctctgggttattttgagattcaaatgtggtaatatatgtgaagtactttgttaagctctgtgttacacatggcatcgatcatctgtatagagaaactacaacactgttttccaagctgtggaagagggtgctgaggggaggagggtagccaagagatgtggtggatcagaatccccacaggctttttcaaactgctatgtatctaccagcactggtatgtgtgtgcatactcatatatacacatatacatttacacataacaattagaaatattttgaaggtagggaccaagagaaataatctcttatctagattcccttctagcatcttcatggttctgtgttttctctcccacatggttttaaactgggatggccaataaaaaatgtcagtttcttagtttctgtatgctcaaagttcacattttaagtaagaatcttcatcaactgtttggccgaggaggtttgctttcaccagttatgctttgtgaaattcctcccaccaattcttaatctagaatatgtctttctctccttggtgctctgtctttcctgctgcatgtggtctgaaaaaggttctcctgctgtgtaagcattatggatgcctcagcacttcctctgttgcacattaaacttgaaggcagttggggtgtactcactgttggatcgtttctgtaccactgtgtgtctgttctctatagacgtcctggttgctggtcattgtctcttagtgttccatgttgacgtaatcatatgcattttatttgattatgatactctggcgcagactagggaagagatactttggtgaaaatatctctgtataattgtatttaagaactattcagtcttatttttaaaaatatttgagattcaagaagaaatcttttaaggtcaagtgttgaaatcaaacacatcctagaacccattcattatactcctaggtatctacccaagagaaacttatgtgggcacatggcaatctggacaagaatgatgatggtaacattgttgatgatagcacatagttgttggaaacaaagttcacaacaggagagtggataaattatcttagagtcaaacaatgaactactgctatgggaaaatgcatgaattagaggaaaatgtatcaaaagaaataaatgtcaaacataatgtggaatagaaaaatgcaaatttcaggatatgatcaatatgttgcatttatataaagtttcggaacatgtaaaacaatctttattatatatggatacactgtttatatggcaataatacataaccctgcaagagagtgagagacacctaattcaggatagtagttgcttttgaggaggttagagaggaattgtatggaaaagaattatacaggggcgtccattgtgtctgcaaagttccattgctatctgtgtttttgtaatgcctgaaatatttcatgaaacaaatcaactgtaataaaaaggattcaaaatgcatatggaatatgatctacaacttttgaatgttattggcaaaggaaagaacatggatggatttcatagtggttctcttaagtattaggctcatagacggtttttaatttttatagttttctatatttttcaagttttctacaatgaaatacgtattattattattattattattattttttttttttgagactgagtcttgctgtcacccaggctggagtgcagtggcgcaatcgtagctcactgcaacctcctcctcctgggttcaagcgattctctggcctcagtctcccaagtagctgggattacaggtgcctgccaccacacccggctaatttttgtattttttagtagagacagggtttcaccatgttggccaggctgatcttgaactcctgacctcagataatctgcctgctttggcctcccaaagttctgggattacaggcgtgagccaccacacctagcaactgttagaatttttaaagccaaagaatgttatttaatattatgtaaaatcatgtgatgacaaatagtgatttttcaaagactgacagaatagtctacaagttaaccacttcctagtcagaagaaattctcatggggctagagacagactggatcaaactgcagagaaataaacaattttgatttgttgttattttccgtttatcttattcctaggacttcatacatcttactgttcgtagaacagatttctgtttcttctcagcttaggtatcacttttatagaaaactaccccttccctctctacaacacacttgcccctaatgctgggttatgtgaacctctagtaccttatccttaatgctgttgtgtcactgtattgtaattggctggtaatccatctatctctgctggagtttaagctccacaaggatgagatgtaggtattttgttcatcattgaatcagtgccaggacctagcacaatgtctgacacatagaaggcactggtaaataattcttgaatgaatgaatgaatgaatgaacgaaaggtcacaactgaaggggacattctggaaatggagccatttgatgggaaactttaaactaggtttttttcactcatagatacaagaatggtaattaggctgagtaataagatgtgagtaattctctctggaactcttaagtggaaaagtgaaaagccagagctttagatgctggtgccttactagaaataatgaatgagaaaatggggaaataaagaaagtacacccaagatagagataaataatatttaaaaagtttaaggtggttcctccttcattttatatgattttaagtgttgtttttaaatctggccttaaatgatatgatttcttaatattaggtatgttttatcagtattcattttttaacatacgtatagaaaaatgcaatgactgtatagcttcataaattttcagtgcacagcaacccagaagcccacccaacttctcctaatcactgcctttccccaaagataactactacacccagagtttttgtttttgtttgtttgttttgagacgcaatctcgctctgtcgcccaggctggagtgcaatggcgccatctgggctcattgcaagctctgcctcctaggttcacgccattctcctgcctcagcctcccaagtaggtaggactacaggaacccgccaccacgcctggctaattttttttctatttttagtagagacggggtttcaccatgttagttggggtggtcttgatctcctgacctcgtgatccgcctgcctcggcctcccaaagtgctgggattacatgcgtgaaccacagcgcctggccgagtatttgttttttaataatacaatctttatagaatgcctcaactcattatgtcgctgtttgtatctggagtaagtcagcctgttggcccatcaccatcttccacctagtaatctgaatgaaggcatggtaactttagcccaagaaggtgctccattttattgacaacaaatgtcatactcccaaatcatcatttaacttttattgcagagatcctaaactatgggtgtgtagaaaggtggagaacagaaatttacttatatagtttatttggcatgagctctggtttccttggatattgaagacctcaaaaccgctgggtctgttatttaagagatattaaaatcaatttgcttcccagtttcaacattgtctttcttaagacaatagctggataccattctttcatttatgccacaaacaactatccagtggcctactgtgttcctctttcagggcaaagcatgatgcaatgcaaggcattgtggagtatgtgatgattaataagcatactttttgccttaaggcacttataaccaagtaagcaaaatgaaatacataaatataaaacgatacaggcatacctcattttattgggctttgctttattgtaatttgcagatactgcatttttttacaaattgtaagtttgtgccaaacctgcattgattgagcaagtctgtcgggaccattttttccagaagcatatgctcactttgtgtctctgtgtcacattttggtaatttttgcaatatttcaatttttttcattattacatctgttatggcgatcagtgatctttgttgttactattgtaattgttctggggtgccatgaaccacactcatatgacagggaacttaataaatgttatgtgtgttctgactgctccactgaccagctgttcccccatctctctccctctcctcaggcctctcttttccctgagacacaacaatattgaaattaggccaactaatagccttacatggcctcttagtgttcaagtaaaaggaagagttgcacatctgtcaccttaaaggctagaaatgattaaacttagtgaggaaggcatatcaaaagccaacttaggccaaaagctaggccacttgtgccagttaaccaagttgttaatgcaaaggagcagttcttgaaggaaattaaaagtgctactctagtgaacacacaaataataagaaaatggaacagccttaatgctgatatggagaaagttcgaatggtctggatagaagatcgtactagccacaacatttccttaagccaacgcccaatccagagcaaggccctaattctctccaattctatgaaagctgagggaggtgaggaagctgcaggagaaaaattggaagctagcacaggttggttcaagagatttaagcaaagaagtcatctccagaacatgacagtataagatgaaggagcaagcgctgatggagaagctacagtaagttatccagaagatctagctaagatcattaattgatgacggtgaccatactaaacaatggattttgaatgtagagaaacaaccttctattggaagaagatgccatctaggactttcatagctagagaggagaagtcaattcctggcttcagagtttcaaaggacaggctgactctcctgttaggagctaatgcagctggtgactttaagtggaagccagcgcttatttaccattctgaaaatggtaattaatggcctcttaagaattatactagatctactctacctgtgccctataaatggaacaacaaggcctagataacaggatggtttactagatattttaagcccactgttgagacctactgctcagaaaaaaaagatccctttcaacatattaatactcattgacagtatacttggtcacccaagagctctgatggagatatacaaggagattaatgttgtattcatgccagctaacacaacatccattttgtagtccatggagcaaggactaatttcaactttcaagtcattagtttaaaaatacattttgtaaagctatagctgccataaatagtgatttatctgatggatctgggaaaagtaaactgaacaccttctggaaaagattcatcattctacaagccgtaagaatatctgtcatctgtgagaggaggtcaaaatgtcaacgtcaacaggagtttgaaaggagttattcctttttttttttttttttgagacggagtctcgctctgtcaccaggctggagtatagtggcgtgatcttggctcactgcaacctccgcctcccgggttctagcaattctgctgcctcagcctcctgagtagctgggactacaggcgcgtgccaccacacccaactaatttttgtatttttagtagagacggggtttcaccatgtcggccaggatggtctcgatctcttgacttcatgatccaccctcctcagcctcccaaagtgctgggattataggcatgagccaccacacccggccggaaggagttattctaaccctcatgaatgactttgaggagttcaggatgttcatgggggaggggactgcagatgtggtgaaaatagcaagagaactagaattagaagtggagcctagagatgtgactgaatggctgcaatctcatgataaaacttggacagatgagaggttgctttttatggatgagcaaagaaaaaatttttttttcttttttgagacagggtctcgctttatcgctcaggcttgagtgcagtggtgcaattatggctcactgcagccttgacctccgggctcaagtgatcctcccacccagcctacagcatagctgggactacaggcttcagccaccacacctggctaatttttgtatttttttgtagagacaaggttcaccttgttgcccaggctggtcttgaactcctgggctcaagcaatcttcctgccttggtctcccaaagtgctgggattacagcgtgctgggctgaaagtcgtttcctgagatggaatctcctcctggtgaagatgctgtgaggattgttgaaatgacaacaatatatttagaatattccctaaacttagttgataaagtagcagcaaggtttgagaggactgactccaattttgttttttcatttttgatttttttaagagacagggtcttgctctgtcacccaggctggagtgcagtggtgtgatcatagctcactgtagcctcgaactccctgactcaagtgatcctctcacctcagcctgccaagtaggtgggactatggctgcgcgccacagcgtgactcaaattgtgaaagatgctctgctgtggataaaatgctgtcaaataccattgtatgctacagagaaatcttttatgaaaggaagaattgatcaatacaggaaacttcattgttgtcttattttaagaaattgccacagccatcccattcttcggcaaccaccaccctgatcagtcagtcagtagcgatcaacattaaggctagaccctccactagcaaaaagatgacaactagctaaaggctcaggtgatcattagtttttttttttttagcaataaagtattttttgaattaaggtatatgcttttttttttttttttttttttttttttttttttttttgagatggggtctcagcttgttgcccaggctagcaggctagaatgcagtggcccaatcatggcccactgcagcctggacctcctgggctcaagtgattctcccacctcagctgggtagctgggactatgggtgtgcaccaccacactctgctaattttttttaagtttttttttttttaaaacacaggatctcactgttgcccaggcttgtcttaaactattggcctcaggtgagcctcctgccttgacctcacaaagtgctgggattacggatatgagcacatgtgcctggcctgtttttttttttttttttttttttttttttttttttttttttaagacagtgctttcctacacttaatagactacagcatagcataaacataacttttttatgcactgagaaaccaaaaaaattttgttactgactttattgtgatacttgctttattgtgatggtctgcaactgaacccacaataagcctgaggtatgcctgtactttaaagtagtcttttagaagtgatctaaaataccaggagggagggagagatttttttgatcgggggaataagaacaactgtcccaagcagcaataggtaagatagcattctaatcaataggaatggcacagccaaagtataggagtggaaaagtacagtaggagtgaggaatgtttttgtgaagcattagacacagcagagaacgtctattaataggttgaaaagagccctgagtgctcatttaaggatttgggactttattcaacaagcagtggagagtctgatgtttctgaggagaaaggagccatggctatcaaacccaaatttcctatcagttcctgattcatccaccagccaaggttactgtaccatccttgaggttgaatgggttgggccaactttgaggattagtgaaatcatttgttattaatgaagtggttaacaatagttaaggtaccatagcaattatgaacaatcttctgctttaatttttcctataggatcttttgctagtcaggtcttctctgagtagatgagagagataactttgaatgtctcagggaaaagtgtaagagtaagttttgttaatacaatacaagtgagattggaaatgcagttggctctccatatatgtagtttctccatccatagattcaaccagctgcagatcaaaaatatttgagagaaaaaacagtaaacataacagtaacaatttaaaaatacagtgtaacaactacttacatagcatttacatcatattaggtattataaggaatctagagattatttaaagtatataggaggctgtgcataggttatatgcaaatactacaccattttacataagagacttgagcatctggggattttgctatctaaaggggtagtgggagtgtgtgtatcttggaaccaatccccaatggataccaaggaaggactgtatattcttatcttactaccactgtgcttgtcccattttaacatttttttcctgaatgagaaataccatactgtacagaaaaagcacacagaatggagagtcaactcctgagtctaagtcctagctctgctacttattatctttgtgaccctgagcaagttacttaacctttgtgatcttgtcatatggtggtcaggagattccagttggaaagtagttgtgaaagcgttattaacaaaattgggatgtatgttgccttgcttgtatttggagatagtgtctcctgtaggcattgttgacattgtttatagatgcttctgaaaagaccaatgcgtgtcccacatcttatccactgtgcggaaaggcagtgtgttatagaaaatcactggggcggagagcagaaacactgaattccagcccacaccctcgccctggcagtgtaactcaaatgtaactacgacctcataagctaccctgcttttgtcttaaggtactgtaaaaacaaaccacaacagtcacaaaaacaacagaaaccacacacacacactcaaatccaattccaagagacttaaagaacaatgcagatctttactgcagaactgttgaaaagaagatgcattggacattctactactagacttcttaaaaagtactttttgcccgcattgtatactagttgtcctatctcctttttttctttctcctgcaccatgctctgcagcgtcagccacattcctagattgctccttcctggtctttttatcatcgccatcattatcattatttggagagttttccactttctcttcctacctattgttcttttcagcatgaggaaaatagctaattgaaacaagaaaggaatgatctcttcttagaaaatggacagggggccacttaatattttgtgcctgtgtcactggctgtttgatacaaggcaacctctttaaccccaaacaccttttctatgaagctggatctagcaatatttgctcctttaaactccaagtaaggatgagttgaggtaaaggataaggaaatgctctgtaaatgtgaaagtgctacacaaatatgaggttgcattaatttgtagcctcagcaactgtttgcaatttgtcactctgtcagctcaatctcctaagatcctaacaagtctgaaatcttgttcaggaaagttccactctgtttttaaacatggctggccacaatggattgttgctgccattcatagctaggctactgtgtatttttatatttatagttgtataccactttgtaagcttttgacaaacattctctcatctgatcctcacagtaccctgtgagatgattttatcacccccatggttgaagctgtacctcaggccttcccatcgtttttgcctcttctgcttgcagtcaccttatttgagcaccactacagctacttgggtaacccatcttctcttcccctgtaaaacttcatggaagtgtgagtgatgcttctgaatttggtcagtaggatatcctccagtaaccatttagtgggtctgctcttcggcctgttagcgattgattcagcaaatgaagagcaaaaagcacacaagcctgacatggcctttgtgatgcctgtatccagacctcttagccacactgaaggctgggtggcctcacagcttttgatttggagtttgatactgctttaatgccctattgtttaacccagtttcacagtaatcttttttggtcattgaagcaacctgagtgttccctgagtagcaaaacttcatgcgagctgtcagttctcttgttgtcattagaagtcttcatcttgttttcagagcttctttggagtaggaaagaacttgatcttagtcattgtcaaagatgtcatggtacggggaaaccatgtggactttggagagaggctgctcccaatgtgatcctaggaattcctgtattttcttcgagttcgagtctcgctttctttcctcacttgtaaaatgaagctgtcaatgcctgcctaattaggttgttatgaagattaaatgtaatgtttatagagtgcctgtatatattattataacgttcataggtattacacctgctttgcagacaggcccataaccactttgatgtatcttctttacttctgtgtatttgggcatgtctatttcaagagaactttattaataattaactccagatctatattgttgaaagaattgccattgaattctggagttgaaagagagccaattgttattttgtataatagtttagagccatccttatactctgcaggagaggaatgaggtggacttgaggctcattcctgcttcaaccagagcagaaatacttttgtctatattatatattgacatttcgtgtaagatatgatttgaaaaagacttccactgattgaaaacaaaaaaactttaaaattttaatgcaactttcttcctgtccaacacagaaatctattttccaacatgacagacagataatgacttaggagagtgtatcctgataccaggtttaaattctaagctttccaatgcctggaatgtagtagacactcaagacgttctcattgaatggataaattcttggtcattgttgtgttaagggtcaactgaatagtgctggacctttgttctagcctgtgtgtgtgcattttcattactttattcaacacataattatttgatgatagattttgagtcaggatattattccttctgatttaagcctctatgttttttgaatcttggtaatcctgtcagagaaaccaactttgcttagtctgtcagattccaggagagctcattagttaagtgttatgatctcatagggctggaagggatcttagaagggatgcagggaagggaaagagtgctgatcacaagaagtagtgtggtgaagttgttaagaacacagaccttggggccaaacagcttgggtttgaatcccactgtgggacgtagagcaaagttacttaacctctttatgcctcagttttctcatcagtggtatgggataagaacaataactacctcagagggttgttggaagcatcaaaagaattatatataaagctattagaactgtgtctagcaggtagttagtggtccatattaaaaacaattattggaagtattattaagacctacttcagacggagtctcgttcactcagtgctcaatggtgcccaggctggagtgcagtggcgtgatctcggctcgctacaacctccacctcccagcagcctgccttggcctcccaaagtgccgagattgcagcctctgcccggccgccaccccgtctgggaagtgaggagcgtctccgcctggccgcccatcgtctgggatgtgaggggcccctctgcctggctgcccagtctggaaagtgaggagcgtctctgcccggccgccatcccatctaggaagtgaggagcacctcttcccggccgccatcacatctgggaagtgaggagcatctctgcccggccaccccgtctgagaagtgaggagaccctctgcctggcaaccgccccgtctgagaagtgaggagaccctccgcccggcagccgccctgtctgagaagtgaggagcccctccgcccagcagccaccccgtctgggaagtgaggagcatctccgcccggcagccacctcgtctgggagggaggtgggggggtcagccccccacccggccagccgccccgtccgggagggaggtggggggatcagccccccgcccggccagccgccccgtctggcaggtgaggggcgcctctgcccggccgcccctactgggaagtgaggagcccctctgcccggccagccgccctgtccgggagggaggtgggggggtcagccccccgcccggccagccgccccatccgggaagtgaggggcgcctctgcccagccgcccctactgggaagtgaggagcccctctgcccggccagccgccccgtccaggagggaggtgggggggttcagccccccgtctggccagctgccccgtccgggaggtgaggggcgcctctgcccggccgcgcctactgggaagtgaggagcccctctgcccggccaccaccctgtctgggaggtgtacccaacagctcattgagaacgggccatgatgacaatggcggttttgtagaatagaaaggggggaaaggtggggaaaagattgagaaatcggatggttgccgtgtctgtgtagaaagaggtagacatgggagacttttcattttgttctgtactaagaaaaattctgccttgggatcctgttgatcggtgaccttacccccaaccctgtgctctctgaaacatgtgctgtatccactcagggttgaatggattaagggcggtgcaagatgtgctttgttaaacagatgcttgaaggcagcatgctccttaagagtcatcaccactccctaatctcaagtacccagggacacaaacactgcggaaggcctcagggtcctctgcctaggaaaaccagagacctttgttcacttgtttatctgctgaccttccctccactattgtcctgtgaccctgccaaatccccctctgcgagaaacacccaagaatgatcaattaaaaaaaaaaaaaaataataataaaataaaattaaataaaaataaaataagataaaagtaaaaaaaaaaaaaaaagacctacttcatatgtatcagctcatttaaccctcaaatgaggaaatggtggcccccaggaacacattcaggaacacattagtctacaggatgccactaatggattttaaccaaggaagttaatttatcagaattatatcttaaaagtcactcttgggacaggtagactgaaaaggaatgaaactggaggcagtgagaccagttagacggttgctatggtaactatacaagaagtagagaggctttttctggaaagagttagatggtaaatatttcaggcgttgtgggccacacagccttttgcaactactcagctctgctgttgtaacacagaagcagccacagacaatacttacataaatatggctgcattctagtaaaacttgacttacagtgtttcagtattggatgacaagatttgaagatggatggtgccaatgacagtacaacaatgtgaatgtattccatgccatggaactgtatacttaaaaatggctaaaatggtaaattgtatattatgtatattttaccacaaaaaaactttgcgagaagaaatgaaacacttttcagttttagaacaatgggcaggaaaaattgtttgcatcataacttgaatcaaggaaactggtgaagaaaatgagaataggtgtggaggcaacagttagagactacaaagggttttctgtttgcgatataaagggaaaggctcttgtgccttgtggaagaaataaaagaaacatggaaagcaagttaaatttgaagaaaaaataggtgatgggggaatggggaggtaaagtagaggacagctttgagttgtagtgtctgcagggccagggagatggaagcggatactagtagcttggtagctatatggatgtatgtacaaggtggaaacctgtcttcattttggactgatggaacttgctcagagaagctttgggatacatttataataattgtaatcattggattgacttcattaaataggatccattcttaagaatctttcttagtgtttttagattaaaatatctaagtactaacttggtggcatgcaccagtagtcccggccacttgggaggctgaggcaggaggattgcttgagcccaggagtttaagtccagcctgggcaacacagtgggaccccatctctgaattaaaggaaaaaaaaatatccaagtactcaaagtgtaagtgcttcacaagtatctcctgatagttacttatggataatcatacatttatatccacactaggcctctgtcaaagtattctatgaacagatccttcaaagggtttctggggggaagacatacatgtaagtcaaccaagagtgactatctcaaaaggattaggaagagaccagttcttgagaatcagaggcaaatagctcagctttgggtaaattgggtggagtgagacgtaggaaggaaagacttcagaagtggcctcactaggtggaaatccagggagacagatgaatgcacgggcagttccccattaacatggttgcagacattcaccaggctgacgaggtccctgagacctggtaggagggagagcagatcttgatgttatcaagagaggctgaagactgcagtttttaatgtttaaaatgattaaaactaagctttgacaaaattgtttttatttcaatttctagaactgttatagattttccaaagtgaaatctgtaagtaacaaagtagaaaattggcagcgtagcatctcgttgtcattggagaagggaggagcccagggagtggggacagtgtaaaccatccatctggctgcatttttctcttctcctatgataagagccacccaccttcctcccagtgcagagctgctcctctggctaattccaaatgagctatatatcaacatgcaaagagctgtctcgcagtcctcagagtctagctatagttctgtaccaagcttcttatcttcttttgcttcttgctattaaatctttcccttgaggagcaaagatggtggcgtgaggtgtgctgctgcaaactggcctctggggaggagaagggtgggggccactgactcatgggaggccaaatacatctacaggattgggggaggttctttatcacaggaagaatttttttcataacctttagatgcttttacattcatctcaaaaacaaaattctgaactcagaacttggaaaagatagagtctcactatgttgcccaaaggctgaagagcagtggttattcacaggtgtgaccatggcacactgaggtctcaaactcctggcctcaagaaaccctcctgcctcagcctcctcagtagctgggactataggcatgccaccacccttggtgtgtaaatgtgtctctatgtagtctcctagagtgggtagtcctgtttcttttattaagccactttctctattcttgaaatgtggatgtattagtccgtttttacactgctgataaagacatacctgagactgggaagaaaaagaggtttaattggactcacagttccatatggctggggaggcctcagaatcatggcaggaggcgaaaggcaattcttacatggcagcagcaagagaaaatgaaaagatgcaaaagcggaaacccttgataaaaccactagatctcgtgagacttattctctaccacaagaacagtatgggggaaaccacctccatgattcaaattatctcccaccaggtccctcccacaacacgtgggaatcatgggagtacaattcaagatgagatttgggtggagacacagctaaaccttatcattccacccctggcccctccaaatctcatgtcctcacatttcaaaaccaatcatgccttcccaacagtcccccaaagtcttaactcatttcagcattaacccagaagtccacagtccaaagtctcatctgagatgaggccagtcccttttgcctatgagcctgtaaaatcaaaagcaagctagttacttcctagatacaattgggggtacaggtattgggtaaatacagctgttccaaatgggagaaattggccaaaacaaaggggttatatagggcccatgcaagtccgaaatccagcggggcagtcaaattttaaagctcccaagtgatctcctttgactcaaggcctcatgtccatgtcacactgatgcaataggtgggttcccatggttttgggctgctccacccttgtggttttgcagggtacagccttcttcccagctgctttcacaggctggcatcgtgtgtctgtcttttccaagcgaacaatgcaattggtcggtagatctaccattctggggtctggaggacggtggccctcttctcacagctccactaggcattgccccagtagggattctgtctgagggctccaaccccacatttcccctctacactgccctagcagaggttctccataagcgtcctgccccacagcaaacttctgcctgggcatcccggcatttccttacatcttcttaaatctaggcagaggttcccaaaccccagttcttgacttctgtgccctcgcaggctcaacaccacatggaagctgccaagtcttggagcttgcaccctctgaagccttggcccgagctctacattggcccctctcagccacagatggagcagctgggacgcagggcaccaagtccctagactgcacacagcatggggaccctgggcccagctcacaaaattattttctcctaggcctctgggcctgtgatgggaggggctgctatgaagaactctgacatgccctggagacattttccccattgtcttggagattaacattcgctcctgattccttatgcaaatttctgcagccagcttgtatttctcctcagaaaatgggtttttcttttctatcacatggacaggctgcaaattttccaaatttttactctcagcttcccttataaaactgaatgcctttaacagcatccaagtcacctcttgaatgctttgctgcttagaaatttcttctgccagataccctaaatcatctctctcaagttcaaagttccacagatctctagggcaagggtaaaatgccaccagtctctttgctaaaacgtagcaagagtcacctttgctgcagttcccaacaagttcctcatctctgtctgagaccacctcagcctggaccttattgtccatatcaccatcaggcctctggtcaaagccattcaacaagtctctaggaagctccaaactttcccacattttcctgtcttcttctgagccctccaaactgctcccatctctgcctgttatcaaattccaaagttgcttccatgttttcaggtatcttttcagcaatgccccactctactggtgccaatttactgtattagtccgttttcatgctgctgataaagacatacccaagactgggaagaaaaagaggtttaatttgacttacagttccacatggctgaggaggcctcacgatcatggcgggaggcaaaaggcactctttttttttttttttttttttgagacagaatcttgcactgtcgcccaagctgcagtgcagtggtgtgatcttgttgggagcaggccccccaaaatctggccataaactggccccaaaactggccataaacaaaatctctgcagcactgtgacatgttcatgatggcaataaagcccatgctggatggttgtgggtttaccggaatgagggcaaggaacacctggcccaccccacattgggcaccacttgccgagaccagctcggtcggggagaccctaacccagcggtgctagaggaattaaagacatacacacagaaatatagaagtgtgaagtgggaaatcaggggtctcatagccttcagagctgagagccccaaacagagatttacccatgtatttattaacagcaagccagtcattagcgttgtttctatagatattaaattaactaaaagtatcccttatgggaaatgaagggataggccgaattaaaggaataggttgggctagttaactgcagcagcagcatgtccttaaggcacagattgctcatgctattgtttgtggtttaagaatgcctttaagcggttttccaccctgggcgggccaggtgttccttgctctcattcccgtaaacccacaaccttccagcttgggctttatggccatcataaacatctcacagtgctgcagagattttgtttatggccagttttggggccagtttatggccagattttggggggcctgctcccaacagatctcggcctgctgcaacctccacctgctgggatcaagtgattctcctgcctcagcttcctgagtagctggggttacaggcacccgctaccacgcctggctaatttttcatatttttagtagagatggggtttctctatgttggccaggctggtcttgaactcctaacctcgtgatccatccacctcggcctcccaaagtgctgggattacaggcatgagccactgggcctggctggcacttcttacatggcagcagcaagagaaaatgaaaagatgcaaaagcagaaacccctgataaaaccatcagatctcaggagacttattcactactatgagaacagtatgggggaaaccgcccccatgattcaaattatcttccacctggtcattcccacaacgtgggaattatgagagtacaattcaagatgagatttgggtggggacacagccaaaccatatcagtggatatctcttctctacatattacctctatgatttggaagaggccacttttctttttgttccacctcccagattcaacactgagagctcctaggattgcagttatcctgccaatttctccagggaagaaagaagcagctgacatgggtcttctgctactacttaattaattataaagaaactattttttaagctgaagctgatgtttcacttgaaacaggtgcttagctggtggtatttgtgaattcttgactttttgttccaaacaagaagactaaataaatagcaagtatggatgagaaaaaaaatcattcccttgagcttcagaagcacatatccttgcctatatgatgcctccatttggaggtaccaaagcacctcagactcagcatgttcaaaagtagtcgtcatcttaatctgaacactctgcatcacatcttattccttgtctcggtgaatttcactacctttcatccctttgctcatgatttgggtgtcttccttgatttttcttatatcttaccttcctcttttagtcagtcaacaaaactgtccagtacatcatccctgctgctgctgcctggcttaggctgtcatctctcagtggaattgctacaatagtcttcttcctggtcttcctcactctgatcttattcctccctcgtctcattctatacactatagctagaataaccttcctaaaatgtaactttgatcaaatcattttctactttgaagctttcattatctccttattgccttcagaataaaagcaaaactccttcccatgagagcagggctttgcgttcctgatgtggtacttgattacctgttagctgattttctgaaatccacttacactgaactgcttgcagtacccaagtggcttgatctctcttgtctgtagtctcttaaatatgctatttcttcttcctgcaaagctctacccattttcttcccataacccaccctaccagcccatctgtgggctttagctaagatgttactttctccaggaagctttccttgatctctccaaacttggttagatgtcttgcttgtatattctgtaaaagcctgttttactttattggaagatttaccatactgtactttgcctgtttacctgtctactacctcagaagactataaggtcctttgaaggcaggagtgagctattattcatcagagtatcatttatggcatatgttggggggcatagtaaatttttttctctcttttaaaaaagattgtagtaaaatatccataacatttaccatcataaccatttttaaatatataaactgggccaagcgaggtggctcacgcctgtaatcccagcactttgggaggccgaggtgagtggatcatctgaggtcaggagtttgagaccagcctgaccaacaaggtgaaacccagtctctactaaaaatacaaaaattagctgggtgtggtggcaggcacctgtagtcccagctacttgggaggctgagacaggagaattgcttgaacctgggaggcggaggttgcagtgagccgagatcatttataaactttttcatcatcccaaactgaaactctgtaccaactaaatagtaacttctcattcccacacagcccctgattacccctattctgctttctgtctttatgcatttgactatttttggtacctcatataagtggaatcatacaatatttgtccttttgtatcttgtttatttcacctagcattttttcaagattcctccatgttgtagcatgtgtcaggatttcttgcctttttaagctacataaatatttcattatgtgtatatttcacattttgtttattcattcatccatttgtttatctcatagacatttgggttgtttccaccttttggcagttgtgactaatgctgctaagaacatgtgtgtacaaatatctttttgagttcctgctttcagttcttttggaattgctggatcttatggtaatcctatgcttaatttttttgaggaactgcagtaatgttttctatagcagctgcaccattttcctttaccaccagcagagcactagagtctgaatttctcaacatcctcacccatacttgttattttctgtgtgtatgtgtttttaggtaagaaattttgtttgtgttttaatattagacatcctaatgggtatgaagtggtatctaattgtgattttgattcatattacctaatgattaatgatgctgaacatcttttcatgtgcttattggtcatttttatatctgtttttggagaaaagtctattcaagtcttttgtctatttttaactgggttttgttgttgttgagttttaggagttctttatatattctgggtattaattctatcacagatacaatttgcaaatattttctctcattctctgagttgccctttcactctcttgatagtatcctttgagcaacaaaagcttttcattttaataaagttccatttatctattttttcttttattgcctatgcttttggtgtcatgtctaagaaatcattgacagatccaatgttatgaagcttttcctctatattttcttctaagagttttatagttttagctctgacatttaggtctttgatccattttgagttaatttttgtgtatggtataaggtaaggatccaacttcgttcttttgcaagtgaatatccagttttgccagcaacatttgttaaaaagattgttttccccattgaatggtcttggttcccttgtcaaaaatcatttgaccatatatgtgagggtttatttctgggctctttaatctattccattagtctatatgtctgtctttatgccagtaccacattgttttgattactatagatttgcagtaagttttgatatcaggaaatgtgagtcctccaactttgttttttttcaagtatgagacaagatctttttaattttccccttaaatcttttgtagatatggggtctcactatagttgaccaggctggccttgaacacctggcctcaagcagtcttcctgctttggcctcccaaaatgctgagattgcaagtgtgagccactgcacccagcctgagacaagaccttagtgctacttgctaccttttatttgatcttccttccaacatatttttttgtgtgttctagcataaagtgtttgctttatttatgtatttattgagacagagactcactctgtcacccaggctggagtgcagtagcatgatctcagctcactgcaacctccacctcccaggttcaagcgattctggtgctttagctacccaagtagtttggattacaggcatgtacaccacgcccagctaatttttgtatttttagtacagatgggtttttgctgtgtcggccaggctggtttcgaactcctggcctcaaatgatctgcctggctcagccccccatagtgctgggattacagcactttgtaaggcatgagctactgcacctggccctggagtgtttgctttaacagcccctcctagatgacttctcttcctttcattcctgttataaccgcatttcaatactggttttcttttcctctttcatgccgtatcccttaatttggattctactttgtttgttttcaggccacagttggcattttaaggtcttttttttttttcttttttagagacaggtttagacacaagagttgtgtcacccaggctgtagtgcagtggcaccatcatggttcactgtagcttctaactcctgggctcaagtgatcctcccatctcagcctccccagcagctgggaccaccagggggacccagataaatttttttactttttgtagagatggtatcttcctatgttgcccaggctggtatcaaacttctggcctcaagcgatcttcccatctaagcctcccaaagcactaggattacaggcatgagtcactgtggctggttgcattttaaggtcttaatcctcctggacaaccaaaagaagagagctctacccatttgtttgtccgtatctttgagtctagttcaatctcttttagaactgccagagccaaaagttgcttttgttgttgctttatcatctgggctgcctgtgtcatctttagcactgcaggaagatgcacaatgatatagtagtggtatgatggacaaggtcattgacttagagattgggtgttttagtgaaaagaacattatattggaagccaagaaatgtgggtcatagctttgcatctgtcactaattggcagttagacaattcagcctctcttatataacttatggatgaagcaaattgaactaggtggtctctaagttccttctaaatctaacaagtttgttactgtgcaagatgacttcctcagaaacagaagacatttaagcttgttttaatggatgaatagaacttaggtaagaaagaaagatgtcaggaggtttaaaataaggaaatgaaaatatttttaggagtcagtgaaagtaacagtgaccagacattttattaagtgatgctgggtcaattatataaccacaggaaaaaaatgaaccttgtcctccacttcacaccatatgtaaaaatcagttcatatggttttcaaaactaaatgagaaaggtaaaataataaagattgtaaaacaaaggagaatacctacatggccttggaataggcacagatttcctagaataaaaaagtactaacatgaaggaaaagactgatagattggatttcaattaaattaagaactacagtttatcaaatgacaccattaagacaggtgaaagagaggaaatatttgaaatattatatctgacaaatgacttcaatggagaatacacaaagaactcctacaaactagtaagaaaaatgcagtttaacccaataggaaaaagtgagcaaagacttaaatcagcacttcacaaaggaggatgtctaaatggccaatcaacctttgagaaagtgctcagtatcatcagacaaatacaaatttagacaagagtgagccaacaaacatgagaaaatgcatcagtatcattagggaagtgcaaatcaaaaccacagtgaggtaccactttacacccattaggatggcagcaattaaaaaaaaaaggtaagaaaaaggaaagtaacaagtgttggcaaggatatggagaaattgtaaccttcttatattgctgttgggaatacaaaatggttcagccacaaaatgcaaaatagttcaggaaaacagtttggcagttcctcaaaaaagttaaatgtagaattaccaattccactcctaggtatagactcaaaggaattgaaaataggtactaaaataagtacatgtgtgtgcactgttcatagtagtcgaaaggtagaaagaattcaatgtccatcaacagatgaatgtgtaaacaaattgtggtatatacatttctgggatatatattttggggtttttttggttttattgttatttttaattggcacataatatgtggtacagtgtgatattttgatacatgtatacaatgtgtgtaatgatcaaattagggtaattggcatatccatctcaaatatttatcatttctttgtattaggaacattcaaaatccacttttctagcaatttaatccacttttcaaaatccacttttctagcgtttccctgatgataagtgatgagcatttttacatatacgttttggccatttatatgtcttgttttgagagatgcctattcagttcatttgcctatgttttaattgaattatttagtcttttgctgttgtttgagttccttatatattctggatattaatcccttgtcagaggaatagttcacaaatattttctcccattctactggttgtctcttcactctgctgattgtttcctttgctgtgcagaagctttttagtttgatataatcccatttgtctatttttgcctgtgtttcctgtgcttttgaggtcttctccataaaatctttgcccagaccaatgtcctgaagtgtttctcctatgttttcttctagtagtttcatacttacatgtaagtccttaattcattttgagttgatttttgtatgtggtgagagatagggttccagtttcattcttctgcatatggttatccagttttcccggcaccatttattgaagaaattatccctccccaatgaatgttcttggtgcctttgtcaaaattcagttggctattaatatgtggatttaattctgtgttttctattcttttccattggtctgtgtgtctttttatgctaataccatgttgttctggatactgtatctttgtagtatattttgaaatccaatagtgtgataactccagctttgttccttttgctcaggattactttggcttttcaaggtcttttgtggttccatagaaattttaggattgttttttctatttctgtgaataatttcattggtattttgataaggattgtattgaacctgtagatggcttttggtagtatggttattttcacaatattaattcttgcagttcatgaacatgggatgtctttccactttttttcgtgatctttgatttttcatcagtgttttagttttcctggggagatctttcaccttcctggttaaatttattcctaggtcttactatttttttggtggctattataattgggattgctttcttgatatctttttctgctagtttgttgtggatgtatagaaatgctactgatttttgtacgttgattctgtaccctgcatgaaataccatagaaaggaatgaagtactgatacatgctacattatggatgagccttgagaacactatgctaagtgaaagaagtcagacacaaaggctcacatattgtgtgagtccatttatatgaaacatctagaatagataaatcagtagagagagaatgaagactgatagttactaggagctgatggcaaagggtggaggctgaaggttgggaatggagagaaactgcttatgttttagaactagataactggtagtgtgaatgtactaattgccactgaattgttcaccttaaagtggttaattttatgttatgtaaatttcatcccaataaattacttttttaaaaaatacgagatgctgctatacccaccagagtgggtaaaattcagaagactgtcattccaagtgttgatgaggatttggaacaactggaaccctcttatattgctggtaggaatattactaaagctaaatatctgccattctctgacccagcattccactcctagctatatgccaaaaagaaataaaggtatggctaccaaaagacacctacaagaaggctcttagcagcttttattcataatagcttaaaactataaacaacttagatgcccatcaaggtagaatggataaattgtactacattcatacatggaatactatgcagaatgaactactgctacacatataagaagatgaatttcacagaaatagtgttgggtgaaagaagccagaacccaaagagtatgtttatagtctacttatatgaggttcaggtagaagcaaaactaatatatggtgataactgtcagaatagcttttgctaggggtggttattgatggctcatgagagctgtctgggatgctgaaagtaaattttctacatcttgatttcagtggtggttacacaggcacatacatatgtacaaaataagctgtgcacttaaggtttgcatactttattgctttattgtattataccttcaaaaaatttaaaactcagaatacagttaccagcgcaggtgttcttgcagagaagccatagaagagaatgtttatgatgcatacggttgtagaatgccttgagtggcagtgaaatggcagaaatttggaatctgtcctataactggtaagtttgagggagggaaatgacttaatgaaagcagatttttagggaaatggatccttggaaatatgcctggtggactgagggctggtccactagtggggagactactgcagtaggacctgaattagggcaatactattgaaaatggaagctcttaaggaagagcaattggaaaggctgatgactggttgggtatggatagcaaaaggggacttggcagagataactgagattccatgctggatggctggtccagaaataagaaagccaggaagaagaatcagttttggagtaagatgaatagtttttaaatgctttagtttgaaagtcatagcaggccactagagacagctgtgggactcaggagaacaatcagggccagtgagagaaatctgaaaattaccaatgtacaaattattttgaataaatgagatccctaaaagactgtaacagagaaatttcagaaggccagcccttagagaaggaagagttgatgaggaagaggagactgcagctgtagttcaaccaggataacatagtgtcacttaagccaaacaaagagagtgggggtggagatgtaaagggagaagatacaggaaaaggtacaattgaagatgggatgaagagcaggggtaatcgagaggacacagtcttggagaagacaggaagaaacaggatctgaaagcaaagacagagaacttagccctgggctgctgtgaagaacccaggtgcctgcaattagatcacaagaatttgtcagcatcttcagtgacttactcttgcagtgttctttgtcctctgcgtaggaacagagagtgacctaggattgagaatgagtgtgaatgtaggagccaaggagtcttcatgcatttggtctcattatgggtttcaggcataatcttattcactgtgaaagggatacaaaagaagtttaagacctctgggaatttattctctaaaaggtacaattcagcaatgattaagtgccaaaacaagtttatttagtgtgtttcaaatcttcttctggctctataactttgggcaagctacattagttttctctgactctgtttctttatctgttaagtggtgataataatatctgcctcatatgctgctctggagattgaacgagttaataccagcaaggtgcttggaataatgcttgaaacatagtatgtgataaatgttagctaaggtcatggtggagtgctgtaggagtgtagaaagggaaataattagtcaaggctgagttggtgggaagactgtggaggagtagggatttattggggggcaatgcaaggatgggtaggagaagaatgaaggaaaaggaattttaggttggggaaagcattgtaactagtcttagatcagtaaagccataacatctatgtgatttacttactaaacaaaactattcctgttagggagtagtgggacatagcaaaggaggataaataactgatatttattataatgaaagaagttctgagatggggcaccagagcattttggttgtgatttttatcctggccctgtgactcagggcaagttgtctgatgtcttaggaccccacctttcctcatctaaaatcaaatggactaaatttgtaaaatccttacagctctgaattccttagataattcctacatggaaatgatttctctgaccccactgtctgtaggatggtggtggtttttgcgcagcgtgaagccaactggtctatgtggggcaaacttgtggttatggcctctttagccccctgctgtgagtgagcaagctgaccggccctcatacagacactacacactgtcttgcaacacttagcaatcaccactgctccagagatctggaatctcagcaggtggatttgtggaaggagctcttgggtatctcattcccaacttatttagagttaactctactactgttctcttgaggagcctcaaggtttagttgccttgaagtggaccttgagggctaagcaaaaggggaaagaacaatctttagtcttaaaaaaaaaattcctggattgttcttatggtgttcacaaaactaaagagaaaaaagcagtaccatgagcacccaatgaactgccagcaaagccctaagcctttcacagtaaacttacctgctcaggtcattttgtgcttgaaactgaatactgctgcttttgaaaccagggattgattcctccattttactgcatgtgtttaaataatccaggactcttataaacataaatggaaaatgagcttttcaaataccatgcttagaaatcattggctctcataagacattacttcagttttctttttagaagggaatttttgtttgcaactgttctcacgtctccgtccttcctggtttgagcccctctcaggagtcgcacgcaggcccttctgctgctgctctgcctgcagtgctgcagtgctgttgcatcagaggaagttctctaggattccactcccatttgctttttctagttgctaagcaggtggcacttagaacagtatctgccctctgagcttgatggttgaagaagatacttttggacttttatgctggatggattttacacaacttgatgattgaaaaataacttgaaatggaagaactaaagcagatggaagcgattttatctatattttgtgtctccaaagtttgaagggtttattttgttttcagaaactgaaatttgagaagaatgacaatttatcctcatcttgcccttgaaaggaagagtttttctttggggagtggagctttaggaaaggaaggtgacttgaactgtaaggacttcacagctaatatatgcccctgggtaatgttacatagtccttatttgtttagaatagaagttccttgagggagtagaatatatcctttttgtcatatgtggttcctaatagtgagttgcatctgagtgaatataataaataactagcaattcaggagactcctgatctttgatctcccttggcagatcagtccatgccacagatttcctgaaaagaaaaataagcatgtcatggaagaaactatgtgttttttttgcaaaggttgtgaattaaggttgagagtgagatggtttagtctggcctctttttagagcagtgattgattaatgctgtctgtgttgctgtttcttccagagctgacctgtcctggacgcagcataactaacgaagctgctgcaggatgagaagatggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaagtgagttggaggaggaggagcagctgcagatacctgtttcctaacaggcttagggagatgggggagaaagaactgtgtctctttgccaaagtttggataagtagttaaccttttgtttcagttctggcttgtggggattatttttcattttaattgctactaagggtcatttgtggtcttggcactcatcaactctattaataagacttctgattttggctccaggccagatcagaaaaaggggatgtaaatgaaatgggactatttaaaagagtctttaaaagatgggatagtaaaagatagtacagcctccctctctcaaacacacattcaaaattctatctttaatataaagatgttgttactattgtgacatcagtagagtagtgtggtggagatggtgtatttgctctcttgtctagctccctttgtgacattagagcatagctgctggggacaggaataaaactgagtaaagcaaacagccaatgaggacagacccaggtcattggccctgttacacagttgtctggattgattcttccagcactgattcttgcagtagagtcaattctgttattcatgcaagtagggagctcaagtgaaagaactactaatcttcatttggccaagagtttcaaacttcctgaacaaatatttgactaaatcttcagctactttctcatgattatcttctgaaacaaggcaccaagtagagaaacaaagcctcctgggtgatccaccagctaaataattgaatcttgaggactggcatcagacaccgttttagggaacccctcatttgacagattttttttttttttttttttttttgagatggagtcttgctctgtcgcccaggctggagtgcagtggcgtgatctcagctcactgcaacctccgcctcctggtttcaagcaattctcctgcctcactctcctgcgtagctgggactacaggctcagagcagctacgtaatagaaaaaagtggaaccattagtctgttctttcactgggaaatctagaaagtttaatgaacaaaatgatatgatgtaaagaacggtagattggaagtaagaaatcctgattctaccataatttaccatgtttattatttactttttttttttttgagacagagtctcgcactgttgcccagacaggagtgcagtggtgcggtctcagctcactgcgacctctgcttcctgggttcaagcgattctgctgcctcagcctcctgagtagctgggactacaggcgcccaccaccacactcagctaatttttgtatttttagtagagatgggatttcaccatgttggccaagctggtctcaaactcctgacctcaagtgattcacccgcctcagccttccaaaatgctgggattacaggtgtgagccatcgcacctggccttattatttacatttttattttttagtaaatgtattgtccaagctaccacagataatttatggggaaataaatgaagaaaaaaaccttctcaaaactagtttgtaattttcggcaagacacttcatctctctggacctaaactattttttccaactcaaatgcaaattaatttctcattttctttttgttttatagtttttattttttatttttcctttaacttgtattttaagttcaggggtacatgtgcaggatgtgcaggtttgttacataggtaaatgtgggccatggtgatttgctgcacagatcatcccatcacctaggtattaagcccggcatctattaactattcttcctgatgctctccctccccccattccctacctctgacaggccccgatgtgtgttgatccctcccatgtgtccatgtgttctcatgattcagctcccacttataagtaagaatgtgtggtgtttggttttctgttcctgtgttagtttgctgaggataatggcttccagcaccatccatgtccctgcaaaggacatgatctcatttctaatttctcattttcttaaacctgacaggagatatgaaaaaatatatagagctaatagttgttagagcccaattttcctttgcatactctttttccctggttcctgctctatagacagcctctttccattcccaatgcctcagatttcagtggcctggcttgagagtggggacttaccaaagagagtgggtctctggacctgatgggtgtcattcattggataggccaaattgttcttgatggtgctccattagaagactgattcttcatggtagaaattctgacttcagccttcggcttcatcacaagggtaaattgttcctggaaaggataactttaataattttttttggtaatcttgaaaaaatatataccaccatacagaaaagctcataaagtataaatacatagcataatgattacaaactaacagcatgtcaagaaagaacattgctagcactttagatatgttcattgtacttttaaatgaaggatattttacttgattaaaaataatattcttgctcaagttaacttgcaaatctttaccagcagtgctgatacttctataggggtggaaaaagtccacctccattctcctagggtgccagctgggtcttagaattaaattgacataacatagattaacaggagaaaagcataatacatgctttatgtagcacaggaaccctcatgaagaaatgaagaccgtaggctgggcgcaatggttcacgcctgtaatcccagcactttgggaggctgaggtgggtggatcacaaggtcaggagtttgagaccagcctgaccaacatggtgaaaccccatctctattaaaaatacaaaaattagctgggtatggtggcacgggcctgtaatcccagctactcaggaggctgagtcaggagaatcacttgaacccgggaggtggaggttgcagtcagccaagattgtgctactgcactccagcctgggtgacagagtgagagtctgtctcaaaaaagaaaaaaagaaagaaatgaagacctaaagaagcagagtcagttatttatatactggattggacaaaaaagtagtaaattgtgaagaagcaactaaattatgtgggcaggcttaaaagataagacttatttaacaaggtcagtacagtattctcttggtctcaccttctcgcccttgaagataagcatgttgcctttccttcaggttcagggagtgtcttccacacaggaatttcatcttccactcttaagaaacaacaagaagatcagagtgatttttttgcacccgctgttatacaaatgtctttaacttagtcaatatgccagaatagcgtatcttaaccccttcacttcttgctaactaggccttgtactaaattagcaagatctcttctacaaggaagcacactgggaattaacccacaaatcaaaaagtcataagggtagaaatgactttaagaggacattcctttctgtttgttcttcaagggctgccttaaataattcttcgcaaaatagaatctcttttctttgctaaatgataccagtggaatcctttatcttttcctcataggtctcattttctaactgatgaattgtctttgtgactctcacaaaaacttgtttcttatttccttccctctttctcctagtgggtaagagagaactcgttttctattagtgaagttctggttttagctcggatttcacactcccaagggacacagggccctaaaagttgtgcaccacgttttcgagatactgttatttattccagctggtactggtttggctatcataagtatctgaggtccttgttgggtttcacactgactttttcagctggtccctgcaaacaatctgaatcatcctctctttgaattgcctcagacttctcagggctactttacattttctttgtctcttggtacttcgttctaatacctcctttctttggctttcttgtttttcagctgtttgctacaaataattgtttaaggaacatttaaatttttaaattcagtttttaaaataggtaatatatttaagtggttaaaaattcaaaggtacttggtaaataagtttcctttttacacctgtctctcatctgtctatttcctatctccctgcacaggcaaccactgatagtagtatcttccagagtttctttatgtgtatgcaagcaaaaacaaatatgtgttttatggaacttgaactccattaaaagcctttgttcttgtttttttcctgttgttttcttaaacctattgaagtcttaattttgttttttgtttttacttactattatttttttttttggagacagagtctaactccaagttggagtgcagtgatgcaatcataggtcactgcagcctcaacctcctgggcaacctccatctctgtctcccaagtagctaggatcatacgcatgcgccaccacatctggctaatttttgtatttctcccacttttaaaagtttttcttgtttagtgaaaaattaaggactttttctctgttgcacaaagtgcctgtcttcactaaaaaaatgctctccaatgcttaataatttgttgaatacaaaatggccatatatactcctaatgactatcttaagtaggcagccaggaaatctgtaggttaagctgaaacaaggccttcagaattgactcctatccctttttaatttcttagaactgacagttgatatatgtgtcttttttgtttaatgggtataccgtgttacaatcatataaacaaaaatttaaattacaagattgatcaatgtgtttttaaatagggcacactcctattgcattaaaaaaattatatatggccgggcgtggtggttcacgcctgtaatcccagcactttgggaggctgagtcaggcagatcacttgaggtcgggagtttgagaccagcctgaccaacatggagaaaccctgtctctactaaaaatacaaaattagccaggtgtggtggtgcatgcctatagtcccagctactcaggaggctgaggcaggagaatcgcttgaacccgggaggtggaggttgtggtgagccgagatcacgccatcgcactccagcctgggcaacaagagtgaaactccatctcaaaaaaaaaaaaaaaaattatatatagcttcctaaagttcagtttaccttcaacttttcaggtgtacctttttctgcaaagagatatctgcttattagaataatagaaaattataatttgatagccttagacatcatctagttcatcctcctcattttataaattttatgaggtgaagtactttgtcaaaagcccacaccactgttggtggtataaccaggactagagctcatgcctcctgaccctccatttattacaacatatttctagttccccacttttctaattttttattaattgattctcagcctcaattggctcagttctgctgaagctgggcagggaaatagaagaaaaataagctccattttcatttattccttaaaaatccctgtggtttttaggacaggtcctaaaaagtgcagtggtaagagcagtgcttctgggggaaaggattcatgttagccccctgtattgtatcttctcaagtcaaggagctctgaggtgtgctattccagggacttgggcttattatcaataccgccagttattcaactggacatgtgatcttgaggcaatttgtttaacctttctaccacttgttactttttaaaggaagagtatttcctatctgccatagtccatccctaacctatggtaggatagtatacaatcatgtgtcacttaacgatgggggtacgttctgagaaatgagttattaggccatttcatccttgtttgaatgtcacagagtgtacttacacaaatctagacgatgtagcctactgcacccctaggctatatggtatagcctattgctcccaggctacacacctgtacagcatgttaccatattgaatgctgtagacatttgtaacaccatggtatttctgtatctaaacatagctaaacatagaaaaggtaatgcattgtgctatagctacaactttatgacagatacaaggtcactgggcaataagaatttttcagcctcataatcatcttatggaactaccatcccaaatattgttgaccaaaacattgttatgtggtgtctgactatagtggaaagagtgcctaactgggaatataaaaccttgttttagttccggctttgcttcatgtttgtacttggccaaatcactttaatttccttacctacaatatttgcaaaatagagttagacccatatctgtatctttctaattgtatttcacacttttgcctgtcacatattgtattttagctgtggcgaactacttcctgtttccttagtgtggaagatccctccacccacccaccttcttctggtctccctctcttcctgttggaatgtactctgtcttttagttttaccttttgatattctacccacacaggcccagcccagaggcctctttttccttgtagcctaacttaattatcttacacttggagaactgattactccttttaaatttttaaaacacattaggatcttcagtcctacattggataagaagttggtttcaggtttaactcaggttagatgtggagggtacctgtgttcccaataatgtttgcaattttctccttatttcactaatattagtgccatcagcttaagtgatacatacacacgcagccctatatgagctcctggcagaaaatccttgccaaaactgtgctgacacactttcaacaggtgtgcagcacagagaccattacggaaggatttatgttcattaggtgtgatttgaagtattgctaataagttgtatttcctgcttttgcacaataagcctaaatgctgctttctgttttaaataaaaactacatattttaaaactttggtttattttgttgttaaaaattttttcccaacaatataaggaaacaaaatgatgtttcagttattttatttaccaaatcaaattgagataaatgctttgagacactgggaggtgggcccttctcaaggggatgggtcattgatgggcacgtatggtcttgtcccctctctaagtatggctacctagaggagaaggaatttaattaagcattcctaaatcagatttctcttttgtagccttggcaatagaccaaaagttgggaaatcttatttctgatcccatccctgtgtaattccaggtgatcttgggcatgctatttaaccatttaaggtctcaattcctccaatctagaaaattaagattatgctaacagctaacatgtcacttattacaggttgcagtatagaccatttctgtaagaaaaatccaggtcatctaatttgattggaatgtggggaagtgactgaggaacagtggaagatgaggctggggagggtgagctggagcaaatcctggagggcttagaatgttagactcagaggtgtggtgtttgtttgagaagcagtagagaactactgaaggttctggaatgggagtaaggatacgtgatgatagctgtgcttagagaggtgaatgggacgacaatatgtaagctgggccagaaggaaagagaactgaaacaggcagatcagctatgaaataatgaaggtctgagttacagtggtgggagtggaaattaaggggattggatgaattcaatatgatttatgatagattatggagggttaaggaaggaatcgagcatgcaattattcatttgacaatttgataaattgttactgagtgttttctatatgccggggctctgttccgtgcccttgggaacaaaacagacaagccatatacattgttggaacttagtttttttgttagggaaacacataacaaacaaatacggaaataaaattattactgataatgatacgtgcattaatgaagataacatgttccagctactttagaggcttatggtgctctaatttggtacagagactaaacttggagaatccagaagaatcaaagattatatggtctctcaaatattcagtatcttaaataggacttcagcaagtactaaattataatctttaaaagagccaattttgagagcatttgcccctcttggatttttacacagacagaaataaaatatacttgagcatcagtgtgagctattggggtgcttacaaagaaacccagcattttctccaggtttaactcgggttagatatggagggtacctgtgttcccaataatgtttgcaattttctccagtcctaagttctcatgcaggtactaagctgtgattccatagttctgagccacatagggtatagacatagacagcttttacctgaattctaggatgtgtgttattagccagtcacctcagccataaaattccactgattggtaattttaggaattagtttctgttggtttctccagggtgtgatttgtgcaatcttatcataacaaaaatttacctgtggaatgggactatttaataatggccatttaaatgttaaagaatgtttcaagattatgataatatcgatcatctctgacaataattaaacatattttaagtgctttcttagttaaggagagaaaactgtgttattattttacagagggatgcttctcttttgaattgtttccaaattgacaaatgagttgtaaatgtttctttacatataacactatttgacacaacttaagatcaggattgtcccatcatatgacaatttctgctaactctgattttaccagaagaatttttgatacaagctctgaagatgcatttctattccagacactgttgagcctgcaaagtttggcctatggaaccaagttggaataaaagattctgctgatttcagagaatagggtgccccatctactgttcaagaccaatccctcacccctgtctttgaccctgtgcctttctacctcctctggaatttcactcctttggttaccctttctcttttctgtattttccgtctttcctctcatataggttcctttcccatagtctgtaaacacactcaaagcctctcccatcctaatgaatcctcacttgacctcctgtccccaggtagtctaattgcttcccatgccactgaaaattgtaaagaggagggtttactcaccacatacacatatctacccactcctccatccttgaagcctgactgcatcctcacccctttcatgatattataagatcttcccaataacatctcaaatggccaagtctaagattttttttaagttcttaactcactaaacctctctgcagtgtttgacaccatttattattccctgactcttatatctctttacttccttggcctaattttctttttatctcaaatgtttccttctctttcatgttctcaacctttccctaaaatgtttatattcccccagattcagttcttggcccatgttcctctcagttggcacattctccctggatgatctcatccagcctcagttcgaggactcctgactctagcctagcctagaatgttccttaaatattcaacaccttgccagtcatctgtttctggattttcccacaggttactcagtatattcagaattgaactcattttcttcccctcaaacctgcttatcttcttttatctactttcctagttgatggcaccagcaaaattctagatatcatttttgactccccaattttcttcagtccctgaggattgccagtactagggatatctcagtataatgatgcacgctttcacaagtatgaaacagttgtggctccagaaacattaacttgcttcctcaagttccctcaactaatatgtggtagagtcaggtttctgttccagggatctgtgaatttcaccttcatggtctttcttccatgccctgctgtctatatagagcatattgcaggatgatttttttggttttgtttcctatcatttttagagaattttctatcattttgcattgcctcaatatgaagttagtatagtcagccctccatacccatgggttccacatttaagaatttggggaaattatttggggaaaaaagcacatctgtactgaacatgtacagacatttttttcttgtaattattcccaaacaatatagtataactatttataaagcatttacattgtattaagtattataagtaatctagagatgatttaaaatttactgatgtgcataggttatgtgcaaatacaatatcatcttatgtcagggattgagcatctgtggattttggtatccctgggagtcctggagccaattccccgtggatactgaggaacaactgtatgttgttgtgtttagttcatttgttttataccataccttcagcatggtatctgagacatagaaaaggagagaagacttttcaaactaaaattagtgaatttttatgggtagatgagctaaggggtagaaaagtgaccgtatagcttcaactgggcacccagaagaaagttggatatagttaaaggttaggtgattttagctatagtatttctttatgaaagaagagattcatacattcaacaaacatgtatgaactgtctaccttgtgccagataggcatcacattatgtactgtgcttgctaagctgagaaaatcatagtagatggaagataaacccctccccttcacggatctcagggtccagtgagagatactggtatgtaaacctcaataacaatattctgtgttaactgctctaatagaagtaagtacaagtgttatagggacacacaggagggaaggacaattctattgaagagagttgaatggggctgtcttcatagaggatgtgatctttgacctggacctttaaatattaaagtatggttaaaaaaaaattcacctgtcagagaagagatgagagagtagttcaggtgtgaggaacagtgcatataaaggccgagagacatgacacaggtttaattgtttaaaatagtgagtgtagctgtaactgaagcatagagtgtggtttggtaagacaggagataaagcctagaggcaggttggagccaatgagtgcatcctaaggggttttgttttctctcacttccagagttcccaggttggaaataacctttgggacccattttgagctagtgcagccttgctctggattcttgctctaacttgtgcacaagtgtccttcctgcaggcttgtgccaagtatgtggatccaaattaacagaaatattagccccttcttgtcatcttcttttttttttgagagagagagtcttgttctgtcacccaggctggagtgcagtggcacagtctcggctcactgcaacctccgcctcccgggttcaaatgattctcctgcctcagcctccctagtagctgggattacaggtgcacgccaccacgcctggctaatttttttgtatttttagtagagacggggttttgccatgttgaccaggctggtcttgagctcctgacctcaggtaatccacccgcctcggcctcccaaaatgatgggaatacaggcgtgagccactgcacctggccagccccttcttgtcttctaatggccactccgtgtttggagccaaagagctgaaggaatggcagtactgtgtgatagcagtggcagcatcataaccacatttaacgagtgcctgttctgtaccagccactgtgttaaggactccttgtgacccttcatttaatctttataacaacctgataaaattggaatagtgattgatggcacccactttatcaataagaaaactgagccttagcaaccttaacataagttgcctaagatcatgacagctaatgagtgacagagctgggtattgaatccaggtctttctggcctcaaagcctgtaaaccactctgctttgctgctttctaagagagaggagagtgagaatagattctagaatatgaattcagtaaatagaggggttaatatgctttacataataagcctgcattttgctcttgcttagagcaactgtagtctgtgcactttaagaggttaaagtagaaggtgatcaaagtcaaatttatatcgaaggacaacaaggaagaatggccatggaggagggtcagacctgccctgccctgggaagcagcattgtgctggctcagcattgaagcaggtgcacagtgctgctgctctgcccggtatggcttcctgtgaccagaacgtgagcaggaagggtggcagctactccaaatgtaggttggagagcatggggttacaaggtcaaagcaaatgaagaagtgtatgcagaagcacctggaaaactgtaaagccacaggcagatgtggtgctagcattatcttcatcaaactgaacttggagaaaagaccataaacagcacatgtagggggttccgttttgcaacagttaaagttattaagtagaggagattcgttcattgatctgagggttggaccagatgacctggtgcagtaaaaagagtttagaactagaagccgggagatctagtccctggcaactcccccgccccgctcctgctcactagtgctgtgaccttagtttgggcagagcattctgtgcttggtttcttgtttgtgaaacgaatagttagatttgagtaatagttttgaagctttttaaaattgtagtatgccttgataattaagagtataggctttgtagttagattgcctcagttcaatgctaaatttcaccacataatagctgtgttgtcttgggcaagttacttaacctcctgcctcaattttatcatctgtaaaatagtacctacctcatagagtagttgtgagacaattattatattaagtatcttagtagggtgcctgcctgccacatagtaaatgcttaataaatattgaattttattatttcttcaaatagactcataggtaaaattatcggttactaagaaggaatttttaaaatacgggatttagtggtctcttttgctccattctcagtatcgtgtaatagaggcgtggaaaatgatggttctaaataatcacacagctcaaatagaagtacacgcctagggaagaattttgctggtgtctttatgtcctgctttggtttagtatttctaaggggagctggatagtaccaggacacgggtactctggaaatgcattccagttatgcctttgctggagactttcctgggacacactggggctatataatataacctatattgtaattttacttgaggaattcactaggcagattagcataaatcaggattcttttcaacatggcatagtcatttttacataacaggtcatttgtttctgaacagcttcctagcctgatatagaatctgtttagattctgcattatattttccgttattaaaccagggaaattttgaaaagacacttatttgttgcttgctatattggctgtatgcatactcagaaatgaatgccaaattgctgttgattaatgaacaactggccagttgcctagttcagaaagaggtgcataggcctgggggaataaagtacatatattttgtttaggcaggcaagaatttaaggccagagaagttggctaaaaatgagaggaccagttagttctagaaatcaaaacagttaagatctagggacagtcataagtcattgccagtaatagttatagtcagatagactcaaaggttcccagctgccctcttgttgattagctaaacctctgagcccctaggtgttcattatagccagaagctcatcagatgcctgcagattacctttgccctagttgagatgttgacctctcaggatggggagaattggttgagtgaagagctgctctcctgggccatgtgtttcacagcaaaccagggaatggataataaatatttctcgaagaagctttctcttctatgatattctccacccctgcattgagtactcttctatataaagtcttttcatatatatgcatcctttattgttttgttttattttattttattttattttattttattttattttattttattttattttaccaagacagggtcctgctctgtttcccaggctggagagcagagctcactgcagtttcaacctcctaggctcaaacgatcttcccacctcagcttcctaagtaagctgggtctacaggcacatgccaccattcccagcaaattttttttattttttttgtagagatggagttttgccatgttgcccaggctgatctcgaactcctgggctcaagcgatcttcccttcttggcctccaagagtgctgggattacaggattacagatgtgagcccatcctgtttttttttaatttacactttttgtgtggctaaaaaaattatttcagtcttgggatggggtggagacatgctaataaactagctgtactgcagtcctgtgatgactgtgtgtgttcattgttttgcatttgcagtatgctaggatttgtttggtatgcatatggacgtgctctacctggccttgcacggtgttccttcccctaatagtgggccccaaagagaagatatcttaccctgagttaacaatggcaatgcttctacattgtgtgctccaagccccacattgctaattagaaatgaaaagcctgtgcttttcagaaggctcattttatgccacatgacacatttattaatactccatgccataactcgttaagaactgttctgccgctcttaatggaccttgtgctgaaacacattcagtggctgttgttgtaaactgatatttccccttgggcagtaaccgcaatccaatttcaatttcccttttaactcatttacttcacaaagatctaatttgagacagattaggtaaaatattattggacttcatgggcaccaagtgattgttgtttaagaagtattgtaaaaacccaaagcttgctgtcctttccactatatctaggctttgaaggatctggcgttagagcgtagtgttgaacgagagaggacaaatgtccttttctggattcccaccagaagtttaacgatgattacaaacgtttaatcagccatatgaaacatatatacatatatctctctcaaagtcaacccatcaccagtcattcctttccattctcccatattcactgttctcctgagaacaggggataaaagtaggggagatagtgcagggaaaagcattgtgtgacgcagttctgccttggggctgctctatgctcccccctgctgacataagtatggtactatcaagcactgttgaccctggacttgacagacttcaacataaggaagcagaataactaactcaagagagttctacaaggataaaatgtgagagaataaataataataggctttctcctcaaatttatggtttgctgctttgttgcacttaatcctggatgctctgtgaattttctaaatcgtagaattacagatatcagtgctggaaaggacctgagtatgcccatcaggcagccctctctcttattttatagacgaagaaaatgagatctagagacatgcagtggcccagccaggccatacagttaggcagaggcagggaagaaggagaacacaaatgttttagcacatgacctgagagatgatctagtggttccccaaatctggtgagcacagattcctggattctgcacacttatttaatagaacacctggagaagggatttatgactgtgttttttaaaaagctactcagcctaggctgggcatggtggctcacgcctgtaatcccagcactttgggaggctgaggcaggtggatcacgaggtcaggagatcgagaccatcctggctaacacggtgaaaccccgtctctactaaaaatacaaaaattagctgggcatggtggcgggcgcctgtagtcccagctactcgggaggctgaggcaggagaatggcatgaacccaggaggtggaggttgcagtgagccgagatcacaccgctgcactccagcctaggcagcagagcgaggctccgtctccaaaaaaaataaaaaaataaaaaaaaataaagctactcaccccaggtgcagtggctcacagctataatcataatcccagcactttgggaggtcaaggcaggaggattgcatgaggccaggagttcgagaccagcctggacaacatagcaagaccctgtctctacaaaaaataaaaataaaagataagacaaaatagaaagctagtcagatgattcttatgcagtcagtctggacttagtcaggaactaatattagtgtatcccctggggcagatcagttttgccatctgtttttgtatggcccacaagctaagagtagtttacattcttaaatggttggatgtgtgaaaatgatacgaaatttaaatttctgtgtccataaagttttactgaaacacagccactcttcttgggttgcatgttgtctatggctacttttacaccacagttacaggggtaagttgttttaacagacactgtatggcctacaaagcctgcagtattgactgcctgcgccctttcagaaaaagcttgctggtgccgggtatattacttatctctttctcgtgtaactactgcaagccccagtattaaaagggaaattcctaagctttgagtacttgtgtttgtccaaagactatcttgccttttttttttttttttttttttgagacggagtctcgctctgttgcccaggctggagtgcagtggtgcaatcttggctcactgcaagctttgcctcctgggttcaagcgattctcctgcctcagcctccagagtagctgggactacaggcacatgccaccatgtcgggctaatttttgtatttttagtagagatggggttttaccatattggtcaggctggtcttgaaccgctgacctcgtgatccactcacctcgacctcccaaagtgctggacttataggtggactatcttgcttttcaaaaaagatccattgtgaaaaactgcatgcaaattatctgtaaatctaattatttcaataataaaacatgttttattgtaatacaatttcctactaaagtatgggagatttaccttcatttcaggagtttagtgtgctagatgtttatttttgttaacagaaaaactgtttttatacttgatgaagatataagatgtaaatatttagaaaggggtgatggtaaatgagttttattttaagtgcaacactcagtacccaaattaattaggaaatttctatttcacacatcaaatgtccttagtaaagccttctgcttacaaaatcatagtttttgaattcactaaagatgcttagtttcccccagaatcatcagtctcccacttgacagctcatgactgatcctaattcagaatcctacctcctagtttttcttcaaccaaggtgtaacagtttccatttcctaatggcttccttttgtttttcttcttctccttttccctcacctaaaacaacataagcatttgttcatgaaccaatcaacagacatttatgaagcacctgctgtatgccaagcactgcaggcactgattcagtgataggaaataccctccatgctacctctagacctatatttccatcacgaccaaaccaagagacaagaaaaatggaaagtatagagtacattactttaaaaaatcctgccaggcgcggtgactcacgcctttaatcccagccctttgggaggctgaggcaggcggatcatccgaagtcaggagttcaagaccagcctggccaacatggtgaaaccctgtctctactaaaaatacaaaaaaattagccgggcgtggtggcaggtgcctgtaatctcaagtactggggaggctgaagcaggaaaattgcttgaacccgggaggcggaggttgcagtgagccgagattgcgccattgcactccaacctgggcaacaaaagcaaaactctgtctcaaaataataataataataataaataaaaaataaaaatcctgttgtctgtctcctaaggagaggttttgtaggtgatattgtaaaggcctggcccggtggtttttaggtcttggggcatgggccagtgcttctttcattgttgcttagctgccctatctaccccggggactcaaatttctcattcaccagagaatttgtctcccacaccaacttcattctttgccacgttgccactgaactgcctcccaatctggcacagtggaagctgttgagaggctcgaagctcggcataacatgctgctctttgctgcgtgacctttgataggtggtgttcctctcctgagcttttgttaagtgtatagaggataataattcctccttcacccatacttcatgggaacctcatgaggttgaatgaggaggttatttaaaaggctattttcatagagggagttgctgtagaaacacagagaggtacaagtacttcagagaaggggtttgggggaagccctgggctgtttgtttctttgagaagccaagatatggaagagctatagctcagccctcgatatgtttcagatgaaccctaggtctcctacttctctttgcttttaggattaggctttttctgcagcatatcccaccagaaccagtatgctccattacaggaagaatttcactgcagtacaatgtcaaacactagggatggtgtagtgggcgtttgtagaatgaatctagctcaatacttttccattgggaagagaatgacgtgactctggtagattaggaaaggaaagaatagaagataagaaaagaactttaaaaaacatcactaatactgtgcattgggagtacataggctgatcattattttggaactgtggagggaagcataagattggttcctgccctcaaagaaccaaaagcctaattgtaggggatggaggttaaaaaaaaaaaggtgggggcttttcataaatgacctgtatcaggttaagaaggacctctattactagtttgttgagtacttttattatgaaaggtgttgaattttgtcaaatgcttttttctgcatctattaagatgatcatgtggtttttctcctttattaatatgttgcattacattggttgattttcaaatgctaaactatctttgcattcctgggataaatcccacttggtcatggtgtgcaaccctttttatatgctactggatttggtttgctggcatttttgttgaggctgtctgtgtctatattcataagggatgttagtatagagttttcttatgatgtctttggttttgtatcaaggtaatactggcctttagaatgagtttagaaatgttctcttctattttgtgggagagtttgtttaattctttaaacatttggtagaattcatcagcaaaaccgtctggtcctgggctcttctttgtggaggttcattttttaatgcaaggtacagaagagtatttatggaacaatcccatgtgagagagagactttttaaggacatagatgtatgcttttatttacatagaaaatttctagaaggatatacaaaaaaactggcatcgaaggttatcattaggtaatgagatggaacttggtcttatgttccttttttcttttaaaaaaattatgatcatgcattgctccaaaatttaaaaataaaaaactacttaaaatgactagcatttatttggccatttgctctttcactcacacagtgagatgttaggactgtgtatctgccactggcctgtctccttaggaacttgcagtctagtaggcccttgcatctctcagcttcacagtctgctctccctcttccctattttattctccatcaacactgaattactggtcaattccctaaaaaaaacctgcagctacatgctactatgctttcatacatgtgatttttgttcccttacctttccatcccttctcactaaatctgcccaattaatttttcttaattcttgtttgtcctttaaagccccttccaaatattagttaccttcctttgtgaacttcccagctcactctcttttttatttcccagccaaggggaaaaaaatctctccctcctgctgcctctcatacattatgcatatttttgttattgaactttgtacttaccctgcatcataattactagctcacatatgtgtcttccctaaaagtacagttttattcagtgttgtgtcctggtaattagcacaatgcttggcacagaatagatgctcaaaaatattttaattcaattgaattcagaacacagggcattctgggtagaatgagccacataaacaaaggccaacgttatgaattatgagaacctgttccgaggacatgagctagtttggctagatgggaacattcatactgggaaatatgatttaattattttggcaaggctgatattgggggctcttgaaagccatataaagcagattgaatattggtaagtaaatattgattgaatgaagaacatttctcacttccttccagtctttgctgaaatctctcttctcaataggcttatcctgaccacctctttaaaactgttgcctctttattcacccatcccctgactccagcattcttgatcttcctctccctgttcttcctccttttcccatagcacttgtcatcttctgtgtagcacagcctatatgatttactcattcattacagtttttatttattgtctgtctttcccactagaatttaaactcttcgggccaagactatcttgttcactgaccattcccaagcacctagaacactgtctgacacatagtaggtgtccagtcaatatttgttgagtgaatgaatcctaaattaatgaacttcagtatcgtgtacagagctggcatagaagcttaggtatgattcaaatggcctgtttctgaaagaaacaagtttattctgtaaaactgccaagattgcatcagctatttgtattataatctgggcagggcagagggagggaggtggggcaaacattgtgttaagtttgcttgtttaaaaaccaagttgggaagtaaaaagtattaaagacttttgagtttatgtattccttacctagaaatcattgtttcatgtggtgactcatacccatgctttaataatttctctctctttctttctccacagacctttgtagtattaaacagagggaaaactctcttcagatttagtgccacgcctgccttgtacattttaagtccttttaacctgataagaagaatagctattaaaattttgatacattcatatccttttcggcaaatgtggagtgagtgcggcaattgcatggttgcttgtttacctttccaatatttgacaactggattatatgttaagttttttactgatagtgtagttaactagcccatcagtcagaggtttaagagtgaactcctgccacatcccaagctaagcttcagcaaattattattttttttcttaggatgatattgtgggaatctgaaattttgtttggtatcattgtatggacaaattaaaataacctttgttgaagtcttaactttatttagtactcagacttcagtgatttctttctgtggccaactattcagcttaatatattaaaagccattattagaggtttttgaaagtccttcttgagttacaaatagttggatgaatgctttagaaatcagctttgaatttttaaaaaattttttgagcagtgagcttagaaagtgagatagttctcagatgaaataacctccttgttacagaggtttagatgcagattgtaggataaataggaattttttctggtatttttactcaccttaaatagaaaaatgccaatttattcaagtggcctgagttacaccttattaacatatgagagtgcatgtggcaaatgaaaaattatttgatggatttagtgatgttgcatggtggcttgaaacttccatggctggtcgatatttaacagcatgtagtcccatgcttcgaagattactgtatcctccaagtaggatgaggaagccagcagtggcaacttggtgaattggtttagaagcttttgccacattttattaactcttgagaatttagcaacatggttgtgaaatctgtagtttagaagcaacacacttctgaaacctgaaagtttataaggaaataaataattcacagtggacttcttcctttctgttggatcagaagatggagtgttttttctcttctggttccaaaaagcatgaagacaaatccagctgtaatgaagaaaaaaaaatccagctgtaagaaatattttctctttttaaaactccactggtagaataaagatagtgaatgccattaacaagagagagtgaattagtggcaagtgggatatggctcccaggctctaattggctctaattgtctaatatgacaattagagtttgagtaacttgtgctgacctaaaatagtttaatttttctcagagataagaacagaaaggcctgagaaaaattctggatggaaatgaaatggcagtatggtttgctatccataaacattcctggacactttcagaatggattttgctctattacctagtcttcagaagtgtggtggtgcagatgtccttgaaatctttacctaacacgccgcagctggtttcccacattggacatattcaacacatatgggctgtattggctgatgccttgcccccaaagacaattcagccctttagaaggactagggaaggctgggtatgatggctcaggcctgtaatcctagcactttagaaggctgaggatgtaggatcacttgagcccagggcttcaagaccagcctggatgacatagagagaccttgtctcttaaaaaagaacaacaactatggaaaaaaagtgaaccattgcatgtagattttcaaataactagctatataagataattaacttaccaataaatgaatacctagtcatatagatttaattgattttcttatctgacaactttggttgagaaagtgcaaaggtattatactaagtgcaaaaaacatttagaggcttagtctgtagttgagctgaaaagtggttaggggaacatgtttcatccaaagggagtagaacagtgacggcccttttgaaagaaggagaatagctgtccttccccatccatccacagttcacagtggcatgctgtgtttatggtggtgaccacagttttcattgtcaccttgcatttttctcttctgtgcttcatctcctttcaggttattgatttagatctgatacccaatggaaatgtgtttgttttttgagaaaacaaaaggcaggccagattttaatattgccccttgactcttctctacagtatttagcatgatcattatgtgcactattttgaccaactgtgtattcatgacttttagtaaccctcctgactggtcgaagaatgtggagtaagtaactcatttatgtgtgtgcttgtttgttttaaatatttttagtcactaaatcttgctttgccacagtttacccatcaaggagaaaaaaaattagttattgcctctctttatttcacagagaaggctgaaagtacatgtggaatttaggaaatagccctccctaaaagtcaactctagaaattactgcccttcactccaccctttcctgtgccaggcagtgccgttgccctgcagccccatcaccatcttgtatcctcagttcctggtgatactgttttctccaattttaggtttccctgttttttgacttctctaaggttaaaaacagctctggggggcctccttaccttcttcaaaatgatcatgtactagatagctgcaaagttaacttccttggaatacagttcaggacccagtacaacattccaaaggtcttagtttgtgcatttcagtaagtttcattcaaagcatttctttctcctcctcacttccttcctgctgcattggctgtgccctttctgttttgtctctcttagttgaagcaacacttcaggcaagtgctaacttaaaaggttcatttaaagtttctgtccagaaattacctcaagctattcatttctttgacaggtacacgttcacagggatttatacatttgaatcactagtgaaaatcattgcaagaggtttctgcatagatggctttacctttttacgggacccatggaactggttagatttcagtgtcatcatgatggcgtaagttctccccttactttattggtgttctgggtgtgattctgtgtgtggagttgtactcctgggtctgtttgtaggtgtgcatttgtggacacagctgtatgaggaattaatggataaccatgtaaggccctgtgggcttatgtctatgattcttgcttgtgagggcctcttgtggttcaggcgatgataatgtgattatgtgggagaaggcaagcagaggctttacagctgggagagaaaggaggaggtgatttcccattggcactctctcgtattttattcctccttgtaacgatcacggtttgcacacctatcattacaaggcctctgtgaagacttgaaattttttagttgtaaactttcagctgaaaattagtttcctgggtatgtcttaccgtatgtaatttaataaggctggccaggagttgcagacttcagagcacattttagagtcaaaacatgagccaaggtacccacactggcacttcctgaagagttactaaccagaatttcgaactattgctcaagatcagtgtaactccacattggtatgttactgctcttgctcatttgtgttgttttagcaataaaaatgtatctttgggaaacttagctgatgatagaagagttatagcaggctttaatatttttatttccaggttgggaaagcgttttgtggtttgagagtgtagtgggggtggtgtataggttttgcatgtatgcatgatgtgtaagaaagagtgcatttaaatatgtattgatctgagcatctggtggaaagatgtatttcaactgtaaaatgtgaataaatgatatgcatcgaatacttgaaactgaaactgactttctgttatataaaggcaatgtcttactttctagcattatgaatacacattagtgttcatttaaaatttttaagtgtccttgctaggcacagagcttgtaattaaaatgagcacttaacacaatatttgctctgatcccatgatttggattttcctgagccctgcttttgtatgtcacaggccaaaacttcgtttatagacatgactacaatcatatgattacaactcctgattaagtcatatatacagacatttagtcaattttgccattgtcaactagggagttactttaatgtagatttgtttttgtggggctcctgctgcagaaatttgtctccctctcttaagaaattgttcgcaaaacctgggaacataagtctttgatacctcctggccttcctttttactaaaaaatgtgaatgggctgacagtaaagccattctgattgggatcagcacattactatgctgattaaaggagtaaatcccaaaaacctcctttctaatatacttttgaattcttcttgtccaattaaaaacaaatgttccttaaatgtttgtattttgaagcttttctttttcaaggaaaaaaaatgaaaccatagcttgtatataagaccctttcttccccccattctcaaaccctcgcccagtggtaccattacaagttaactttggtttgattctgcaggtatataacagagtttgtaaacctaggcaatgtttcagctctacgcactttcagggtactgagggctttgaaaactatttcggtaatcccaggtaagatggtccggggttggtgttaggtgttgggatagggccctgacgtgacgtattgtactttttgttttgttgtggttttgttttttcctttggtgtttgtgtttgtgtctgtgtttgtcacttgtgtctgtgtgtgacctcccttactacagatatgtgacagagtttgtggacctgggcaatgtctcagcgctgagaacattcagggttctccgagctttgaaaactatctctgtaattccaggtgagaaaatttgtacataagactgactttgccacatctcctctttctcctccattcgttttgtccaccattctaaagcagcatcagtacagttgataatggccttgcattctgcatgtttttcctgggagacattctctggaatggccgttgggtcctcaggcagttttctctgagtgcttgccaggcatgcagcccccattccatccatgctattgaaaaagcacgtgcttaagaatcacttggatctcaccatatttgaaaaaattgtcacagagctagctgtctctttgtatataaatcatttgcttcctggctccatggaattacagtaattttgacatttgccatcatttcattgcttgtgatttctaggcttcctatcgcatggtttgctgatgggacgttattcaaagaattatacatttactattactatattaatgaaattttttaatcacagaagagatctacccagttaactaatccagtatcagagacaaatttaagagacttggagttagtgaactgcacttaaataatcactgtctatcatgttctactgctgtctgtctcactagaataagaaccagtagacatctactgtacatattcagggttcattctcagacataaatcccaatgaaagcttatgcaggcactgttaaaaaaaaatcaaaggtaagataagatacaactaaaggtatgggcaacaaaaagtatagaagtttgagggggaaaaacagaatcagtaggtgagttgagagttagttgattgacaacctctttaaaatttgtgcctttttctttttggttctggttgagctagcccagtggttcttaaccttttgaaggttttggatccctttgagaatcagatgaaagttcacagaccccccccgaagcccatccatggaccccaggttaagattccttaatttaatttaaatcattgctactgggtaatacttatctttctaaagaccagtcttgaaatcttaaaggaaagatattttagttagtattgtgagcctaagatatactggcagtaacttggcagaggtgaagactgtgttgttgctctttggactttagttgtggtcatgggttttcaaggaagactccgtagagaccaggctttccacagcagtttcgtagaaataggaatgtggagaaagaacagaggcatcctattcttatcctattcctattacgtggctcttccccttgtggctgactcagaaatatctctctttttaaaatttatttttattttctagagacagggtctcactctgttgcccaggctggagtggtacgatgatggctcactgcagccttgacttccagggcccaagtgatccttccttctcagccttccaagtagctgagaccacaggtgcatgccatcacacccagctcatttttaaaaaaaaatttttttgtagagacagggtctccctatgtgttgcccaggctggctgccaactcctgggctcaagcaatcttcctgcatcagcctcccaaagtgctgggattacaggcgtgaaccactgcatctggcccagaaatatttctttttcccctttttaacattatctcaatagcagatagagagggaagcacaaagatagaatcctggagagggcagactggaatgtggtttactgagaacctcaaagaaggtaaattcatcttcaccacttacaaatttgggccatggagttggccagtaaatcagtgccctaccaaagatgaattcctatatttttagcgatggtagctgactccctatcaaccaaaggctgggtgtcagttggagtatatacttcttggcaaaaaactacaaccttaacttaccaagatttagttcctagccattttaaatctgcttaacagaaagctcagctcattctccagacagcctcagatagctcattgatggtggcctaatctttaggcaagccctgtcttattttttgaggcatctggaatgccacatctttactttcctttttcctatggtgtctctcctcctctggccccttttaaatatgatcttgtgttttctgctcacttccctcagcgtactctgcaaggcatctgtctctttatgttcctgtgatctcaaaacttctctcccagaattaaattggaaagctgtattctattcactaactagggcagcagatgctgtctctagtctagctcatagcttctgtctctgtctctgctatatggttaacttttgaccatggcatttctgcaagataactcttgattggtgtcacgttatttattctttgtatatatccttaatatgtcatgttatttatcctttatatatatatatatatctggaatatatgcagtatctaacacagtggtgcctggcccatagtagtgctccataaatgattgtagtagcagcacttatgattataaagaaacctaagccaatagaaagggtttgtatcaagatttagacatcagcttcacagtttgaaatagagtcccagattcctatttctagaagcacccccaattgagctggctttgggaggctaactggggtggaaaataacattaagggatagtaaagcgaataatagatcactgtcagtaaatggtatatttggttcttgaattaaaagtgtggctctcagtctcaatctttttgctcccgattctatgttttagagttagcaagagaaatccagtaatgaagtttccctcatgggaagttttctctcatcccatgggaaattctgaaattgctgattatgctttctcccatagagggctgttgtgctattcctacctgcctgctatgcagtctccttccaaggcgggagcaccagtgtatgagtcagtggatctgggccatctcttgcttggaagtatgtcattttgattcttgtgtggtagagctggcccatgattcccccttagtaaaaaccattgttctgtataccaagttttctcgtggatcacttcagtggaattgttgttctctttgtcttctcctcttcccagtttggttatagactactgccaagggtgccaaaaatagaatgtaagtgagagcaaccactgacctggagcctgcaaggtctcacaaagctccctgtgtatcaacttcctttatgagagcttttcctttcctagagcactcacccacccccaacctgggctgatactgtccttcctctttgtgtctttggtagatattctgagtatgtcagggccgccaggactcctgaagattggacagtaaggtgcatcagttagctctgcccttttctgtaagaaatgctttgctttacagctttcttgtagctcagaaaagaccaaacccaccttctgaccctttttactttacttgacttggatacatactatttatgctttccgttttgctccagaggcagcaacttggagaacagtgtacacaaggtctgtgctaaaagggtcggttatagaggagaccctttctaaattttctttagtctggaatttgtcaggttcaccttgacattgaaatattctgcctaaaagtttatatgtcttaaaactttgttttcactttgtatttgtctgagtcccgcataaagtgaacttctaagtgactctgtcagaagttttgtcctctgcatgctttgatcatgtgccacagggctgacatgcccaaagaaagggtgggaatctcgaaagagtttctcaggttcttcttagcagcatttaatagtataagtactaatagaactggttgagagaaactcacttgtcagtttcttttcatttctgtcatttaaatggctcccccaaagcactgcttatctgctgctcattagcacttatttaatttcattccaccttctttagaattgtactttcttgattatttttctcttggcaattttatttctcagctttcatttctgttactttcactcctaaccagtgggatcagacaggaagcagggaaaatgataggcctttctttttcccatcccagtattcacaaagaaagcattttctttaaacgtatactttgtgaatgtggcaatttctgctgcataatgatgggagatataaacggttttaagactatatcctgactcattgtctcttttaaactattctggttattctcttgcctttgtatttctccttttcccacattgcttcttaggaaagataaagtttaacagtggtcctaagtgaaaaaacactaaatgctcacaggatcccactgacccatctttgagagctagtttagttttggtcacactttgtgatgtgaatcaggtctcgagtaaataaatctagagtcccccaatctctttcaaattactgttgctctaaagaatcttcaagagctatcactgtaggctgggtgcagtggtacacacctatagtcccagctaatcaggaggctgaggcagaggatcacttgagcccaggagttcgaggctgcgttgagctgtgaccacacctgtgagtagccactgcattccaacctgggcagcatagtgcgatcttgtctcttaatgaataaataaataaatattatcatgaggattgcagttgcagcaggaaaagtcaacacagcacactactttacccctatgctttgtctgaacccaagcctaactctgggaacagggcagttgttctttccctgggttgagaactgcttgttattgctactgaggcctgtttcaatattaactttgtaggggaattagctcacattgcttcaaaccacttagatttctcaacctgagctacaaactagggaatatgaattagctttatttgtttattttatttatacatatatattttttgagacggagtttcgctgttgtcacgcaggctggagtgcaacggcctgatctcagctcactgcaaccgccacctcctgggttcaagcgattctcctgcctcagcctcctgagtagctaggattacaagcatgcgccacaacgcccagctaatttttgtatttttagtagagatggggtttcaccatgttggccaggctggtcttgaactcctgacctcaggtgatccacccaccttggcctcccaaagtgctgagattacaagtgtgagccaccgtgcctggcaaattaactttatttataatctggccatccagtggtggaagactttgaccccaatatgcttgactgatttttctggacatagcacataggcaggcattgttcatttgttcacaggattcctgggcattgtgaaacgtgacaggagattcagaagtacctttggacatagagtagaattcttcacctgcttcttaaaattccttaaatatccagctttccctcttgtcactgaccgttctctacattgaatccagctgtcttttcttggcctgtggtaaatgtattaactgtttctactcagacacgtaccaaagcgagctaatgttagccttttccaagagttctcactgtccaatttgcccccacgtggcttctgttgcacactctgttgtgtaggagtcttttgaagaagcattctgctgttttggataattggttttcagtcagtatcttgcttgttctggaatgtgatgttgtgagccaatagaataaaaagacagtgtctctgtatagccagaatgatggcattctacattctggcttgtgtcaggtcaggaattctactaacaactttttgcctttattgacaggtgttgatggtacgagggagcgctttcaggcagtcacagctggcaagactgtagagcttgagcttaggaaatgaattgatggcatcatctccataaattggcattgatgtctgcaaatatggccatttgcaatttatcctaatgcaaggaagaagagtcttggctgagaccaatgtatagttcagtgactgtttattatttaatattgctgtgtgatcatgtgccttcctttacctgaaaacatgaagccgcatcacctttcttcttctttttcctttgccttctcattttctcttggtgatctggatgggaatcttggagagtaaaccttttagattgctgtagttagtgttatttagtgattatccttgcttaattaagatttatctgaccctctgtgaagtataagcagctgaataccatttccaattaagtttctactttaaacatcttaaaagctaactcaccaatgagatgatctaagccccagacttcaaagctgaggcttgaaaaagtgtctttttagcttccttgactcatgtttacacatttctttagcaataacatctggtttatggagaagagatgttaaaccagctgattgtgagcatttgggtccatctccaggaaagtgaattccatgcaggcttgacatatcagattagtgggggtcatctctggttgcaagtagcaggacacaggaataattcagggaacccaagggctggaatgcacttgtgcttctgggaagccagtagaaaccagaatgcagagccagaaagccacaggtacaaggaagtactttgtctccatctctcttctctccacgtgtccatttctctctctcagcatactaactgcctttactgctcagtcacatcttaatataatgacagaatatggctaccacttagcttccaaatttatttattaccattctcaccacatgaaaatattactcaactgtctcttatcctgttccaaattcctgtaggaaaggatctggatttaggtcagatgtccacccagacatctgcaactactgagagaaggtgaccttgtacagacatggctgctgagagctcattcttaaggaacaggggatagttggagctcatggcctagaatttccacttatgtggacctggaactgtcaaagagctcccagcccaggaaaatcagattctactcatgctaaaattgtatgcttttcaaattatttacttacaaactcagtggtttggctattttgtagtattatactcttgttactaatacaaaagacagcttacagctcatttctcaattcagtaaaatagcaatggtaaagtcctttaacagtcatctggtgtttggaaaggtgattgtctgaatcgtctggagagatggctgtataggcttcataaaaaagtccaggaaaaagaaactgtacaaccatttttagtgacccattcaagtattattttttaaatcagtctatgactttgaaaacagtaaacttgcgtaattttagaagcctaggattgaattattgattcaatataggcccactccaacccagtcaaaatataatgaggacttctctgttccttagcaactaaatgaaaataaattccattacactggaacaatttttatttcttgatggctgccttctactttattccaccagtaccttcttacctagctgtgtgattatgatacgatttcccagggccttaggtttaaaagatagcttgaaatttcaattacttaatctcaatcaaagaaacatctctgccttattttgaaagacttttggagaagattcttcagcttctccactactgcccactgctgtgtagtggtgtgtttatccatgttctgtagatgagaaaatgagactatgagaagtaaccttaaatttgatttgaaacacaataggtcaggccgggtgcagtggctcacacctgtaatcccagcactttgggaggccaaggtgggcggacgacctgaggtcaggggttcgagaccagcctggccaatatagtgaaaccccatctctgctaaaaaaaaaaaatacaaaatttagccagggcatggtggcgggcacctgtaatcccagttactctagaggctgaggcaggagaatcgcttgaacccagaagacggaggttgcagtgagccaagatcgtgccattgcactccagcctggacgacagagcgagaatccgactcaaaaaaaaaaaaaaatgaaaaagaaacacaataggtcataagtagtgattgagccagggccagaataccagctttctgattccctgtctgatgctctttctgcacagccctgtgaggagaaaaataatcagattataaatgctttgtttcctggagcccaagcaagcgcttcataatccctttgtggccacagtctccatggaaggctcactctaagaaagcacaggactctgcatcctttatctctacagtctacctcttggttcttttttctttaattcctcttagatcccttttaagatatttgtgtaagaggagagaactttctcattcatttactctcccaataaaaatgtattaagtggctgcctttgtgccaggtcttgtgctaggcagagcatccatttctattattggactcagaaaattctattgggctcgactttgaaaagtctaggtgattacttgcaaaatacagcaaaagaagaatcatgtccataccacccattagatcaccattatatgaggttagccctgagcagtgccattttcccaatcatgtccctttgaggagggtaccatatgtaaaatagtgataacaagtgttgagtgccagctatgtgccaggaactttactaggtattttatataaattatctatttaaacctaacaacaacccaatgaggttggtattgtaaatccatttttatagatgagtagactgaggttcagacaataatggtcaacatttgactcatttgctatgtactaaatgctttgtgaagttttttttgtttttgtttttttgttgttgttgttttgtttttgagacggagtctcgctctgttgaccaggctggagtgcagtggcgcaatcttggctcactggaacctccacccccgcaccgggttcaagtggttctcccaccttagcctcccacagtagctgggattacaggcgtatgccaccacacctggctaatttttgtatttttagtagagatggggtttcaccgtgttggccaggctggtcttgaactcccgacctcaggtgatccacctttcttggcctcccaaagtgctgggattacaggggtgacctactgctcccagctgctttgtcaggtgttttatatgaaccacacttaatcctcacagcagccctatgctactcaactataattttccctactttaaagatgaaaaaacagattctaagaggttagaaggcgtattgcgtaagtcagacaagtaagtggcataacttagttaagattagtgattatctacccaggcacacccttcaaagccaggattaggattcccttgaatttcagttcagtggtcttgccttcagaagtgagcagggatgagattcttcctgaggcctgtgtgtcaaggcttagcgcctgcctcttgctcatcctggtgcattggctaccaagagtgctcagtgctttgttggccgtatcctatggatagcctgatatctgggcacgtaaagactcatttggacccaaaaagcatgaacctacaaccatatttttctgtctcttctgtcactgatctctccatgtgcaattaagaaatacaccttcagtctacatttgagccagtgtcttacatttaaactttaaattaaaatgtttttaatgtttttgtttttaatttaggacagaaaaaagatcattgtagaaagcagggaatgtatgtgatacagaagaagtatatttttaaatggaaaaagacaaagtataaatactggattaataataaccttttcaataccttggcatttccagaatccttgggaaaatattattgtaccttctcatttattattggcttttaaacttcccagtggttttctttctaacctggaggcctcctcaattcctgatttttccatttgtttctttgacaaatatttactaaatacctactgtgggctaggcactatagatacagctgtgaacaaggtagatataaaaaatcaattataaccatgacaaatgctacagaagaaagtataagatgctactgaaacactagtaacaatggagaggaagaagagttgcaaagtcagagaaggctttctggaagaattgacgtttaaactgggccttgaaggatgagtaggatttaggcacagaggacaagataatatagcagggagcattccaggcagagggaacagcaggtatgaaggccctatggcaagtaagagctgggctcatatggggaaccaaaaagtagtcagagatgggaaagtgttcccaggaagaaaggagccacatgttggcagaagaatctttcttttgctttttaagatgtattcacttaaaaatggatacttctggaatattgataattttgctgacctattattattctcatggcataaatataaagtggccatatttcaagagtggtgctgtggggaggagtagcagccagtggctaagagggaggcttggggaggtggggagggctttgaggtgcctctgattccggggattctgtctcctcaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaagtaggtggcctcttctctgcaagaggaataggaaaagtctattcctagttcacatggtcagaagctaccacagggcttaaaaaagtagttggaggccgggcgcggtggctcactcctataatcctagcactttgggaggctgaggcaggtggatcacctgagattgggagtttgagaccagcctgaccaacatggagaaaccctctctctactaaaaatacaaaattagccaggcgtggtggcacatgcctgtaatcccagctactcgggaggctgaggcaggagaattgcttgaacccgggaggcggaggttgcggtgagccaagatcacgccattgtactccagcctgggtgggcaacaagaacaaaactccatctcaaaaaaaaaaacaaaaaaaaaaagaggtagttggagctggaaaggaatggcagtgctactagaactagccttgtaaaattattcctttcctcgacgtaggattgtgttcagaagctctcaaacatcagccctctctctccctttttcgcatgtacccctaaacttttgctttgaaattcaggcttagaagcttgataaaaattatttttttccctagctgagttacttgggatatttacacatttagtccagaatgtatctatggaactcaaattttcttaatactactaagactattgcagtcgatctagtctagggaatcaggtgatctttaaggcactattcaatcctattacagttgagtagttttaatcattttatttgaggaaccaaaggaaaatttgatgtagtgatacttctttagactcatgaaaaaagttagagatgtcactattagtcattcctgagaaaataaagtcagtttagtaaccataagaaagtgttgcaaaggagatttcggggacatatgctggaaaggaaaacatgccttgtaatttaaaataggacctagtatctagtgaccaatgagctctaaaatatctttgataaagacacgttgtaaggatagaagaatagagtggtataaaggggtgtacaactcagaaaacttacttttaggtccctttgattcaagtaccagaatctggcatatagatttgaagtatgaatggaagttgggtgtctgtgaaaactatattgcatcaagaactttgggatagcacttttaatttgtaaatatatataagaaaatcattaataggaaagaattcttatcagctgtcacagctgttggaaaagatttatgagctaaagtcaataactttctgctggggctagttaggaacagtgtaaccttattctctcattcacttaaatctgcctgttcatttcctgcctcttcaaacttttctagcaaatttctacacagttcctggcatgctggaacctttactctgtgggaacagttctgatgctgggtaagtagctcacctagttttattctctttccttaaaataatgtacctgttattagtagggtcaaaataggcaatatgtttacttttctttgtgctcaagtagtagaccttacaattgcatctgacctatcggttggcatagattttctttgtttcacatttgtctatctaaagttgacatttcactagtaagtgcctgtgcttgtagtctcaagccaaaatgaaagaatccaaggatgaaatcaataacaccagatagcttacctgcctcttggggactcctctcatcctatagtacaatagagagcaaagataccatcttagtgatggttatggatccaaggatgacccaaaatatgcttgacctgacagcatagaggaggaaaaagagtagcaacgtagtatttgagtcctgatcctgatacttcctgctctaagacctttggcaagccatttaacttacctgagccttggtttttttcatccataaaatggatttatttaataagccatgctctctcttgctcacagggttactgtaaggatccacttacattacatgaaaatgctttgtaaacagagggctttataaatgtagggtggtagtgtcattttgcagagtatattggtcctctaagaactggtgttttaacatgaccacaatgtctagaaatccatttaatttctgtattagactactagagattacattgaaagaaatatgagatgaatctaaaagcaggagaaatggtgtaactgaggtaactgaaatccccccttgacttaggttgtcacagtgagtgtaaagaaaacaaatgattaagaggaacttctggccaggcgcggtggctcacgcttgtaatcccagcactttgggaggctggggcaggcagatcccgaggtcaggagttcgagaccagccaggccaacacagtgaaaccccgtctctactaaaaatacaaaaattagccagacgtggtggcaggtgcctgtaatcccagctactcgggaggctgaggcaggagaatcgcttgaacccagaaggcagaggttgcagtgacccgagatcatgccatggcactctagcctgggcaacagagctagactctgtatcaaaaaaaaaaaaaaaaaaaaagaggaacttctagccgtttgtcatttgggataatagctgagttaaatttgattgaattgcacattgatatgaatctagaacagtacttctcacactttggcatgcataagaatcacctggcatacctcttaaaaatgcagattcatagatcccatatccagaacttttaattcctagtttgggtaaggatgcaggaatctgcatttttaaagaccatgccaggtgattctgaccctaggctgtccccagaagttggttagctgcttatttccttcttctttttcttctaatttgttggcctggctctatcaatggttatttattatctccaaggtcatggagtaaatagaattatgttatttattatctccaaggtcatggctgggtggaattatgttgaaaagtcctgaacttccctttctttctttaggcaatgcccagagggataccagtgtatgaaagcaggaaggaaccccaactatggttacacaagttttgacacttttagctgggccttcttggcattatttcgccttatgacccaggactattgggaaaacttgtatcaattggtgagtaatacctcttttcctttggccatagagtttgcatgagcttatatggggttggggaccttctgtgtgagagacatttagtgaagacattatacaagatctaactgttgtgtttcccacagtcagctggtttttgatattaagtgaattctctcagttctcatccctgtttctcgaatgctcagtcatgtatagctgtagactcttacagccttgacggcctcttgtctggcctgagaatctgctatctctatctacttcctattgttcatcctctgcagcatatctccttttggatatttggggttagggtttgttttttttgttttgttttgttttgttttgtttttgaggtaggtactacaggcatgtaccaccacacctggctataatatttctactttttggaaagttgggatttcactctgttgcccaggctggtcttgcaacttctggcctcaagtgatcctcccactgtggcctcccaaagtgctggaattacaggcgtgagccaccattagggcttctgtttaagaaaaattaaacacctctttgtgcaaggcactatatagatggaatttccataaggaccttggagtatatcaacatttttgagatttgtaacacatttggatttgttcaagcaggttgtacagataaaaataaggtcagtaacattctgttgggctacactggtgactctggacaagtcatttaactttgctggttgtaatgaagttagtgtaagtgattgctaagtgtcttacagcttagaaattttatgacagtccattgttttctctttgtgtcatttggtgataccagttactttgaaaatgaaaaacacgtcccctcaggcatgaaggaccctctttaagggaagtttctagttcaaatatctgagttaattcttacctttctgttgcacagcctagagttccctcaggttgaatgtttctcatgaaatatttgtgattgatgctgtttctttttttttttcttttttgagacagagtctcgctctgtcgcccaggctggagtgcagaggcgcaatctctgttcactgcagcctccaaacaattctcctgcctcagcctcccgagtagctgggattacaggcatgacgcacgcacctggctaatttttgtatttttaatagagacggggtttcaccatgttggttggccaggctcgtcttgaactcttgacctcaggtgatccacctgtcttggcctcccaaagtgctgggattacaggcatgagccaccgtgcctggcccattttgtggttaacagaataaagatgaaaactgtgtaatagtgggttgcatgcacataacctaaaacctagtttccttctgcctttttttaacctcccccactatcctttatgtcatattgtcatatccttttaaaactcatcctattgcaaaagaataaggaagggtgtcactcaagatggggatttcctggccggacacagtggctcatgcctgtaatcccagcgctttgggaggccgaggcaggtggatcacttgaggccaggagttcaagaccaacctggcaaacctggtgaaaccacatctctactaaaaatacaaaaattagctgggcatggtggcacatgcctgtaatcccagctactcgggaggctgaggtgggagaattgctttaacctgggaggtggaggttgcagtgagctgagatgatggcactgcactccagcctgggtgacagagcaagactccacctacaaaaaaaaaaaaaaaaaaatgggtccgggcgtggtggctcacgcctgtaatcccagcactttgggaggccgaggcgggtggatcatgaggtcaggagctcaagaccagcctggccaacatagtgaaaccccgtctctactaaaaatacaaaaaaattagctgggcatggtggtgggcacctgtaatcccagctactcgggaggctgaggcaggagaatcgcttgaacctgggaggcagaggttgcagtgagccaagatcgtgccatcacactccagcctaggtgatagtgtgacacttcatctcaaaaaaaaaataaataaaataaaaagatgggatttccttcaacttggacctgatttcagagacataatttgtctgttaatgctgtttgaggatctgtagataaataaactgcagtcttgtattcttttctttatacttgtcttagcccaaaatcaaactttttttatgaaagaagtgtatggggaatatttagaaatgagaggggatttttttgtcacaatggttgggagataactactgccatttaatatgagttgaggataagtgaaaccattcaaccattgggagtaccaagtgcccacaataggaaggagtactgcacaaggaaacagtcctgtacaaaatgcaaatagtgcccttacagaggaacttggcccattagctgttttcagcccggttcatttggtacaagtgactcagaaaatggcctttgtctttgcagactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggttggtgatgaattctttgcaatagaccttcctgccagatcatggtgactaagaccccatctgattttcactgcagttgatcttttctaggcatattttcaaatagaggccattggtctgatgaaaataacccaagtaacaactgcagatggttgctccaattattccatttgtcttaacagccaaattgtttgttcatatcatcttttgttgaattgctcaactgttcagtgtgcaaccgccatgatgactgggaagaaaaaagtgaccagggcagtttatatagtgtttcacataagcatattctttttcttgggagaaatgttaagattcctgtggtcataaaaagtattatttctaattaaaaagaagatacaggcaaaatattaaagtaaaacttgaaagacatttgtgatctcatcaccttaacacttaactttctcatccttatctaaatgtacatgtatttatacatttttaggacccataatatataatgttacttagcattcatttccagacttgctcagaagggatcctctggtaccaaggcagcagtcaagttcttgtctgaaatgctcaagagaataactcccaaagttctcaccactgggttctgctctgttcacttagggctgtgtgccagaatacacagaaaccctctaacagtctaggtttcctgcctccttttttcctcctttcccttcagtagggaaatgttctgtactaactggttggctttgggtggctccagttaattgccctggtctggcttccctgctgtggcttctttcctaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcaggtaaactcttcttttttctatacctttttcaaaaatgtattttttattaaggtaaaatgtgtatatgtatattataccatctttaccattttaagtgtaccgttcagtgttaagcacaattatattcttctttttcctctttcatcccctctatcccctcttcttcctggcctctagtaaccaccagtctactctctaccttcaagagatccacttttttagctcccacgtatgagtgagaacatgctacatttgtctttctgtgcttggcttatttcagtgaacataataacctctggttccatctatgttgctgcaaatgataggatttcattctttttatggctgaatagtattccataatgtaaatatactgcattttctttatccatttatttgttgatgggcacttaggttgatttcatattttgtctattgtaaatagtactacaataaacatgggagtgcaaatatctatttaatatattgactgactttcttttagttatataccccataatgaaattgctggtgtattattcagggttctctaaaggtatagaactaataggatatatttatatatctattagtatataaatatataaaggggagtttattaagtattaacttacacaatcacaaggtcccacagtaggctgtttgcaaacttcaggagcaaggagagtgagtctgagtcccaaaactgaagaacttggagttggatgttcgagggcaggaagcatccagcatgggggaaagatgtaggctgggaggctaggccagtctcgcctttttacgtttttctgcccgctttattttcactgacagccgattaaatggtgcccaccacattaagggtgggtctgcctttcccatcccactgacttgactgttaatctcctttggcaacacacctcacaaacacacccaggatcaatattttgcatccttcagtccaaccaagttgacatgcagtattaactatcacattggattatatggtaattctatttttagttttttgaggagcctccatactcttctccaaactcattgtactaacgtgcattcccacaacaggtaaactctttgcattggctcaataagcctaaaaacatctggtttttctaatcccacttgatttttgttttcccctttggccctatgttttccaggcccatccctgcatcctcatttctacctcactatgctccatattgttctcctctgggaggaagtgttaccagcacatttggaacattctccattctaatcctgccctcccttctctgatgtaaaagccaaccctagactatgtgatcacctttggcagacttgcttggcagtaaagtatatgtcacaccagcaatcttcatgttctaaaacatcagggagagcttttctcctctaagcatcagaatttagctcaaacatctctgtttaagaattgcatttaattataggggataataataataggagtcatgaaacaggacatcttatttgagcatggcaagttctactgattaattattctggatatgtagtatcacactgataagttagcctatggtagattgaaagcctccagtttttcccaactttgataaataaggaaactaagcaaaaaagagaagagaagacttagggtaattgttgagcattctgttactatagatgtcatcacataaataggtatttttgaatttcagagaacaccaccaaaatctaattcttaggccatttaatttcccattttaaagaaatgctggaaagttggtaggtatagaatagattatagttactgtttatggcagtatatccagtgtcttttcttttttcccagagatgattcattttcccacttgcatctctcactttccggttcagtcttttgccagtcttattcatttcaggggatgagcaaaatccctcatgtccatccctttctttccattccttaaccttatcttagttttggctttacaattgtattattttgttctactgcgtttgtccactgcagttttgttttctgtagtctttctccttctcctttgtattttatatactgtcaacagattacgtcctaaaatgtagttctcatcattcatgtagtaggcatttatggagtacctactacatatcaaggattgtactaggtcctagggcttaaaaggtgattaaaacatcatccctaccctagagagcttacagtacagtgagacagctgaatacactaaaaaattatgtagtagcatgtgttattccataattgagatatgctgggggtgttagaaaagcacagatctatcccaacatggagattcagtaaagattttctgggaaaggtaaatttctgaactgaagcttaaaggatagacaggaattaggtagtgtatgtgtgtgttggtgagggaacaataagaaaaaagggatggtctaggtttagaagggaagcatattccagtttagacatccaaagtttgaattatttgttggataaccaagtacagatttttttagacaaggataggcaaatgatagctcagggaagaggtcctgagctgagaggtggtgattgaaaccatgagacttgtggagattactcaaagagaatgtgtcagttggaagaatagtgagccaaaggcatgaccattgctgttcactggcatagagatggagttcaggaaagaaaacggataaacagtgttagagaagctgagagggccaagagttcagtgaagtcaggagagtagcgatgttggagaaggcagggccatgatcaatggattgaaatttaacagagatgcttagtgagctcagaattgaaagtgtccatcggatttggcaatataaagtcattgaggactttaaagacatgttttagtccagagtaaggaaggatgccatgttgagtggggagtggagacaatgagtatagacttctcatcccagcttctttaaagagtgtgggtggaaataagcatgtttatactttgtggaagaaaaaaaaacagaaaagggagacgtgttagaaaatgaaatagaatagatagaagtagagcacaatcttggaagataccgaaatagatgtggtttttaacagaaaagggacccttgttccctgaaaccttaagaaaacagggaaggatgaggtggataaatttgtagaaaaggagtaggaagttgaggaagataacccctgacagtttagtgttttgataatgttgggaattaaaaatcaaggagtttgagatcagcctgggcaacatagtgagacctctgtctctacaaaaaatgaattaattttttgataatgtagggaattaaacatcttattgaagggaatgagaggttgtgtttggaggtttaaagaaaatattgacagtttagaatagtttttaagaagaatgggagagggaattcccctaatcagattcattctatttactaattaagtcccaactccttaccctggaattcatagcattccccatccccttagtacggttacaacctatgtgttcattctccagtctctgtgccccatcaagcaccctatacctgaaccagaatgagctactagatatttctcatatatgatgtttgttttctaccctctgtggtttgctcaagaaacaccctctctaaaatgtcctgttggccagcacagtggctcacacctgtaatcccagcactttgggaggccaaggtgaaagaatcatttgaggccaggagttcaagaccaccctgggcaacatagagagacccctgtctctaccaaaaaattggccaggcgtggtggcccatgcctgtagtcccagctactccggagtctgaggcgggaagatggcttgagcatgggaggttgaagctgcagtgagctgtgatcttaccactacactcctgcttgggcaacagaacaagaccctgtctccaaaaaaacaagaacaaaaacaaaccaaaaaattaaaattaaataaaatgccctatttttctgtctttgaatttctgcatcatagttattctcaatatcttcctttcaagaaaccttccctgactctcctgttttctagttggaaatcttcattttatctttctgttatgttttatatgtacctctttatttgttgctttttactctacatatagaaagtactctacatatactctgtgtgcatatttatttatgctcatatcctcttctctctaaataaagtgttaaagtcaaggtctgcttcttagcacatggctagatattgttagacaaatgaatgcaagacttttagaaatttgatttaaccctttctgagagacttctttatagcctgaagccatctctatctcaccttcacctctcctttcttctagttcagcctcttttggggaaagtatgatagtagctttgttgtaattttacccttgtcttgagagactcactgagctatgaataccttagcgctgttaacgctttgctattgaatggtctctgattattctataggctttagtacaaaggtacactgattgctccttaactcaaaaaataaaaccaggtttttgagatgccctgctgtgcatgaaaatattcacttagtctaatctctccattcaaaaaaaatagtaaattcctagaggccaaggctgactgccaagagtagcttaagatgctacaagggatagtagtattctttgtcccaggactttcctgcagtttctctggaagagtgactgaggaaatcgaggcctagaaagaatcacttataactataactagttatagaaggttatataactagtaagtgttcaagccaagatttgaacccaggttcatccaactccagagcctgtgctcctaaccaccccttctttattcctgcacacgtacatgatcatctcttctgttagtttaacaatgaatgtggcttcagttttctcacaacatagttcagttatgtgtcagtttcttttatttccatagcttctgagttcttttttttttttttagaaattttgagatttttttcatttgaaggtaatcgtaatgctattaaattcacaaatgctaatttaaatattcaatcctatttatctaaaacacacattgcaaacacacaaattatctattctctccacgtcagtacccattcatatagtggtttggaaatggggagaatagattccccttaaactgcaagtcagcaggtgtttctttacagttaactttagcaaaattcatacaaaatagtaattaacaatgatctttacttgttaactcacaaggaaacaccttcaaaactgcattttgttaaagtttctgtactaaaatgtagaaaaactgaactacataaatattgaaaagttaaaaattccttaatttttaattcctggtaccactaccacaatttacagggcaatatacctgatgtaatgaaaagaaaaagaaaaagacaaggctacaacagataaaaaacctcaggaatgtacatctaattgacactacattgcattaatcaatagctgcacttcctgcaaactgtggctatggtagtcctgaacaagaagggtttcctgtttaagctgcagtaacttttctgactatggatcatccttccttctgtggcagatttttacagttcctctaatgcatttgggacgactgtctcaaagtaacctgcagctttcctgacagctcctcgcgctctctcctgctgagaactgtagcccttttctgctgtttttagaaccttctgctactatatccaccacttccaccactagatgcataaccaccaccatagggactgcccgagcttcttccaccaaaacttccccctttcatgggtccataatttgattgctgttgtccactataatttctaaaatcattatagttcccaccaccaccatagttaccaccgccaaaatttcctccttcattgtaaccatcatatcctccaccaccgctaccatatccaccacaccaccttggtttccatatcctggtccaccaccaccatagccccctctactactataaccaggaccaccagcatagttgccaccgtcacttccaaatccattatatccaccatcacctcctccataactacctctgctgccaccacctccaccaccatagcctcctcttccaccaaagtttccaccacggccaaaattacctccaccacctccaaagcttcctccgcaacccataaaattgccagatccacctccacgacctgtccgtgatccagcggactgcatctcttgtttagaaagggcctttttcacttcacaattatgcccattaatagtgtggtatttctgaacaacaattttatcaactgtatcatgatcatcaaaagttacaaaagcagatcctctcttttttccactctgcctctcttgcataacttctatggtttcaatcttgccatacttttcaaagtagtctctcaaattatattcctttgtatcttctgtaataccaccaacaaaaattttcttcactgttagatgggcaccaggcttcacagaatcctctctagaaacagctctctttggttccactacatgcccatcaaccttgtgtggtcaagcacacattgctgcatccacctcttcaacacaagaataagtcacaaaaccaaagcccctggaccattttgtttgggggtctctcattaccacacaatctgtgagtgtgccccatttctcaaaatgtcctcttaaactatcatctgtagtttcaaagttcagactaccaataaacagttttctcaactgctctggttcctttgggtcatggccctcttcctgccggtggcaatgtcgacggctggagtcggactgggggcgaccaggcggtggttttacctccattttgagcctgagttcttcttgagacatctcctgctcactcatctccgacctatttctctatatttttaaggctcagggcagtttataaatattaagttgagcctattttttcaaagtattttaattgctctattgcagcagttttcaaactttctggtcttaggacccctttatcttcttaaaaaattttgaggacctaaaaacctttctaaaatgtgcttttaatttctcatattagaaattataattaagaaaattaaaaaatatttactaattttaaaaatatagtaatgataaacccattacatgattaacacaaattatatatttttaagaaaaatgaactgttttccaaaaccaaaaaaaagggtttttttttttgtattacatttctgcaaatctctttaatctgtaatgtaatagaaaacagttgaaatatcatactggcttatgcatgcattcagtttgttgcagtatgattttctttggttaaggtatttgaagaaaatcaagtttcatacagatatgtagtggaaaagggaggaatattttaataaccttttcagataactgtgaatgttcctccttgatgctgttccaaaacttaacagtgacaattatttaaacagttagttgtgacgtggaaactataacagtaactttgtactctgttatagtaaaatcccttgctccattttgtgccatagatctttaacctgtgaatgatttctagcatcatgcattgatcatttgagaaatttcctgagtttatgcaaatcttccaaatacacacaagattttaaaactatatttattattaccaccaatctcatcataaaactatttaaatattatgaaactgtcaggctcacagtgtcactgtgtgcttatgagaagatgagtgataaaagcacatatcatctcagtattattaggaaagaattttgacctcatgagccttccttaaaaggtctcagggacctccgggggtctccagaccacactttaagaaccagtgttatactagaaagaaaaagtgaaatatggcatggaaatagaacatgtaaaatcactccttcatcacattccaaacatcccagtcatgtaagcctcatcttgacagacaatttacgtgtatcccctgagatccctagagataatttttctgtggaatgcaaaagttacactctcaaatccagaacatcccagagccattctcccgagcaaacatctaaacctaagttggaattcatagtttaactgtgactgcctgaaaaggactaaaagctcctggttggttgacaggaagattttgaaaagagggagtaaggcctactcatctcataaactacacaggatcccaggatttaatagagccatggatcctgagtcatcactgaaaacctctagtctaggaaaattgagattgaggaagggttggggtttcagccctcaggatgtagctccttagggaatgccctaaatcaggattggatcctgggtacaaggagaaatggagcaagtcacctttacccctttcacatttgtccattgcagactactcttttgcaaagagtgaaacaaggaattagaaaataatagaacagtttttcctcatattatgtattatcttaggatcctgatttttaagtgttcataggttcactgaattggtcataaccttcatcactgtcttatcatccttatcactatccttaaggttaagaatagtctgatgtattagatctcatgtagtatatgatatttcctcctggcctccaagtttcaagagggcagggaccatgtcatatgtctcgttcaccactgtatccacaccacttagcacacaataggtatttggttaaataaaattaaattttggccaggcacagtggctcacgcctgtaatcctggcactttgggaggctgaggtgggcagatcacttgaggctgaggcaggagaatagcttgaacctgggaggcggaagtttcagcaagccaagattgcaccactgcacttcagcctgggtgacagagcaagtctctgtctcaaaaaaaaaaaaaaaaaaaaaaaaattatataaaattaaattttattgtaaaatgcataaaacctctttcctaatgtgaaggcctaatgtaccaggtagtatcagcacccagcattgacctaaggcaaaattatgaattcagtgtagtttagcttgctgtgagaagtgaagcagtgaccccgtgcaggtgtggaacaaccctaaaatgggtggctaagctgcagtacctaatctaagtgcatgttaattaagttaaacattgtagttactatgtgcaaagcactctgggagatataaggatgaatcagtcaatgtgcccttaaggagtttgtagttggttaaaaatgagaggtagaacatgtatataaagaactgttgtaaaaagtagagcgtaggctggatacagtgactcacgcctataatctcagcactttgggaggccaaggcgggaggattgctttagcccaggaatttgagaccagcctgggcaacatggcgaaaccctgtatctacaaaaaatataaaaattagccaggcgtggtatggcacatactgtgtggtgtggcacacacctgtagttccagctacttgggaggctgaggtgagaggatcacttgagcccaggaggttgaggctgcagtgagtgattgcactattgcactccagcccaggcaacagagtgagaccctgtcttgaagaaaacaataaaaaataaagtagagcataaatattctgaaagggggtttggtgctatggaaagattaaggaagttagtagaggaagaagcagaagggctggatcatggagaactgagaggatttccccagctgaaaatggagcgtgcactggaaacagaggaaataaagacctgcggtagggtggctgcatggtagggtgtatgagaaaacagtgaaagatgaagtagaataagtaaattagagtcaaagctgggagggtcttgaatgccatttttatgacggtgtgatagtgttaaaggtgattgtggcatttagcaaaggggccatgagtacctgagtcctgtgttttcctggccatatttctccgcagtgacgccctcaagagagatatatgtgcccaaggcaccggttattaacacctgggcttgtacgctgaacctcagtgttctatgttacgcttgccttcaaacttgtttttcatgtacagatcaaccagtccaaagcccacattcccaactacctcctttatctaactcactcactaagccaatatttcccttccctaaatcaccccatagccagataccagacaagtagttaccacctctgtaggccagagccttccaaaattattcaaactccccaatcctaaacttgctcagacttacctatcctgcctcatcccttccttcctgaggaaaccacaggctcaggctaaactttccccctcactccttctgcttcctgaccaacacttatatttccccgtatggccctccatggcatggcaagcccactcctcttgggaacggaagtaacgaactcttctttcaaaggcagttgcctctgtgtctgtcaccttaccgtgatctgattaaaacaaaatcccaagtacatcttaagacaccatcttctggccagccacattgtttagagcagtgtttctcaagttacctgtggtaaaggactcatttctatttttcccaattcttcatgaactgatactattgtaaaatacagtaacaatcaatttctagaaaaatcatcacattcttagatgttatgactgtgtctaattgttacaaaagtttctaaatgcccctctcagtttccttacttgtctcattgcagaccaataacaagtcatgtactagctgcctgtgaatcacaccgagcagagctggttcaggggaattaatcagtgattcaaagggggtgcccaaacatacatgtaagaagaatagtaagcttaccaatccagcaacatggaagaaggacgtgcaagggcttcctctgttatgtgctcctttaccacacagcgtatgtgtccttttaacacacagtgacaacatctactcataaatgaggctcagtcaacattctggaatctaaccaatgtagctccttctctctggtacaaggatgaggcagaagaacaaaataagtcagggtcatcctgttttctagagacctatatatagcatatggcatgtatctaacaatgacatttatatataattatctatgattagtgcttttcttgatgtggtccccagtgatcaaattcatttattgtcttagaagttcgtgggttgcttaaagccaaagcaggaaaagcctacataacacgtgaaagatacaggtgagagcatggaataatatgcaactagaattatccaaagaaaatataccaatctggggacaggggctttgtacattacaagaagagacaggatctaaagtatagaaccagaaacggaattgtagccaacaaaaagataagggcagaaatgttaaagaggagccccatagcccttccagtgttaacatcaaaagtattaacaggctgagcgcagtgcctcactcctgtaatcccaggactttgggagaccaaggcaggaggatcacttgaacccaggagttcaagatcagcctgggcaacatacagaccccatctctattaaaaaaaatttttttttaattagccaggcatggtggcacatgcctgtagtcctagctacttggaaggctgaggcaggaggatgccttgagcccaggggtttgaggcatatttctgttttacatatttctgttatgagtgaaacagaattataactggggatgttgagatgttttaatttcaggagattgttgcccttgcaatacatactaatacatttattaacagtgaaaaatgggatcatgctttttcaaatgaaaataaatctaatttagccaattgctctgacattgaggactgactgctcattagatcatgtgattgaacattttcagaaatgagctaaatctgtgactttaaggtagattttgatgaaaatatatttaaatcatgtgataagataaaatcatattaaaaataaatgttggcaatgatggaaagacattaataatattttgatcatttcaaccatctattgggttaaacgagcttcctttaagtacaagagtaactcatgtgagaataacccacataattattagtaaataatacagctttttgatgtcctcagaaatcaagaaagggaatgtgtgtaggtaacaaatcttttggcaaattgagttttcagttctttgctttcaataaaattggaaaaagacttaattgccagccgatagattattacaataaattttgctgatagtttgctatatatttttggtatataacttggaaggagtccaaagaattgagtgacattactataacaaagttcttccattccaagataatatttatgtgaataaggttttagcacataaaattgattattagaaaggtggggaggattgatgttgagccatgtctcattctaataataagtaatactaccaacaaatatttaaacaccaattgaaaagaaaatctcatccatcttattaagaaatgcattttaatacaattttctttcttaagtctattacctataaaatgtgtaatatatttctatagcttgatcaattgtattcataagaatattaataaaaactcaatccaggagaattatttaaatgtcttaaagactcatgctcatagaaaatttttaatagtaagtacagtttatttataattttttgtataatttgttggaatcatagccaacaaaaagataagggcagaaatgttaaagaggagccccatggcccttccagtgttaacatcaaaagtgttaacaggctgggcgtggtgcctccctcctgtaatcccagcactttgggaggccaaggcaggaggatcacttgaacccaggagttcgagatcagcctgggcaacatagagaagtaaattcaggggtgagcagtaaaatacattaaaacataaaagtgtacattcaaataaaattccctagggaatatggaaaggaagcatgaaattgaaggagaagaataaataagaaaaaatttatgctaaagaattcttggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtagcgggcgcctgtagtcccagctactcgggaggctgaggcaggagaatggcgtgaacctggcaggcggagcttgcagtgagccgagatcgcgccactgcactccagcctgggcgacagagcgagactccgtctcaaaaaaaaaaaaaaaaaaaagaattctttaagtaaatttctttaccacaaactttacggtaaacaagagttttatttaaaaacatcaatgccggatattatataacttacagctgtttgagaattatatactttacaactatgtaaacttttgatgaaaaaaatatgtttaaaatggggtggatggaatactatgcagccataaaaaggatgagttcacgtcctttgtagggacatggatgaggctggaaaccatcattctgagcaaactatcacaaggacagaaaaccaaatactgcatattctcactgataggtggaaattgaacaatgagaacacttggacacaggttggggaacaacacatactggggcctgacatagggttgggggaggggggagggagagcattaggagaaataactaatgtaaatgatgagttaatgggtgcagcacaccaacatggcacatgtatacatatgtaacaaacctgcacattatgcacatgtaccctagaacttaaagtataataataataaaagaaaaggaaaagtaaataaatacataaataaaatggggtgggagggggcatatataattttcaactgaactaaaaggcttgaaggccactgttggagtagtagttgaccaggacatgtacttggagagaatggcaaatgatacaggcacgttttaaattctgtctttttggccaggagcggtggcttatgcctgtaatcccagcactttgggaggccgaggcgggtggatcacctgaggtcaggagtttgagaccagcctggccaacatggcgaaaccctgtctgtactaaaaatacaaaaattagccagggcgtggtggtggatgcctgtaatcccagctactcaggaggctgaggcaggagaagctcttgaacccaggagctggaggttgcagtgagccaagatagcgccaccacactccagcctgggccacaagagcgaaactccatctcaaaaaaaaaaaaattatatatatatatatatatatatataatatttatttattgttatatatataatatttatttattgttatatatatgataataaataaattctttttgcccagtgtggcgtagtctaactgaagttaaagacacctcctctgagcccatggtaaaaagaaatacacagccatgtgctttaccctggataagttgtgtataatgtgagataaaagcatcttagggatgcgtgcaagaacagtgggtagacgttttgaatccgtaactggtcaagcacaggtgagtaacaatgggtgatgaactgtagcagtcaacatgcagaagtgctttctggaaacacttgtgttctgatcacaggagtgcgagggtggggccggctgggaacctaaccactttgctcagtataaaggtccacactcccgtctcatttccccgtccctctctctttccctgtctgcccctgcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggtgaaaattgataaggcagctaccgatgacagtgtaaggaagaacacaaatagatcgaggctaggcatggcagtctcccccgctccttcccctcctctttccctgctctgcccatcccacttggtctgtctggaccccactcctgttaacaccccagccatttctgtgttcttcctggcctatttctatttctctcttcagttttccccctattactcaccctgtcttctcctttcctttattctttgctccattttccccatcttccctccttgtttctgacattccttctcccctatcaactccttcctttattttaccgttttctttctctccatctttatctttgctagtctgtgtgcttgtgtttccctcctcccacctgctttgactgaatgcctctggccccggagcctgcccggatggaatgagaatgccatggagccaccaccaatgaccaagtctgaaccggtttggaactaccattgcttctcttcccccacgtcttctaccctcctagggaaacagcttggaatttagagatcttatcctttaaatttcaaaaaagtaattgagttttgaataatgttctaagtctcctctgcaagaatgagatgtgctttttctaaagaggaatgctcatgtgatgtcagggtgggaggtgtgtgccagcagagccaggcaaggagaagttaaagaggccaataggaaactggacccagacttttaaaagggattgaaattgaaataaatcaaaggaggaaaaagtgggaggttggggcaagaacacattttatatgggggaggaaggaaaggggaagcaaaagatgaattttaaatgtaatcaaatgtcaggctctggaggagttgcatataatagcttcacaaaatcagttatctagaggtttggataatggtgcccctctttgggaagagccatctcctaacatttctttaaattctaagctgtttgtatcttttttattttaaaattcttataaccttagtgatcctaatccttcatacctctaatttctaactttcctcagtccctcttcagctggtataaactccttgagatagcatgttagtctatttctttctaacacttcagtttcttctaacacagcatgaagatagccaatcctttaattagcatgatgcatgtatggaggaaaactggacaatcatggataaagtttattgaatgtatagataaaatatcaaaaggcaatttgtatgtgtttgggattgtctagtctcgcccttcccttttatttaaaagcaaaatacagccgggcacagtggctcgcacctgtaatcccagcactttgggaggccaaggaaagaggatcacatgaaaccaggagttggagaccagcttggcaacacagtgagaccctgtttgtacaaaaaataaaaataagaaacattagccaggcatggtggcatgtgcctctatagtcccagctacttgggatgctgaggcagggggatcacatgagcccaagagtttaaggctacaatgagctatgatcactacatttcagcctgagtgacagagtgagactctatcaaaaataaaataaagcaaaaatactctcccccttatgtttgtatagctttttataagctacaaattactcttacatgcattatttaatttgtcctaaataaaaattctgggctgggagtggtggctcacgcctgtaatcccagcaccttgggaggccaaggcaggtggattgcttgaggccaggagttcgagacaagcctggccaacatggtttgaaacccatctctactacaaatacaaaagattagccggatgtgggaggctgaggcaggagaatcgcttgaacctgggacagggaggttgcagtgagccaagattgcgccactgcactccagcctgggcaacagagcgagactccatcttaaaaaaaaaaaaatctgtaagggtgactaagtgaagattaatatttctgtttttgagatggggagaaggttcagagaatttgtgttcctttgggtcttacagctcatcactgatagagccagaatctcagacccctcagagaccctaatcttccgtgtgccagttaattttaatttgttctctgcctagacacttggccgttgctcttctcaagacctacttatttaagatcagcaacagtaagaacaataataatcctctggtttctctttagattgtataaatctttcactttttacaacaataataaacagtgggccgggcacggtggctcatgcctataatcccagcactttaggaggctgaggtgggcagatcacctgaggtctggagtttgagaccaggctggccaacatggcaaaacctccatctctactaaaaatacaaaaattagccaggcatggtggtatgcgcctataatctcggctacttgggaagccaaggcacgagaatcacttgaacctgggaggcggaggctgtaatgagccaagattgcgccattgcactccagcctaggcaagaagagcaacactcttgtctcaaaaaagaaaaaacaaaacaaaacaaaacaaaaacagtggctccagatagttggccaaaataaatgttatagatacttatctttacatttaatttttatttgactttcactaattctgcgaatagggataaattttgggctccacagttagataattatgttctccgaaaaaacaagagataatatcaatggcaaatgaaggatggatcaacatgggcagtagagttggggcaattcaaatgtttagaaccaaattgaattttgatatactggcagtttttaaaaatatttaagccaaataatgcagcttgccttttctctgaataaaaaggtattagagaaggtattcagaaagaaggagcttaaacttcagctgaagagagatgatcaggtttaggggttttgtttttgtttttttagcaccttctgttacactgtgtgggagaaaaaaattgagagtcagttaagcagatggctgtggaatgtaaggctttctgtactgctttacactgtggtctataaaggtctgcatttgggttttgggggatctacatacccagcaccctataaacacttaggaggttctctgtagtgacctcaagtgaggatggtgacagacttgagaattcccaaagtcaggaggtcacagtggcaggaagatgaggtaggcaagaccatatcattcccaagggcctcataactaggaaaaactatttttatttatgtatttatttatgttattagaagccataaagaaactgttatggctagttgttaacggtgtgggaaaatgtctaagttacattaataggcccaaaggaagaaaacagcaaaacaagatgcatatagtttgtgtggtatgataccaaattatataaagacaaactacatagagaaagaagatgaccagccaatagttaacaatagttgcctttgagagaagaaattatggacttttttcttccttatacttttctgttctttctggttttctgtagtgagcataatgattatagccatcatatattgagttcttattatactatgctgagcattttatatacattatctcattttattcttataatcactctattcagtaggtggttttatcctattagcagatgagactcagagcgctgtagtgacttgggaattggaccttaaaccatggtctttctgataccaaaaggaacttcctgacagcttaaatattagctcctttagaaagtgggaaaaacagtcacttatcttagaaggacacagtatattaagacttttatattattcaggattataaatttaattatgaatattaagagattctaattgcttaatatctcttaaattaactagaagcccaagacagatgaaaattaatacctcttggggctaggatttagtgattgttactagcaaacattttggatgcatccacagccaaggattaacctgagcaattagaatgcatccagttgtgtatactcacgcttcacagagtggcataggagccactgatagtgcacgcactggtctcacacaatattcctgcttaggctatgccaaatctatattttttccttcaggcttaaattaagaaataggctgtaagaaaaaaagacattcagacatcacttcattagtctcatcaaaaattattttatatcttcattagttattcagttgtgattgagtgttgagattttttgatcattttggaatttcacctttgctttttgctgaagcagaagataaagggactttttcctgcgtctccatctctgagctgggaaagtaggtgagaatcatttctcttcactcattgccactagcaatccagaaatcacatgcaattgagaaatcatgttgaggcagcttgcaaatgtataagcattatctcatttaatgcttacagcctggaccttatgaaacagatactgctattggctgcatctgactgaagaggaagccaaggcttagaggaataaagtaacttgtccaagatggcacagtaagcagtagagttaggactgaatccaagcaatctaactcctaagcttttaccattaaccattacaatatactaactctgaagcccaagttctgtatttgtgcatatgatcctgggaaattctctaagccattttcttctctgtaaaaagagataaaaggacctctgttatagggctattgtgaagataaactataacatatatatgttattcatatataatatgcttcatatataaagtgtgaagcatattatgtggcccattcttgctgttcagaaagccaattttcattccttttcttacacatttttggccctgatagctctgagaagtaaacagtaccgaattgcttgacttggagaaggtggggctccttctaaaaatcctttgtttgagaaagagaaattattgtacaattattttaaagcaaaacactatgcaaatttgagaaaggcttgggaaggggattatggctaggctctaaggatacttataaaatgccataagttttaattccaagaaaggtgactggctaataaacgtccagttatgtatttccatgtgaaaacaatttagagtcaataagaagtcaaacttccttgggcagatctctagaaatgtgggcagaatctgccatctgcagctattgtaggatatctctttgaaaaggactgctgtaatttaggaggttggaagtcagtggctaaatgcccaaataaacagcatttgggttaagagaaatggaacaaacttgtacggggtttaacttccaaagaacttacttaaggttttcagttgcttcctcagttactgagggtttttcctccaaggcttcttacaggttgaattaaaaaaaaaatacaaggctcctaactttaaaatgttaattctcctccccactcaccaacaacacaacacaaaacaaaaaaactatacacctccatttgcaaaaaaaaaccaaaaaacactgaaaagatagagaaggggagggaaagggtatgtgatctttcctctggcccttggatctcaacatagttggaagaagccctcttcccactgccaggtgatgagaattgtgcagctgagcctgtaacaaaggtacatctcacggaagcccaagcagaattctaggtggaagctgactatgagaagctgacagtgaagctgctttacctgggagagaggctcctccagattttcttcccttcagttgtctgatttaacataaatagtagctcatgcctgtaatcccagcactttgggaggctgaggcgggcagatcgtttgaggtcaggagtttgagaccaacctgaccaacatggtaaaaccctgtctctactaaaatacaaaaattaaccaggcatggttaatctcagctactcgagaggctgaggcaggagaatcacttgaacctaggaggcagaggttgtagtgagtgaagattgcaccactgtactccagtgtgggtgacagggcgagactctttctcaaaaaaaagaagaaaaagaatagccaagaacctagccttgaaatcccagctttcaaaacctcacttagggaagagggcctgctcacccgtgttggtgtaccctaagtttaacagattagattttcctggtagtacagatatattgttctctagacaccatcttagccccctctcagcacttttagtgatgtccttcatagacaattttagtttgtgtcatgttagttaacactggtgaatatatgtttgccctctctctcttcatgaatcagcctattttgatgaatactatgagacatctacaaatggacaatgactagaccatgtaaaaaaatagaacgttgacccatgacctgcccaagaaaccatcccccttatctacaataaacagtccaagaagccagcctgttataagtcaaacttgcaggaagccaggttgctgtctctgatgacaatccaggaagctaagtaataatttctgtaacaatagtcccctaatgagagccaggcatggtggcccttacctgtaatcccaacgctttgggaggctgaggcaggaaaattgcttgaacccaggagtttgagactagcctgggcaacatagtgagacctcatctcttcaaaaaaattaaaaaattagctgggtgtggtgtcacgcacctttagtcccagctacttgggaggctaagatgggaggatcacttgagcctgggaggtcaaggctgcagtgagcagtgatcactgtactccagcctgggcaacagaggaagaccctgtttcccaaaaaaaaaaaaaaaaaaaaaattcccaaatggccaggacttgattcataactgacagctttccttaatttttgttcccattttcaacttagattcaaccagagacagccaaacatgcaccccccaacccatcacacgagatgtcccactttttattaccttgactacagcttccccatgccaacagcctccattcaagccacatttcaagccttcccttttctcacctgtaaatctttgccactcctctgcctgcctttgagtttctgccaaaacacaaatggcagttatggtggcggacttcctcagtatagcaaggtctaaatagcgtttgcttttctcatttggttgatctttatttccacaatacagataatattttatacatattttatactccatatttttgcattccatatttcttctcaaaggaattctgcataatcaaattctggtaggacattgggtaagccgcacccaagagttaaccaaagttgtgggtaaaaaggaggtggagaaggagttatctgagcaattgaggatcattccctaaacccatcattaccataagactctcctaaaaagtactctcctacccgtttctaatcagttccttccacttcaggcaaccacttctgactcgtatcactatagatagatttgttttcttttgtatgtgtagactcatacaatatgtatttgtatttgactgctttcaatcaacaagatatttttgagacatattcatgttttatataccagtagttcattccttgttattgctgagtggtatttcattgtataaacatatcccaatttgtttattcattctcctgtcgatagatgttccggttgcttccagtttgcaactagtgtgaataagcttgctatgagcattcttataaaactcttttagaacataaacgttttcatttatattgggtagatacatcaaagtgaaattcctgggtcatatggtaaatgtatgtctaatttataagaagttgccaaacagttcctcagaatagttacaccatttttttatcccaccaacaatgtattagggttacacttgctccacatcctagccaaacttgctgttgttagtctttgattttagcattttaatgcatataaaatgttgttttattgtgccattaatttgcatttcctgatgactaattatcttttcctatgttttttggtttctcatacatcttctttaatgaattgtttattcatatcttttgccccttttttgactttttattattgatttgttttttatgttccagatacaagtcaaatataaaaatgtttaatgttaatgaagttcagtatatcaatttttttcttttatgatatggctttttgcatcctgttcaaggatgcctacccccaagaacatgaagatattttacttctataagttttatggttctgagttttacattcaggtctccaatttatctcaaattaatttttgtgtgtgaaatgagatacaagtcaacctggatagatatccagtctgttagcaccatttgttcaagagactttcctttctccattgaagtgacttggcaccttggacaaaaatcaattgatgtatatgtgtgggtctatttctggactctgttctgttcccttgatctgtttgtctatgcttatgccaatatacatttgtcttgagtattgtaactttaattcttgaaatgtggtagtataagccctaagaacaaaaaataatatttttaagattattaaatagcatctctagagattattttcctattctagagcctttgcatgtccttagaaaattttatttaatttcagatttttgtgggtacatagtaagtatatatatttatgaggtacatgagatactttgatacaggcatgcaatgcttaatgatcacatcatgaaaaatgggatatccatcccctcaagcatttcttctttgtattacaaacaatccagttatattcttttagttattttcaaatgtagaattaaattattttcactacagtcaccctgttgtactatcaaatactagctcttattcttactttctaacaattttttttgtacccattaactacctccatctccccgctacccttccccactacccttcccagcctttggtaaccatccttctactttctatcctcaggagttcaattgcattgatttttcgatcccacagataagtgagaacatgtgatgtttgtcttcctgtgcctggcttatttcacttaatataatgactttcagttctatccatgttgttgtaaatgacaggatctcagtctttttttatgtctgaatagtactctgttgtgtctaagtaccacattttctttctccattcatctattgatggacacttaggttgcttccaaatcttggctgttatgaacagtgctgcaacaaacttgggagtgcacatagtgcagatagtgcagttatctctttgatatactgatttcttttttctttttcttcttctttttttttttgagacaatctcactctgtcgcccaggctggagtgcaatggcacaatctcggctcactgcactgttaacctctgccacctgggtcctatgttcaagcgattctcctgccttggcctcccaaggagctggaattacaggcatgtgccaccacgcccggctaatttttgtatttttagtagagacggggttttaccatattggccaggctggtttcaaacccctgacctcatgtgatccacctgcctcagcctcccaaagtgctgggattacaggcatgagccaccacgcccgactggatatattgatttcttttcttttgggcatatacccagtagtgggattccggatcgtaaggtagctctatttttagttttttgaggaacttccaaactgttctccatagtggttgtactaatttacattcccaccaagagtgtacaagaattcccttttctccacatcctctccagcatttattattgcctatcttttggttaaaagccattttaactggcatgagattatatctcattgtagttttgatttgcatttctctgatgatcgtaatgttaggcaccttttcatatacttgtttgccatttatatgtcttctttggagaaatgtctattcaaatcttttgccagtttttaaattggattattctgttttttcctatagagttcttggagctccttatatattctggttactaatctcttgtcagatggatagttttcagatattttctcctattctttgggttgtctgttcactttgttgattgtttccttgctgtgcagaagctttttaacttgatgtgatcccatctgtccatttttgctttggttggctgtgcttgtagggtattattcaagaaatttttacccagaccagtgtcctggagagtttcctcaatgttgtcttatagtagtttcatggtttgaggtcttagacttacctctttaatccattttgatttcatttttgtacatggtgaaagacaggggactagtttcattcttctgcatatggatatccagttttcccagaaccatttattgatgagactgtcttttccccagtgtttattcttggcgcttttgctgacaatgagttcactgtaggtgtgtggatttatttctgggttctctattctgttccattggtctgtgtgtctgtttttatgccagtaccatgctgttctggttactatagctccgttgtataatttaaaatcaagtaatgtgattcctccagttttgttcttgttgctcaggattgctttggctgttctgggtcttttgtgattttatataaattttaggattttttttctgtttctgtgaagaacaccattggtattttgatagggattacattgaatctgtagcttgctttgactaatatgaacattttaacaatattgaacaatccacgaacatggaatatctttccagttttttgcatcctcttcaatttctttcgtcagtgttttatagttttcttgtagagatgttttatttcttttattaattactaggtatttaattttatttgtggctcttataaatgggattactttttttatttctttttcagattgtccacctttggcatatagaaatgcttctggtttttgtatgttgattttatgtcctgcaactttactgaatttatcagttctaagagttttctggtggagtctttggatttttctcagtataaaatcatatcacctgtaaacaaggataatttgacttcttcctttccgatttgtgtgccctttatttctttttcttgtccaattgctctaagtaggacctccagtactatgttgagtaacagtggtgaaagtgagtatccttgttgtgttccagatcttagagaaaagctttcagtttttccctatccagtataatattagctgagagtctgtcatatatggcttttattatattgaagtatggtccttcaatacctagttttttgagggtttttaaccacgaagggtgttgaattttataaaatgctttttcaacgtcaattgaaatgatcatatggtttttgttgtttattctgttgatatgatgtatcacactgattgatttgcatatgttgaacctttcatgcatcccagagataaatccaacttggtaatgatgaatgatctttttaatgtgttgttgaacttggtttgctagtattttgttggggtttttttggaaatgtatttttgcatcaatattcatcagagatattagcctgttgttttctttttttgatatttccatgtctggttttagtatcagagtaatactggcctcatagaatgagtttggaagtttctctcctcctttatttttcagaataatttaaataggattgacattggttcttctttaaatgttggttaaaactcagcagtgaagccatcaggtcctgagtttttctttactaaatggctttttattacagctttgatcttgttacttgttaattggtctgttcaggttttggatttcttcatggttcagttttggtaggttgaatgtgtctaggaatttattcactttctctagattttccaatttattggcatgtagttgctcatagtaaccactaatgctactttgaatttctgtggtatcagttgtaatgtttcccttttcatttctgattttatttatttgggtcttctccctttttttcttcattagtctggctaaaggtttgtcaattttgtttatctttccaaaagataaacaacctcttgtttcattgatcttttgtattttcttcattccacattcatttatttctgctctgatctttattatttcttttcttctactaatttggggttttgtttgcccttttctagtcaagatgcaccattaggttattaatttgaagtttttcttcttttttgatgtcagtgctgctgagaccagctcggttggggagaccctaacccagtggcgctagaggaattaaagacacacacacacagaaatatagaggtgtgaagtgggaaatcaggggtctcacagccttcagagctgagagccccaaacagagatttacccacatatttattagtagcaaaccagtcattagcattacttctatagatattgaattaactaaaagtatcccttatgggaaaagaagggaagggccaaattaaaggaataggttgggctagttaactgcagcaggaacatgcccttaagacacagatcgctcatgctattgtttgtggcttaagaatgcctttaagcggttttccaccctgggcgggccaggtgttccttgccgtcattcctgtaaacccacaaccttccagcttgggtgtgagggccatcatgaacatgttacagtgctgcagagattttgtttatggccagttttggggccagtttatggccagattttggggggcttgctcccaacgtgtctcccttctttgatttgcaaagagataaaagcaagggcggctttgtcacagtgagctacttcttgcaggagtcagaatctgcatctgcagactatacaaagaaaaacaacgtagattaaaagcacagtcatcattgaaatcacagagcttccaagtgtttttatccattttaatggattactagctgctaatttgtctgcagcgcctttaagcactccagttcctggcattaaggtcaggtgtgcctgggatgctttaaatatttgttctttaattttgcaatatccaaaggcaagtttgtagagtgtctttgtagatggtttttattttttcctaaattttgatcttattaagagctattaatagtttccacaaatccttatgtttagctcccacaacgagccctatcatttgaggttgaggtgccactataccaccatggttccagataataggaacttttgccgtactttttggtatttctaccatgtgatcagtttgttcagatcatctgaacatagtctggccatggcacgcagactgagaggtgcaattcaagcttaacattcccttaggggaccaattaataatgattccataggaatcgttgtgcagcacctctgcctgttttgcaaagcaatcttcctaaacaagtacgttcattttttctaactgggtccaatcctgtttacaaataggtttttgagggtggtatgcctcaattataggagcagatttattatggtaaataccgagatcagaaagcatgtgtaactgtgtcatagagtgattgcatccaggcattattaccggcccttattgaaggaatactcacgacagtggtgataaccactatcatagctaccattaaattattcattgtgactggttgtcccgctttcctcaggttttcttcctccatctctgacagcttcttgatctgtccccaggtgggtggctgtggttgacgggtgttgctcgtaactgttggggtcctcctcagcatcggtctcgacatggctgcaaccggggggtcctcgggatcctcctggagtctcttcctcggcatctggctcctgataaggtttcaggtgtcttgatggtatccaaatcggctgttgattttggcctggagaaatacaagcataacctctaccccaagttattattttacctatttcgcacctttttgttattggctctgtccaccaaatcagttgttctgtttctgtctttgcagctggtttctgtagatgctgttcagctgctgatagcatctggcctttgggcaggctcaaaaaatttaaagttaataatgctaggttcagttgcatttgtgggggttcagtattctctgtctctccctttctgcttttgcaactgctgttttagggagagagattcattctttccactatggcttgtccttgagaattgtatgggataccagtaatgtgtttaatattccacatagagaaaaatgtagctagagcttggctagtatagcctggggcattctctgttttaatagaagcccatcaccacaaaacactgcaaaaggtgacatttaacacgggcagaagactcttctgattggcatgcagcccagacaaagtgagaaaaggtgtccacacatacatgtacataagctagtctcccaaacgagggaacatgtgtggcatccatttgccaaatagagttaggttacagtcctcgagggttaactcctcctgtaaaagatgaggaatgtaccatttggcaagttgggcatcactggataatagctttagcttcattccaggtaatgctatatctgcatttgagaccagaggcattgacatgggttgaattgtcaaagtgtccagcattagatattgcaatagcaactaggccatcagccatttaattcccttcagtcaaaggtcctggaagaggtgtatgagctctaatgtcagtgatgtaaaaagggtgcattctactcctaactgctatttgcagttgggtaaataaagtcatcggttgttcatctgtatgaaatcgtaactgagaattttcaattaaccgtgtggagtgaaccacatatgaagaatcagaaatcacattaataggcatttcaaaagcagtcaatacctcaattacagctacaagctccgcttttagagctgaagtatagggcgtctgaaaaactttaccttttgattcaaaataagaagctttaccattacgagacccatctgtaaaaacattctcaacaccttcaattggtttaaatttagttattttagggagaatccaattagttaatttcaaaaaactgaaacagcttcgttttaggaaaatgattatcgagaatactcacaaagtcagctaaacgggtttgccaagtaagactatttataaaagcttgctgtatttgtgccttcgtgagagggacaataatttttccaggatcatatccatgtaatttaacaatccgagttttcccaatccctatcattgtagcaatttgatctaaataaggagttagagtccgtgaattagtatgtggaagaaaaagccactctactaagtcctgttcttggacaataacaccagtaggtgaatgctgagttgaaaaaattagcaaatctagggtcttctctggatctgttctatttatttgagctttatggacttgcttctcaattagttgtaactctgcctctgcctcctttgttaattgccaagggctagtgagactaggatttcctctaaggatagaaaacagattactcatggcataggtaggaatgcctagagcaggtcgtatccaattaatatcccctagtaatttttgaaagtcatttaatgtttttagttgatccctacgtatggttactttttgtggcacaatggtagtgtcatttactcaggtccccaagtaggagtagggagtagtagtctgaattttgttaggagctataattaaaccagcacgagaaattgaattttgtaagtgatcatataacacttagcaggctgcaggttgtttactgcaggaattgtaaatgcaaaccattcacagtgttgctcagctaaagggatagtaaagaaacagtcttttaaatctatgactgttaaaggaattgatggcttttaagtcagttaacattctccatttacctgattttttcttaattacgaaaactggagaattccaaggggaaaatgttggcgctatgtgcccattttctgattgttcagcaagcaatttctctaaagcctccaatttttctttacttagcggccattgttctatccaaattggcttgtctgttaaccattttaaaggtataggttctggaggcttaacaatggccgccatcaaaaatgatttcctaatctttggtgggaactttgtttttccactttaagcggttctttcaaaccatgcaaatttttttctagtcccataccagggacataccccatttcatgaatcatatgttgactttgagggctatataattgttctggaattagaacttgtgctccccattgttgtaataaatctcttccccataaatttataggtacagaagttataattggttgaatagtcccaggtcatccatcgggcccctcacaatgcaaaatataactactttgatatacttcagtggctttaccaactccaactatgttcagttgagcggattgaattggccacgtggacagccagtgctatagagaaatgattgaaatgtccgttcctgtatctcccaaacctttacatttccttccctgaataattatttcacaggtaggacgtttatcagtaatttgatttacccaataagctgctttgccttgtttatttgtgcttccaaatcctcctgttcgtttaatttcactttttcccattcccacatatggcacaatcaggagctgtgctatgcgctctcctggctctgctttccagggaacagaagtagatataacaatttgaatttccccattgtaatctaaatcaatgactcctgtatgtatttgtacgccttttaaacttaaactaggccttcctagaagtaatcctattgtccctgctggcaagggtccacagactcctgttgggaccttttgcgggggttccccaggcagaaggctcacagcttttgtgcagcataaatctactgcagctctaccggctgtggtgggggacagacattgtacaggggtgagggaatggcctgagctggaaatgccctggtttagaatggggcccgggacaggcccctcatggtgtttcccaaaatcaggttcccatctttatcaaacttagagtgacactgactagtccagtgttttcctttttttatatgttggacctatttcagactcaacagttttcctttttcctctatctggcggcctgacttgctgattttttctacattcttttttagtatgaatagcttttttaaattctttgagtaatttaaaaggaaaaggctcaaatgtagctgtaatatttccctgttgatctggggggtttgttctaacagggaaccgccaaacctctaaatcaccctctcgtatagcttgctgaattcctgcctgaatagaactaagagccgtcactcgaggtgccgctcgaagagtcactggggcaactactttttgcccaatgtcctccggaaaagaaagatccggggggtcattttcttcaaaataataaggagggggtgcagaagggtagggatgaacctctccttcctttgccactttagctttagctggcaaataaacctgctctgtaacctcttctgttacttcactattctcttgttcctcctcattatcagtgtgaaaaagttctaaggtgaaacgaaccagaccccatccctgtcccattgttaccctgaggcatccgagctccccttcttactcaccatggggactgctttaagagtactcgggtgtcctccagctagttttctgttccaagcatcgcttcagcaaccctttgacctggatttgagcccccacgaatggacgccacttgcgcgagaccagctcggtcagggagaccctaacccagtggcgctagaggaattaaagacacacacacacagaaatatagaagtgtgaagtgggaaatcaggggtctcacagccttcggagctgggagccccaaacagagatttacccgcgtatttattaacagcaaaccagtcattagcattgtttctatagatattaaattaactaaaagtatcccttttgggaaactaagggaagggccgaattaaaggagtaggttgggctggttaactgcagcaggagcatgttcttaaggcacagatcgctcatgctattgtttgtggcttaagaatgcctttaagtggttttccaccctgggcgggccaggtgttccttgccctcattcccgtaaacccacaaccttccagcttgggcgttagggccattatgaacatgttacagtgctgcagagattttgtttatggccagttttggggccagtttatggccagattttggggggcttgctcccaacaaggtgctgatagctttaaacttccctcttactagtgcttgcactgtatcccataggttttgttatgttgtgtttctgctatttgtttcaagaaacttttcagtttccttcttaatttcttcattgactttgacccactggtcattcaggagcgtatttaatctccatgtgtttgtatcatttccaaaattcctcttattgatttctggttttattcaaagatgcttgatattgtggtaaaaaatgctggatatttcaaattttttgagtattttaagacttatttgtgacctaacatatggtctgtccttgagaataatccatgtgctaagggcaataatgtgtattctgtagcctttggatgaaatgttctgtaaatatctcttaggtccatttgttctattaggtcatccttagtgtaggttaagtccagtgtttctttgttgattttctgtctgggagatctgtccagtgctgaaagtggggtgttgaagtctctaactgttattgtattgaggtctctttctctttgagacaaggtcttgctctgttatccaggttggagtggagtggtgcgatctcagcccgctgcagcctcaaccttacaggctcaagtgatcctcctgcctcagccctctgattagctgggactacaggcatgcaccaccatgcctggctaatttttgtatttttttgtagagacagagttttgccatgtttcccaggctggtctcaaactcctgggctccagaaacccacccacctggacctcccaaagtgctgggattatgggcatgagctattgtgcctagcctgaggtctatctctttagctataataatatttgctttacatatttgggtgttccagtgttgggtacatgtatatttataattgctatatccttttgctagactgacccctttatcattatgtactagcgatctctgtctcttcttataatttttgtcttaaaatctattttttctaatgtaagtatagctactacttctgctcttttttggtttccattggcatggaatatctttttccatccatttattctcagtctgtgtatctttataggtgaagtgtgtttcttgtaggcagcagatcattgggtcttattttttcatgcatttagccactctatgtctttttattggagagtttagtttatttacattcagtgttattactgataagttgggacttactcctgccattttgtcatttgttttctggttgttttgcggtcttcttttacttcttcccttccttccttgtcttccccttagtgaaggttattttctctggtggtatgacttaatttcttgctttttattttttgtgtatccgttgtgtgtttttcaattcgaggctaccatgaggcttgcaaatattattaatgtctcataatccattattttattttatttttatttatttattttttgagacagagtttcactcttgttgcccaggctggaacacaatggtgtgatctcggctcactgcaacctccacctcctaagtgattctccagcctcagccccccaagtagctgggattacaagcatgtgccaccacgcccggctaattttgtatttttagtagagacgggctttctccatgttggtcagactggtctcaaacttctgacctcaggtgatccgcccacctcgacctcctagagtgctgggattacaggcgtgagccactgcacctggcataacccattattttaaactgatgacaacttaacactgattgcataaacaaacatgcaaaaagaaaactaataaaaactgtacactttaacttcatccctctgctttttaactttttgttgtttctctttatgtcttattgtactatgtcttgaaaagttgttgtagttattatttatattggcttatcataacactgtaattatggtatgtgggagtttacatgccacaattacagtgttacaatattctgtgtttttctgtatgcctactattaccagtgagttttgtatcttcagatgatttcttcttgccgttaacatcctctcttctttcagatggaagaactccttttagcagttcctgtaggacaggtctggtgttgatgaaatctctcagcttttgcttgtctgggaaggtctttatttctccttcatgcttgaaggatatttttgccggatatagtgttctagggtaaaagtttctttccctcagcactttaaatatatcattaccactttctcctagcctgtaaggtttttcactgaaaggccagctgctagatttattggagctccattgtatgtttttttttttcttttctcttgctgcttttaggatcctttctttatccttgacctttgggagttttgttattagatgccttgaggtagtcttcttttggttaaatctgcttggtgttctataatcttcttgtacttgaatgttgatatctttctctaggtttggcaagttttctgttattattcctttgaataaactttctatccgtatctctttctctacgtcctttttaaggccaataactctaagattttcccttttgaggctatttgctagatcttgcaggtgtgcttcgttttgttgttgttgttgttcttttttcttttgtctcctctgagtattttcaaatagcctgtcttcaagctcattaattctttcttctgtctgatcaattctcttattaagagactctaatgcattgtttagtattcagttgcattttccaactgtagagtttctgcttgattgtttataattatttcagtctctttgttaaatttatctgatagaatgctgaattccttctctgtgttatcttgaatttccttgagtttcatcaatatagttcttttgaattccctgtctgaaaggtcacatatctctgtttctccaggattggtccctggtaccttatttagttcttttggtgaggccatgttttcctgcatgatcttgatgcttgtagatgtttgttggtgtttgggcattgacaagctaggtattttttatagtcttcgcaatctgggcttgtttgtgcctgtccttcttcagaaggctttctaggtattcgaaggaatttgagccccgagtccaataatgctgtggtttttgcagattcatagaaataccaccttggtggtcttggataataaagatctggaagaattctctggattacgagacaaagatttgttctttttcctcactttctcccaaacaaatggagtctctttttctgtgctgagccaccttgaactggagtgtgttgatgtaagcacccctctggccattactgctgagactgctgggtcagacttgaagccagcacagcacagggtctcgcccaaggcccgctgtaaccactacatggctgccacctgtgttcactcagggccctagggctctatagtcagcaggtggtgaagccagccaggtttgtatcctccccttcagggcagcaagttcccccaggtcccaggtgggtacagacatgttgtccggaagccagggattggagcaaaaagccttagagatttcttagcactttgggagtccaaggtgggtggatcacctgaggccaggagtttgataccagcctggccaacatggtgaaacccatcaagaggacagggaccatgttgtatgtgttgttcgccactgtatccacaccacttagcaaacaataagtatttgtttaaataaaattaaattttagctgggcatggtggctcacatctgtaatcccagcgttttggaaggctgaggtgggaggatcacttgaggccaggagttccagaccagcctggataacatggcgaaaccttgtctttactaaaaaatacaaaaattagccgggtgtgcttgtgtatgcctgtaatcccagctattcaggaggctgaggtgggaaaattgcttgaacctcagaggcagaggttgcggtaagccgagatcatgccactgcattccagcccgagcgacagagcagactctcaaaaagcaaaaacaaaaaaccttagagatttgcctgatgttctattctgcagctatgctggcactcaaaccacaatacaaagtccttcctgctcttctctcctttttccacaggcaggaagcctcttcctgtggccaccaccacaagtcattcttacatacacagatgttagtgtagtaatgcagcaaggaattcccagggaaatcggttttttttttcctgtatttcttctgtagctttgccaaacaaacaaaacaaacaagaccccaaggtcactgatccaaatctgaatcagtgaaacggtctgatgaaacactttttttttttttttttgagatgaggtctctatgttgcccaggatggtctctaactcctgggctcatgtaatcctgccgcctccaccacctgagtagctgggactacaggctaatgaaacactttgataagataaatcgtaatctgctaggaactttcttctgttcagtccttaaaatctgaagatgtttagaacagatgggctttttgatgtagctctccagaggtaattattttctttttaaaacagctaggatcattctcttactgatcagacatatgcttctccagcctcatacaatctcccctgtcctctgagggctgctgattgtctctgtcctcttaccttcaccatcttagaaggtagagccaagtctacatgggtgatatggtacagtgaacaacatggttactaaagagctggattagttttaagccaccccattaagcctgaactgatgtggaatagtcacaagaacgtaagtcagggacatgaagaaaccctttgctgtaatctaccataccagttattgtccagggtggagcatgtaatcatattttttataacaatctgattatactttctgccattttttattgggaatttgaacattgcaggttaactcataaaactgtgaaagggcaagataaacataggatacaatatatttaattaagatagcaaaacctttctgtactttcttacaatggcttgtaggagatctggagcagatcttcaacttgctaatattcagttaggcccagatttggtatttggaagaggcagtttattagaataatggtgctcctactcaaggaagaaacagcatctataatacttagtgtgatcagggacttgactttaatgaaatttcaaatagaagtaaatttgacttacttcgtcatgtggaccaattctagccaaatattgtggatttgagattaggaccagaaaactcagggtctacttcggatgacagtaccccaggaggatctggtctttctactctatgcttggaatgaaccaggtgagatgtttgggtcccagccagattcttggggtcattgtagattcacacagacttaaatgacttcatcttatcacccacagaaataaggatgaaccatagccaattcatttcattctacttcttgaagtgaataggactgtaggatcaaagttaaagactgtaatgaagatcacacactggactgtgtatcaagtaaggcccagatttacctttatagacttaactcactctatttgcttttctttttttttttttaaaggctacaactgaggtggaaattaagaagaaaggccctggatctcttttagtttccatggaccaattagcctcctacgggcggaaggacagaatcaacagtataatgagtgttgttacaaatacactagtagaaggtatgtgccctataatgtcagtccaaccgctgagatataaatatgtaatgtgcatggtaaatataatccctgtcccttggtctgagttttgcaaagactgtctctgagctttaggaatggaacgtattcctatatagaaactagaatcacaaattctcttcctaaatttacatgcagagaatcaaatatatgggcttcagatatgctgccagcattgtacacaggtacgtaagagataggaaaggacctgcatgagtgacctagaggagctcaagacccctgtcattgtcatcatgaggagtcagtggggtgctgtaaggcataaaggaatgtagactgaagaactgcaaagatacaaaatcatagaatttccctcatggtctctaccccaaaacataccttcctttttcctttttaactacttacagcccttttggttttcccttaaggaagaatgtactgtgcgttttgattgcacctagttctgtctctcttcaaacacccccacatatccataaataaatttgatgtgtgactaggtaagttgaaacccttcttactttgagtgctccatgccacagggaaagagcaaccgctgttgaatcatagcacgtctgcagtaatgttgctgtcagtgcctgagttctctgtgttaggtgggttctctgtgacacaacctggcgtgctgaggaatacctgaagattttccattaggccacagtccccctagcaagaatcagctcctctccagttatggtctgtagtccaggatctgcatgctttctttagaggtaaattcagtgacagatttcctctcccaagattctgatggattctgcacctatcttcttcattggatttatgttgatttagctgagttgtctcattgtagatctagactattcttgaggcctcaaattgctattcttcagaatgcagatgcctggaataaccaaagtccctgagacaggaaaactcaaaatggtcccagtatccttgagacaagttatttttttagtcatttaaacccagtgccacttctactctgtgtgtatgtgtgtgtgtgtgtgtgtgtgtgtgtgtgtgtgtgtgtgtgttggggagggggttgcagtggagggagagtccctagaaaagaattctctaagggtgcttacttccccacatatgtgcaaagaatgagactataactagactgaccttgagaatatctcaagtaaaaaaatgaattaactgctttcatctgccttaacaacctctgtttattcctgttttctttaattaccatcggggttattgtctgatagaacatcttcaagctttgtagcttatactttgagattgtgggcaaataactgcaggtcctaagaattccaagctaagaaatcctagcctaagaactttggctaattttattattcacatagagaccaagacaggatttctctgggtatctgtggcagttgagaaagaaaaactaacaacctccaacaggtgcattttattattggtagagacatcagaacagcagaactatcaatacagtgcaacagttatttatttattactgcattggcttagcacaggtcccagcaaaagtagatgctcaaacaatctttgtcaagcaggcattggctgtgcttagaatggtaggagtgctggtgctgcagggctcctttcggaggccttagagctttcatccctttactgagttggatacaaaccattgggacgttttaaaattctcttttctgcctcttctccctgttctttctctttctgtcctgctgtgcttattctccaccctttacccaccatcacacacattaaaaaaaaaaatagatgtaagggtagctcaggagacagagacagatgtagttgtgttcctgaataggaaaatattattccagttttttgtcctctttcctaagccctgattttcacatagttatacctgtcctgccatttcctgctcaggcattccgcctaacctctccccttgtatttgacccttttgttgaagctgatgggacagaactgctataagcaaagagcaatgaatggctgaccggaatcacgtaagagggaatgaaacatccatgtctgtttaaccattggtccctctgtatttaatgcttgctacagcattaaaagccagcattgaacgaattgtagttcttgctctcagactttcagttaggaaactttgacattattttaatccaaagattaaaataattattatttaaaacccaagaggcaggctacgacttacctaatagtttagaaaccacagtggagaattctagaatcctgtcaataattagaaaaccctgcctgctctgccccaccccattcctattgcaaacatgcttaccaggatccagcacatttagtagagttcaccactagcagagctataagctttggggatatttcagaggactgggcgtggggtgtaggtagagtagtatagtctcactcttcaaagcagtaaagctgtgggagaaggggagatcgtggtgaagtaagaaagaggaagtagtgatgtacttgacattattgatttttaatatcaaggaagtacttgacattattgattctcaggtaaaacaattccaaaaactcccctagaggtaactggccatagctattgctgccactatttatgtattcttttatttaactgaccagcatcaagtgtcagtatagttacttggctgatatcagcctttcctgtgtggcagtaaccttagtgagggtgggggctcttcatcagtgtatccttggtgcctagcacattgcagatgcttaaaagttgttgaactgagttgagggaagctcctgccttcagaaacctcatggtctaccaaagccactatcttggtgaaattgtgaggtcctttctgaaaacaattcccagtacccctgaggttaagacccaaagtaacctctctgaaaatgtccaaaagcaaagtaagagaaaccccacaaacctttggacatctcacccctagatgttaacccattcccccaagccaaatactgggccattccaggcatgtggaaccagtaacagtttgcaaagtcataagtcacttgataagccttcttattccccaaatgatcttttctagctcctgaagctccacattgtcttactttttgtaggcgtgaaagtttatcctgaggatcttcccttctcagtaaatgtggctaagagatgtctggtatccctagaggaattttcttggtgccctgtagggcaggaaaacccctttttgctgtgtggcaagactcaagacctagttcattttaaaacattacaattgtgtcaactgtcataaatgtgagcagtcgcagcttcaacctccacgtgccaatgagcaacagtcgtttggagaagggaggagataaaaaagggaacggaactaaatttccaaatatgtactgtaagaatttatggaaataagagaaccttgtcattttcttttttgggggagaaaactatatttaccttaggaagggagggagttctctgaggaagaagccacagcagattttcctcacagacttcctttcccacgcagcctcccacgtggcgcttacaggggtccttaaatctgaaaaaccaaggcaaccattttcttagcaaccagggaaccaatcagaaactcagccaaaaccaccttgtctaaaataaagcatcgccatccagctggttggaaaagggaacgtaggctaatttaaggcagccatcttggttttggtgaaatgttagggaattttttttttccttaaatgatttgatagtgtcagcagaattgaacagagagagagtagctgttacagccaaccctgtagaatcaatacctcacattctgacaggttttcaaagttcacctcttaaaaagcatcctcttagctcagggtccttggaattttgtaatcctggcagttgtatttcatgcttaatttgaggagagctttgttatgtagggcttcatacctagattgttgtaactgccagttgagtgtctgtctttgatatctgataattcttttgaatctttgctttggaaatgacctttgagatcgtctagtccagtgatttttcagtctgtgtgtatcagagtcctgggattctaggggagtgcctcaggagaggaagaaatctatccaagtgggctcctgcttcatctagggaaattcctggtgtatttcatgtattgggggtcatgtgagatcttgtttgacaataacactttctgtttctaaaagagacggagaagataagacaaaggagagaaagaaaaaggaaagaagatgatgacccagggagggaaggaggaagaagctgtagaaagaaagagaaaagcaaaggatgagagcaggaaagggcaaaggggcaagaaagaaggagacaggaggagacagaacaaggaaggaaaggtgaaaaggggagaggagtgtgacttgtatttctaactcaaccatgagctgtaaacttgttaagggtggagacagtaacttgtacttctttgagaacatagcagatgtgttttacatggtggcttggtcgatgatgctggtcctgctaatgaagataataataatagcaaacacttccatggcactttctatctgccagggtattctggggcactttgtatatattttagctctttaattctcacagcaaatcaaagtaacttgttcacggtcccacagttagcaagtgtctgagctagtatttgaactcaaatatgtctggcttgagtccatacttttgatggcttctctgacaataattgaataatatagtgtataatagaatacctaccatttattgaggatttaccctgtgccagatactatagtgactgctttacaccagcatctaatttagtcaaactcattcagtcctgtaacctgtaaagtaaatgccttgttttacagctgaggcagctgagcagagaaatcagctaccttgcctgggatcacacaacttgtaagcattcaagccagttgaggccagggagtcagacctgagaggccaagctcttgacctctgtgctcgactcccataactgatgggttgagtttcatttataactagatcattattaacagggaggcaacttgtgtggatcatgcttcctctctaccttagagagaaggaaaacacttaggaatgtcagcgctcaaaacaaccttgattccatattaataacagtgataatgactgagagtgagtagtgtgtccccctggttttcttgctgtgattgaggggccatctttgttcttacttactgtagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggtaagatggaacactgtctcccgatattaggattggaatgtgttttgcctgtaggatatctttttctggtactgaaagctggagtttctttgaaaagaaagtaatagttaattggaagacaaatgcttccacatatgccaggacaccagctgcttttctgctcactgttgtgatccctttcctctttctgcagaactttgaagacgtcatttaggagctattggcagaaaaccaatgttggcgacagaatgggaggcctggtacagtgggttggtttcagcagcctgagccaagtacttctggtttttttcctcattctgtttctcacattgattcctgcattgctccagcaaatcagttcccctttcaccttaagtaacatctaccacaaaataggggagctcttatagtacactacatgtaactatggatttgcctgctctgtcaggccttaattacctaaatggggactatttatgggaaagttttcactcaaggctggtttccccctccaatccaacttctaggccattaaagtatgtagttttaaaaaaaatagattacaaagtgtgtattctgaaattaagtagctgtgatttgaggattatgtgctttgtttactacaaaaatcaggagttaattgaattttgatagatagtaaagaattagtgagaaagaatcccctaggtcacctgggccccctaattccttaggtgttaaccacaggcatgtccaaatctggctattaccgtttattaatgatttgagcatgatttctgcccagtttctcttatgtgtgctctctcactctctctctccctctctcacacacacacacacaccacacaccacacagttttgaccttaagtcctattactatactattagagcactacaatatcattttcatttctcattctcaaaacacatggcaaagagatcctttgtagcccattgtgttccctggtatacatccaagatgattctgtgatctttccagtctccatgtctgtggttggtgacatgctccaggctctctgcaatgatgtaaacccttgtgtgcagtccagtggaatgcccggagtcttgaaatgggacactcccacagactgtccaagaagtctgctgactggctctgaaaggtgcctgtcagtgggcacatgctgtgactggctgacacagtaccttggccatcaagcagaattttaaccctgtcaacaatatcatttagatataagcttgattttagtagccaagagttaagtaacaagaaatatgtcttgataatttttattgttgatgttggtggttctgttttccctgtagagttttgtttttctcaaaatctttacttttccttactttttacagaaccagtctttgattttcaagggattttgagaggctttttcacctatccccactagaactccagtcagaccaggctagcaaataatgttatttttcctttcatgaaaatttagagatttaggatgacatctaaattagaattgtaagtatgcagaagactttgctcagaaccatcagtaaaaaaagaggaaataagagaaggtcaacagctaatattcaggaactccatgagacaagccaacccagaaccaagttctagaataagaaagctcttaggtggcaagggttttcaaacggcaagtaaatgtacaattagcagtggggagggagcttagtatatagtccagcatactttgtactagatgtgagggtaaaaggaaagaaatcaagaatggtgcctcgatgtgggggtttaataactgagggaaaagtggaatgactgcaaaggaactggcttgacgtataaaatcaagagttctgttttggacatattaagtgtctattcactgagtggagatgccaaataaggaagttgggtacgtgctggacctcatgagtgttttctggacaataccagaaactcaaagatatactttgttgccaatcaacacgagcgctgagattggcagaaagaacacagaattggaagtcagaagatttgggttttcattcctattagatatcgtataaccttaaataagtcattgatttctgtccagaaatgtttcacatctgtaaagttaagataataccatgccatcactggttgggaagataaaatgagaacctatatggaagtctttgggatagctaaaagtatcttataaatcttaggaactgtgttagtataattcaataggagaattttcaaagatttggagaattgggtagaggctttgactgaacctgagaataagaggatggaaaaaaaatgagactgacttgtgaaataaagtatgaccttgtcctgtggttggaatgtgaggcttttcctatagccctaattgtgagttaagttgtctttgtctgtagacatacagaaagatgtttttaaagaaagatgagaacagtcttctgcaaattgaagatccctgcctcttctaccctccccaaaagagaaaataatagtaggaagaaccttagagataattcaatccaatccacatgttttatagaggaaattgaagcccagggaagctgtgtgacttatacagcctctatgttatgtagtagataaatgtaatgattcttttcctgccttttttgcaactaactgaatacaatagtaagtttatgaggtgactttttaaacattctgaatcttacaggaaagtttcaagtacagtacaaaaaacttcttttttttttttttttacaataaaagccttatcacatagccttatcacccccaaatgcttatagtgtgtattttttatacacaagtacattctcctacataaccatagtataactgtcaaaaccaggaaattaacattgatacattacaatgttgtgaagtaaagtatgaccttgtcctgtggttggaatgtgaggcttttcctatagccctgattgtgagttaagttaagggtctaattctcaggccctattcaagttttgccagtgatcccagtggtatcctctgtaacaaaaggatgcagttttaggattaccccattacatgtagctatcatatctctctaattttcttcagcctggaggagttcctcagtctttccttgactttcatgtcttgacattttgaagactacaggtcatttgttatgtagaattccctcaatgtgggcttgtctgatgcttccttatgattagatgcaggttctgcattttggtaggaatatcagataagtgagactatcttcttcacgttgtatctaccagttggcatgtagcttccatttgtcccactactggtgacgttcatttaatcacttgattaaggaggtgtctgccaggcttttcatctgtgaagttacgctttttccatttacagtaagtccccacttaacatcctctataggttctagatttttggaaactgtgactttaagctaaatgatatactgtataacaaaaccagttttaccataggccagttgatataaacaagagttaagttcctatggcatatagtacatcatgtggcttaaagttgctgttttcaagaacctattgaggacattaagtgaggacttactgtatacttaataggtatttgtgaggaagaccatttgaaacgatttaaatttcctgttcattcagattttgatttcttcctttatttatatcagtatggattaatggttttctattttatctgagttacaatccatttgatgtttagacttcctctgatgtggccagtggaaattccttcaagctggcttctgatccctttcacatgtcttcttcacttcctggctttctggtaccacaaggtattccaggatactttcgtgtttttccagccccagggctggaatcagccatttctcaagaagccctgagtcttttagtggaaaaatggcatttagaagtagagatctaggctgagtatcagctgaggcaggaggatcgcttaaagtttgagaccagccttagcaacacaatgagaccccatctctaaaacaaaatagaaaactaaaaaaaaaggaagaggtggagatctgagatctaggtatgcatattgctatgggagcagcatgcagcccctcttaggacatgatgagctatttatattgagctagccgtggatattgcaaaccatgagttgatcctgtcacttccaattctaatccaacacttcaagattctttctagttttctccctttctgtatttgtaactcctgcaacaataagaaacctcgttgccactatctttggtatacttatttgatcaaaaagccactgttaccttttatagcatgggtaccctcctcaccctgtataggctctgggtgccccccaccccttcagcacccgagccgtgtcctccatgtggacacctgctcacactcctcagcttggaacccacaccagtccaactcacacatggtttgccccaccctgcttagaccccgatgcctcacagtaggccttcgcctgcatggacatccttctcaccccacaccagactctcccctgacatagaagctctgtttatcctgcacagactcttaacacccctcaccaggccacccctcgtcaccctgctcatgctgccatctgtctgtgtgaacactcctggaagttctcacctggcccattgactttatcccaaactaggcgacatcctcctcaccctgatgagctctcacaccctcactgtccacctccctgtttggacactttcctcaccccactcgggatctgacaccccacatcaggccttcccctacatgagtccctcatcagctcacctggctctggcaccccttgtgagtcttcccctgcagggagtccctcctcagcccactccgggatcttctcctggagatgctatctttaccttacttaagctctgactctctctgctaagtcccccaccccctcaagtgtggacgtcctcccaccacacttgggcctcaacacctatgtggggtagccacgcctcataccacttccaactgcccatcctagactgttgcccaaagcggatgcctctccttggcccacaggctcagacagcccatgccagcctgctgccctgcatggacctcctcgctcccctccatgtctgactctccttgctgggccgtgcccttcctggggttccagcactgggcagcagcctccctctaccggcatgcccacctacagccttcaggactgagtggttcaggaggaaggggatggaaagaagagctagggtttaatctgacatataagtttaagttctcagattctaagcaaaagcaaaaaagcagaaagttattttcctccttcaccaaacataaatgaatgttatcatttcttaattataggttacctagattttcagctaaaaagaattcttttcagattttttctttcatctgtgcttctatagccccatatggatacttctttttaaatacttgtcacatgatttttagttattgttctgtcccctcctggttgggtaaccaaccaccttaatttgcagaggcctgaggaagcccttgcagttttaaaactgggacagtccttgtcaacaggtacgagttgatcaccctaactggagatgcaggatgcaggactttcggtgtgaaaagtggcaaaccaggatgagttacttacattctggcgactgagttcgtaaggaaccattatttttcatggtcacattttttttttcatgtctccattgcagtacacatagaacttttaatcaaattgaacttgattaactttgggcgaggatctactgcagaacaggagctgacaagcccttttctgagcacaaagagaggagcatgccagacgctcgtcactttcaaataaggctgaattttttctgcaaatgcaatgagggctgaaggaggtgtgtgacttctccccagaccccatagtgacgccaaagtgaaatataccccaatcttaggactatataggccttacttgttgacctggcattacaagttatcaggcatatgaccacaggctagtccaaactagactaggcatggctcatgcctgtcatcccagctctttgggaggccgaggtgagaggattacttgagcctagaatttcaaagctgcagtgagctatgatcatgccactacattccagcctgggtgacagagcaagaccctatctcaaaaaacaaaacacaacaaaacccaaactagaaagatatgatgactgtgaattcagagtattttatagagaatagagaaccattgttttatagaatattctgaggagatcaggactcctttatactcttaaataaaagtgctatgtctattgatatttatcatgtttgaaattaaaactgagaaaatttctaaacattcattttgtaattaaagaattacaataatatagctaggtgagtcccagctacttgggaggctgaggcaggagcatggcttgaggccaggaatttgatgccgaagtgctccaggaacacggctgtgaatagccattgctttctagcctggctagaacatagcgagaccccatctctacaaaaataaaaaaattaaccagacatgcagctcatccctataatcccagctactcaggatgctgagatgggaggatcacttgagctcaagaattcaagactatcctggacaacatagcaacaccctatctgtaaaataaatatcacaataatacacctatcacacatgaacataaataacatattggtgtggggaaactatattttttaagaaaaactggaagaatggcattgttcgacattttgcaagtctctttaatgtctggtttgtttgtttgtttgtttgttttgttttttgaggcagagtctcactctgtcgcccaggctggagtgcagtggcacaatctcggctcactgcaacctccgtctcccaggttcaagcgattcttctgcctcagcctccagaatagctgggattataggtgcccgccaccatgcctggctaatttttgtatttttagtagagatggagtttcaccatgttggccaggctggtcttgaactcctgacctcaagtgatccacccacctgggcattccaaagtgctgggattataggcgtgagccaccgtgcctggcctcatgtctggtttaatagaaggtagttagattctctttttctgtttctacattcagtctgttgtgatatgttattttagctaaagtataggaagaaaagcttgcctcacacagatatgtacagtagtcctctcttatccaagggggatacattccaaggcccccagtggatgcctgaaacctcagatagtaccaagccatacacacacacatacacacacatatacacatacatgtatgtatgtatatgtgtgtgtgtgtgtgtatatatatatatacgtgtgtgtatatgtacagccacgtaccacataacaacattttggtcaatgacagactgcatgtctgacagtgataccataagattataatggaacatatataaaaacttgttatatgccatttgatattggccttgcaggtcaagtaagagaaatgattgatgttaagtaaaggtgctgggacatttggttttccatattaaaaaatatatatatataaatgaaaatatatatataaggcctaggtttgtgtaagtacactctttaatgtttacacaatgactaaattgccaacaacacattatccctatcattaagcaatgcatgaccatactgggttttgtgaatctgataaccaagatggctgctaagtggctaaagggcaagtagtttatacagcatggatacactggacaaagggatgattcacatcctgggaggatagagcaggacagctcaagagttcatcatgctactcagaacagtatgcaatttaaaacttatgaattgtttttctagaattttttatttaatatttttggactgtagttgatggtagctaactgcaactgcagaaagcaaaaccacagatggggggtacttctgaagttggaaaagagcggaccatgtgcttcctgagagggccttagggactctaaggatcctcaactaccctttgagaactgctgcataagaacataaattctaaagtgaatatttgatgacattcaggagaacaacactgtctaagtaatgttaccacaacagagtaccatagactgggtaatttataacagacagaaatttatttggctgacatttctagagactgaaaagtacaagggcatggtgccagtacctggtgaagacctttgtgctacatcatcccatggtagaaggccaaagggcagaagaagctaacagtgagagagcaagagagggccaaacttgcttttatcacaaagcccactattgagataaccagcccactcctgcaataacatcagtccactcatgatggcagagtcctcacatgacctaatcacctcttatcagaccttgctgcccaacagtgttgcattggggattaagttttaatacatgaactttgggggacacattcaaaccatagcagacacttatatataattgctgatacactgcatgtaattcttccctttctcctctttgagaagcatatcacagtgcaaacaagagcatcataggataaccttaaatctgctcttctcaatttttaaaaaagcaattccttcacacagctaatagttataggtgtattttgacacacacccaactgatggcaacataccatgtgttgaagcaacatggtggctgacctctgtcacagacaatttctaatgcctggggccattcatcctggtgataggcaacaccacataactatgtcattgttgatttcccttaggaaccagagttcccacagtatagatattccatggtcaatcagccagtgcttcttcatgagcacttcctgtgtgtttaccaggaaccagtgggataacaaatcaagtctctacttagtgtgtgttaggcaggctagcatttctatgtggagaaacctccgatgggtttacactaagggtgaaatgtatgtcaggatttgatgagctgtctgtactttccctgaactcactcagagggatgttccatctatagatgcttaacttgtattatgaactttgattttacttaactggatctctttgtggaatttttttttcaattttagaatattgcatcttcaatctagaaacatgaggaactacagaaaagcatatgaacaccgaaaacctataggccacttatccattttgatgtattttagtcatcattctgtttttttattgctgtttattaattcagtatatttccaaacagcatctataatctaagtattttttactatttttaattttgttctcagactatatcttcagttttccttaaactgtttcatgtggaaacttggtaaacaggtagttacaagtactaatatattatgacagttttaaatctattgtaaaaataagtcctctcctctaaaaacataatattcaataagagtctgttgttatatggttagcctagaatgaaggtattggttttgtcaaaaccggcattcatgggatttgactttcatcctgcatatcacctcctaatgcctgcaggatatggaagtcatcgggaagtgcaggcactctgtcagcagaaaaatgggagatgggtaacatagggaagatgtcatttttacacacaataagctacaaatctaaaacatttattatgtcattgattatcctattgaaaaaaattaaaaggaagcaccgaagtctccaacaataggaaaattctttgaggatgtatttacccaatagaattttattcactatttatttatttatttatttattattatttatttattattatttatttatttgatagggcttgctgtgtcgcctgtgctggagtgcagtggcatgatccactgccctccaggttggctcactgcaacctctgcctcccgggctcaagtgattctctcacctcagcctcctggtccctggtaccataggcgcccaccaccacgcccaactaatttttgtattttttgtagagatgggttttcaccatgttgtccaggctggtctcaaactcctggactcaagtgatctgcctgcctcgaccaccagaagttttgggattacaggagtgagccatcatccccagccgccagtagaattttatacaggcagtaagagtgagagttgggagaaccgtgtagcaacatggatagacttaatgataagtgaaaaatcagacccaagatattatgtattctatgattttccaactctgtcaaaatacctgtgtatttgaaaaaaaaaacagaaagaaaatacatgaaaatgatagttgtatgcaaggtggtgagattatagatattatttcctatattttgcacattttggcaatattgtatgcttttacaccttgtggcaaatgtctgggtttggtaagatataatgtcaaattagcattaatactttatttttctaatcataacgcgagtgatttttttaagcaaacatcacaagtattatatacattcaaaatgactgctgaccctttgaaaggagttgcctggtgtagtattgccattgcttagcatattttctcaactgcactcttgaagagtttaaattgagtggttctggcaacaaagatgggaactttggggctggggacttcattcaagaggagaataagattttcctgcatcaggtggggtgctcatgagacatccaggtattgatatcttcagatgcagaatttaactaaggttaactcttgaggtttcttggacatgttgttttcaaaaataatgataaaggcattctctttctactatttatttttcttaccccctgcatccccctatttaggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattggtattccatatttctccaatttcttttaacatttcctatcttgcagctactagaaagagttctgcataaattaatttgatgatttaaaaaatatattagctgtattttactcccaattccagttatttggcttagctactaaaatactaaaaactttcctaaaatacctgatatggaagttagttgttaaaaatcaataagcaaacagaaatcaattttaagaagctctttagaaaatgctgcttaatctaagagatgtctaagatgataagtgaaaaataagacccaagacaatagatgaagaaaatgaactcccccaaggcccagtgtgaggtctaatttgggtttgctagtgaatctatttttttaaatctgaacatctccttgaaatggcaagaatttctccttgttcaaagatgactttcttcatttcttctgtcatggtagcccttccaaaaaatgagaataaaataaggtttttaaatacagatggttcccaacttatgatggctcaacttataatttttccgctttacattctgcaaaaaagcagtgcacgttcagtagaaaccatactttgagtacccatacaaccgtcctgtttttcactttcagtatagtattcaataaattacatgagatattcaatactttattataaaatgggctttgtgttagatgattttggccaactgtaggcgaatgtaagtgttcagaacatgtttaaggtaggctaggctaagatgtgatgttcagtaggttacgtatattaaatgcatttttacttacagcagtttcaacttaaaatgggtttattgggacataaccccatcataagtcaaggagcatctgtattatattgaaattagttgttgaacattggctttattttctctatcattagctaaaaaggagtttgtattaatgtatttgaagaagttagtgtttaagttactcttaatgacttaaaatgactctgaaagaatagttaatggtatcttcaaaacagctgtccgtaaacataagcgtaagtgatatcttgattaactcctcgagaaactgagttatcctgggggtatcccactcagacaaaggtcactactgcttccctggcattagaattggcttgggtttttcactccctattgggaagataagcacttggcaactgaaaaggtttaaagtttcttcattgagatattcagcagttctaagaaagaactggaaaaagtgaaactatttgtaaaacagagatttcactcagtcaaagtcacaaataactgccaaatttttgaaacctctgtttactactctttggttaggatccagtccacagtggacttttttgagaatagttattacctaggtgacaaaagaaaggattgagagtagccttagaacttatattcttaataaattaaaaagccactctgttgaaaacaatatgagtggcccagatgtctgcatatagggtaacacctggtaaatattgaactctctgataagataacttttcaattaaaaaaaagttaaaattgtgctgttgccttctaccaaaattcctattacattctatgttagttatttgttagcatatgacatctaatgatgatctcataattataattttattggccacactgccatagcctttatgcgttataattctcaggctgggccaggcacagtggcttacacctgtaatcccagcacttcaggaggccaaggcgggcgcatcacttgaggtcaggaatttgagaccagcttggccaacatagtgaaaccccatctctactaaaaatacaaaaattagccgggcatggtggtgcacgcctgtaatcccagctacctgggaggctgaggcaggagaatcacttgaacccaggaggcggaggttgtagtgagccgagatcacgttgctgcacaccagcctgggcggcagagtgagactctgtctcaaaaataaataaataaataaataaaaaataattaacactctgttagccaagatcttgtgattatttattacagaatatagatatttcatgattgacaggtactaaaagttctgtcctctgaataacagtataggctcttttcttttttttttttttcttagacatagtctcattctgctgtccaggttgaagtgcagtggtgtgatctcggctcacctcaacctccacctcccagattcaagtgattgtcatgcctcagcctcccaagtagctgggattacaggcctgcaccaccacacttggctaatttttgtattttttattagagacggcatttcaccatgttggccaggctggtctcgaactcctggcctcaagtcatctgcctacctcagcctcccaaatgctaggattacaggcatgagccactgcacctggccagtataggctcttttcataagatgactgatgtatgtggaactgcccctgtttgagagttgtgcttttcctccaagttattccaagcatatcacaacggttttttttttaatcagccattgattatttattggcaagcaaaataggtatttagaattttttaatgtttgcttttttaaggagatggatttttttttaaactgtccttcccatactagtcttttggtttcctacaaatcacattttatttcacttgtcctgtgagaagtcaagggaaaattagtaggaaagggatatgttcatatgtgggttttcaaaaagcagactgactcagatctaaagtaagggactaaggcagtattcctggcttctagttacaattttttcagctactctgaaattaatcagttttcccttatttatgtctcagtttctccccatttagtctgagacactgttagacagtcctgggaattttccaagggtgctcaatatatttaatctgatcttcctaattttatgtttttattttagccatgtggtgagaaaattgattgagtatcatttatttttttgtttgggttttttttttccttagctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggttagtactaatttgtagatatttttgttctacaccctgaatattctacccctggcccagaagcccaagtccttctactgcttagtccttctactacccgtcatgtttgttgatttttattctttcttggcccagaaaccctaaggtacaaatgaaggagaggagagagatacttacagagtgcctacttagtcatgacccccacaacttgtcaacccttttcctagagttaggaaagcaaactcagcaaaattaagtgactttcccaaataaatatagggcttgtatttgaacctagactttaaagtctgtgcccgttcgcctacagaagccttcctttttcttaacgacaatttctcatctgtggtgctattgacatcttgggctggataattctttgttgtgggctgtcgtgtgcattgtagggtttagcagcgtccctcctggcctctacccactagatgccagtagcatcctgagagctgtgacaaccaaaaatgtctgcagacattgccagatgtccctggggggcaaaattgccctccattgagagccactgctctaaaccatcttagagttttatttaatttatataaatgacaaatcactttattgctttactttgatctgtccacctgctctcttctacctctgtctttagaagagtcaccaacagatcttaacaccttgcttcttaaagtgtgatctgaggactagcagcaacaagatcactggagagtgtgttagaaatgcagaattctaggccccaaatcagaactactgaatcagaatctgcatttttaacaagatccttgtgtgactcaaaagtacactaaagtttgagaaaggactatcttacagatcaggcccctaatcccacctgcaagtgtagggcaaagaggactggaagaagacaagcttatctgtcccttaacatgctcatccacattagttctcttcctttttctgccacagagaagcatgacagtaaaaggctcctactcaccagcctctgaaccagctacaactattctgttcttccatttgacctacaaaaaaaatagctactgtaatgtttaattcttcctattctccattcccagccacctttccctcagtcctaatccctctagtcttctcactacttcagtgactataacaatggcttcccggtatgtagcatgatcaacattttcaagccctgactttgccaccctttcttcactctcctgttcttatttgtctcaaagacatctccccatggctgtctccagatacctgagcacttgataaatatcagctgttaccactaccccctgatgacacttttcaggttcccctttgctcagtagtccttctgcccatctagtttctaggaacctataactttggagtattttctgtctcttttctctccactcccagcaaatatctgatgtgtcttaaatgcatctttcttcatagtatttctgtggatatctttcttcatagtatctaagatctgccccttcctttgcatttctataattcattcagtactttataaaaatgtggtctcaagaccacctgcatcataattacacaagtttcactcacaaaaaaaggccaagtcccaggctgtataccaaatctactgaatccaaatatctttagagtaggctaaggaaatatttttaacaaacatccacacccctcactccctgagattctaatgcatgctaagatttgggatttctgtagcctaatccagacccttatcaattcatgcatgtaacagcctcctaacttctgcccttcccctctgtatgtagttagtctgaatgactctttcaaatctctgtcatcatctgtgactcctttactcaagaaccagtagtggatctaattaaataaaattcagatccctcagcctggccttcaaagccctctccaactcacccgcattttaccaaccatattcctttttaactctacaaatggtgcagataatccgaacacttatagttcatttattgtttccaccctcccactctgcacatgactgttatctcctcctaaaatgtatgccttactcttcttcccaagtatgacaaagtaaggaccaagtgtaaatcaagaagtgagcacctctcccactcctaaaaagtaagtcttaataaaaataaattaattaaccttaatgttcaatgtagtaaaataggagattaaaacaaaaataaataaagcttgtatctgatggcttgacatttggctatgcaaaaaaatataaaagtcaccatcactgtcttgctaaagcgattttatttttataaatgttaggaaaacaaaatttgttcttaaaatatgatgacatgaagtgtatttcagttctaccttcttggttttttgtatgaaaggatggcacttcaagcctcatttttttttctgacgtgggtcctctcaaactctgctcatgaatatgtggcatctgtacactttgtgaccctccccagtaatgtatattcacctttttattttggggaaatgtccttccaaaatggatacagcctttgttttttctgattataagagaagtaatatattttcgggtacatatatatgtatgtaaaactataaaagacccaagaaaagaggtgctaaactccttgtattacaaccaaaaccaaacacattcccacctggtaacatgagcattaaaatactacgtgggccaaacacaacacaacagagcctctttgagtctgtcacgtgaagtccattggcttccagtttgcaaccctgagttcgatggataacttttctgcatttgttccttttttccaatgctactggcataggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagatgtaaggtcccagcctagaaacagccttgatcctgtgtgagagcaaacagggtgctgtcagccttggggattggttcagctatgggcttggtagagagattctggaataaccaatttacttggagaactaattccaccatcccagtatcattctaggcctttcctaaatgaggtggaatcctaagatgatcctggacccacctgtgtcacttctggatcatctgggtcagaggctgggataagccctcgagtacttccctgccattcatctgagttatagccctgaaaccaacctatagccgcatttagctttgggtgggtcattgccgtccgtgagtaggttttgatgttatatcccatctggacagaagttttgggggcccgtggatatggtttgcttatcgcgagttgtagtaagaattctagagaggagtcagagctggcccctcatccccaccagaggcagagttctcagtgtggagtgggcatgttgcctcagagctgcctgatctcccttttccttgcgtgtctagaaactagatgacaccagctcctctgaaggaagcaccattgatatcaaaccagaagtagaagaggtccctgtggaacagcctgaggaatacttggatccagatgcctgcttcacagaaggtgaaaggggatgaggagcggatgggctggggacactcgtgttgctcacagacacagttgtgccagggctagtggtgggtgaggggcagctcagtggctatcttcaagaactttgctccccaccccaccattttgaaggaaacttagattgtttcccctatttgtatctgacaagtatataacaagtttctattctcacacttttcatcatccattttctcttggcatcagcccaaaaatttccattagaatgtcctgttctgtcatggctgtgcctgcctcctgactacccttttctcctccctctcactgcttctttgtaggacatcttcttgactcctgagggccttgccccattagcctttgcagcatagccccagccctgccaccctctccatttctgaatcagacattctgattcagccactgtcctcctggctgtgtggtggggcagcacctggcagggcctgggagatggaggagagggcaggtctgggcggggcacgtttccacggtctgacccgcctctcccgctggtgccaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggtgaggtccaggggagagtgtgaggagggattggctggggaagggtgtagagaagccagtgggaaaaggctgaggccaaagcccagtggctctgaaaacagattggctgtggtcccaagaagaatgatctgttaaagtgctttaggctgctgctttgagcctgtgggattagaatccatacttgttcacattgtgatcctcttagcactgtccctctcatgtgtccccactgcctggtttaccccatccttagcagcccctaaaatgaacttccctctcttacctaataccccagatctcttgttctttgcttttcagttacccaggatgggagggaggtgttattcctccacttactgtcccttcacagcctactggatttagagaacccatggcattagcaaatgtttagagtcagatctctggtacaaaggttatttatattcacattgtcctaagtgcttattaatcagtgaaacatacaaggccctgaacacacaggctcctccactgctgagactaattatcttggcatttgcttttatttttagctgggttataactgaggagacctggacagggtgtggtgtgagggagttgcttgctggttgaatagttttgaggactgccgggactagagacagtacacactttccttaagggaaaggtgaagaatgcataatgttgaagtatatttaaagaaggtgggcacattcatgtaggtgtggaatcaaagcttaatctggaagcaaaaaaaaaaacatagcaagggagaaaccccaaattcagtgttaaagattttgacagtgacttgggaagtttcactgacttgtgaggcaggagaataagaaaagctgagagaggcagcagtgtgtattaaagacaagcccaagggtgaagaagacagctttcatgaaaagaggaccatgggaagtaaggggaaggtttacacaggaaggtgtcaggattaaagtaggaggcaaaaaagatggcttggtagccactgatctgagtgaaggggagccagaccaccccgcctgcccctgggccacttgttgcatcaacgagcattcacacaacacttcctgaggtgccgcagaggcacccaaaatgacagcatggtctctgcccttaagaaaagagaggatcagactaagccgagtgattggagaaaggcgttttattattgctgtcattgttattcttcaaatctagtgaacaaaaatctaaacagtatgacccatcatttggctcttctctttcccatctctacaaaaaagaataaagcagtagtgaactaaaggttttgaactttctttactgctcacctttgacagtttttatttgtcttaatgattaagcaaaggatatttggttcagtacctgcagaccctgtgcaacagtaggcatgagatttaagaataacaaagtggcccggtccagtggctcacgccttaatcccagcactgtgggaggccaagacgtgcggatcacctgaggttgggagttcgagaccagcctgaccgatatggagaaaccacgtctctagtaaaaatacaaaattagccgggcatggtggtgcatgcctgtaatcccagctactcaggaggctgaggcaggagaattgcttgaacctgggaggcggaggttgcagtgtgccgagattgcaccattgcactccagcctgggcaacaagagcgaaactccatctcaaaaaaaaaaaaaaataacaaaatgaagccacagctaaaatgtccaagaagaaactagaacgtggttacaatgggactaaagagggtggggattagggccctcagtgacatgagatcagtcttgggcatctcacataccttggattatagaagggatttccaacagatggggttaaggggaaacctgacatggaagctgttgcctttttgattgagcaaaggggatttatttgcaccgagaccctgagtaggactgtggaacccatgacccctgtcccccccactgcctgttgatgaaagtggagccgggcacggtagctcacacctgtaatcccagcactttgggaggctgaggcgggcgaatcacgaagtcaggagattgagaccatcctggctaacacggtgaaaccccatctctactaaaaatacaaaaaaaattaaccgggcatggtgtctcacacctgtaatcccagcactttgggaggctgaggcgggcgaatcacgaggtcaggagattgagaccatcccagctaacacagtgaaaccccatctctactgaaaatacaaaaaaattagccgggtgtggtggcatgtgcctgtagtcccagctactcggaaggctgaggcaggagaatggcatgaacccgggaggtggaggttgcagtgagctgagatcgcaccactgcactccatcctgggcaactgagcgagactccgtctccaaaaaaaaaaaaagagagaaagtggagcacctgatatccatgtgcctccattttggaaggtgacgctgcagacaacccaattctattcttctggagctgttcagagcaagcacttcccattgtatcacagaggacttcctccccacctccacaggttgtcctatgctttagcaattccctcctcttggcaccatacctgtatagtccaccactctgcagatgggaatgctcacaccaccatataagggattatcccattcatgaatgccaaagaaataaaatagtgataagattttcttcacctgagtatctcccacctgtatgaccctccacccatactgtggtgtcaaggaactaaaaggaggaaagattttgtcatttcttttatatatagtattggtgaggatgtggagaaattggaacactcatatgcttctggtaggaatgtgaaatgatgcaaccactttggaaaacagtttggcagttcctcaaaaaattaaacagttaccatatgactcagcaattccattcctgggcatatgccctagagaatgaaagcatgtctacctaaaaacccatacacagatgttcacagcagcattgttcacaatagaaacaatacacatgtccatcaactgatgaaggataaacagaatgtggtatatccatatattggactattactcagccgtaagaaggaaaatctagagagagaaagtaggtgagtggctgcctagggctagggggttgggggtttagggggtgatagctaaagggtgtgggggttcttttggaggtaatgaaaatgttctaaaattgattgtggtgatggatgcacaactctgaatatactaaaagccattttaaatggctgaattttatgatgtgtggattgtatctcaataaacctattaaaacataaaaagcctgtgtagtcggggagtaaagcgaaggagacagggagctgatgacaggccagcccatgtgggacggctccctgaggatagcagtgctccctgtggcctgcttgcctttaggcactgtcataggcagctgctgcctctcttttaggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtaggtgtcttgctttctgtttcccaccccttccagcaggaacacagaaacaggggtctctctttttctaatggcagtagctgccctgggcccaggttatagatcaagatgtagatgtaggtgtgggtaaagttcctttcatagcactgagatcttcatattaggaaggggaactctgaagtccctgcatttgctccagggggactaaccaaccaactctttagtctggtgggtttggcttaacttagattcagagattaggagaagtggaatcactggtctcaaaggcccttgttattaggctggggacaggaaagttcttgccccaggccagcaggatgtcttctttttgtggctgggagaaggggtgagagaatgagatgggggctgttgggacgaaggtggtagtgggatctgtggtctctcctgctgaccattcccatgaacacttccatccaactcaagtcttgataccggactcatccaaacaagtcagtttattaggtgaggggagttcatactgaaataaaatacccccttcttcccacacgttgccattctacagtgatcatgaaacttgagaaatccttttttcctccaggttccaagactatctccgaggtttgctggccatgacctcctgtgttttttccaggttggaagtgacatcctctctttctcaccccaccatcacttgtcccttgtcagctttactaaggatgatgactggtctcttattctatgtaataactaaagcttctttctttcagccaagtcagcattttaaagaaaggatgaatgggatttgtaggaccagggcctgtggaaaatttacttccttctgagggctgttaagtgccaagaaagtagaacggtggagactagactggtcatgtagaatgcaggtttcattggctgcatggtgaactacctgtagagtacctctgggatccaggatagagcagcatagaaatctctctgatggcaagtgcccaatactgagccatgatttctgctctataaatggatttcttttccaatacattgtctgcaatttccatggttaatgacccccttcccagaactgtcactgtgccctcagttgcatccccatttcaaaaagccagcctttgggaaatgcctaaatgaggtgaggaccccagccctagagggtaccaacgtgagaccctcgcagcgagagatgttagcatccctccagcaactgttgataccatcctgtggtggaaggacctgtaccccccacagacagaaagctggagctctaagacttaaagaatgggattgagataagacccagagagccaaagtgagactgggccagttctctttctggagaaagggcaagtgccatagagtcttcatttcacaatagctcctcaggactacctggcgtcaggtgggacacctgaactcaaactggtactcccagctgcctggggaggctgtgatttgaaacagagcttccccaccatgacctaccctcaccagaacacgttcttctcttttccctagggtgagctacccagaatttgaggatattgatcaggtatcccacatgcccctgctaaacaaataagctgctaaagcactaacagtcctacacattagcatttatttattttttgagatgaggtcttactctgtcacccagcctcaagtgcagtggcgccatctctgctcactgcagcctctgcctcctgggctcacacgatccttgcacctcagcctcccgagtagctggggctgcaggcacacaccaccatgcctggctaatttttgtatcttgggtagagatggagtttcaccatgttgcccaggctggtctggaactccttggcctcccaaagtgttgggtttatagacatgagccactgtgccaagccagcatttatttcacaaactttttttatggctatacgcaaggtggaagggggtaaagagcataggacaggcatgcagccaactgacactaattcataagctcctctagcttttgggggttgcttgatcattacattactgtggtgcccaactggctcctgtgaagacctctgtgccagttattagtaaataatatagttgaatgccattatctgtgtagaattgctccagaagcaataaggagaaacagaggttatccttgttgcaagcatgttcccagcccctggggagcttacactagcttccatagcaactatgtcctgactccaggcttgtgctaggtggggggacattcccaggccatcacagtggctccaatcgctgccgcttttgaattgtggatgaacaattagtcttggactgtcacctgaacagttggaactgctggactttttggtgtccatttgagtaaagtgtctgatttctttggcattttagaacatccagaaggttaggtcctaacttagcaccatccactggtgacttgatgtctgagtctgttactcatcgcatttgtttccctaattgattttcattcttccttgttgcctcttaaagctgtgagtacttttcagagtcatgggattatccagatgacttccaaggttaattccttttatttataatccatatgagttcccaaacaaggaaaacattctgttttgctcaggtccttggctgcattggagattgtgtcctgtaaaagaattttggcatcttttccccaggacatttatcccactgtatgtggagatcagcaagcctgtcagttctggaggtggggacttgcagagactacttgccttggcctgaattcagactgctgaaaacattccccgtttgaaaatattcacatcattttggaaaaatcacctaatgtctttccccctttttttccagcctgcaaccatagatgccctgaatttattttttttttcatttttaaaatttttttaggtaggtctcaccctgctgcccaggctggagtgcagtggcacaatcatggctcactgcagcctccaagtcctgggctcaagcagccttctcacctcagcctcctgagtagctgggactacagacacgtgccaaggcgcccagcttgatgccctgaatttgatgtctcctcaattccttttttttgttgtttgtttgtttgttttaggtttgattctttccttaaagaatactttaagcaatgttaggattatctccaggaggggcttaacagagactcagaggattataagagagccctcaactactaaagaataacccagataagacccttattgatcctgtcctgggccagattaatcagctgagtaacagcttatctcccagcaacgtttagagccagccactgaattggcctttagacctcctccttcattctccacactgtgggcctatttgttatcagataagtcacccaccctgaagagctgggatcttcttaccttatatggtgcctatagccactcaaagcttagtttcttgcaatctttacctgacaaagtttcgtaagatgaagctgacccaggggactgttgagtccaggtacttggttgagaaagaatagcttcccttcagatatacttattaaggcctcatgaagaatctggcaactttcaattcttttggagttaagagttggctgcacttggcaataatagggaagtagatacctccatataaattcttattgataatgatggatagatggagagaaagggattagttggtgctactatgttctagatagggcttttgtggaccatagtttaggcagacattgaactctggaccatctgccttttcatgaaaattagggtagcctaatagggcaacatttacttccggatttgctttggacattgcccagggcttcagagaagataactacactggtggattatgagcacatgcatagatcaaggtccatatccctttttattttatttatttatttatttatttgagatggagtttctctctttttgtccacgttggagtgcaatggcaccatctccgctcactgcaacctctgcctcctgggttcaagcaattctcctgcctcagccttccaagcagctgggattacaggcatgcaccaccacgcccagctaatttatttttgtatttttagtagagacggggtttcaccatgttggccaggctggtctcgaacttctgacctcaggtgatccacccacctcggccacaacacctggccatacctgtttttaaatcgaaacctttaaacctgctcatcctgacccttgctaggtcatacaccttccgcttctaccccctgccagtcatgctgcttaattataaatgtcttcatttttttaaaaattgtgtgtgtgcaattcacagacacaggatcaaagaattaaagccctaggatagagataaaaagcagcaaatgaaactaatttgtagattgtttctttttttttcctctctaaattttctttaaaatatcatcaatttaagtaagctaagctgatatgcaaccaccacttcccagtaggaagaatttaagagctgttgatggaaatatcttaacctatcctagaatttgtgttgtaagaaacccagaacccagccaggcatggtggctcaagcctgtaatcccagcactttgggaggccaaggcaggcgaatcacctgaggtcaggagtttgagaccagcctggacaacacagtgaaaccccatctctactaaaaatacaaaattagccgggcatggtggcacatgcctgtaatccaagctactcacccaggaggctaaggcagggagaattgcttgaacccaggaggtggaagttgcagtgagccaagattgtgccattgcactccagcctgagcgacagagtgagactttgtctcaaaaaaaaaaaaaaaaaatagaaacccagaccccaaacattctggagtttgactaactaaaggtatttttaggtctgctctcccctgaaaaattctgtaactctcagctcaggcccaggaaggcataaatgtagttagcatggtgttgagaaagcatcgtggtaactgctctgagagctctggtagaacaaggtaagtttgcctctggcagagcaggaaagtgtttgaccatacgtctaagcctggaggtctgcaggcttcttccccctggtacggcaacatattcaactctggattgtccagatagtcctgttggaatgttgttgtgtaacctgcccactggggacgtgacctgtctgtggaagaaaaagcattggcctgggagtcaggcagtggaggtctttaatcttggctgcagaactgccagtgtaaccctgggcaaattcttgtgtacgggtctcaatttcctcatctatcaaattttgttttccaataattcttccaactgttattctaatgataaaagttatagaagattgacatggaaatgacagacctgaggataattcattttggactgttctggaaggaaagaatgagaattataaggaaaagtgtggattcagttgtccagactcacccaagttcttgtggccatggaggggtcctggttgtagttaaaagtcagaaggttattttagttaaaagtgagagatagaaggatgtatttaaaagatagagatagaactcacttaagcgagttctcatggagccacagttcttgggaactgcaagtcagccatggtacttattgaacgttttcattgttgttaatgtcgtgacgcatcagattctcaaacaaatggttttttgtcaaccaggagtaaaagagcttgtccctcttggaaagccaatggtgaatgagctgaggagggggcagcttgtgtggagtatgtgtatttatctgtattcttttccataggtaccattaaatttgtctggcttaatttaatggggacttctgggacctgcagagactgtaaagggcgagggtaaggctcttgtcgtctgttcaggctcctctccttccagtggaacttcctcactctctccctctcctgtcagcctctccccttttccaacttgtgtgtatgttctaccgccttctcgattttctcttccctgttgttttttcttctgcctctcaaccacctttccttccaggtcatttgagtcaagttgaaataggcagctggtgttacccacttcctctacctggggggcccaatctgtgtctgcagtgctaggggctttattctaacactctggaacctctgttttctttctttttttttttttttttttttttttttttttttttttttggttaccttttttgtttttgtttttctcttctgtttctgtgtaggtctctttagtcagccttatagctaatgccctgggctactcggaactaggtgccataaagtcccttaggaccctaagagctttgagacccttaagagccttatcacgatttgaagggatgagggtaagatactaagagcagctgatccttctgcatgccagtggaaactgtttaagcatgctagaactgatcacatggtggaaagatcattcaaacacgaattctgtgatgcagctgctgatttgatcctccactctctcccccacacctgccccgtgcaaaggggaattgcgttaagctgcctcatcacattaatattggccccacccccaacaacatgccatcgcagaacatgttaacattggctattctcatttggcctggcttacctgtagcactcggggcggctttctgggctgttgatttaatagggagaatcaccaggtgcaaagaaggggaattgtaacccacagacccactgaaaagcacaagcttcaggcagggagagtgctgtgtaggggcatcagccaggtggggactgtggccttggggtcactgagaaagcagctgagaatgggcttgaagatatgacggcaggcctggaattggggatggagcagaatttggcatggccctgggagaagaaatgcttttctttctattcagattcctcttattgcaccttgttgttctcagcccagtcccaaatggactggcagcctctgtctgatagcaaagcttttttcctcacccccaacccctctgcccatgctgtatggtcccctaacactgtcaaaatgacaaccaggtatgatgctagatgactgaatatcagctagttctcctgaaatttcttgatacctcctattcctgctttggtgatactccacccaaaacaatacaaggtgttgtcaccctaccaacttagaaacaaggtgccttcttggtgccattttcttgtctcttttggtttaaacaattgtgcgtgtgcgtgcgtgcacgcgcacgcacgcgcacacacacacactaaaaagtcaatttcttaaaatcaaaggggcaactggctttggtggtaatagttgtcctgtggtgatcagagagaggtctgttatacttaacgagaccaagaaaactcgcttcaattcagatgttgaatacatgcaggagagcaaaccagaggatatatttttaaatgtgcaaacgtcagaatgtaagaataaaaattaaactgttacgatagtctacgtgaagcaagaccgattggaaaaactagataccaggacagtgctgctacgttcattcctcatctgtgatccctggctgttactccgtgaagccacccacagttgtataggaagatgaaaacttaattgggacttgggaattttattatgttaatttatccagacaaatgctttttgcaacaaattcatgtttcttctgtagcacagtcacagtttggtgtactaaatgtaagctttctgcaactctcctgaaaccatctgcaggaagggcagttccacagtagtgtaaatttattttaatttttcaaaaaacttgggattttaagactttgtcccacagataagagaggttttagaatgtgcgcaataaggtcattgctatgctcaggctaaatttaggaagagagtttattacacaagattcagtggtttagacagccacggtgagattgaaaatgtggttaaaatgttcttctctgctccctgcacctttgcatgttttccagagggagctaataaaatgttttaataaactatactggttttgttataaagttgataatccactttggtgactgaaaaactgaggacaagatgcaaccaaaaaagtccactgaagacagtacttgtcctgagctcctccgcatccccttacggtgatccctgatcttccaaaaagagattggtaatacctgaagaaatccccccatccccctacaaaagaaatgcccatctgacccatctgacgcttgttactaactatttccatctagatgctcattactgtcactcctgtccaggcagacagaagcagtcctcatggccgctgaggccagtttgctcatgaggcagttatttgaataacctggatgcaggagccgttttgggagccacacctcttccctttagctgccccttctttctgtacctttcctggaccctcagtttccagtcccctaaaaactttcatttgtcaacttgaaagtagtttaggagcttggaggtcccgaatgaataatcaaaacagtagataattttatttgaaaagcattacgatgttttatttgaaaagcgtctacatcaagatttttttcggtaagataatatggtcaagaacaagtttgaacccaaagctggaatttaaaacaataaatgcattaagggaagctttggaataagagtgccacctcatttgattgactccagccaactaacagacagtattgaccatggctggttggacaggtgaaaagggatcagggatccattcctgccctgtgggtttcctagcccttctgtgggagagtaactgcaagaagaaaagattggatttggctaagcgtatagtaagtcccccagtaataaacagaattaggtacacatgttttatttaaatgttgtttttctatggggcattttttttttattctttggttttggttcttctatatgccaacacttgagagattacctttagaatattttaaataagaaatcttcataaactgaacaccatccatgcagatgggctcaactgtcactgtgctgctccaattacagactcagagcttcgcttgagagggagcatatgacccaagcctgtcctggggcactgacttcagtctcctcttcttcctcaaaccaaagaccaggaaaactgaagcacttgtgctataggcaaaggaatacttaatgtagttctttgcttttctctcctccatagtaatcttcatttaaagatttcgctgtgctgttaaatttttgagggtatataatattagtgccatgaaacatgcatgcatttttgttgattgtcagtacatagttgtccattaaagccatgttccctctgccagatattaaaagccagctgacagattttatagtatttattgagcaacaataatgtccgagacattatagagaagacaaaaatactgtaagaattaatatccattggaattttctcatcaattaaatgggatttgtatcaattgccaagtataaacttccattgataaaagtaactttttatagattactaaaacctagctttgagtgatatttccatgtctagttcttaacaataatagatttcatgataattaaaggcaatgtagagtaactagagaacttgagaagagaggatcaaattcattaagtgactaccagtggaaatttgaattatcatttataatatatctttagtgtgagagttcaaaataagaaatatgatctcttgcccagtggttgctgagaataacagcacaatgatcattacatatggagcacagttaaatagtttctgcttaacatattgcagcttatcagctaatgttcaaagcaaggaagaattattagtacgtttaatcaactctgggctgggcatggtgtcttatgcctgtaatcccagaacttttggaggccgggatgggtggatcactttgaggtcaggagtttgagaccaacctggccaacatggcagaactccacctctactaaaaatacaaaaattagctgggcacggtagcacgcacctgtagtcccagttactcgggaggctgaggcaggagaatcgcttgaacctgagaagggcctccccttctcagtgagccaagattgcaccactgcactccagcctggtcgacagagtgagaccctttctcaaaaaaagtaaataaaaagttttcctctggttatgattaaagtacatttgcatcttataaaactttggattttataggattttatatcatatcagagaaaaaggctctgaatatttttgtatattgattcatttgatcaaatggatcaatatgtaaaattttctatatactttatgagaataaaagttcaaagagatcacgttaatgtttcttccctatggtaaatattaacccatatttaactgtaacccatagtatgtgggacaggttagactgggtttgagtagaagcatagtcagaagagttcctggaggaagtaggttctgaattgagattaaaaagcagcatgccttggtttcaagtctttggggcctacttcctgtgccacatttgtaatggtctaacagaaagtcacgatctcccacctaaggtctgctgtgaaatgctgtcacatagacttgagcttttgaatctttacctaatctactcccgtattgtttgagaaattatttttccatttgtggatcatcctccttttgcctgttcttttgcacatttcattgctttttggtgctcccgccaaagggtgagcaactgtagaatgaggaggtaaattcaaatctgggtacatgacaaatttggtaacataaataaatatgaatatcattctccaaaaaaagtgaagattttccagatttcagatatcctggctttccttgctcaacttaataaacaaagtaataaacactttaatgacagagctgtcagatctagaagagatttcttggtctaagaattaagaaataactgcctctcagtctaaaggccctaatatatgaattggagaaacacaaaatgaatattttaatctaaaaggaagagtgactccttagcagaagaatatcattgcattttttacacaagatagaacaggtagctctgatcagccacatgtcctgatgagtgacaatgcagtctaaggcaaaaccagaagataaggaaaccctaaactctaaattgtgtgctgctctgactgatactgtaaagcaaaatcaaagttgaaaacaacccagaaactttgattcctaaggggtttttttcttttgctttttttttattcctgagcttactgtagtcagtgactttttaacaatatgccaatttttttagtttcaagagcagtaatgatctaacccaaaactctcttaaaaaccttaagtcatccctatttctaatacactgtgatgagtggagtcaagccctggaatgagcagagagacgacagcattagccaagcagggagcgactctgatttaaaagcctacaaagtcagaataaaaaggctggagaagtataatgaaccttgagtgaaagcacatctgttcagattttattttatactgtttggtagagggaatatagctttttttttcttgagcctgcaaaatatgcatgctcagctattgcctgaaataaaataactggccttgcgtagtcataaaagagacattatgtactgttaactggctctctattaaccacaacgggtagttctctgtgattttgaacaattataccttgtttttcagagggggaatgtctgttgaaaggcaactagcaagctgaaagttactcatattttcccattgggcatattttatgaattgaggaagagggagactggactaagaaataatttttcagtcccaggctgatgcataggaaagaagacctaggtcagcttggctggggaatttgtgttagtcaaaataaaggtgtaactttttaaaattccttcaatatagtcctagacttagaaatttttggtcctacttgtcagtctgtccagttactctaattccaaagggaatgtaatgtttccatacagaacaagccacacaatgtcattcccctcctggaaatcaaaaaatgtgcttgctctcatttccacccaacactgagcaacctccccttccaatgcaggtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagtaagtagtgtttttgtttttgttttttcagttctgtgaaattcaggcagctagcacaaatcccatttggcttcttctcaaccctacttctagaatcaggattattctcaataagtccaagaaaacagtattgttccccaaaccagtttcagagagatacctttcctgagtattctctgacttctacaagatctcactcaaaggaaatctctcattgtgctcaggaacagagctgtgcccagcaacaaataaagattcctgcagtgtaataaagtggcagagagatcatcttcccttcccggtcagagagtgcagttaagggcaaaaaaaatcaagataagcacatagacaaaaaagtaatggcaattgtgatggagcaaaataatagtacatactcaccacactccagttaacttaggaaaaaaccatggcagtgccttggtgccacactcacctcagctccaccccagagaaaggtatcagattaatgtatcacagtaaggaaggatttaactatctagataattttcttttgcctaatttcattctttcgtgcttagttttcactgcctctctcaccagtgtttcagaatgtgtgacaagacaacataatgcagtgaaagctcgtaggctttgaaattagactgttatagatttgaacatactctgatacttactggatatgtaactttggacaaattattcagcctctctgattctctgtttcttcatctattaaatatagataatgacactttgcatggttgttaagaggatttgtgatttttaaaaatttcttagcacaattcccagcatataaccatgcttaattaatgaaaattgctattaagtatttttgttactatatttccatgcctatatcatacagttcttgggagactaactgcaataacagatgaagctctcaacctaaccttacatatagtaagcactcactgtcaactcatagtagaaggcaaagttcatggaatgaactgtagtttttcctaccccaaagcttttttgctgttgtatcccagttcaaaaaattggacccacaaattgaatccgcattggagcatctccagaaaaccagtgcacatccacaagccagtcaaacatggtagtcacagagtccctgccatatgcaagatgctgtgcttgacagtacagtactagggccctcagatgaataaagaccttcctgtgccctgaaaaaaccttagaatctggttgagtacataaaacacggtgaatagtacatggattatccatatttgtttccaaaatcagtctagatatagtagactgaaggagtagaattgggaagggactgtcaaggataaagctggtattgctactacataggctatacatgtgtatattagtaaagggtaagtctgatcttgtttttctgttaaatccaactatgagtttttccaacccccatcgcttaacacctttttttttttttttttttttgcttaccacctttttaaaaaatgattcctcagtggaatggaatgctaagttccaagcattttcagtagattcctgccattcactcataaaagttttccctaactttaggcaggcttctcaggcccatgtttttgagtcctttcgtaaagccttaatcccattaccattttcaacatgtttatttgccaaactttgttgttattttcaggaacaaagccctcctgggacctgtagtaacaaacagaaaattctgtgtccaagggaggctgcacccaaacccctgttctgtgttctcactgaacctaagcctggcctttgggacccctgcctagactcccatccactccctttataggcaccgtctaatgactgactctgtttgccaggcaaccttcaaaggctggatggacatcatgtatgcagctgtagattcccggaaggtaaggatgtacatggcgaaaataccaccttctcagatggctggaaagcaagcagcatggtataccgagcccaccaagaaagaggaagggatgaaggaatggaggagttgacgtttctctttggaacatttattgcttgtgttattgattatagatcattttcctcgtctcagtaagtcatcactctcttacattggcccccgatgattagactgttgattttttacgtggccccagtgaagctgtctatgcttcaatcctgcttcatagcaaaagtatggctcctagcagacagccctccctccctgtggcctcatgatttgcatgttctccacatgagtcagaggacgactagtgtcctctcaaggcaggccatgccagtgtagagaaggaggcaggcagaaagaatctagggctctcccaccccaagatgtttagcagctcaaatggtcacagttacggctgatggccttggcgtgtcaaactccaaactaggagctgattctcttccttttatcctgtcctttgacagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagataggtctcctcccctcattgccagtggttggaagtcagcccagataagaggcacctttgtccctatctctagaaagaaaatgtccctctttctttctctaaaatctatattgttagctcactgtgaccctggcccccatacgttagccccaacccactctttctgttcggggctggattttgcagcatgtagtttaccacacttactagcagtcttttattcttgaggcctattactggtcttggtctcccaccagaagcaggtaggtcagagagtgcagtcgagcaacagagatgaaaaggaaagaaacagatgaatagctatcgcaagaagtagaaactgtctagacttgtgattatctagtcaatctctgagcacaagcaaattgggatttgagagacccacctttggaaagtttacttccttacttgttctagatggttaacaagttttacaaagcaaattgaaaaaattgctcgttgctcattttgtaagtcaagtctatcccagcatactccaagacaccctccaaattgctttcttgcaccagaatttccaaattttgggtatcatctaaaataaattggctatcagccaggacacaaagatctttttattctaaaatgtgttttctagaatgtatcaggaaagctggcccccagaaaatgagactctgagatatgactgccttcagagtcactgctctcaagtttaccctttctgagggtctataattggatagtccatttcctttctaccttgggactggaagatcttagaaatcctgagctaaaaacaaaggcaggtatctctttagagctgaatgattatcgcgggtctaccccactctgggacagctattagctacaaaggaaagggataggtctcccctcagttctcagtattgaaccttaggtccaaacccatagcatgttgagagccagttgtaattgtctgttttcttcttccctcctttacttcggaggtcaggacatcttcatgaccgaagaacagaagaagtactacaatgccatgaaaaagctgggctcaaagaagccacagaaacctattccccgccccttggtaagtgcattgtgcaggctgaggccttggtgagaacccatataggaaaagtactagtagagttactgcaaaggaaggaaaaaggtaagtgattggctgctttgtgcctaatcctctcctcaccacccatccaaaagatctgaataggacagcagaagaacagggattaaggcatgggggcttttacatcaatcctcgacctatcctctgagccttccttgggcagccaccaagaatcagcatgtgaaccacattggctttggccctagtagagagatgtttttagagacaaccaaaagtcaacaagagaaaatgaaataaagttgctctctttcctgccagttcaagataggatttggtcataattttttcagcttctctctgctcaataaggatgtctttgcaggatgtatctagaagttaaaatcatgcttctggttatttgtgggagcagaagagggtaaaactatgggagtctggtgccctgattgcctctcttgagctgagccttttataaaacagattagggggctgagattctgctccattgttccattggtgatggttgtcttgtttcagagaaggagaaggagataccctgctcctgattccatatatccggttggatgggacatcacccccaaaagccaggcccaaagttccttcttctgtaacttccatttagcaactttaaaaaacagacacaacaccaccaaaaaaacccagcagaacctttaatacaaaagcttccctgataatctaatttgtagaaataaaagtagagtgactatgtttgaatcaagagttatagttccagaacgttttctcccctcacccccacacgccgcacgtggtacatctaccgcatcaacttttgaaggaaaagaaattctagggctttgcagggcagattgaaaaccaccaagttaaagtaatcctggaaataagcaccagcccaagtatctcagtacttcaaacaaacaaacaaacaaaaaagcaaggaatctcaagaaagagaacatttggggttttttatttccttgatgttaagccttcattttgtgatagaaatgggactaaagggaatagaaaagggttaacatttctaaagttctgtttagtactttccatatgggaacctgagtgtattttcttccacattaaaatataacttccagcctaggcaacataatgagaccccatctctacaaaaaaattttgtaaaaagtagctgggcgtggttatgtgtacctgtggtcctcactacgtaggaggctgaggtaagagaattgcttgagcttggaggccaagactgcagtgagccatgaccgtgccactgtactccagcttgggcaatagggagggaccctgtctcaaaaaatacaaaataaaataaaatattttaaaaagtaggaaacattaataaagagtgattaacattcatttagcaaatatttatcagtctttactatgtaccgtgcactaagcattatgtgatgaacaaaaggtagactgctgtcatggagcttataatagttggggagatagataataaataagtaagcaaacagctacaataaaatgtggaagattgtaggaaaaaaattaactagagggctataagtggtggatctactttaatatagtagtcagagaaagcctcactaaggagggtctacttgagtggatgagaaggaactggctgggcacggtagctcatactgtaatcccaacttgggatctagaggcaggcagatcgcttgagcctaggagttctagaccagcctgggcaacacagtgaaaccccatctgtacaaaaaaaaaaaaaaaaattagccgagcctggtagcatgcctgtagtcccagctactgagaaggctgaggtgagaggatggcgtgagtccaggagtcagggctgcagaggatatggtaagctatatcatgccactgcactccagcctggggaagacagcgagaccctatctcaaaaaaaaaaaaaaaaaaaaaaaaagatggaaccaaagagctggggaaagagattcctggcagaatacacaagaccccagaagcagaaaacagtgtggccagggggtggcgagccagaggagggacattgatgagcagagtcagactgtgccaggtgtctccccgtaagccaggttaacaaatagggattgtgttgcaagcccagtgggaagctcttgaaaggttttaagtaggggaatgatgtgatccatttgttttttgttttatgttttgagacagagtctctctctgttaaccaggctggagtgcagtggcgtgatctcggcccactgcaacctctgcctcccaggttcaagcaattctcgtgcctcagcctcccgagtagctgggaccacaggcgcctgccaccatgcccggctaatttttgtatttttagtagagacaaggtttcaccatgttgaccaggctggtcttgaactcctgacctcaagtgatcaattttgggaggcctcggcctcccaaagtgctgggattacaggcgtgagccaccgcacctggccatgatccatttgttttaaatcattacacttgctattatatgaggactgcattggatgagggcaagagaagaagtgggagaccagttaagtggccatacaagtccagatgagagatgatggtggctcaggttagcatagagccaataaaaatggaatgaagtggatgaacccaagttataattttggaggaagaatcaacaggcccttcagtggatcagatgagggagaagaaagagacaaagatgatgcccagattcctggcttgagagcctggatgaataattgagatgagaggaacaagctgggtgaggggcagggaacagacttggctctgctttggacttaactgtgagaggcttggacatggtaagtttgagatgcttatgagtcatcaagaagaagtggcaagaaagcaactcaggatcagaggaaaggtatgggctggagaagaaaagcttaagcataagtacataggtatcacgtaagcagcaggaatagatgagctctcatagagcaaaaatttagagaaagaagggattgagagaagaaatggctggcagatgagaccaaaaaaggagcagctgggtaagtaggacgacagccagagtgtgtggtgtcacagacgcaaaagagaagtgggtcagctgggtatagtggtgcacacctgtaggcccagcttattgggaggctcaggcaggaggatcacctgaggccaggagtttgaattgctatgtgctatgatcgcgcctatgcatagccactgcactccagcctgggcaacacagtaagacccgtctctaaaaacagaaagaaaggccaggcatgatggctcacacctgtaatcctagcattttgggaggccaaggaagacagatcacttgaggccaggagttcgagaccagcctggccaatatagcaaaaccccgtctatgctaaaaatacaaaaaattagccggacgtggtggtgtgtgcctgtaatcccagctactcgggagactgaggcatgagaattgtttgaacctaggaggcagaggctgcagtgagtcaagatcgcaccactgcactccagcctgggcaacagaatgagactgtgtctcaagaaaaacaaacagaaaacagagagaagctggtaactttggaaagctcttggtgaggtaatattgatgttggtctccatccatgctcctgatagagcagcagagctgaccagtattacagaaaaggagggagagggtacctcgattagcagggtgacagcttccataggttggcttggaaaggttttcatgaatcttttatcttttccttcccttcctccccagaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggtgagtggggttggggaagtaggcgaggggaaaggggacctgactgattgtgaggatgagatttcccaggagaggaatgaatgacacaggtctgaagtgcaggcaagataccctcatagcttaagtccatgtgtgcccctgagtcatctcgggggcctgcctagcaggttccatggtcagtgagtttctactgagtgttctcggtagtcttggattataaattctgtggctcagggaaaaaagaaaaaaaaagaagattggttcttgcttttaaggagtttaaagtctaacagggaaggtaaaagaaagttgtcattaaaaaatgtagcacaatagaagtataaaatgaaagaaggaatgattgctaagttggccattaatgaaatagtctaggtaagttctggtggtgatggccatggtagtgctgctgttactggcagggcttgtttactgagattttactgtgggccaactacagtttgagagtttcttcacctgtgttatctcatttgattcttattacatcccaggaagcaagcttctttattatctggactgtatacatgtccacagccactcgctaggaggtggtggaggcataactcgagttgatcccaaagtccaagttttttaacagtgactgtgtgcattttgcttcaacctaaaagtgtggggggatgatgtcctggcaggcaggcagaaggaaatgccaccctgttgattgccctcccaaaatgtttccttgccttcctgaaaggcatcttttgtgataaaactcagccttgtttccaggtaagtttgactcatttacactagagagagtgctcgagacccatgggaccagccccagagctccagcggccatgctgggcctagtgtagtggttataaggtaccagaaggcaggggtctccccttggcctgtgccaccctctagttctgtcctccagagcagcatgtccaaaagtgtgttgatatgtggaacactaatcctagacatttctcctcaagaaaagaatctcatgagggaattactttaggtaacactgcctttaattttcctttgaggattcacagtgcacattactgaattctcttggaaatcctacggtaaagaattctgggccgggtgcagtggcgcgtgcctgtaatcccagcactttgggaggcaaggcgggcagacaacttgagcccaggagttcaagaccagcctgggcaacatgataaaactctatctctgaaaaaaaaaaaaaaaaatgagccaggcatggtgatacgtgcctgtagtcccagttacttgggaggctgaggtgggaggattgtttaagcccgggaggtcaaggctgcagtgagctgagatcacgccattacacttcctagatgacagagagagacctagtctcaaaaataaagaattctgtttaaatgtaacatagcatcccacctttattagaccagtttcctttttttcagtaataataatgatagctcacatttaaatggaacacattatgtgccaggtaccataataagtgctttaaatatgttaattcatataatcctctcaacaaacctctaagaatcctgaggaactgaggcacagagaggttcccaagacatgtggctggtaagtgagcaagtggggattcaagcccagcccctggcctcaagtccattctcctaatttctcacaaaacacactttggacaatgctacccttgtccactgctcttgaaactttcctggccacttatcgttgagtgacacagaaaatccacagaccaaatcataccccctattttcaaatggtactctaaaaagaggaagatgtttttgatgatggggatttttattaacattaatttgttcagaaacaaaagtatagaaatcatctttttaaagtaagaaatgtattagagttttgtagagggacaaaactaataggatagatgtatatatgaaagggagtgtattaaggagaactgactcacgatcacaaggtgaagtcccacgataggccatctgcaagctgaagggagccagtagtggctcagtctgagtctcaaatcctcaaaagtagggaagctgacagtgcagcctttggtctggccaaaggccagagagcccctggcaaaccactggtgaaagtccaagagtctaaaagctgaagaactcagagtcttatgttcgagggcaggaagcatccagcgcgggaggagatgaaggccagaagactcagcaagtcagcttcttccatcttcttctgcctgctttttctagctgagctggcagccgattagatggtgcccacccacattgagggtgggccttcctctcccagtccactgactcaaattaaaagtaatggcaaaaactgcaattacttttgcaccagcctaatctctggtaacacccagaaacgatactttgcatccttcagtccaatcaaattgacacttaatattaaccattacaagtccaccccttgccaacttgaacccatacacatctcctgaaatcatacttaatctccaaataaatacaataataaggtcctaattacgcctaacataatacagctatccttcatacaaccagaagcgcactaatccttaacctgaatgctattacataaagttaacaacacttaaatgctgatacgaagtcaacaaatcttacgtcacatgataaaggaaaaagaaaagaactaaaatgaagatattttcctagtacaagtgtatacatgcacaaacatgttcttaacaaaatgaggaaatactcatgacaatagtcttcatttctgcaactggtcacgtggtcgtagctggtattgatgactaccttctactactcattctatattcactttgccttcagcaagcaccacggctggttgtggttatttacctggtggagtgacccaaatcttcattcctaaagggtttggggcatttgtagtcctacctggattgggctgttgtagtttcccattgaccttaatcacagggcatgctaatactaagagacgccctaagggatctcctgtattccatgcatactcttccatacctccattgtggagcagtagactgatttcatcttgatagtctgggtcagtcaccccagccaacactataactcccttcttagactgttgacttagaggtagcccaaagtggctaggtggccatcttaacttcaggtttaatggaatcattgttgtgtctcctagtgacagcattcctccctctggaactaagacctttaggccagcagaacataatgtcgcaggaacaggaggcaaaaattttgctagccggtcactaggggtgatggggagtggtgccacttccatttccaccccttgattcctggacccatgaatcctggctatgggagaaacagtaccatatattggacactgattcaaagcatacatggccttctggagaactttgccccagccctgcaaagtattgttacctagttggcattgtaatcatgacttcaaaaggccattccaccatgctatcaatccagctgcttcaagatgatgaggaacatggtaacaccagtgaattccatgagcatgaacccactgccgcacttcttagctgtaaagtgagtgccttggtcagaagcaatgctgtgtggaataccatgatggtgggtaatgcattccgtgagtccacagatggtagtcttggcagaagcattgcatgcaggataagcaaacccatatccagagtaagtgtctgttctggtgaggacaaaccgctgccctttccatgatggaagaggtccgatataagcagcctaccaccaagtagctggctgatcaccccgaggaatggtgtcatattgagggctcagtgttggtctctgctactggcaaattggacactcagcagtggccacaaccaggtcagccttgatgagtgggagtccatgttggtgagcccatgcgtaacctccatccctgccactatagtcactttgttcatgggcccattgggcaatgacaggggtgcctggggaaagaggctgagtggtgtccacagaatgagtcatcctatccacctgattattaaaagcctcctctgctgaggtcaccctttggtaagcattcatgtgggacacaaatatcttcacagtttttgaccactcagagaggtccatccacatacctcttccccaaatttgtcaccaatcatgcttctttgaagtccctgaccatccagccaaaccatttgctacagcccatgaatcagtatataatcacacatctggccatttctccttccatgcaaagtgcacagccaggtgcactgctcgaagttctgcccactgggaagatttcccttcagtgctctccttcagggatgtcctagaaaggggctatagtgctgcagctgtccacttttgggtggtgcctgcatttcgtgcagaaccgtctgtaaactaggccctagtcttctcttcttctgtcagttgatcatagcgaactccccatgaggccatctctgcaggctctgggagagaaggtagggtggcagcagtggggaccatgggcatttgagctacttcctcttgtaacttacttgtgcctttaggacctgctcgagcccaatcaggtgcataccatttccatttgacgatggaatgctgctgtgcgcacccaacttcatggctagatgggtcagagagcacctagttcatgagaggcggttcaggccacatggtaactgggtgacccatggtcaaacgttcagtttccaccaaagcccagtaacaggccaagagccgtccctcaaaaggcgagtagttatctgcagaagatggcagggccttgctccaaaatcctacggacctcctctgtgattcacctatgggggcctaccaaaggctctaaacagcatccctatctgccactgatacctcaagcaccactggatctgctgggtcatatagcccaagtggcagagcagcttgcacaacagcctggacctattgcagagccttctcctgttctaaaccccactcaaaactggcagcctttcaggtgactcaataaatgggccggagtaacatactcaaatgaggaatgtgttgcccccaaaatccaaataggcccactaggcgttgtgactctttcttggttgtaggagaggccaaatgcaacaacttatccttcaccttagaaggaatatcttgacaggcccctcagcactgggcccctagaaatttcactgaggtagaaggtccctgaattttagtctgatttatttcccatcccctggcacccaaatgtctcaccagtaagtccagtgtgtttgctacttctcgctcactgggtccagtcggcataatgtcatcaatgtaatggaccatcatgatatcttgtggaagggaaaagcgatcaagatctctgcgagcaagattatgacacaaagccagggagtttatatacccctgaggtaggacagtaaagatatattgctggccttgccagctgaaggcaaattgcttctggtggaccttatggacaggaatggagaaaaaggcatttgccaaatccgtagctgcatatcagataccaggagatgtgttaatttgctcaagcaatgaaaccgcatctggtacagcagctgcagttggagtcgtcaccacttggttcagcttatgataatccactgtcgttctccaagatctatctgacttctgcacaggccaaataggaaagttgaatggggatgtggtgggaatcaccacccctgcatctttcaagtccttgatggtggcactaatctccacagtccctccagggatgcagtattgtttttgatttactgtttttctaggtagaagcagctctaatggcttccatttggcctttcccaccataataggcctcacactaccagtcagccaatatggggattctgccagttgctaagtatgtctgtgtcaattatacattctggcactgggaaaatgaccacaggatgaatccgggacccactgtaagtcagacctgagctgaaacttcattaattatctgacctccataagcccttactctaactggaggaccacagtgatgttttgggtcccctggaatcattgtcagctcagagccagtgtccagtgtccagtagtccccgaaaggggaccagttaccctggtaaaagaccggaggtctccttggggaagcatgggagaaagattagcagtataaattgtcagtagtgtagtggggtccttcctcaaagggaccaggcctcccctttattcaaggggaatattattctgggtctgtacgctggttcaagtctggaaattggttgagggggcatgattctctatttttataattcaaattagccttttgtccacttgacctggaagttttctgcttacacaaatcaagaaagaatgtagtaggcttcctatcaatttcacttctaggaacaccatgattaattagccaatgccagagctctacactaatcagactattctgaatgctgctttgcctctgctatccattatggtagctacgcccaccttgcctttgacagttgagtgcccccactcagcccctgccaccttgggatccaattattcccattgcatttaaattatccagttaagtgactgcagttcccactgtaagatctggcatacagggaagagcaatcacagagctcttcaaggatgcaggtgctcccctcacaaatctgttttgcaagctattaagggtatgtcttctggaccctaccagttgggatgagtaggtctaaggtgactaatccactctagcattccaatctccctaagcctttggatcccttcctctacattaaaccaagggagatcaggcatttccagcttactcacagtgggccatcttttgatccacgtttcagctaaccaagcaaataaactattagaacctttttaactccccgagctgcgatattaaacacagaatccctgcttagtgggcccatatcaataaattcaacctgatccaactttatgttccttccaccgttgtcctgcaaccttaatatccattcccatacctgttctccagatttctgcttatacaaattagagaactccagcaggccaggcgccatagctcatgcttgtaatcccagcactttgggaggctgaggggggcggatcacttgacgtcaggagttcaagaccagcctggccaatatggtgaaaccccgtctctactaaaaatacaaaaattagccgggcatggtggcatgcacctgtaaccccagctactccggaggctgaggcacgagaatcacttgaacccaggaggtggaggttgcagtgagtggagattgcaccactgcactctagcctgggtgacagagcaagactctgtctcaaaaaaaaagaaactcaagcagttcttttggagtgtagctcatctcgtgagtcacactctgaacctcacttctagagagctgccatgacttgagtctagttacaggtctagaaacaaacaggggtgttgggggtgggttcagaagagaatcagcattgtcttgcttcaggggagaccactgttgcctcaagcattgcagggttaatctcagacaaatgtggaaaggctggtggcagcctgggtgggggaagggatattgccaccactgggggtgaggaggctgttttctctggcaaaaaaggctgtcagagtttacaagctcagtgtccccagcttcatcagggtcctcccacacatccctattccaagtcgcagggtcccattcttttccaatcagtgccctcactttaacaatagatacctagggaggctacgcatgcacctttccttgcaggccagccacttgcgtggtaagaacttgtgtctgattttccgcaaattcagccctttgtctacaggagataagactcagggaaatcttagaagacttgaggctcagtatgtgctctggagctgggagttagaatccctgagctcatccttttctttcattactttctccagtgaacttaggagaaccaaccaacttcattatattccttggttctccacatatggtcaaatgtattacgtacagagtcactaaactccttgcctctcaagaacgacgaatcaggaatatcaaatgccatctattttgcataactctctaaacagttcacaccagggactatcagtgttctccatactatcagaagtagagtccttagcatttttgggtctaatcagattaagcagccaactccagaaaccccaaaaccaactaaagaaatctattcttaaaattctgttcctctagacccactcctggtaccaaaatctgtcagggttttctagagggacagaactaataggatagatgtgatctcacatgatcacaaggtgaagtcccacagcaggccatctgcaagctgaggagcaaggaagccagtagtggctcagtctgagtcccaaatcctcaaaagtagggaagctgacagtgcagcctttggtctgtggccaaaggcccgagagcccctgtaaaaccactgctgtaagtccaagggtccaaaagccaaagaacttggagtctgatgttccagggcaggaagcatccagcgcgggaggagatgaaggccggaagactcagcaagtcagcttcttccaccttcttctgcttgctttttctagccgagttggcagccgattagatggtgcccacccacattgaggatgggtcttcctctgccagtccacagactcaaatgttaatctcctctggcaacacccagaaacaatgctttgcatccttcattccaatcaggttgacaatattaaccatcacaaggaattggggtaaaactagctctgaaaccacctacagtaccccttaaatggtgtttctactgcctacattactgtctgccccatttctccctttttctatggggacaaacacacattcatccttcaaaacctacctcagccatcatcttctctctgaagccctacttgcccaccatatatgggctgcaagtatcacatatacttgaatatatattatataacctgacttctttggtcactggttatgatttaattgagaagctgagaaatcatcatgggtgggtcctggctaaggtagagattttcttgaaggctcaaatatcccatggaattctagctcctgaagaggctccaagaggttacttgacccgctcgctgaacttaccgcatcttgtcttttcctgaaaagctaagattcttcaaaggggattctatagccttctgcagttaactgccccagtgtttatgcattcttatgagtgagaaatgtttccttaaattaaatccctcatgtcagctgaagctcccattgtcttgttagtgcctggacggcaagagaaggtactgaacgaagagaacatgtcagtgccttcccttcggttgtcatttcccagacttttcaactcccttccttctgtggcccctcatttgggtcccctcattgctctgtagctttaaagaacaagtggcctggttgaggtgaatccctggacgaatataactagaaaaaggcgctccgatttgtacactttcaccttctgcctttatatcctaatgccttggcatctttacctgctgatactggtttcttttctttttctcttttcttttttttttttttttaaagacagagtctctgcctgtcgcccaggctggggtgtgatctcagctcactgcaacctctgcctcccaggctcacgcaattctcctgcctcagcctcctgagtagctcgggttacaggcatgcgccaccgcgcccagctaatttttgtaccttttttttttttgagatggagtctcactctgtcgcccaggctggagtgcagtggcgtgatctcagctcactgcaacctctgcctcctgggttcaagtgattctcatgcctcagcctcctgaatagctggaattacacttggctaatttttttgtatttttagtagagactgggttttgccatgttggccaggctggtcttgaactcctagcctcaagtgatctgcccacttcagcctcccaaagtgctggattacaggcatgagccattgcgcccagcctgatactggtttctgattcacgacttgcagcacaaaatctatcagctcagagttgaaagtcactcctgagagtccctttagttttctaggctagtttggtagactagggaggtagggctatgggaaaccaaagatgttttttcagagcaaagaagaagatactttgaaaattgctagcattagagaataagtaagacatcaaagttaaaggataatgaggttatgaggtgtagccaggacctctcatttattgtttgtggatatggaaatttgattccattgtggagagtatcagtatcatcttggaaaatcatatgtgcacaaatctggaaaaaaaacctaatcattcttgagtagaaaaacaagtaaatgaggacctattcataaaagggactaccactcaattgttagaatgagtaatctaaaattatttcaacatgagggatcagtctcacaattgtaatgttaaatctcaaaaacaaggtagagacctaaatagatggacctcataagacaaatacagcctggatgtggcagttcacacccgtaatcccagcactttgggaggttgaggagggaggatcggattgcttgaacccaggcattcaagaccagcccgggcaacatagcaagacccccatctctacaaaatatttaaaataaataaataagacagaaacagtataatttcctaaccatagtaggaaattatatatatatatatgattcttacatagtaaatctataaaggcacacactagaatgatacataccatcttcagcattgtggatgcttcatctgggaatgatgaaagaggaagaaaaattggtaaaatgctagcctctattaaatctgtgttccattccagttactcaggaggtctgaggcaggaggatcacttgagcccaggagttcaagaccagcctgggcaacatagcaagaccccatctcaaaacaaaaaacaacaacatcaaaagacaaatgtgtgtgttagttatgtgtgttaggcacataggtgtctattttattgtttacttttctatatgtttaaaatgttactttttttttttgagacaagaatcttggtctttcaccaaggctggagtgcagtggcacgatctcggcttactacaacctccacctaccaggttcaagcgattcttgtgcctctgcctcccaagtaactggaattacagatgtgcaccaccatgcccagctaattttttattttcagtagagacagggtttcaccatgttggccaggctggtctccaactcctggcctcaagtaatccacctgccttggcctcccaaagtcctgggattacaggcgtgagcccccatgcccagccaaaatgtcactttttgagagttcagactgaatagtaaggcacttattctgcaaagccctttgaacctaagggttccacaatgccaggtaaaaaaaataaagagaaagggtgtctcccatctcaataacataacttcttctattcctcttcttaggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttagaggagaacaaaaattcagtattatacagatctaaaactcgcaagtgaaagattgtttacaaacttcctgaatattatcaatgcagaacagctgtggagactctaacctgaagatctataccaaacgtcgtctgcttaccacgtaacacagctgcatcttgagcagtgacctgccaagggcaaaggaccccgctccctagacttacagattttctaatgcttgggcaggtggttactgcatgttccacatcagtcaatgcaacttaggacaaaactaaccagatacagaaacagaagagaggctgccgggaccagcatatttccgttgcagccaaatggattttattttttcattttattgattctcagaagcagaaagcatcactttaaaagttcgtttgttcatgcaaactatatttgcattcttacattagttaagctaagcagcaaaaagaaaacacacacacacactcacatttagcccatgtcatttaattgtcagtttctttgacataaagcgcatcttctccacatgggcttcacgtggtttggagatgggtgggggaaaacaatcaggtttcttcaggctgaggaggacttgctcaggccgattccaaacattgtgctcgttcaatgcgtagaaatgatttgcatgatggcatgccgtgatcagaagtcatgcatgagatccatacaccacaggacactactaatctagtcccttgcactgggtcagcctttggacaggacccagccctgcaccgttcactgtatttggagaaaatggtaagagttccataccggctacaattctttgagttcttaaaagtccttcatacaccttctgggtagggaaacaaccaactaattgactaacaccaccaacaacaaaaaacaaacccaatccaacaagcagatggatccgttgcgtgtatatgtttaacagacatctctaacatacagccattgttgcacattttgcaagatgaactatttaatgctgctctgtgtccagtacatgggggagactttgatcccaaatggcttgtactatttatgtcactgtaaaaccaaatcctagggctaaaaaaaaaattcatttgtatcttgcaatatttatgacgatcgtttagccttcatactggagaattgacacttatttggggtagagataaaagtgcttttcaaagtagcaccagttatctctaggggtaatttggggaacttaaaatgacctttcattgggagttatggggtgctcatttcatttcgtatcatgtcctctgcattgtggctttctccaggcatgggtcgataccgcgaggggttcagatattctgaactgacctctctcttctgcagaggggtcatcgcatactcacacactgcatggctccccctgcctccatcacatttctccactgagcagggcttcccttgcccacagaggtgggtgagggtagcagcattggggttacggctgtggtttctttcacttattattagaataatagtcactggtgagacctgaggtagagatggagccgcctctgaaccaagcccacttggtttctttactgcactggttttcatgagaaagcaatttaatattaattctttaggatgttccagcttccctcagtgcattcccttgcagccagttttgctatctgcttagcgacctgcccattgtcattgaagtgtggcttctgggggagcgtcatatcttccattccatctcctgctttaggaaagacagtttttagtccaggggtttctcacctaatgactggatttcaaagccaagatgctgcagttgaatgtgtcaggaagtctctatacaaagagtgaaaagagactttagcatgtgaaccaaatccaaataaattcttcctgaaccatacactagagggggaagaagaaccaacccataatcagacacaccgatctgccttgcagtagaagcactttgaaggtaatttcacagtccaccagactagttttgcatagaacaaaccacagtaagtcggcaagcagagctttctatcttgttggaccactagaaactgtccagctctcttaagcctgcttctgcagcagcatccgtagaatttttgtactacacccaataccagactgggaaggcctatctttcagtgaccctgctaactgctaggatgaatgtatagtaacatgtacaggctacattgtaaacagcacaaaacagaagctgacatgtgtggttattctttttaagagaattataaatactgtaatatataattttattttattggcatgcctttttgtaaatacaaattattaacttattaaataggaaatggcttctggcttatgccattgtcatgtttatttttattttttatacttttctttcttcttagaacttagatgctgtcagccaatgtacatccccaaaccagacagacagacaaaaaattttttattgctaatggtcttttccaaaacaacaaaaaatatatatttttgttccaatgtgcaataaattctaaccacgtctatgcaagatttggctaaacttaataattgttcttaggtcttgagatgggcaacagagctgtaagatcccgccaagtacaccatgtgctcatgctagtgatgtcattaagctactagggcatagtgatgagatttttctgagatctaaccgtttccctccgctagctcctgaccccagcccagcaggctgtctcctcagtaacacatgggccttggtatctggcacccatcagccagcctcataggaaagaagccacctccgctgtatttgaagtttcactttttaaaaaaaaaatgtttcccacctttttatccttcacccactgtccttccacctgctcactcactcactctctcacccatcctgctccacacttctttggtagtaaatgacaccatcaccagcagtttacacccgcagttaacaggcatcgaactgaaagaatcagtgatgacgtttttgtgcatcttcgctgagtgggtaagtggcaatgctttgccaaatattaacgaagccaatcaaagagcagcgcagccacagtatcactgcacatatatatatagatatataaatataatataaatatttattttttgtagccaggtccttggtgcagtatttatactccacaatccaccttttaaaaaaaggacaaaaagcaaaaagatgtgtgatgctgttaataatttaaaatgcagagccaaagccactgagcagcagctgacatggttgctggtttgtgtgtgaaaaacattttccccctcttctttccttaacctccttgttgcttaggatgaggagactctgtaatttaaagcagaggacgagaggaggaagaaacttccatgttaacatggcctactctcactcctttctaaacgagccacttttggcgtcagaagttgactggagagagataaacaaactcccagtcaaagcccctaaagcgacagtgcccagagttcttttattttttgctgcaaccaacgtaaacctgtaaaagaccaacagtgaagattagtgtattagtgaatgcatggaggccaatgctgccctaatgagacgtgataaaaagtccatcactcctctaagccaaaaggaaacaaaataaaaatgaccctttttactgtacaagggaagcctgtcttggtgtgtttgttgcttgactatcccaattcttgagtcctgggaggggtctgggaattgggaccttagcacatttggggaaaggaggggaatagagggaagagaaatcaaaggggctctcgcaaagttctggctaagaaggagagattggaagaaaagcaaggtcctgaccccccccagatccccactcacccattgcatgtggcattccccactactggccggtgcttagaggcccataaaacctctcttcacctaatccacctggcagagccctcagagatgacgtcagccaacccagctggactggccttacttctgcctttttcacctgtatgttaaaaaacaaaaacaaaaaaaccctatccctgtgccccaaagctagggcatgtgcgatgcccaacagcctccactctgcaagctcatactgggccctgccctccatcgccccagacaggcccacagacgagggctggtctccttcacatttgtgtgggggtgggggcacgcttcctcttccttctctccctgtccccctttggggacatccctctctaggcctaggccatgtctcctggttcaggaacactctctcaggaggatctcgaagtgcccactctagtccgctccagtagctccactctggagcgagatggctggtaggtcagtccccatcctcctcccctcggcccccagcctccaggcctgctgtggaggtgagatcgccatgggtcatgactttgtgacttgagtcctgggcatcttctgtaatgatgcctctttcttttctcagatgttgcccacagtttagcaaaaagcttcgttcattttcaggtactccccaccccctaaagaattggctgcaacttgccagccagggggacatcaggagagaagaggaagtctgggttgggaggggcccaagttttgcaacagagtttccattgttcagctctgtgctttcttggattccctttccaagattaccaagatttcctccattaaaactcctgaggcaccccctgcccctgccccccactgctgtttgctgtacatagaggctaagtggggtggggtgggtgggtgtgcacatgtggtgtgtgtgtgtgtgtgtgaagaatgtatataggtaaaggggagtgtggtccagtggttccagaaaagggacagaactgtgttctgtccctagcttgaccgctggctcgctgtgtggccttgggcaagtcacttaacctctctgtgcctcagtttccccatctgtaaaatggggataataatactgacctacctcacaggggtgttgtgaggctttaactaaatgttttgtaaagcgttttgagatacagaggcaaaggcgctagggaagggcaaagtattattggacttaaggaagctgaaatggtaccataaatgctgctattctgagagccattttaaactgcactgctccaaccacccccgcttctcatcaccaggacagcaggtcgagaaaatgtctttcctttcacttgcttctggggtttgtgattattcaagacaattttttccccttgctgtatctccttccctcacctcctcgacttgttccctgttctgtttatgcggaaatggcagaaatgcttgagaaatgagaatgtgtaagtggattggaagtttatagtatgaaatttcaattttactttgtactgtacatctttttactttagaatttgcaataaagggttacgtcaatcttgttttcaactctc